SEQUENCE LISTING (42 sequences)

SEQ ID NO: 1 - RNA, 20 nt, synthetic construct
  source 1..20: mol_type = other RNA; note = DOCK8 sgRNA e3g4
tccacagggtcataaaactg

SEQ ID NO: 2 - DNA, 8922 bp, synthetic construct
  source 1..8922: mol_type = other DNA; note = pFb-e3g4-L-shortHA
gacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccaggactctagctatagttctagtggttggctacagcttgcatgcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtactagttctagaggaaaattgtctccgttccctagttcactgtatttgggtaaacaaaacatggcctcggagagcctttgcgggagaaattataaaaattcagcaaagctttgtgatctcattttggatctcctcaggattcctcatcaagttgcaattaaggagagaaaaattccgccttggggtgggattttaattactcatcatgttatgacttgactttctgtgctgcatttttttccatatcactacttctgtgtttgacagctcctccaaagagtcgctccgttttatgccaggagcgaagtacttactaagtcaaaggaaagcaaggcaaacatctactattgcctttgtgcttttaccagaaaactgggtgagaacctccttttccttttccctgcccctccagcctcaattctatgaccctgtggagccagtggactttgaaggacttctgatgacccacctgaacagcctggatgtgcagctggcccaagagctgggcgacttcaccgacgatgatctggacgtggtgttcacccctaaagagtgcagaaccctgcagcctagcctgcctgaagaaggcgtggaactggatcctcacgtgcgggattgtgtgcagacctacatccgcgagtggctgatcgtgaaccggaagaatcagggcagcccagagatctgcggcttcaagaaaaccggcagccggaaggacttccacaagaccctgcctaagcagaccttcgagagcgagacactggaatgctctgagcctgctgctcaggccggacctagacacctgaatgtgctgtgtgacgtgtccggcaagggacctgtgaccgcctgcgatttcgacctgagaagtctgcagcccgacaagcggctggaaaacctgctccagcaagtgtccgccgaggacttcgagaagcagaacgaggaagccagacggaccaacagacaggccgagctgtttgctctgtaccccagcgtggacgaggaagatgccgtcgagattagacccgtgcctgagtgcccaaaagagcacctgggcaacagaatcctggtcaagctgctgaccctgaagttcgagatcgagattgagcccctgttcgcctctatcgccctgtacgacgtgaaagagcggaagaagatcagcgagaacttccactgcgacctgaactccgaccagttcaagggcttcctgagagcccacacaccatctgtggccgctagctctcaggccagatctgccgtgttcagcgtgacataccccagcagcgacatctacctggtggtcaagatcgagaaggtcctgcagcagggcgagatcggcgattgtgccgagccttacaccgtgatcaaagagagcgacggcggcaagagcaaagagaagattgagaagctgaagctgcaggccgagagcttctgtcagcggctgggcaagtacagaatgccctttgcctgggctcctatcagcctgtccagcttcttcaacgtgtccacactggaacgggaagtgaccgacgtggactctgtcgtgggcagatctagcgtgggcgagagaaggacactggctcagtctagacggctgagcgagagagccctgagcctggaagaaaatggcgtgggcagcaacttcaagaccagcactctgtccgtgtctagcttctttaagcaagagggcgacagactgagcgacgaggacctgttcaagttcctggccgactacaagcggagcagctccctgcagagaagagtgaagtctatccctggcctgctgcgcctggaaatctctacagcccctgagatcatcaactgctgtctgacccctgaaatgctgcccgtgaagccctttccagagaaccggaccagacctcacaaagagatcctggaattccccaccagagaagtgtacgtgccccacaccgtgtaccggaacctgctgtatgtgtacccacagcggctgaacttcgtgaacaagctggcctccgccagaaacatcaccatcaagatccagtttatgtgcggcgaggacgccagcaacgccatgcctgtgatcttcggcaagtctagcggccctgagttcctgcaagaggtgtacacagccgtgacctaccacaacaagagccccgacttctacgaggaagtgaagattaagctgcccgccaagctgaccgtgaatcaccatctgctgttcaccttctaccacatcagctgccagcagaaacagggcgcctctgtggaaacactgctgggctatagctggctgcccatcctgctgaacgagagactgcagaccggcagctactgtctgcctgtggctctggaaaagctgccacctaactacagcatgcactccgccgagaaggtgcccctgcagaatcctcctattaagtgggccgagggccacaagggcgtgttcaatatcgaggtgcaggccgtgtcctccgtgcacacccaggataaccacctggaaaagttcttcaccctgtgccacagcctcgagagccaagtgacattccccatccgcgtgctggaccagaaaatctccgagatggccctggaacacgagctgaaactgagcatcatctgcctgaatagcagcagactggaacccctggtgctgttcctgcatctggtgctggacaagctgttccagctgagcgtgcagcccatggttatcgccggacagaccgccaacttcagccagttcgcctttgagagcgtggtggccattgccaacagcctgcacaacagcaaggacctgagcaaggatcagcacggcagaaactgcctgctggcctcttacgtgcactacgtgttcagactgcctgaggtgcagagggacgtgccaaaatctggcgctcctaccgctctgctggaccccagaagctatcacacctacggcagaacatctgccgccgctgtgtcctctaaactcctgcaggctagagtgatgtccagcagcaaccctgatctggccggaacacacagcgccgctgatgaagaagtgaaaaacatcatgagcagcaagatcgccgaccggaactgcagccggatgagctactactgtagcggcagctctgatgcccctagctctccagctgctcctagacctgccagcaagaagcactttcacgaggaactggccctgcagatggtcgtgtctaccggcatggttcgagagacagtgtttaagtacgcctggttcttcttcgaactgctggtcaagagcatggcccagcacgtgcacaacatggacaagcgggacagcttccggcggaccagattcagcgacagattcatggacgacatcaccaccatcgtgaacgtggtcaccagcgagattgctgccctgctggttaagccccagaaagagaacgagcaggccgaaaagatgaatatctctctggccttcttcctctacgacctgctgagcctgatggaccggggcttcgtgttcaacctgatccggcactactgctcccagctgtctgccaagctgtccaacctgcctacactgatctctatgagactcgagttcctgaggatcctgtgcagccacgagcactacctgaacctgaatctgttcttcatgaacgccgacacagcccctacaagcccctgtccttctatcagcagccagaacagcagcagctgtagcagctttcaggatcagaaaatcgccagcatgttcgatctgaccagcgagtaccggcagcagcactttctgaccggcctgctgtttaccgaactggcagccgctcttgatgccgaaggcgagggaatcagcaaggtgcagcggaaagccgtgtctgccattcacagcctgctgtctagccacgacctggatcctagatgcgtgaagcctgaagtgaaagtgaaaatcgctgccctgtacctgcctctcgtgggcatcattctggatgccctgcctcagttttatgaccctgtggagccagtggactttgaaggacttctgatgacacacctgaacagcctggatgtgcagcttgcccaggagctcggggacttcactgatgacgacttggacgtggtgttcacgccaaaggaatgtaggactttgcagccctctttgccggaggaagggtaaatagttttctaaaatgtagatgtgattgggattgtcatgattgttttcaataagtgggtaggggagatgccttcaatctgaacttaaaaataaaataaaattactcaatccattcaaatgtgtgggacagctatatgatatcatcatgtaagtatagatagtttttaaattagtttggccagagattttgaaaagctatgtgaagactaagggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggtctgagacaataaccctgataaatgcttcaataatgtaagctctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctactttttctctgtcacagaatgaaaatttttctgtcatctcttcgttattaatgtttgtaattgactgaatatcaacgcttatttgcagcctgaatggcgaatg

SEQ ID NO: 3 - DNA, 9118 bp, synthetic construct
  source 1..9118: mol_type = other DNA; note = pFB-e3g4-Rflag-shortHA
gacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccaggactctagctatagttctagtggttggctacagcttgcatgcctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtactagttctagagttcctgaggatcctgtgcagccacgagcactacctgaacctgaatctgttcttcatgaacgccgacacagcccctacaagcccctgtccttctatcagcagccagaacagcagcagctgtagcagctttcaggatcagaaaatcgccagcatgttcgatctgaccagcgagtaccggcagcagcactttctgaccggcctgctgtttaccgaactggcagccgctcttgatgccgaaggcgagggaatcagcaaggtgcagcggaaagccgtgtctgccattcacagcctgctgtctagccacgacctggatcctagatgcgtgaagcctgaagtgaaagtgaaaatcgctgccctgtacctgcctctcgtgggcatcattctggatgccctgcctcagctgtgtgatttcaccgtggccgacaccagacggtacagaacctctggctccgacgaggaacaagaaggcgctggcgccatcaaccagaatgtggcccttgccattgccgggaacaacttcaatctgaaaaccagcggcatcgtgctgagcagcctgccttacaagcagtacaacatgctgaatgccgacactacccgcaacctgatgatctgcttcctgtggattatgaagaacgccgatcagagcctgattcggaagtggattgccgacctgccaagcactcagctgaaccggattctggacctcctgttcatctgcgtgctgtgcttcgagtacaagggcaagcagtccagcgacaaggtgtccactcaggtgctgcagaaaagccgggatgtgaaggccaggctggaagaggcacttctgagaggcgaaggcgccagaggcgagatgatgaggcgtagagcccctggcaacgatagattccccggcctgaatgagaacctgcggtggaagaaagagcagacccattggagacaggccaacgagaaactggacaagaccaaggccgaactggaccaagaggccctgatctctggcaacctggccacagaagcccacctgattatcctggacatgcaagagaacatcatccaggccagtagcgccctggattgcaaggattctctgctcggaggcgtgctgagggtgctcgtgaatagcctgaattgcgaccagagcaccacctacctgacacactgcttcgccacactgagggccctgattgccaagtttggggacctgctgttcgaggaagaggtggaacagtgcttcgacctgtgtcaccaagtgctgcaccactgcagcagctccatggacgtgacaagatcccaggcctgcgccactctgtatctgctgatgcggtttagcttcggcgccaccagcaatttcgccagagtgaagatgcaagtgaccatgagcctggccagcctcgttggaagggcccctgacttcaatgaggaacacctcagaagaagcctgcggaccatcctggcctacagcgaagaggataccgccatgcagatgaccccatttccaacacaagtggaagaactgctgtgcaacctgaattccatcctgtacgataccgtgaagatgagagagttccaagaggaccccgagatgctgatggatctgatgtaccggatcgccaagagctaccaggcctctcctgatctgagactgacctggctgcagaacatggccgagaagcacaccaagaagaagtgctacaccgaggccgccatgtgcctggttcatgctgctgcactcgtggccgagtacctgagcatgctggaagatcactcctacctgccagtgggcagcgtgtccttccagaacatcagctccaacgtcctggaagagtccgtggtgtccgaggacacactgagcccagatgaggatggcgtgtgtgccggacagtactttaccgagtctggcctcgtgggccttctggaacaggccgccgaactgttttctaccggcggactgtacgagacagtgaacgaagtgtacaagctggtcatccccatcctggaagcccacagagagttcagaaagctgacactgacccactccaagctgcagcgggccttcgacagcattgtgaacaaggaccacaagcggatgttcggcacctacttcagagtgggctttttcggcagcaaattcggcgacctggacgagcaagagttcgtgtacaaagagcccgccatcaccaagctgcctgagatctcccatcggctggaagccttctacggccagtgttttggcgccgagttcgtggaagtgatcaaggacagcacccctgtggataagaccaaactggaccctaacaaggcctacatccagatcaccttcgtggaaccctacttcgacgagtacgagatgaaggatagagtgacctactttgagaagaacttcaacctgcggcggttcatgtacaccacaccttttacactggaaggcagacccaggggcgagctgcacgagcagtatagaagaaacaccgtgctgaccaccatgcacgcattcccttacatcaagacccggatcagcgtgatccagaaagaagagtttgtcctgacaccaatcgaggtggccatcgaggacatgaagaagaaaaccctccagctggccgtggccatcaatcaagaacctccagacgccaagatgctccagatggtgctccagggatctgtgggcgccacagtgaatcagggacctctggaagtggcccaagtgttcctggctgagatccccgccgatcctaagctgtacagacaccataacaagctgcggctctgcttcaaagaattcatcatgagatgtggcgaggccgtggaaaagaacaagagactgatcaccgccgaccagagagagtaccagcaagagctgaagaaaaactacaacaagctcaaagaaaatctgcggcccatgatcgagagaaagatccccgagctgtacaagcccatcttccgcgtggaaagccagaagcgggactcctttcacagatccagctttaggaagtgcgagacacagctgtcccagggctcttcgagcgactacaaagaccatgacggtgattataaagatcatgacatcgattacaaggatgacgatgacaagtgagaaaagccatcttcattcgtggagactgtggccctgcaaccctggagaaggacttgctggtacttaaaaaatgggacatttgccacccaggactgactgtacactccctgatcagccagcactctggaagctttgggatcccaggaaccatggaattattcccaaatggactctgaccagatttttgccatactggggggtggcgggatggaggatgggtactcaggcatgactgcgtatttattaaagtgtgtttttccacaatgtaccaaacaaggcataagcagcttctcctgctgactggccaatcactgcccatctgagagatgatttcctctggcccatatttgaatttattggagtaactcaaattgcctgaggaaaaatggaaaaattatccaccagtcgattcaaactgaatttcactctttataggaaggcagggcaaacttgtaggagtacgaaacattttcaataaatctacaaagggaagccttactacaattccaaaaatcatcatggttggaaatttgggaggagattatttgtgaacttgttacccttttggtaatggtggactaattgctgtatagttatttttgttttattattactgttacattaatttaacatgcatttatagaagaatacattcaaagcactgatgtaggagatacacggtacttggagcagtcagccagaaatcacagatactgctttcacttaaatggaaacaattctccgataatgctttgctttttttcttatgtcactcttgtgtactatctatttttctcctctctgggaccaagtttctttttataaagcaataatatctctgttttcatttcagaacattgtgctgtctgtcagcatatgtatatcagctacaaaatatattcaactttgacttcttttgacaaaggactttaggaaaaagaggaacaaagacattatttgagaattaaattatatatttttaatatgactgtgaccttgactgataataaagatgtaataagaattgcaagctaaattttatgaccctgtggagccagtggactttgaaggacttctgatgacacacctgaacagcctggatgtgcagcttgcccaggagctcggggacttcactgatgacgacttggacgtggtgttcacgccaaaggaatgtaggactttgcagccctctttgccggaggaagggtaaatagttttctaaaatgtagatgtgattgggattgtcatgattgttttcaataagtgggtaggggagatgccttcaatctgaacttaaaaataaaataaaattactcaatccattcaaatgtgtgggacagctatatgatatcatcatgtaagtatagatagtttttaaattagtttggccagagattttgaaaagctatgtgaagactaagggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggtctgagacaataaccctgataaatgcttcaataatgtaagctctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctactttttctctgtcacagaatgaaaatttttctgtcatctcttcgttattaatgtttgtaattgactgaatatcaacgcttatttgcagcctgaatggcgaatg

SEQ ID NO: 4 - DNA, 13207 bp, synthetic construct
  source 1..13207: mol_type = other DNA; note = mCCL-EFS-DOCK8ga-WPRE
acgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatagcacctagatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacgggggggtcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgagacagacgaggtttgcgcttggctgggcatgttccgcggctactctgcggcgcgccaggcccccgctttccgcaccccgcgaccctagaagccaccgaaccgccggcgggccatggccacactgcctagcgctgagagaagggccttcgctctgaagatcaaccggtacagcagcgccgagatccggaagcagtttaccctgcctcctaacctgggccagtaccacagacagagcatcagcaccagcggcttcccatctctgcagctgccccagttctacgaccctgtggaacccgtggatttcgagggcctgctgatgacccacctgaacagcctggatgtgcagctggcccaagagctgggcgacttcaccgacgatgatctggacgtggtgttcacccctaaagagtgcagaaccctgcagcctagcctgcctgaagaaggcgtggaactggatcctcacgtgcgggattgtgtgcagacctacatccgcgagtggctgatcgtgaaccggaagaatcagggcagcccagagatctgcggcttcaagaaaaccggcagccggaaggacttccacaagaccctgcctaagcagaccttcgagagcgagacactggaatgctctgagcctgctgctcaggccggacctagacacctgaatgtgctgtgtgacgtgtccggcaagggacctgtgaccgcctgcgatttcgacctgagaagtctgcagcccgacaagcggctggaaaacctgctccagcaagtgtccgccgaggacttcgagaagcagaacgaggaagccagacggaccaacagacaggccgagctgtttgctctgtaccccagcgtggacgaggaagatgccgtcgagattagacccgtgcctgagtgcccaaaagagcacctgggcaacagaatcctggtcaagctgctgaccctgaagttcgagatcgagattgagcccctgttcgcctctatcgccctgtacgacgtgaaagagcggaagaagatcagcgagaacttccactgcgacctgaactccgaccagttcaagggcttcctgagagcccacacaccatctgtggccgctagctctcaggccagatctgccgtgttcagcgtgacataccccagcagcgacatctacctggtggtcaagatcgagaaggtcctgcagcagggcgagatcggcgattgtgccgagccttacaccgtgatcaaagagagcgacggcggcaagagcaaagagaagattgagaagctgaagctgcaggccgagagcttctgtcagcggctgggcaagtacagaatgccctttgcctgggctcctatcagcctgtccagcttcttcaacgtgtccacactggaacgggaagtgaccgacgtggactctgtcgtgggcagatctagcgtgggcgagagaaggacactggctcagtctagacggctgagcgagagagccctgagcctggaagaaaatggcgtgggcagcaacttcaagaccagcactctgtccgtgtctagcttctttaagcaagagggcgacagactgagcgacgaggacctgttcaagttcctggccgactacaagcggagcagctccctgcagagaagagtgaagtctatccctggcctgctgcgcctggaaatctctacagcccctgagatcatcaactgctgtctgacccctgaaatgctgcccgtgaagccctttccagagaaccggaccagacctcacaaagagatcctggaattccccaccagagaagtgtacgtgccccacaccgtgtaccggaacctgctgtatgtgtacccacagcggctgaacttcgtgaacaagctggcctccgccagaaacatcaccatcaagatccagtttatgtgcggcgaggacgccagcaacgccatgcctgtgatcttcggcaagtctagcggccctgagttcctgcaagaggtgtacacagccgtgacctaccacaacaagagccccgacttctacgaggaagtgaagattaagctgcccgccaagctgaccgtgaatcaccatctgctgttcaccttctaccacatcagctgccagcagaaacagggcgcctctgtggaaacactgctgggctatagctggctgcccatcctgctgaacgagagactgcagaccggcagctactgtctgcctgtggctctggaaaagctgccacctaactacagcatgcactccgccgagaaggtgcccctgcagaatcctcctattaagtgggccgagggccacaagggcgtgttcaatatcgaggtgcaggccgtgtcctccgtgcacacccaggataaccacctggaaaagttcttcaccctgtgccacagcctcgagagccaagtgacattccccatccgcgtgctggaccagaaaatctccgagatggccctggaacacgagctgaaactgagcatcatctgcctgaatagcagcagactggaacccctggtgctgttcctgcatctggtgctggacaagctgttccagctgagcgtgcagcccatggttatcgccggacagaccgccaacttcagccagttcgcctttgagagcgtggtggccattgccaacagcctgcacaacagcaaggacctgagcaaggatcagcacggcagaaactgcctgctggcctcttacgtgcactacgtgttcagactgcctgaggtgcagagggacgtgccaaaatctggcgctcctaccgctctgctggaccccagaagctatcacacctacggcagaacatctgccgccgctgtgtcctctaaactcctgcaggctagagtgatgtccagcagcaaccctgatctggccggaacacacagcgccgctgatgaagaagtgaaaaacatcatgagcagcaagatcgccgaccggaactgcagccggatgagctactactgtagcggcagctctgatgcccctagctctccagctgctcctagacctgccagcaagaagcactttcacgaggaactggccctgcagatggtcgtgtctaccggcatggttcgagagacagtgtttaagtacgcctggttcttcttcgaactgctggtcaagagcatggcccagcacgtgcacaacatggacaagcgggacagcttccggcggaccagattcagcgacagattcatggacgacatcaccaccatcgtgaacgtggtcaccagcgagattgctgccctgctggttaagccccagaaagagaacgagcaggccgaaaagatgaatatctctctggccttcttcctctacgacctgctgagcctgatggaccggggcttcgtgttcaacctgatccggcactactgctcccagctgtctgccaagctgtccaacctgcctacactgatctctatgagactcgagttcctgaggatcctgtgcagccacgagcactacctgaacctgaatctgttcttcatgaacgccgacacagcccctacaagcccctgtccttctatcagcagccagaacagcagcagctgtagcagctttcaggatcagaaaatcgccagcatgttcgatctgaccagcgagtaccggcagcagcactttctgaccggcctgctgtttaccgaactggcagccgctcttgatgccgaaggcgagggaatcagcaaggtgcagcggaaagccgtgtctgccattcacagcctgctgtctagccacgacctggatcctagatgcgtgaagcctgaagtgaaagtgaaaatcgctgccctgtacctgcctctcgtgggcatcattctggatgccctgcctcagctgtgtgatttcaccgtggccgacaccagacggtacagaacctctggctccgacgaggaacaagaaggcgctggcgccatcaaccagaatgtggcccttgccattgccgggaacaacttcaatctgaaaaccagcggcatcgtgctgagcagcctgccttacaagcagtacaacatgctgaatgccgacactacccgcaacctgatgatctgcttcctgtggattatgaagaacgccgatcagagcctgattcggaagtggattgccgacctgccaagcactcagctgaaccggattctggacctcctgttcatctgcgtgctgtgcttcgagtacaagggcaagcagtccagcgacaaggtgtccactcaggtgctgcagaaaagccgggatgtgaaggccaggctggaagaggcacttctgagaggcgaaggcgccagaggcgagatgatgaggcgtagagcccctggcaacgatagattccccggcctgaatgagaacctgcggtggaagaaagagcagacccattggagacaggccaacgagaaactggacaagaccaaggccgaactggaccaagaggccctgatctctggcaacctggccacagaagcccacctgattatcctggacatgcaagagaacatcatccaggccagtagcgccctggattgcaaggattctctgctcggaggcgtgctgagggtgctcgtgaatagcctgaattgcgaccagagcaccacctacctgacacactgcttcgccacactgagggccctgattgccaagtttggggacctgctgttcgaggaagaggtggaacagtgcttcgacctgtgtcaccaagtgctgcaccactgcagcagctccatggacgtgacaagatcccaggcctgcgccactctgtatctgctgatgcggtttagcttcggcgccaccagcaatttcgccagagtgaagatgcaagtgaccatgagcctggccagcctcgttggaagggcccctgacttcaatgaggaacacctcagaagaagcctgcggaccatcctggcctacagcgaagaggataccgccatgcagatgaccccatttccaacacaagtggaagaactgctgtgcaacctgaattccatcctgtacgataccgtgaagatgagagagttccaagaggaccccgagatgctgatggatctgatgtaccggatcgccaagagctaccaggcctctcctgatctgagactgacctggctgcagaacatggccgagaagcacaccaagaagaagtgctacaccgaggccgccatgtgcctggttcatgctgctgcactcgtggccgagtacctgagcatgctggaagatcactcctacctgccagtgggcagcgtgtccttccagaacatcagctccaacgtcctggaagagtccgtggtgtccgaggacacactgagcccagatgaggatggcgtgtgtgccggacagtactttaccgagtctggcctcgtgggccttctggaacaggccgccgaactgttttctaccggcggactgtacgagacagtgaacgaagtgtacaagctggtcatccccatcctggaagcccacagagagttcagaaagctgacactgacccactccaagctgcagcgggccttcgacagcattgtgaacaaggaccacaagcggatgttcggcacctacttcagagtgggctttttcggcagcaaattcggcgacctggacgagcaagagttcgtgtacaaagagcccgccatcaccaagctgcctgagatctcccatcggctggaagccttctacggccagtgttttggcgccgagttcgtggaagtgatcaaggacagcacccctgtggataagaccaaactggaccctaacaaggcctacatccagatcaccttcgtggaaccctacttcgacgagtacgagatgaaggatagagtgacctactttgagaagaacttcaacctgcggcggttcatgtacaccacaccttttacactggaaggcagacccaggggcgagctgcacgagcagtatagaagaaacaccgtgctgaccaccatgcacgcattcccttacatcaagacccggatcagcgtgatccagaaagaagagtttgtcctgacaccaatcgaggtggccatcgaggacatgaagaagaaaaccctccagctggccgtggccatcaatcaagaacctccagacgccaagatgctccagatggtgctccagggatctgtgggcgccacagtgaatcagggacctctggaagtggcccaagtgttcctggctgagatccccgccgatcctaagctgtacagacaccataacaagctgcggctctgcttcaaagaattcatcatgagatgtggcgaggccgtggaaaagaacaagagactgatcaccgccgaccagagagagtaccagcaagagctgaagaaaaactacaacaagctcaaagaaaatctgcggcccatgatcgagagaaagatccccgagctgtacaagcccatcttccgcgtggaaagccagaagcgggactcctttcacagatccagctttaggaagtgcgagacacagctgtcccagggctcttgattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgctaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgttggttttttgtgtgtgcattgtctgagtaggtgtcattctattctggggggtggggtggggcagcacagcaagggggaggattgggaagacaatagcaggcatgctggggatgcggtgggctctatggagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctctcactacttctggaatagctcagaggccgaggcggcctcggcctctgcataaataaaaaaaattagtcgaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggg

SEQ ID NO: 5 - DNA, 7253 bp, synthetic construct
  source 1..7253: mol_type = other DNA; note = 939 DOCK8-CfaN_2
ctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtggatcccctgagggggcccccatgggctagaggatccggcctcggcctctgcataaataaaaaaaattagtcagccatgagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagcacgtgagatctgaattcgagatctgccgccgccatggccacactgcctagcgctgagagaagggccttcgctctgaagatcaaccggtacagcagcgccgagatccggaagcagtttaccctgcctcctaacctgggccagtaccacagacagagcatcagcaccagcggcttcccatctctgcagctgccccagttctacgaccctgtggaacccgtggatttcgagggcctgctgatgacccacctgaacagcctggatgtgcagctggcccaagagctgggcgacttcaccgacgatgatctggacgtggtgttcacccctaaagagtgcagaaccctgcagcctagcctgcctgaagaaggcgtggaactggatcctcacgtgcgggattgtgtgcagacctacatccgcgagtggctgatcgtgaaccggaagaatcagggcagcccagagatctgcggcttcaagaaaaccggcagccggaaggacttccacaagaccctgcctaagcagaccttcgagagcgagacactggaatgctctgagcctgctgctcaggccggacctagacacctgaatgtgctgtgtgacgtgtccggcaagggacctgtgaccgcctgcgatttcgacctgagaagtctgcagcccgacaagcggctggaaaacctgctccagcaagtgtccgccgaggacttcgagaagcagaacgaggaagccagacggaccaacagacaggccgagctgtttgctctgtaccccagcgtggacgaggaagatgccgtcgagattagacccgtgcctgagtgcccaaaagagcacctgggcaacagaatcctggtcaagctgctgaccctgaagttcgagatcgagattgagcccctgttcgcctctatcgccctgtacgacgtgaaagagcggaagaagatcagcgagaacttccactgcgacctgaactccgaccagttcaagggcttcctgagagcccacacaccatctgtggccgctagctctcaggccagatctgccgtgttcagcgtgacataccccagcagcgacatctacctggtggtcaagatcgagaaggtcctgcagcagggcgagatcggcgattgtgccgagccttacaccgtgatcaaagagagcgacggcggcaagagcaaagagaagattgagaagctgaagctgcaggccgagagcttctgtcagcggctgggcaagtacagaatgccctttgcctgggctcctatcagcctgtccagcttcttcaacgtgtccacactggaacgggaagtgaccgacgtggactctgtcgtgggcagatctagcgtgggcgagagaaggacactggctcagtctagacggctgagcgagagagccctgagcctggaagaaaatggcgtgggcagcaacttcaagaccagcactctgtccgtgtctagcttctttaagcaagagggcgacagactgagcgacgaggacctgttcaagttcctggccgactacaagcggagcagctccctgcagagaagagtgaagtctatccctggcctgctgcgcctggaaatctctacagcccctgagatcatcaactgctgtctgacccctgaaatgctgcccgtgaagccctttccagagaaccggaccagacctcacaaagagatcctggaattccccaccagagaagtgtacgtgccccacaccgtgtaccggaacctgctgtatgtgtacccacagcggctgaacttcgtgaacaagctggcctccgccagaaacatcaccatcaagatccagtttatgtgcggcgaggacgccagcaacgccatgcctgtgatcttcggcaagtctagcggccctgagttcctgcaagaggtgtacacagccgtgacctaccacaacaagagccccgacttctacgaggaagtgaagattaagctgcccgccaagctgaccgtgaatcaccatctgctgttcaccttctaccacatcagctgccagcagaaacagggcgcctctgtggaaacactgctgggctatagctggctgcccatcctgctgaacgagagactgcagaccggcagctactgtctgcctgtggctctggaaaagctgccacctaactacagcatgcactccgccgagaaggtgcccctgcagaatcctcctattaagtgggccgagggccacaagggcgtgttcaatatcgaggtgcaggccgtgtcctccgtgcacacccaggataaccacctggaaaagttcttcaccctgtgccacagcctcgagagccaagtgacattccccatccgcgtgctggaccagaaaatctccgagatggccctggaacacgagctgaaactgagcatcatctgcctgaatagcagcagactggaacccctggtgctgttcctgcatctggtgctggacaagctgttccagctgagcgtgcagcccatggttatcgccggacagaccgccaacttcagccagttcgcctttgagagcgtggtggccattgccaacagcctgcacaacagcaaggacctgagcaaggatcagcacggcagaaactgcctgctggcctcttacgtgcactacgtgttcagactgcctgaggtgcagagggacgtgccaaaatctggcgctcctaccgctctgctggaccccagaagctatcacacctacggcagaacatctgccgccgctgtgtcctctaaactcctgcaggctagagtgatgtccagcagcaaccctgatctggccggaacacacagcgccgctgatgaagaagtgaaaaacatcatgagcagcaagatcgccgaccggaactgcagccggatgagctactactgcctgagctacgacaccgagatcctgaccgtggaatacggcttcctgcctatcggcaagatcgtggaagaacggatcgagtgcaccgtgtacaccgtggacaagaacggcttcgtgtacacacagcctatcgctcagtggcacaaccggggagagcaagaggtgttcgagtactgcctggaagatggcagcatcatccgggccaccaaggaccacaagttcatgaccaccgacggccagatgctgcccatcgacgagatctttgagagaggcctggacctgaaacaggtggacggacttccttgactggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagcactagtgcggccgcagatctgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgat

SEQ ID NO: 6 - DNA, 9959 bp, synthetic construct
  source 1..9959: mol_type = other DNA; note = mCCL-EFS-939Dock8-CfaN-WPRE
acgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatagcacctagatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacgggggggtcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgaaaataagagagaaaagaagagtaagaagaaatataagagccaccatggccacactgcctagcgctgagagaagggccttcgctctgaagatcaaccggtacagcagcgccgagatccggaagcagtttaccctgcctcctaacctgggccagtaccacagacagagcatcagcaccagcggcttcccatctctgcagctgccccagttctacgaccctgtggaacccgtggatttcgagggcctgctgatgacccacctgaacagcctggatgtgcagctggcccaagagctgggcgacttcaccgacgatgatctggacgtggtgttcacccctaaagagtgcagaaccctgcagcctagcctgcctgaagaaggcgtggaactggatcctcacgtgcgggattgtgtgcagacctacatccgcgagtggctgatcgtgaaccggaagaatcagggcagcccagagatctgcggcttcaagaaaaccggcagccggaaggacttccacaagaccctgcctaagcagaccttcgagagcgagacactggaatgctctgagcctgctgctcaggccggacctagacacctgaatgtgctgtgtgacgtgtccggcaagggacctgtgaccgcctgcgatttcgacctgagaagtctgcagcccgacaagcggctggaaaacctgctccagcaagtgtccgccgaggacttcgagaagcagaacgaggaagccagacggaccaacagacaggccgagctgtttgctctgtaccccagcgtggacgaggaagatgccgtcgagattagacccgtgcctgagtgcccaaaagagcacctgggcaacagaatcctggtcaagctgctgaccctgaagttcgagatcgagattgagcccctgttcgcctctatcgccctgtacgacgtgaaagagcggaagaagatcagcgagaacttccactgcgacctgaactccgaccagttcaagggcttcctgagagcccacacaccatctgtggccgctagctctcaggccagatctgccgtgttcagcgtgacataccccagcagcgacatctacctggtggtcaagatcgagaaggtcctgcagcagggcgagatcggcgattgtgccgagccttacaccgtgatcaaagagagcgacggcggcaagagcaaagagaagattgagaagctgaagctgcaggccgagagcttctgtcagcggctgggcaagtacagaatgccctttgcctgggctcctatcagcctgtccagcttcttcaacgtgtccacactggaacgggaagtgaccgacgtggactctgtcgtgggcagatctagcgtgggcgagagaaggacactggctcagtctagacggctgagcgagagagccctgagcctggaagaaaatggcgtgggcagcaacttcaagaccagcactctgtccgtgtctagcttctttaagcaagagggcgacagactgagcgacgaggacctgttcaagttcctggccgactacaagcggagcagctccctgcagagaagagtgaagtctatccctggcctgctgcgcctggaaatctctacagcccctgagatcatcaactgctgtctgacccctgaaatgctgcccgtgaagccctttccagagaaccggaccagacctcacaaagagatcctggaattccccaccagagaagtgtacgtgccccacaccgtgtaccggaacctgctgtatgtgtacccacagcggctgaacttcgtgaacaagctggcctccgccagaaacatcaccatcaagatccagtttatgtgcggcgaggacgccagcaacgccatgcctgtgatcttcggcaagtctagcggccctgagttcctgcaagaggtgtacacagccgtgacctaccacaacaagagccccgacttctacgaggaagtgaagattaagctgcccgccaagctgaccgtgaatcaccatctgctgttcaccttctaccacatcagctgccagcagaaacagggcgcctctgtggaaacactgctgggctatagctggctgcccatcctgctgaacgagagactgcagaccggcagctactgtctgcctgtggctctggaaaagctgccacctaactacagcatgcactccgccgagaaggtgcccctgcagaatcctcctattaagtgggccgagggccacaagggcgtgttcaatatcgaggtgcaggccgtgtcctccgtgcacacccaggataaccacctggaaaagttcttcaccctgtgccacagcctcgagagccaagtgacattccccatccgcgtgctggaccagaaaatctccgagatggccctggaacacgagctgaaactgagcatcatctgcctgaatagcagcagactggaacccctggtgctgttcctgcatctggtgctggacaagctgttccagctgagcgtgcagcccatggttatcgccggacagaccgccaacttcagccagttcgcctttgagagcgtggtggccattgccaacagcctgcacaacagcaaggacctgagcaaggatcagcacggcagaaactgcctgctggcctcttacgtgcactacgtgttcagactgcctgaggtgcagagggacgtgccaaaatctggcgctcctaccgctctgctggaccccagaagctatcacacctacggcagaacatctgccgccgctgtgtcctctaaactcctgcaggctagagtgatgtccagcagcaaccctgatctggccggaacacacagcgccgctgatgaagaagtgaaaaacatcatgagcagcaagatcgccgaccggaactgcagccggatgagctactactgcctgagctacgacaccgagatcctgaccgtggaatacggcttcctgcctatcggcaagatcgtggaagaacggatcgagtgcaccgtgtacaccgtggacaagaacggcttcgtgtacacacagcctatcgctcagtggcacaaccggggagagcaagaggtgttcgagtactgcctggaagatggcagcatcatccgggccaccaaggaccacaagttcatgaccaccgacggccagatgctgcccatcgacgagatctttgagagaggcctggacctgaaacaggtggacggacttccttgattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgctaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgttggttttttgtgtgtgcattgtctgagtaggtgtcattctattctggggggtggggtggggcagcacagcaagggggaggattgggaagacaatagcaggcatgctggggatgcggtgggctctatggagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctctcactacttctggaatagctcagaggccgaggcggcctcggcctctgcataaataaaaaaaattagtcgaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggg

SEQ ID NO: 7 - DNA, 7730 bp, synthetic construct
  source 1..7730: mol_type = other DNA; note = 939 CfaC-CF-DOCK8-bGHpA
gaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtggatcccctgagggggcccccatgggctagaggatccggcctcggcctctgcataaataaaaaaaattagtcagccatgagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagcacgtgagatctgaattcgagatctgccgccgccatggtgaagatcatcagcagaaagagcctgggcacccagaacgtgtacgacatcggcgtggaaaaggaccacaactttctgctcaagaacggcctggtggccagcaactgctttagcggcagctctgatgcccctagctctccagctgctcctagacctgccagcaagaagcactttcacgaggaactggccctgcagatggtcgtgtctaccggcatggttcgagagacagtgtttaagtacgcctggttcttcttcgaactgctggtcaagagcatggcccagcacgtgcacaacatggacaagcgggacagcttccggcggaccagattcagcgacagattcatggacgacatcaccaccatcgtgaacgtggtcaccagcgagattgctgccctgctggttaagccccagaaagagaacgagcaggccgaaaagatgaatatctctctggccttcttcctctacgacctgctgagcctgatggaccggggcttcgtgttcaacctgatccggcactactgctcccagctgtctgccaagctgtccaacctgcctacactgatctctatgagactcgagttcctgaggatcctgtgcagccacgagcactacctgaacctgaatctgttcttcatgaacgccgacacagcccctacaagcccctgtccttctatcagcagccagaacagcagcagctgtagcagctttcaggatcagaaaatcgccagcatgttcgatctgaccagcgagtaccggcagcagcactttctgaccggcctgctgtttaccgaactggcagccgctcttgatgccgaaggcgagggaatcagcaaggtgcagcggaaagccgtgtctgccattcacagcctgctgtctagccacgacctggatcctagatgcgtgaagcctgaagtgaaagtgaaaatcgctgccctgtacctgcctctcgtgggcatcattctggatgccctgcctcagctgtgtgatttcaccgtggccgacaccagacggtacagaacctctggctccgacgaggaacaagaaggcgctggcgccatcaaccagaatgtggcccttgccattgccgggaacaacttcaatctgaaaaccagcggcatcgtgctgagcagcctgccttacaagcagtacaacatgctgaatgccgacactacccgcaacctgatgatctgcttcctgtggattatgaagaacgccgatcagagcctgattcggaagtggattgccgacctgccaagcactcagctgaaccggattctggacctcctgttcatctgcgtgctgtgcttcgagtacaagggcaagcagtccagcgacaaggtgtccactcaggtgctgcagaaaagccgggatgtgaaggccaggctggaagaggcacttctgagaggcgaaggcgccagaggcgagatgatgaggcgtagagcccctggcaacgatagattccccggcctgaatgagaacctgcggtggaagaaagagcagacccattggagacaggccaacgagaaactggacaagaccaaggccgaactggaccaagaggccctgatctctggcaacctggccacagaagcccacctgattatcctggacatgcaagagaacatcatccaggccagtagcgccctggattgcaaggattctctgctcggaggcgtgctgagggtgctcgtgaatagcctgaattgcgaccagagcaccacctacctgacacactgcttcgccacactgagggccctgattgccaagtttggggacctgctgttcgaggaagaggtggaacagtgcttcgacctgtgtcaccaagtgctgcaccactgcagcagctccatggacgtgacaagatcccaggcctgcgccactctgtatctgctgatgcggtttagcttcggcgccaccagcaatttcgccagagtgaagatgcaagtgaccatgagcctggccagcctcgttggaagggcccctgacttcaatgaggaacacctcagaagaagcctgcggaccatcctggcctacagcgaagaggataccgccatgcagatgaccccatttccaacacaagtggaagaactgctgtgcaacctgaattccatcctgtacgataccgtgaagatgagagagttccaagaggaccccgagatgctgatggatctgatgtaccggatcgccaagagctaccaggcctctcctgatctgagactgacctggctgcagaacatggccgagaagcacaccaagaagaagtgctacaccgaggccgccatgtgcctggttcatgctgctgcactcgtggccgagtacctgagcatgctggaagatcactcctacctgccagtgggcagcgtgtccttccagaacatcagctccaacgtcctggaagagtccgtggtgtccgaggacacactgagcccagatgaggatggcgtgtgtgccggacagtactttaccgagtctggcctcgtgggccttctggaacaggccgccgaactgttttctaccggcggactgtacgagacagtgaacgaagtgtacaagctggtcatccccatcctggaagcccacagagagttcagaaagctgacactgacccactccaagctgcagcgggccttcgacagcattgtgaacaaggaccacaagcggatgttcggcacctacttcagagtgggctttttcggcagcaaattcggcgacctggacgagcaagagttcgtgtacaaagagcccgccatcaccaagctgcctgagatctcccatcggctggaagccttctacggccagtgttttggcgccgagttcgtggaagtgatcaaggacagcacccctgtggataagaccaaactggaccctaacaaggcctacatccagatcaccttcgtggaaccctacttcgacgagtacgagatgaaggatagagtgacctactttgagaagaacttcaacctgcggcggttcatgtacaccacaccttttacactggaaggcagacccaggggcgagctgcacgagcagtatagaagaaacaccgtgctgaccaccatgcacgcattcccttacatcaagacccggatcagcgtgatccagaaagaagagtttgtcctgacaccaatcgaggtggccatcgaggacatgaagaagaaaaccctccagctggccgtggccatcaatcaagaacctccagacgccaagatgctccagatggtgctccagggatctgtgggcgccacagtgaatcagggacctctggaagtggcccaagtgttcctggctgagatccccgccgatcctaagctgtacagacaccataacaagctgcggctctgcttcaaagaattcatcatgagatgtggcgaggccgtggaaaagaacaagagactgatcaccgccgaccagagagagtaccagcaagagctgaagaaaaactacaacaagctcaaagaaaatctgcggcccatgatcgagagaaagatccccgagctgtacaagcccatcttccgcgtggaaagccagaagcgggactcctttcacagatccagctttaggaagtgcgagacacagctgtcccagggctcttgactggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagcactagtgcggccgcagatctgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaa

SEQ ID NO: 8 - DNA, 10436 bp, synthetic construct
  source 1..10436: mol_type = other DNA; note = mCCL-EFS-939Dock8-CfaC-CF-WPRE
acgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatagcacctagatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacgggggggtcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgaaaataagagagaaaagaagagtaagaagaaatataagagccaccatggtgaagatcatcagcagaaagagcctgggcacccagaacgtgtacgacatcggcgtggaaaaggaccacaactttctgctcaagaacggcctggtggccagcaactgctttagcggcagctctgatgcccctagctctccagctgctcctagacctgccagcaagaagcactttcacgaggaactggccctgcagatggtcgtgtctaccggcatggttcgagagacagtgtttaagtacgcctggttcttcttcgaactgctggtcaagagcatggcccagcacgtgcacaacatggacaagcgggacagcttccggcggaccagattcagcgacagattcatggacgacatcaccaccatcgtgaacgtggtcaccagcgagattgctgccctgctggttaagccccagaaagagaacgagcaggccgaaaagatgaatatctctctggccttcttcctctacgacctgctgagcctgatggaccggggcttcgtgttcaacctgatccggcactactgctcccagctgtctgccaagctgtccaacctgcctacactgatctctatgagactcgagttcctgaggatcctgtgcagccacgagcactacctgaacctgaatctgttcttcatgaacgccgacacagcccctacaagcccctgtccttctatcagcagccagaacagcagcagctgtagcagctttcaggatcagaaaatcgccagcatgttcgatctgaccagcgagtaccggcagcagcactttctgaccggcctgctgtttaccgaactggcagccgctcttgatgccgaaggcgagggaatcagcaaggtgcagcggaaagccgtgtctgccattcacagcctgctgtctagccacgacctggatcctagatgcgtgaagcctgaagtgaaagtgaaaatcgctgccctgtacctgcctctcgtgggcatcattctggatgccctgcctcagctgtgtgatttcaccgtggccgacaccagacggtacagaacctctggctccgacgaggaacaagaaggcgctggcgccatcaaccagaatgtggcccttgccattgccgggaacaacttcaatctgaaaaccagcggcatcgtgctgagcagcctgccttacaagcagtacaacatgctgaatgccgacactacccgcaacctgatgatctgcttcctgtggattatgaagaacgccgatcagagcctgattcggaagtggattgccgacctgccaagcactcagctgaaccggattctggacctcctgttcatctgcgtgctgtgcttcgagtacaagggcaagcagtccagcgacaaggtgtccactcaggtgctgcagaaaagccgggatgtgaaggccaggctggaagaggcacttctgagaggcgaaggcgccagaggcgagatgatgaggcgtagagcccctggcaacgatagattccccggcctgaatgagaacctgcggtggaagaaagagcagacccattggagacaggccaacgagaaactggacaagaccaaggccgaactggaccaagaggccctgatctctggcaacctggccacagaagcccacctgattatcctggacatgcaagagaacatcatccaggccagtagcgccctggattgcaaggattctctgctcggaggcgtgctgagggtgctcgtgaatagcctgaattgcgaccagagcaccacctacctgacacactgcttcgccacactgagggccctgattgccaagtttggggacctgctgttcgaggaagaggtggaacagtgcttcgacctgtgtcaccaagtgctgcaccactgcagcagctccatggacgtgacaagatcccaggcctgcgccactctgtatctgctgatgcggtttagcttcggcgccaccagcaatttcgccagagtgaagatgcaagtgaccatgagcctggccagcctcgttggaagggcccctgacttcaatgaggaacacctcagaagaagcctgcggaccatcctggcctacagcgaagaggataccgccatgcagatgaccccatttccaacacaagtggaagaactgctgtgcaacctgaattccatcctgtacgataccgtgaagatgagagagttccaagaggaccccgagatgctgatggatctgatgtaccggatcgccaagagctaccaggcctctcctgatctgagactgacctggctgcagaacatggccgagaagcacaccaagaagaagtgctacaccgaggccgccatgtgcctggttcatgctgctgcactcgtggccgagtacctgagcatgctggaagatcactcctacctgccagtgggcagcgtgtccttccagaacatcagctccaacgtcctggaagagtccgtggtgtccgaggacacactgagcccagatgaggatggcgtgtgtgccggacagtactttaccgagtctggcctcgtgggccttctggaacaggccgccgaactgttttctaccggcggactgtacgagacagtgaacgaagtgtacaagctggtcatccccatcctggaagcccacagagagttcagaaagctgacactgacccactccaagctgcagcgggccttcgacagcattgtgaacaaggaccacaagcggatgttcggcacctacttcagagtgggctttttcggcagcaaattcggcgacctggacgagcaagagttcgtgtacaaagagcccgccatcaccaagctgcctgagatctcccatcggctggaagccttctacggccagtgttttggcgccgagttcgtggaagtgatcaaggacagcacccctgtggataagaccaaactggaccctaacaaggcctacatccagatcaccttcgtggaaccctacttcgacgagtacgagatgaaggatagagtgacctactttgagaagaacttcaacctgcggcggttcatgtacaccacaccttttacactggaaggcagacccaggggcgagctgcacgagcagtatagaagaaacaccgtgctgaccaccatgcacgcattcccttacatcaagacccggatcagcgtgatccagaaagaagagtttgtcctgacaccaatcgaggtggccatcgaggacatgaagaagaaaaccctccagctggccgtggccatcaatcaagaacctccagacgccaagatgctccagatggtgctccagggatctgtgggcgccacagtgaatcagggacctctggaagtggcccaagtgttcctggctgagatccccgccgatcctaagctgtacagacaccataacaagctgcggctctgcttcaaagaattcatcatgagatgtggcgaggccgtggaaaagaacaagagactgatcaccgccgaccagagagagtaccagcaagagctgaagaaaaactacaacaagctcaaagaaaatctgcggcccatgatcgagagaaagatccccgagctgtacaagcccatcttccgcgtggaaagccagaagcgggactcctttcacagatccagctttaggaagtgcgagacacagctgtcccagggctcttgattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgctaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgttggttttttgtgtgtgcattgtctgagtaggtgtcattctattctggggggtggggtggggcagcacagcaagggggaggattgggaagacaatagcaggcatgctggggatgcggtgggctctatggagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctctcactacttctggaatagctcagaggccgaggcggcctcggcctctgcataaataaaaaaaattagtcgaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggg

SEQ ID NO: 9 - DNA, 7733 bp, synthetic construct
  source 1..7733: mol_type = other DNA; note = 939 CfaC-CFN-DOCK8-bGHpA
ctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtggatcccctgagggggcccccatgggctagaggatccggcctcggcctctgcataaataaaaaaaattagtcagccatgagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagcacgtgagatctgaattcgagatctgccgccgccatggtgaagatcatcagcagaaagagcctgggcacccagaacgtgtacgacatcggcgtggaaaaggaccacaactttctgctcaagaacggcctggtggccagcaactgctttaatagcggcagctctgatgcccctagctctccagctgctcctagacctgccagcaagaagcactttcacgaggaactggccctgcagatggtcgtgtctaccggcatggttcgagagacagtgtttaagtacgcctggttcttcttcgaactgctggtcaagagcatggcccagcacgtgcacaacatggacaagcgggacagcttccggcggaccagattcagcgacagattcatggacgacatcaccaccatcgtgaacgtggtcaccagcgagattgctgccctgctggttaagccccagaaagagaacgagcaggccgaaaagatgaatatctctctggccttcttcctctacgacctgctgagcctgatggaccggggcttcgtgttcaacctgatccggcactactgctcccagctgtctgccaagctgtccaacctgcctacactgatctctatgagactcgagttcctgaggatcctgtgcagccacgagcactacctgaacctgaatctgttcttcatgaacgccgacacagcccctacaagcccctgtccttctatcagcagccagaacagcagcagctgtagcagctttcaggatcagaaaatcgccagcatgttcgatctgaccagcgagtaccggcagcagcactttctgaccggcctgctgtttaccgaactggcagccgctcttgatgccgaaggcgagggaatcagcaaggtgcagcggaaagccgtgtctgccattcacagcctgctgtctagccacgacctggatcctagatgcgtgaagcctgaagtgaaagtgaaaatcgctgccctgtacctgcctctcgtgggcatcattctggatgccctgcctcagctgtgtgatttcaccgtggccgacaccagacggtacagaacctctggctccgacgaggaacaagaaggcgctggcgccatcaaccagaatgtggcccttgccattgccgggaacaacttcaatctgaaaaccagcggcatcgtgctgagcagcctgccttacaagcagtacaacatgctgaatgccgacactacccgcaacctgatgatctgcttcctgtggattatgaagaacgccgatcagagcctgattcggaagtggattgccgacctgccaagcactcagctgaaccggattctggacctcctgttcatctgcgtgctgtgcttcgagtacaagggcaagcagtccagcgacaaggtgtccactcaggtgctgcagaaaagccgggatgtgaaggccaggctggaagaggcacttctgagaggcgaaggcgccagaggcgagatgatgaggcgtagagcccctggcaacgatagattccccggcctgaatgagaacctgcggtggaagaaagagcagacccattggagacaggccaacgagaaactggacaagaccaaggccgaactggaccaagaggccctgatctctggcaacctggccacagaagcccacctgattatcctggacatgcaagagaacatcatccaggccagtagcgccctggattgcaaggattctctgctcggaggcgtgctgagggtgctcgtgaatagcctgaattgcgaccagagcaccacctacctgacacactgcttcgccacactgagggccctgattgccaagtttggggacctgctgttcgaggaagaggtggaacagtgcttcgacctgtgtcaccaagtgctgcaccactgcagcagctccatggacgtgacaagatcccaggcctgcgccactctgtatctgctgatgcggtttagcttcggcgccaccagcaatttcgccagagtgaagatgcaagtgaccatgagcctggccagcctcgttggaagggcccctgacttcaatgaggaacacctcagaagaagcctgcggaccatcctggcctacagcgaagaggataccgccatgcagatgaccccatttccaacacaagtggaagaactgctgtgcaacctgaattccatcctgtacgataccgtgaagatgagagagttccaagaggaccccgagatgctgatggatctgatgtaccggatcgccaagagctaccaggcctctcctgatctgagactgacctggctgcagaacatggccgagaagcacaccaagaagaagtgctacaccgaggccgccatgtgcctggttcatgctgctgcactcgtggccgagtacctgagcatgctggaagatcactcctacctgccagtgggcagcgtgtccttccagaacatcagctccaacgtcctggaagagtccgtggtgtccgaggacacactgagcccagatgaggatggcgtgtgtgccggacagtactttaccgagtctggcctcgtgggccttctggaacaggccgccgaactgttttctaccggcggactgtacgagacagtgaacgaagtgtacaagctggtcatccccatcctggaagcccacagagagttcagaaagctgacactgacccactccaagctgcagcgggccttcgacagcattgtgaacaaggaccacaagcggatgttcggcacctacttcagagtgggctttttcggcagcaaattcggcgacctggacgagcaagagttcgtgtacaaagagcccgccatcaccaagctgcctgagatctcccatcggctggaagccttctacggccagtgttttggcgccgagttcgtggaagtgatcaaggacagcacccctgtggataagaccaaactggaccctaacaaggcctacatccagatcaccttcgtggaaccctacttcgacgagtacgagatgaaggatagagtgacctactttgagaagaacttcaacctgcggcggttcatgtacaccacaccttttacactggaaggcagacccaggggcgagctgcacgagcagtatagaagaaacaccgtgctgaccaccatgcacgcattcccttacatcaagacccggatcagcgtgatccagaaagaagagtttgtcctgacaccaatcgaggtggccatcgaggacatgaagaagaaaaccctccagctggccgtggccatcaatcaagaacctccagacgccaagatgctccagatggtgctccagggatctgtgggcgccacagtgaatcagggacctctggaagtggcccaagtgttcctggctgagatccccgccgatcctaagctgtacagacaccataacaagctgcggctctgcttcaaagaattcatcatgagatgtggcgaggccgtggaaaagaacaagagactgatcaccgccgaccagagagagtaccagcaagagctgaagaaaaactacaacaagctcaaagaaaatctgcggcccatgatcgagagaaagatccccgagctgtacaagcccatcttccgcgtggaaagccagaagcgggactcctttcacagatccagctttaggaagtgcgagacacagctgtcccagggctcttgactggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagcactagtgcggccgcagatctgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgat

SEQ ID NO: 10 - DNA, 8240 bp, synthetic construct
  source 1..8240: mol_type = other DNA; note = 1267 DOCK8-CfaN_2
ctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtggatcccctgagggggcccccatgggctagaggatccggcctcggcctctgcataaataaaaaaaattagtcagccatgagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagcacgtgagatctgaattcgagatctgccgccgccatggccacactgcctagcgctgagagaagggccttcgctctgaagatcaaccggtacagcagcgccgagatccggaagcagtttaccctgcctcctaacctgggccagtaccacagacagagcatcagcaccagcggcttcccatctctgcagctgccccagttctacgaccctgtggaacccgtggatttcgagggcctgctgatgacccacctgaacagcctggatgtgcagctggcccaagagctgggcgacttcaccgacgatgatctggacgtggtgttcacccctaaagagtgcagaaccctgcagcctagcctgcctgaagaaggcgtggaactggatcctcacgtgcgggattgtgtgcagacctacatccgcgagtggctgatcgtgaaccggaagaatcagggcagcccagagatctgcggcttcaagaaaaccggcagccggaaggacttccacaagaccctgcctaagcagaccttcgagagcgagacactggaatgctctgagcctgctgctcaggccggacctagacacctgaatgtgctgtgtgacgtgtccggcaagggacctgtgaccgcctgcgatttcgacctgagaagtctgcagcccgacaagcggctggaaaacctgctccagcaagtgtccgccgaggacttcgagaagcagaacgaggaagccagacggaccaacagacaggccgagctgtttgctctgtaccccagcgtggacgaggaagatgccgtcgagattagacccgtgcctgagtgcccaaaagagcacctgggcaacagaatcctggtcaagctgctgaccctgaagttcgagatcgagattgagcccctgttcgcctctatcgccctgtacgacgtgaaagagcggaagaagatcagcgagaacttccactgcgacctgaactccgaccagttcaagggcttcctgagagcccacacaccatctgtggccgctagctctcaggccagatctgccgtgttcagcgtgacataccccagcagcgacatctacctggtggtcaagatcgagaaggtcctgcagcagggcgagatcggcgattgtgccgagccttacaccgtgatcaaagagagcgacggcggcaagagcaaagagaagattgagaagctgaagctgcaggccgagagcttctgtcagcggctgggcaagtacagaatgccctttgcctgggctcctatcagcctgtccagcttcttcaacgtgtccacactggaacgggaagtgaccgacgtggactctgtcgtgggcagatctagcgtgggcgagagaaggacactggctcagtctagacggctgagcgagagagccctgagcctggaagaaaatggcgtgggcagcaacttcaagaccagcactctgtccgtgtctagcttctttaagcaagagggcgacagactgagcgacgaggacctgttcaagttcctggccgactacaagcggagcagctccctgcagagaagagtgaagtctatccctggcctgctgcgcctggaaatctctacagcccctgagatcatcaactgctgtctgacccctgaaatgctgcccgtgaagccctttccagagaaccggaccagacctcacaaagagatcctggaattccccaccagagaagtgtacgtgccccacaccgtgtaccggaacctgctgtatgtgtacccacagcggctgaacttcgtgaacaagctggcctccgccagaaacatcaccatcaagatccagtttatgtgcggcgaggacgccagcaacgccatgcctgtgatcttcggcaagtctagcggccctgagttcctgcaagaggtgtacacagccgtgacctaccacaacaagagccccgacttctacgaggaagtgaagattaagctgcccgccaagctgaccgtgaatcaccatctgctgttcaccttctaccacatcagctgccagcagaaacagggcgcctctgtggaaacactgctgggctatagctggctgcccatcctgctgaacgagagactgcagaccggcagctactgtctgcctgtggctctggaaaagctgccacctaactacagcatgcactccgccgagaaggtgcccctgcagaatcctcctattaagtgggccgagggccacaagggcgtgttcaatatcgaggtgcaggccgtgtcctccgtgcacacccaggataaccacctggaaaagttcttcaccctgtgccacagcctcgagagccaagtgacattccccatccgcgtgctggaccagaaaatctccgagatggccctggaacacgagctgaaactgagcatcatctgcctgaatagcagcagactggaacccctggtgctgttcctgcatctggtgctggacaagctgttccagctgagcgtgcagcccatggttatcgccggacagaccgccaacttcagccagttcgcctttgagagcgtggtggccattgccaacagcctgcacaacagcaaggacctgagcaaggatcagcacggcagaaactgcctgctggcctcttacgtgcactacgtgttcagactgcctgaggtgcagagggacgtgccaaaatctggcgctcctaccgctctgctggaccccagaagctatcacacctacggcagaacatctgccgccgctgtgtcctctaaactcctgcaggctagagtgatgtccagcagcaaccctgatctggccggaacacacagcgccgctgatgaagaagtgaaaaacatcatgagcagcaagatcgccgaccggaactgcagccggatgagctactactgtagcggcagctctgatgcccctagctctccagctgctcctagacctgccagcaagaagcactttcacgaggaactggccctgcagatggtcgtgtctaccggcatggttcgagagacagtgtttaagtacgcctggttcttcttcgaactgctggtcaagagcatggcccagcacgtgcacaacatggacaagcgggacagcttccggcggaccagattcagcgacagattcatggacgacatcaccaccatcgtgaacgtggtcaccagcgagattgctgccctgctggttaagccccagaaagagaacgagcaggccgaaaagatgaatatctctctggccttcttcctctacgacctgctgagcctgatggaccggggcttcgtgttcaacctgatccggcactactgctcccagctgtctgccaagctgtccaacctgcctacactgatctctatgagactcgagttcctgaggatcctgtgcagccacgagcactacctgaacctgaatctgttcttcatgaacgccgacacagcccctacaagcccctgtccttctatcagcagccagaacagcagcagctgtagcagctttcaggatcagaaaatcgccagcatgttcgatctgaccagcgagtaccggcagcagcactttctgaccggcctgctgtttaccgaactggcagccgctcttgatgccgaaggcgagggaatcagcaaggtgcagcggaaagccgtgtctgccattcacagcctgctgtctagccacgacctggatcctagatgcgtgaagcctgaagtgaaagtgaaaatcgctgccctgtacctgcctctcgtgggcatcattctggatgccctgcctcagctgtgtgatttcaccgtggccgacaccagacggtacagaacctctggctccgacgaggaacaagaaggcgctggcgccatcaaccagaatgtggcccttgccattgccgggaacaactgcctgagctacgacaccgagatcctgaccgtggaatacggcttcctgcctatcggcaagatcgtggaagaacggatcgagtgcaccgtgtacaccgtggacaagaacggcttcgtgtacacacagcctatcgctcagtggcacaaccggggagagcaagaggtgttcgagtactgcctggaagatggcagcatcatccgggccaccaaggaccacaagttcatgaccaccgacggccagatgctgcccatcgacgagatctttgagagaggcctggacctgaaacaggtggacggacttccttgactggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagcactagtgcggccgcagatctgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgat

SEQ ID NO: 11 - DNA, 10946 bp, synthetic construct
  source 1..10946: mol_type = other DNA; note = mCCL-EFS-1267Dock8-CfaN-WPRE
acgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatagcacctagatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacgggggggtcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgaaaataagagagaaaagaagagtaagaagaaatataagagccaccatggccacactgcctagcgctgagagaagggccttcgctctgaagatcaaccggtacagcagcgccgagatccggaagcagtttaccctgcctcctaacctgggccagtaccacagacagagcatcagcaccagcggcttcccatctctgcagctgccccagttctacgaccctgtggaacccgtggatttcgagggcctgctgatgacccacctgaacagcctggatgtgcagctggcccaagagctgggcgacttcaccgacgatgatctggacgtggtgttcacccctaaagagtgcagaaccctgcagcctagcctgcctgaagaaggcgtggaactggatcctcacgtgcgggattgtgtgcagacctacatccgcgagtggctgatcgtgaaccggaagaatcagggcagcccagagatctgcggcttcaagaaaaccggcagccggaaggacttccacaagaccctgcctaagcagaccttcgagagcgagacactggaatgctctgagcctgctgctcaggccggacctagacacctgaatgtgctgtgtgacgtgtccggcaagggacctgtgaccgcctgcgatttcgacctgagaagtctgcagcccgacaagcggctggaaaacctgctccagcaagtgtccgccgaggacttcgagaagcagaacgaggaagccagacggaccaacagacaggccgagctgtttgctctgtaccccagcgtggacgaggaagatgccgtcgagattagacccgtgcctgagtgcccaaaagagcacctgggcaacagaatcctggtcaagctgctgaccctgaagttcgagatcgagattgagcccctgttcgcctctatcgccctgtacgacgtgaaagagcggaagaagatcagcgagaacttccactgcgacctgaactccgaccagttcaagggcttcctgagagcccacacaccatctgtggccgctagctctcaggccagatctgccgtgttcagcgtgacataccccagcagcgacatctacctggtggtcaagatcgagaaggtcctgcagcagggcgagatcggcgattgtgccgagccttacaccgtgatcaaagagagcgacggcggcaagagcaaagagaagattgagaagctgaagctgcaggccgagagcttctgtcagcggctgggcaagtacagaatgccctttgcctgggctcctatcagcctgtccagcttcttcaacgtgtccacactggaacgggaagtgaccgacgtggactctgtcgtgggcagatctagcgtgggcgagagaaggacactggctcagtctagacggctgagcgagagagccctgagcctggaagaaaatggcgtgggcagcaacttcaagaccagcactctgtccgtgtctagcttctttaagcaagagggcgacagactgagcgacgaggacctgttcaagttcctggccgactacaagcggagcagctccctgcagagaagagtgaagtctatccctggcctgctgcgcctggaaatctctacagcccctgagatcatcaactgctgtctgacccctgaaatgctgcccgtgaagccctttccagagaaccggaccagacctcacaaagagatcctggaattccccaccagagaagtgtacgtgccccacaccgtgtaccggaacctgctgtatgtgtacccacagcggctgaacttcgtgaacaagctggcctccgccagaaacatcaccatcaagatccagtttatgtgcggcgaggacgccagcaacgccatgcctgtgatcttcggcaagtctagcggccctgagttcctgcaagaggtgtacacagccgtgacctaccacaacaagagccccgacttctacgaggaagtgaagattaagctgcccgccaagctgaccgtgaatcaccatctgctgttcaccttctaccacatcagctgccagcagaaacagggcgcctctgtggaaacactgctgggctatagctggctgcccatcctgctgaacgagagactgcagaccggcagctactgtctgcctgtggctctggaaaagctgccacctaactacagcatgcactccgccgagaaggtgcccctgcagaatcctcctattaagtgggccgagggccacaagggcgtgttcaatatcgaggtgcaggccgtgtcctccgtgcacacccaggataaccacctggaaaagttcttcaccctgtgccacagcctcgagagccaagtgacattccccatccgcgtgctggaccagaaaatctccgagatggccctggaacacgagctgaaactgagcatcatctgcctgaatagcagcagactggaacccctggtgctgttcctgcatctggtgctggacaagctgttccagctgagcgtgcagcccatggttatcgccggacagaccgccaacttcagccagttcgcctttgagagcgtggtggccattgccaacagcctgcacaacagcaaggacctgagcaaggatcagcacggcagaaactgcctgctggcctcttacgtgcactacgtgttcagactgcctgaggtgcagagggacgtgccaaaatctggcgctcctaccgctctgctggaccccagaagctatcacacctacggcagaacatctgccgccgctgtgtcctctaaactcctgcaggctagagtgatgtccagcagcaaccctgatctggccggaacacacagcgccgctgatgaagaagtgaaaaacatcatgagcagcaagatcgccgaccggaactgcagccggatgagctactactgtagcggcagctctgatgcccctagctctccagctgctcctagacctgccagcaagaagcactttcacgaggaactggccctgcagatggtcgtgtctaccggcatggttcgagagacagtgtttaagtacgcctggttcttcttcgaactgctggtcaagagcatggcccagcacgtgcacaacatggacaagcgggacagcttccggcggaccagattcagcgacagattcatggacgacatcaccaccatcgtgaacgtggtcaccagcgagattgctgccctgctggttaagccccagaaagagaacgagcaggccgaaaagatgaatatctctctggccttcttcctctacgacctgctgagcctgatggaccggggcttcgtgttcaacctgatccggcactactgctcccagctgtctgccaagctgtccaacctgcctacactgatctctatgagactcgagttcctgaggatcctgtgcagccacgagcactacctgaacctgaatctgttcttcatgaacgccgacacagcccctacaagcccctgtccttctatcagcagccagaacagcagcagctgtagcagctttcaggatcagaaaatcgccagcatgttcgatctgaccagcgagtaccggcagcagcactttctgaccggcctgctgtttaccgaactggcagccgctcttgatgccgaaggcgagggaatcagcaaggtgcagcggaaagccgtgtctgccattcacagcctgctgtctagccacgacctggatcctagatgcgtgaagcctgaagtgaaagtgaaaatcgctgccctgtacctgcctctcgtgggcatcattctggatgccctgcctcagctgtgtgatttcaccgtggccgacaccagacggtacagaacctctggctccgacgaggaacaagaaggcgctggcgccatcaaccagaatgtggcccttgccattgccgggaacaactgcctgagctacgacaccgagatcctgaccgtggaatacggcttcctgcctatcggcaagatcgtggaagaacggatcgagtgcaccgtgtacaccgtggacaagaacggcttcgtgtacacacagcctatcgctcagtggcacaaccggggagagcaagaggtgttcgagtactgcctggaagatggcagcatcatccgggccaccaaggaccacaagttcatgaccaccgacggccagatgctgcccatcgacgagatctttgagagaggcctggacctgaaacaggtggacggacttccttgattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgctaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgttggttttttgtgtgtgcattgtctgagtaggtgtcattctattctggggggtggggtggggcagcacagcaagggggaggattgggaagacaatagcaggcatgctggggatgcggtgggctctatggagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctctcactacttctggaatagctcagaggccgaggcggcctcggcctctgcataaataaaaaaaattagtcgaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggg

SEQ ID NO: 12 - DNA, 6743 bp, synthetic construct
  source 1..6743: mol_type = other DNA; note = 1267 CfaC-CFN-DOCK8-bGHpA
ctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtggatcccctgagggggcccccatgggctagaggatccggcctcggcctctgcataaataaaaaaaattagtcagccatgagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctcccctcgaagcttacatgtggtaccgagctcggatcctgagaacttcagggtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggcaaagcacgtgagatctgaattcgagatctgccgccgccatggtgaagatcatcagcagaaagagcctgggcacccagaacgtgtacgacatcggcgtggaaaaggaccacaactttctgctcaagaacggcctggtggccagcaactgcttcaatctgaaaaccagcggcatcgtgctgagcagcctgccttacaagcagtacaacatgctgaatgccgacactacccgcaacctgatgatctgcttcctgtggattatgaagaacgccgatcagagcctgattcggaagtggattgccgacctgccaagcactcagctgaaccggattctggacctcctgttcatctgcgtgctgtgcttcgagtacaagggcaagcagtccagcgacaaggtgtccactcaggtgctgcagaaaagccgggatgtgaaggccaggctggaagaggcacttctgagaggcgaaggcgccagaggcgagatgatgaggcgtagagcccctggcaacgatagattccccggcctgaatgagaacctgcggtggaagaaagagcagacccattggagacaggccaacgagaaactggacaagaccaaggccgaactggaccaagaggccctgatctctggcaacctggccacagaagcccacctgattatcctggacatgcaagagaacatcatccaggccagtagcgccctggattgcaaggattctctgctcggaggcgtgctgagggtgctcgtgaatagcctgaattgcgaccagagcaccacctacctgacacactgcttcgccacactgagggccctgattgccaagtttggggacctgctgttcgaggaagaggtggaacagtgcttcgacctgtgtcaccaagtgctgcaccactgcagcagctccatggacgtgacaagatcccaggcctgcgccactctgtatctgctgatgcggtttagcttcggcgccaccagcaatttcgccagagtgaagatgcaagtgaccatgagcctggccagcctcgttggaagggcccctgacttcaatgaggaacacctcagaagaagcctgcggaccatcctggcctacagcgaagaggataccgccatgcagatgaccccatttccaacacaagtggaagaactgctgtgcaacctgaattccatcctgtacgataccgtgaagatgagagagttccaagaggaccccgagatgctgatggatctgatgtaccggatcgccaagagctaccaggcctctcctgatctgagactgacctggctgcagaacatggccgagaagcacaccaagaagaagtgctacaccgaggccgccatgtgcctggttcatgctgctgcactcgtggccgagtacctgagcatgctggaagatcactcctacctgccagtgggcagcgtgtccttccagaacatcagctccaacgtcctggaagagtccgtggtgtccgaggacacactgagcccagatgaggatggcgtgtgtgccggacagtactttaccgagtctggcctcgtgggccttctggaacaggccgccgaactgttttctaccggcggactgtacgagacagtgaacgaagtgtacaagctggtcatccccatcctggaagcccacagagagttcagaaagctgacactgacccactccaagctgcagcgggccttcgacagcattgtgaacaaggaccacaagcggatgttcggcacctacttcagagtgggctttttcggcagcaaattcggcgacctggacgagcaagagttcgtgtacaaagagcccgccatcaccaagctgcctgagatctcccatcggctggaagccttctacggccagtgttttggcgccgagttcgtggaagtgatcaaggacagcacccctgtggataagaccaaactggaccctaacaaggcctacatccagatcaccttcgtggaaccctacttcgacgagtacgagatgaaggatagagtgacctactttgagaagaacttcaacctgcggcggttcatgtacaccacaccttttacactggaaggcagacccaggggcgagctgcacgagcagtatagaagaaacaccgtgctgaccaccatgcacgcattcccttacatcaagacccggatcagcgtgatccagaaagaagagtttgtcctgacaccaatcgaggtggccatcgaggacatgaagaagaaaaccctccagctggccgtggccatcaatcaagaacctccagacgccaagatgctccagatggtgctccagggatctgtgggcgccacagtgaatcagggacctctggaagtggcccaagtgttcctggctgagatccccgccgatcctaagctgtacagacaccataacaagctgcggctctgcttcaaagaattcatcatgagatgtggcgaggccgtggaaaagaacaagagactgatcaccgccgaccagagagagtaccagcaagagctgaagaaaaactacaacaagctcaaagaaaatctgcggcccatgatcgagagaaagatccccgagctgtacaagcccatcttccgcgtggaaagccagaagcgggactcctttcacagatccagctttaggaagtgcgagacacagctgtcccagggctcttgactggtgtggctaatgccctggcccacaagtatcactaagctcgctttcttgctgtccaatttctattaaaggttcctttgttccctaagtccaactactaaactgggggatattatgaagggccttgagcatctggattctgcctaataaaaaacatttattttcattgcaatgatgtatttaaattatttctgaatattttactaaaaagggaatgtgggaggtcagtgcatttaaaacataaagaaatgaagagctagttcaaaccttgggaaaatacactatatcttaaactccatgaaagaaggtgaggctgcaaacagctaatgcacattggcaacagcccctgatgcctatgccttattcatccctcagaaaaggattcaagtagaggcttgatttggaggttaaagttttgctatgctgtattttacattacttattgttttagctgtcctcatgaatgtcttttcactacccatttgcttatcctgcatctctcagccttgactccactcagttctcttgcttagagataccacctttcccctgaagtgttccttccatgttttacggcgagatggtttctcctcgcctggccactcagccttagttgtctctgttgtcttatagaggtctacttgaagaaggaaaaacagggggcatggtttgactgtcctgtgagcccttcttccctgcctcccccactcacagtgacccggaatccctcgacatggcagtctagcactagtgcggccgcagatctgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgat

SEQ ID NO: 13 - DNA, 9449 bp, synthetic construct
  source 1..9449: mol_type = other DNA; note = mCCL-EFS-1267Dock8-CfaC-CFN-WPRE
acgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatagcacctagatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacgggggggtcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgaaaataagagagaaaagaagagtaagaagaaatataagagccaccatggtgaagatcatcagcagaaagagcctgggcacccagaacgtgtacgacatcggcgtggaaaaggaccacaactttctgctcaagaacggcctggtggccagcaactgcttcaatctgaaaaccagcggcatcgtgctgagcagcctgccttacaagcagtacaacatgctgaatgccgacactacccgcaacctgatgatctgcttcctgtggattatgaagaacgccgatcagagcctgattcggaagtggattgccgacctgccaagcactcagctgaaccggattctggacctcctgttcatctgcgtgctgtgcttcgagtacaagggcaagcagtccagcgacaaggtgtccactcaggtgctgcagaaaagccgggatgtgaaggccaggctggaagaggcacttctgagaggcgaaggcgccagaggcgagatgatgaggcgtagagcccctggcaacgatagattccccggcctgaatgagaacctgcggtggaagaaagagcagacccattggagacaggccaacgagaaactggacaagaccaaggccgaactggaccaagaggccctgatctctggcaacctggccacagaagcccacctgattatcctggacatgcaagagaacatcatccaggccagtagcgccctggattgcaaggattctctgctcggaggcgtgctgagggtgctcgtgaatagcctgaattgcgaccagagcaccacctacctgacacactgcttcgccacactgagggccctgattgccaagtttggggacctgctgttcgaggaagaggtggaacagtgcttcgacctgtgtcaccaagtgctgcaccactgcagcagctccatggacgtgacaagatcccaggcctgcgccactctgtatctgctgatgcggtttagcttcggcgccaccagcaatttcgccagagtgaagatgcaagtgaccatgagcctggccagcctcgttggaagggcccctgacttcaatgaggaacacctcagaagaagcctgcggaccatcctggcctacagcgaagaggataccgccatgcagatgaccccatttccaacacaagtggaagaactgctgtgcaacctgaattccatcctgtacgataccgtgaagatgagagagttccaagaggaccccgagatgctgatggatctgatgtaccggatcgccaagagctaccaggcctctcctgatctgagactgacctggctgcagaacatggccgagaagcacaccaagaagaagtgctacaccgaggccgccatgtgcctggttcatgctgctgcactcgtggccgagtacctgagcatgctggaagatcactcctacctgccagtgggcagcgtgtccttccagaacatcagctccaacgtcctggaagagtccgtggtgtccgaggacacactgagcccagatgaggatggcgtgtgtgccggacagtactttaccgagtctggcctcgtgggccttctggaacaggccgccgaactgttttctaccggcggactgtacgagacagtgaacgaagtgtacaagctggtcatccccatcctggaagcccacagagagttcagaaagctgacactgacccactccaagctgcagcgggccttcgacagcattgtgaacaaggaccacaagcggatgttcggcacctacttcagagtgggctttttcggcagcaaattcggcgacctggacgagcaagagttcgtgtacaaagagcccgccatcaccaagctgcctgagatctcccatcggctggaagccttctacggccagtgttttggcgccgagttcgtggaagtgatcaaggacagcacccctgtggataagaccaaactggaccctaacaaggcctacatccagatcaccttcgtggaaccctacttcgacgagtacgagatgaaggatagagtgacctactttgagaagaacttcaacctgcggcggttcatgtacaccacaccttttacactggaaggcagacccaggggcgagctgcacgagcagtatagaagaaacaccgtgctgaccaccatgcacgcattcccttacatcaagacccggatcagcgtgatccagaaagaagagtttgtcctgacaccaatcgaggtggccatcgaggacatgaagaagaaaaccctccagctggccgtggccatcaatcaagaacctccagacgccaagatgctccagatggtgctccagggatctgtgggcgccacagtgaatcagggacctctggaagtggcccaagtgttcctggctgagatccccgccgatcctaagctgtacagacaccataacaagctgcggctctgcttcaaagaattcatcatgagatgtggcgaggccgtggaaaagaacaagagactgatcaccgccgaccagagagagtaccagcaagagctgaagaaaaactacaacaagctcaaagaaaatctgcggcccatgatcgagagaaagatccccgagctgtacaagcccatcttccgcgtggaaagccagaagcgggactcctttcacagatccagctttaggaagtgcgagacacagctgtcccagggctcttgattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgctaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgttggttttttgtgtgtgcattgtctgagtaggtgtcattctattctggggggtggggtggggcagcacagcaagggggaggattgggaagacaatagcaggcatgctggggatgcggtgggctctatggagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctctcactacttctggaatagctcagaggccgaggcggcctcggcctctgcataaataaaaaaaattagtcgaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggg

SEQ ID NO: 14 - DNA, 3120 bp, synthetic construct
  source 1..3120: mol_type = other DNA; note = 939 DOCK8-CfaN-first half (5' half)
atggccacactgcctagcgctgagagaagggccttcgctctgaagatcaaccggtacagcagcgccgagatccggaagcagtttaccctgcctcctaacctgggccagtaccacagacagagcatcagcaccagcggcttcccatctctgcagctgccccagttctacgaccctgtggaacccgtggatttcgagggcctgctgatgacccacctgaacagcctggatgtgcagctggcccaagagctgggcgacttcaccgacgatgatctggacgtggtgttcacccctaaagagtgcagaaccctgcagcctagcctgcctgaagaaggcgtggaactggatcctcacgtgcgggattgtgtgcagacctacatccgcgagtggctgatcgtgaaccggaagaatcagggcagcccagagatctgcggcttcaagaaaaccggcagccggaaggacttccacaagaccctgcctaagcagaccttcgagagcgagacactggaatgctctgagcctgctgctcaggccggacctagacacctgaatgtgctgtgtgacgtgtccggcaagggacctgtgaccgcctgcgatttcgacctgagaagtctgcagcccgacaagcggctggaaaacctgctccagcaagtgtccgccgaggacttcgagaagcagaacgaggaagccagacggaccaacagacaggccgagctgtttgctctgtaccccagcgtggacgaggaagatgccgtcgagattagacccgtgcctgagtgcccaaaagagcacctgggcaacagaatcctggtcaagctgctgaccctgaagttcgagatcgagattgagcccctgttcgcctctatcgccctgtacgacgtgaaagagcggaagaagatcagcgagaacttccactgcgacctgaactccgaccagttcaagggcttcctgagagcccacacaccatctgtggccgctagctctcaggccagatctgccgtgttcagcgtgacataccccagcagcgacatctacctggtggtcaagatcgagaaggtcctgcagcagggcgagatcggcgattgtgccgagccttacaccgtgatcaaagagagcgacggcggcaagagcaaagagaagattgagaagctgaagctgcaggccgagagcttctgtcagcggctgggcaagtacagaatgccctttgcctgggctcctatcagcctgtccagcttcttcaacgtgtccacactggaacgggaagtgaccgacgtggactctgtcgtgggcagatctagcgtgggcgagagaaggacactggctcagtctagacggctgagcgagagagccctgagcctggaagaaaatggcgtgggcagcaacttcaagaccagcactctgtccgtgtctagcttctttaagcaagagggcgacagactgagcgacgaggacctgttcaagttcctggccgactacaagcggagcagctccctgcagagaagagtgaagtctatccctggcctgctgcgcctggaaatctctacagcccctgagatcatcaactgctgtctgacccctgaaatgctgcccgtgaagccctttccagagaaccggaccagacctcacaaagagatcctggaattccccaccagagaagtgtacgtgccccacaccgtgtaccggaacctgctgtatgtgtacccacagcggctgaacttcgtgaacaagctggcctccgccagaaacatcaccatcaagatccagtttatgtgcggcgaggacgccagcaacgccatgcctgtgatcttcggcaagtctagcggccctgagttcctgcaagaggtgtacacagccgtgacctaccacaacaagagccccgacttctacgaggaagtgaagattaagctgcccgccaagctgaccgtgaatcaccatctgctgttcaccttctaccacatcagctgccagcagaaacagggcgcctctgtggaaacactgctgggctatagctggctgcccatcctgctgaacgagagactgcagaccggcagctactgtctgcctgtggctctggaaaagctgccacctaactacagcatgcactccgccgagaaggtgcccctgcagaatcctcctattaagtgggccgagggccacaagggcgtgttcaatatcgaggtgcaggccgtgtcctccgtgcacacccaggataaccacctggaaaagttcttcaccctgtgccacagcctcgagagccaagtgacattccccatccgcgtgctggaccagaaaatctccgagatggccctggaacacgagctgaaactgagcatcatctgcctgaatagcagcagactggaacccctggtgctgttcctgcatctggtgctggacaagctgttccagctgagcgtgcagcccatggttatcgccggacagaccgccaacttcagccagttcgcctttgagagcgtggtggccattgccaacagcctgcacaacagcaaggacctgagcaaggatcagcacggcagaaactgcctgctggcctcttacgtgcactacgtgttcagactgcctgaggtgcagagggacgtgccaaaatctggcgctcctaccgctctgctggaccccagaagctatcacacctacggcagaacatctgccgccgctgtgtcctctaaactcctgcaggctagagtgatgtccagcagcaaccctgatctggccggaacacacagcgccgctgatgaagaagtgaaaaacatcatgagcagcaagatcgccgaccggaactgcagccggatgagctactactgcctgagctacgacaccgagatcctgaccgtggaatacggcttcctgcctatcggcaagatcgtggaagaacggatcgagtgcaccgtgtacaccgtggacaagaacggcttcgtgtacacacagcctatcgctcagtggcacaaccggggagagcaagaggtgttcgagtactgcctggaagatggcagcatcatccgggccaccaaggaccacaagttcatgaccaccgacggccagatgctgcccatcgacgagatctttgagagaggcctggacctgaaacaggtggacggacttccttga

SEQ ID NO: 15 - DNA, 2814 bp, synthetic construct
  source 1..2814: mol_type = other DNA; note = 939DOCK8 - first half (5' half)
atggccacactgcctagcgctgagagaagggccttcgctctgaagatcaaccggtacagcagcgccgagatccggaagcagtttaccctgcctcctaacctgggccagtaccacagacagagcatcagcaccagcggcttcccatctctgcagctgccccagttctacgaccctgtggaacccgtggatttcgagggcctgctgatgacccacctgaacagcctggatgtgcagctggcccaagagctgggcgacttcaccgacgatgatctggacgtggtgttcacccctaaagagtgcagaaccctgcagcctagcctgcctgaagaaggcgtggaactggatcctcacgtgcgggattgtgtgcagacctacatccgcgagtggctgatcgtgaaccggaagaatcagggcagcccagagatctgcggcttcaagaaaaccggcagccggaaggacttccacaagaccctgcctaagcagaccttcgagagcgagacactggaatgctctgagcctgctgctcaggccggacctagacacctgaatgtgctgtgtgacgtgtccggcaagggacctgtgaccgcctgcgatttcgacctgagaagtctgcagcccgacaagcggctggaaaacctgctccagcaagtgtccgccgaggacttcgagaagcagaacgaggaagccagacggaccaacagacaggccgagctgtttgctctgtaccccagcgtggacgaggaagatgccgtcgagattagacccgtgcctgagtgcccaaaagagcacctgggcaacagaatcctggtcaagctgctgaccctgaagttcgagatcgagattgagcccctgttcgcctctatcgccctgtacgacgtgaaagagcggaagaagatcagcgagaacttccactgcgacctgaactccgaccagttcaagggcttcctgagagcccacacaccatctgtggccgctagctctcaggccagatctgccgtgttcagcgtgacataccccagcagcgacatctacctggtggtcaagatcgagaaggtcctgcagcagggcgagatcggcgattgtgccgagccttacaccgtgatcaaagagagcgacggcggcaagagcaaagagaagattgagaagctgaagctgcaggccgagagcttctgtcagcggctgggcaagtacagaatgccctttgcctgggctcctatcagcctgtccagcttcttcaacgtgtccacactggaacgggaagtgaccgacgtggactctgtcgtgggcagatctagcgtgggcgagagaaggacactggctcagtctagacggctgagcgagagagccctgagcctggaagaaaatggcgtgggcagcaacttcaagaccagcactctgtccgtgtctagcttctttaagcaagagggcgacagactgagcgacgaggacctgttcaagttcctggccgactacaagcggagcagctccctgcagagaagagtgaagtctatccctggcctgctgcgcctggaaatctctacagcccctgagatcatcaactgctgtctgacccctgaaatgctgcccgtgaagccctttccagagaaccggaccagacctcacaaagagatcctggaattccccaccagagaagtgtacgtgccccacaccgtgtaccggaacctgctgtatgtgtacccacagcggctgaacttcgtgaacaagctggcctccgccagaaacatcaccatcaagatccagtttatgtgcggcgaggacgccagcaacgccatgcctgtgatcttcggcaagtctagcggccctgagttcctgcaagaggtgtacacagccgtgacctaccacaacaagagccccgacttctacgaggaagtgaagattaagctgcccgccaagctgaccgtgaatcaccatctgctgttcaccttctaccacatcagctgccagcagaaacagggcgcctctgtggaaacactgctgggctatagctggctgcccatcctgctgaacgagagactgcagaccggcagctactgtctgcctgtggctctggaaaagctgccacctaactacagcatgcactccgccgagaaggtgcccctgcagaatcctcctattaagtgggccgagggccacaagggcgtgttcaatatcgaggtgcaggccgtgtcctccgtgcacacccaggataaccacctggaaaagttcttcaccctgtgccacagcctcgagagccaagtgacattccccatccgcgtgctggaccagaaaatctccgagatggccctggaacacgagctgaaactgagcatcatctgcctgaatagcagcagactggaacccctggtgctgttcctgcatctggtgctggacaagctgttccagctgagcgtgcagcccatggttatcgccggacagaccgccaacttcagccagttcgcctttgagagcgtggtggccattgccaacagcctgcacaacagcaaggacctgagcaaggatcagcacggcagaaactgcctgctggcctcttacgtgcactacgtgttcagactgcctgaggtgcagagggacgtgccaaaatctggcgctcctaccgctctgctggaccccagaagctatcacacctacggcagaacatctgccgccgctgtgtcctctaaactcctgcaggctagagtgatgtccagcagcaaccctgatctggccggaacacacagcgccgctgatgaagaagtgaaaaacatcatgagcagcaagatcgccgaccggaactgcagccggatgagctactac

SEQ ID NO: 16 - DNA, 303 bp, synthetic construct
  source 1..303: mol_type = other DNA; note = CfaN
tgcctgagctacgacaccgagatcctgaccgtggaatacggcttcctgcctatcggcaagatcgtggaagaacggatcgagtgcaccgtgtacaccgtggacaagaacggcttcgtgtacacacagcctatcgctcagtggcacaaccggggagagcaagaggtgttcgagtactgcctggaagatggcagcatcatccgggccaccaaggaccacaagttcatgaccaccgacggccagatgctgcccatcgacgagatctttgagagaggcctggacctgaaacaggtggacggacttcct

SEQ ID NO: 17 - protein, 1039 aa, synthetic construct
  source 1..1039: note = 939 DOCK8-CfaN first half (N-terminal half)
MATLPSAERRAFALKINRYSSAEIRKQFTLPPNLGQYHRQSISTSGFPSLQLPQFYDPVEPVDFEGLLMTHLNSLDVQLAQELGDFTDDDLDVVFTPKECRTLQPSLPEEGVELDPHVRDCVQTYIREWLIVNRKNQGSPEICGFKKTGSRKDFHKTLPKQTFESETLECSEPAAQAGPRHLNVLCDVSGKGPVTACDFDLRSLQPDKRLENLLQQVSAEDFEKQNEEARRTNRQAELFALYPSVDEEDAVEIRPVPECPKEHLGNRILVKLLTLKFEIEIEPLFASIALYDVKERKKISENFHCDLNSDQFKGFLRAHTPSVAASSQARSAVFSVTYPSSDIYLVVKIEKVLQQGEIGDCAEPYTVIKESDGGKSKEKIEKLKLQAESFCQRLGKYRMPFAWAPISLSSFFNVSTLEREVTDVDSVVGRSSVGERRTLAQSRRLSERALSLEENGVGSNFKTSTLSVSSFFKQEGDRLSDEDLFKFLADYKRSSSLQRRVKSIPGLLRLEISTAPEIINCCLTPEMLPVKPFPENRTRPHKEILEFPTREVYVPHTVYRNLLYVYPQRLNFVNKLASARNITIKIQFMCGEDASNAMPVIFGKSSGPEFLQEVYTAVTYHNKSPDFYEEVKIKLPAKLTVNHHLLFTFYHISCQQKQGASVETLLGYSWLPILLNERLQTGSYCLPVALEKLPPNYSMHSAEKVPLQNPPIKWAEGHKGVFNIEVQAVSSVHTQDNHLEKFFTLCHSLESQVTFPIRVLDQKISEMALEHELKLSIICLNSSRLEPLVLFLHLVLDKLFQLSVQPMVIAGQTANFSQFAFESVVAIANSLHNSKDLSKDQHGRNCLLASYVHYVFRLPEVQRDVPKSGAPTALLDPRSYHTYGRTSAAAVSSKLLQARVMSSSNPDLAGTHSAADEEVKNIMSSKIADRNCSRMSYYCLSYDTEILTVEYGFLPIGKIVEERIECTVYTVDKNGFVYTQPIAQWHNRGEQEVFEYCLEDGSIIRATKDHKFMTTDGQMLPIDEIFERGLDLKQVDGLP

SEQ ID NO: 18 - protein, 938 aa, synthetic construct
  source 1..938: note = 939 DOCK8 - first half (N-terminal half)
MATLPSAERRAFALKINRYSSAEIRKQFTLPPNLGQYHRQSISTSGFPSLQLPQFYDPVEPVDFEGLLMTHLNSLDVQLAQELGDFTDDDLDVVFTPKECRTLQPSLPEEGVELDPHVRDCVQTYIREWLIVNRKNQGSPEICGFKKTGSRKDFHKTLPKQTFESETLECSEPAAQAGPRHLNVLCDVSGKGPVTACDFDLRSLQPDKRLENLLQQVSAEDFEKQNEEARRTNRQAELFALYPSVDEEDAVEIRPVPECPKEHLGNRILVKLLTLKFEIEIEPLFASIALYDVKERKKISENFHCDLNSDQFKGFLRAHTPSVAASSQARSAVFSVTYPSSDIYLVVKIEKVLQQGEIGDCAEPYTVIKESDGGKSKEKIEKLKLQAESFCQRLGKYRMPFAWAPISLSSFFNVSTLEREVTDVDSVVGRSSVGERRTLAQSRRLSERALSLEENGVGSNFKTSTLSVSSFFKQEGDRLSDEDLFKFLADYKRSSSLQRRVKSIPGLLRLEISTAPEIINCCLTPEMLPVKPFPENRTRPHKEILEFPTREVYVPHTVYRNLLYVYPQRLNFVNKLASARNITIKIQFMCGEDASNAMPVIFGKSSGPEFLQEVYTAVTYHNKSPDFYEEVKIKLPAKLTVNHHLLFTFYHISCQQKQGASVETLLGYSWLPILLNERLQTGSYCLPVALEKLPPNYSMHSAEKVPLQNPPIKWAEGHKGVFNIEVQAVSSVHTQDNHLEKFFTLCHSLESQVTFPIRVLDQKISEMALEHELKLSIICLNSSRLEPLVLFLHLVLDKLFQLSVQPMVIAGQTANFSQFAFESVVAIANSLHNSKDLSKDQHGRNCLLASYVHYVFRLPEVQRDVPKSGAPTALLDPRSYHTYGRTSAAAVSSKLLQARVMSSSNPDLAGTHSAADEEVKNIMSSKIADRNCSRMSYY

SEQ ID NO: 19 - protein, 101 aa, synthetic construct
  source 1..101: note = CfaN
CLSYDTEILTVEYGFLPIGKIVEERIECTVYTVDKNGFVYTQPIAQWHNRGEQEVFEYCLEDGSIIRATKDHKFMTTDGQMLPIDEIFERGLDLKQVDGLP

SEQ ID NO: 20 - DNA, 3600 bp, synthetic construct
  source 1..3600: mol_type = other DNA; note = 939 CfaC-CFN-DOCK8 2nd half (3')
atggtgaagatcatcagcagaaagagcctgggcacccagaacgtgtacgacatcggcgtggaaaaggaccacaactttctgctcaagaacggcctggtggccagcaactgctttaatagcggcagctctgatgcccctagctctccagctgctcctagacctgccagcaagaagcactttcacgaggaactggccctgcagatggtcgtgtctaccggcatggttcgagagacagtgtttaagtacgcctggttcttcttcgaactgctggtcaagagcatggcccagcacgtgcacaacatggacaagcgggacagcttccggcggaccagattcagcgacagattcatggacgacatcaccaccatcgtgaacgtggtcaccagcgagattgctgccctgctggttaagccccagaaagagaacgagcaggccgaaaagatgaatatctctctggccttcttcctctacgacctgctgagcctgatggaccggggcttcgtgttcaacctgatccggcactactgctcccagctgtctgccaagctgtccaacctgcctacactgatctctatgagactcgagttcctgaggatcctgtgcagccacgagcactacctgaacctgaatctgttcttcatgaacgccgacacagcccctacaagcccctgtccttctatcagcagccagaacagcagcagctgtagcagctttcaggatcagaaaatcgccagcatgttcgatctgaccagcgagtaccggcagcagcactttctgaccggcctgctgtttaccgaactggcagccgctcttgatgccgaaggcgagggaatcagcaaggtgcagcggaaagccgtgtctgccattcacagcctgctgtctagccacgacctggatcctagatgcgtgaagcctgaagtgaaagtgaaaatcgctgccctgtacctgcctctcgtgggcatcattctggatgccctgcctcagctgtgtgatttcaccgtggccgacaccagacggtacagaacctctggctccgacgaggaacaagaaggcgctggcgccatcaaccagaatgtggcccttgccattgccgggaacaacttcaatctgaaaaccagcggcatcgtgctgagcagcctgccttacaagcagtacaacatgctgaatgccgacactacccgcaacctgatgatctgcttcctgtggattatgaagaacgccgatcagagcctgattcggaagtggattgccgacctgccaagcactcagctgaaccggattctggacctcctgttcatctgcgtgctgtgcttcgagtacaagggcaagcagtccagcgacaaggtgtccactcaggtgctgcagaaaagccgggatgtgaaggccaggctggaagaggcacttctgagaggcgaaggcgccagaggcgagatgatgaggcgtagagcccctggcaacgatagattccccggcctgaatgagaacctgcggtggaagaaagagcagacccattggagacaggccaacgagaaactggacaagaccaaggccgaactggaccaagaggccctgatctctggcaacctggccacagaagcccacctgattatcctggacatgcaagagaacatcatccaggccagtagcgccctggattgcaaggattctctgctcggaggcgtgctgagggtgctcgtgaatagcctgaattgcgaccagagcaccacctacctgacacactgcttcgccacactgagggccctgattgccaagtttggggacctgctgttcgaggaagaggtggaacagtgcttcgacctgtgtcaccaagtgctgcaccactgcagcagctccatggacgtgacaagatcccaggcctgcgccactctgtatctgctgatgcggtttagcttcggcgccaccagcaatttcgccagagtgaagatgcaagtgaccatgagcctggccagcctcgttggaagggcccctgacttcaatgaggaacacctcagaagaagcctgcggaccatcctggcctacagcgaagaggataccgccatgcagatgaccccatttccaacacaagtggaagaactgctgtgcaacctgaattccatcctgtacgataccgtgaagatgagagagttccaagaggaccccgagatgctgatggatctgatgtaccggatcgccaagagctaccaggcctctcctgatctgagactgacctggctgcagaacatggccgagaagcacaccaagaagaagtgctacaccgaggccgccatgtgcctggttcatgctgctgcactcgtggccgagtacctgagcatgctggaagatcactcctacctgccagtgggcagcgtgtccttccagaacatcagctccaacgtcctggaagagtccgtggtgtccgaggacacactgagcccagatgaggatggcgtgtgtgccggacagtactttaccgagtctggcctcgtgggccttctggaacaggccgccgaactgttttctaccggcggactgtacgagacagtgaacgaagtgtacaagctggtcatccccatcctggaagcccacagagagttcagaaagctgacactgacccactccaagctgcagcgggccttcgacagcattgtgaacaaggaccacaagcggatgttcggcacctacttcagagtgggctttttcggcagcaaattcggcgacctggacgagcaagagttcgtgtacaaagagcccgccatcaccaagctgcctgagatctcccatcggctggaagccttctacggccagtgttttggcgccgagttcgtggaagtgatcaaggacagcacccctgtggataagaccaaactggaccctaacaaggcctacatccagatcaccttcgtggaaccctacttcgacgagtacgagatgaaggatagagtgacctactttgagaagaacttcaacctgcggcggttcatgtacaccacaccttttacactggaaggcagacccaggggcgagctgcacgagcagtatagaagaaacaccgtgctgaccaccatgcacgcattcccttacatcaagacccggatcagcgtgatccagaaagaagagtttgtcctgacaccaatcgaggtggccatcgaggacatgaagaagaaaaccctccagctggccgtggccatcaatcaagaacctccagacgccaagatgctccagatggtgctccagggatctgtgggcgccacagtgaatcagggacctctggaagtggcccaagtgttcctggctgagatccccgccgatcctaagctgtacagacaccataacaagctgcggctctgcttcaaagaattcatcatgagatgtggcgaggccgtggaaaagaacaagagactgatcaccgccgaccagagagagtaccagcaagagctgaagaaaaactacaacaagctcaaagaaaatctgcggcccatgatcgagagaaagatccccgagctgtacaagcccatcttccgcgtggaaagccagaagcgggactcctttcacagatccagctttaggaagtgcgagacacagctgtcccagggctcttga

SEQ ID NO: 21 - DNA, 105 bp, synthetic construct
  source 1..105: mol_type = other DNA; note = CfaC
gtgaagatcatcagcagaaagagcctgggcacccagaacgtgtacgacatcggcgtggaaaaggaccacaactttctgctcaagaacggcctggtggccagcaac

SEQ ID NO: 22 - DNA, 3480 bp, synthetic construct
  source 1..3480: mol_type = other DNA; note = 939-DOCK8 2nd half (3')
agcggcagctctgatgcccctagctctccagctgctcctagacctgccagcaagaagcactttcacgaggaactggccctgcagatggtcgtgtctaccggcatggttcgagagacagtgtttaagtacgcctggttcttcttcgaactgctggtcaagagcatggcccagcacgtgcacaacatggacaagcgggacagcttccggcggaccagattcagcgacagattcatggacgacatcaccaccatcgtgaacgtggtcaccagcgagattgctgccctgctggttaagccccagaaagagaacgagcaggccgaaaagatgaatatctctctggccttcttcctctacgacctgctgagcctgatggaccggggcttcgtgttcaacctgatccggcactactgctcccagctgtctgccaagctgtccaacctgcctacactgatctctatgagactcgagttcctgaggatcctgtgcagccacgagcactacctgaacctgaatctgttcttcatgaacgccgacacagcccctacaagcccctgtccttctatcagcagccagaacagcagcagctgtagcagctttcaggatcagaaaatcgccagcatgttcgatctgaccagcgagtaccggcagcagcactttctgaccggcctgctgtttaccgaactggcagccgctcttgatgccgaaggcgagggaatcagcaaggtgcagcggaaagccgtgtctgccattcacagcctgctgtctagccacgacctggatcctagatgcgtgaagcctgaagtgaaagtgaaaatcgctgccctgtacctgcctctcgtgggcatcattctggatgccctgcctcagctgtgtgatttcaccgtggccgacaccagacggtacagaacctctggctccgacgaggaacaagaaggcgctggcgccatcaaccagaatgtggcccttgccattgccgggaacaacttcaatctgaaaaccagcggcatcgtgctgagcagcctgccttacaagcagtacaacatgctgaatgccgacactacccgcaacctgatgatctgcttcctgtggattatgaagaacgccgatcagagcctgattcggaagtggattgccgacctgccaagcactcagctgaaccggattctggacctcctgttcatctgcgtgctgtgcttcgagtacaagggcaagcagtccagcgacaaggtgtccactcaggtgctgcagaaaagccgggatgtgaaggccaggctggaagaggcacttctgagaggcgaaggcgccagaggcgagatgatgaggcgtagagcccctggcaacgatagattccccggcctgaatgagaacctgcggtggaagaaagagcagacccattggagacaggccaacgagaaactggacaagaccaaggccgaactggaccaagaggccctgatctctggcaacctggccacagaagcccacctgattatcctggacatgcaagagaacatcatccaggccagtagcgccctggattgcaaggattctctgctcggaggcgtgctgagggtgctcgtgaatagcctgaattgcgaccagagcaccacctacctgacacactgcttcgccacactgagggccctgattgccaagtttggggacctgctgttcgaggaagaggtggaacagtgcttcgacctgtgtcaccaagtgctgcaccactgcagcagctccatggacgtgacaagatcccaggcctgcgccactctgtatctgctgatgcggtttagcttcggcgccaccagcaatttcgccagagtgaagatgcaagtgaccatgagcctggccagcctcgttggaagggcccctgacttcaatgaggaacacctcagaagaagcctgcggaccatcctggcctacagcgaagaggataccgccatgcagatgaccccatttccaacacaagtggaagaactgctgtgcaacctgaattccatcctgtacgataccgtgaagatgagagagttccaagaggaccccgagatgctgatggatctgatgtaccggatcgccaagagctaccaggcctctcctgatctgagactgacctggctgcagaacatggccgagaagcacaccaagaagaagtgctacaccgaggccgccatgtgcctggttcatgctgctgcactcgtggccgagtacctgagcatgctggaagatcactcctacctgccagtgggcagcgtgtccttccagaacatcagctccaacgtcctggaagagtccgtggtgtccgaggacacactgagcccagatgaggatggcgtgtgtgccggacagtactttaccgagtctggcctcgtgggccttctggaacaggccgccgaactgttttctaccggcggactgtacgagacagtgaacgaagtgtacaagctggtcatccccatcctggaagcccacagagagttcagaaagctgacactgacccactccaagctgcagcgggccttcgacagcattgtgaacaaggaccacaagcggatgttcggcacctacttcagagtgggctttttcggcagcaaattcggcgacctggacgagcaagagttcgtgtacaaagagcccgccatcaccaagctgcctgagatctcccatcggctggaagccttctacggccagtgttttggcgccgagttcgtggaagtgatcaaggacagcacccctgtggataagaccaaactggaccctaacaaggcctacatccagatcaccttcgtggaaccctacttcgacgagtacgagatgaaggatagagtgacctactttgagaagaacttcaacctgcggcggttcatgtacaccacaccttttacactggaaggcagacccaggggcgagctgcacgagcagtatagaagaaacaccgtgctgaccaccatgcacgcattcccttacatcaagacccggatcagcgtgatccagaaagaagagtttgtcctgacaccaatcgaggtggccatcgaggacatgaagaagaaaaccctccagctggccgtggccatcaatcaagaacctccagacgccaagatgctccagatggtgctccagggatctgtgggcgccacagtgaatcagggacctctggaagtggcccaagtgttcctggctgagatccccgccgatcctaagctgtacagacaccataacaagctgcggctctgcttcaaagaattcatcatgagatgtggcgaggccgtggaaaagaacaagagactgatcaccgccgaccagagagagtaccagcaagagctgaagaaaaactacaacaagctcaaagaaaatctgcggcccatgatcgagagaaagatccccgagctgtacaagcccatcttccgcgtggaaagccagaagcgggactcctttcacagatccagctttaggaagtgcgagacacagctgtcccagggctct

SEQ ID NO: 23 - protein, 1199 aa, synthetic construct
  source 1..1199: note = 939 CfaC-CFN-DOCK8 -second half (C-terminal half)
MVKIISRKSLGTQNVYDIGVEKDHNFLLKNGLVASNCFNSGSSDAPSSPAAPRPASKKHFHEELALQMVVSTGMVRETVFKYAWFFFELLVKSMAQHVHNMDKRDSFRRTRFSDRFMDDITTIVNVVTSEIAALLVKPQKENEQAEKMNISLAFFLYDLLSLMDRGFVFNLIRHYCSQLSAKLSNLPTLISMRLEFLRILCSHEHYLNLNLFFMNADTAPTSPCPSISSQNSSSCSSFQDQKIASMFDLTSEYRQQHFLTGLLFTELAAALDAEGEGISKVQRKAVSAIHSLLSSHDLDPRCVKPEVKVKIAALYLPLVGIILDALPQLCDFTVADTRRYRTSGSDEEQEGAGAINQNVALAIAGNNFNLKTSGIVLSSLPYKQYNMLNADTTRNLMICFLWIMKNADQSLIRKWIADLPSTQLNRILDLLFICVLCFEYKGKQSSDKVSTQVLQKSRDVKARLEEALLRGEGARGEMMRRRAPGNDRFPGLNENLRWKKEQTHWRQANEKLDKTKAELDQEALISGNLATEAHLIILDMQENIIQASSALDCKDSLLGGVLRVLVNSLNCDQSTTYLTHCFATLRALIAKFGDLLFEEEVEQCFDLCHQVLHHCSSSMDVTRSQACATLYLLMRFSFGATSNFARVKMQVTMSLASLVGRAPDFNEEHLRRSLRTILAYSEEDTAMQMTPFPTQVEELLCNLNSILYDTVKMREFQEDPEMLMDLMYRIAKSYQASPDLRLTWLQNMAEKHTKKKCYTEAAMCLVHAAALVAEYLSMLEDHSYLPVGSVSFQNISSNVLEESVVSEDTLSPDEDGVCAGQYFTESGLVGLLEQAAELFSTGGLYETVNEVYKLVIPILEAHREFRKLTLTHSKLQRAFDSIVNKDHKRMFGTYFRVGFFGSKFGDLDEQEFVYKEPAITKLPEISHRLEAFYGQCFGAEFVEVIKDSTPVDKTKLDPNKAYIQITFVEPYFDEYEMKDRVTYFEKNFNLRRFMYTTPFTLEGRPRGELHEQYRRNTVLTTMHAFPYIKTRISVIQKEEFVLTPIEVAIEDMKKKTLQLAVAINQEPPDAKMLQMVLQGSVGATVNQGPLEVAQVFLAEIPADPKLYRHHNKLRLCFKEFIMRCGEAVEKNKRLITADQREYQQELKKNYNKLKENLRPMIERKIPELYKPIFRVESQKRDSFHRSSFRKCETQLSQGS

SEQ ID NO: 24 - protein, 35 aa, synthetic construct
  source 1..35: note = CfaC
VKIISRKSLGTQNVYDIGVEKDHNFLLKNGLVASN

SEQ ID NO: 25 - protein, 1160 aa, synthetic construct
  source 1..1160: note = 939 DOCK8 - second half (C-terminal half)
SGSSDAPSSPAAPRPASKKHFHEELALQMVVSTGMVRETVFKYAWFFFELLVKSMAQHVHNMDKRDSFRRTRFSDRFMDDITTIVNVVTSEIAALLVKPQKENEQAEKMNISLAFFLYDLLSLMDRGFVFNLIRHYCSQLSAKLSNLPTLISMRLEFLRILCSHEHYLNLNLFFMNADTAPTSPCPSISSQNSSSCSSFQDQKIASMFDLTSEYRQQHFLTGLLFTELAAALDAEGEGISKVQRKAVSAIHSLLSSHDLDPRCVKPEVKVKIAALYLPLVGIILDALPQLCDFTVADTRRYRTSGSDEEQEGAGAINQNVALAIAGNNFNLKTSGIVLSSLPYKQYNMLNADTTRNLMICFLWIMKNADQSLIRKWIADLPSTQLNRILDLLFICVLCFEYKGKQSSDKVSTQVLQKSRDVKARLEEALLRGEGARGEMMRRRAPGNDRFPGLNENLRWKKEQTHWRQANEKLDKTKAELDQEALISGNLATEAHLIILDMQENIIQASSALDCKDSLLGGVLRVLVNSLNCDQSTTYLTHCFATLRALIAKFGDLLFEEEVEQCFDLCHQVLHHCSSSMDVTRSQACATLYLLMRFSFGATSNFARVKMQVTMSLASLVGRAPDFNEEHLRRSLRTILAYSEEDTAMQMTPFPTQVEELLCNLNSILYDTVKMREFQEDPEMLMDLMYRIAKSYQASPDLRLTWLQNMAEKHTKKKCYTEAAMCLVHAAALVAEYLSMLEDHSYLPVGSVSFQNISSNVLEESVVSEDTLSPDEDGVCAGQYFTESGLVGLLEQAAELFSTGGLYETVNEVYKLVIPILEAHREFRKLTLTHSKLQRAFDSIVNKDHKRMFGTYFRVGFFGSKFGDLDEQEFVYKEPAITKLPEISHRLEAFYGQCFGAEFVEVIKDSTPVDKTKLDPNKAYIQITFVEPYFDEYEMKDRVTYFEKNFNLRRFMYTTPFTLEGRPRGELHEQYRRNTVLTTMHAFPYIKTRISVIQKEEFVLTPIEVAIEDMKKKTLQLAVAINQEPPDAKMLQMVLQGSVGATVNQGPLEVAQVFLAEIPADPKLYRHHNKLRLCFKEFIMRCGEAVEKNKRLITADQREYQQELKKNYNKLKENLRPMIERKIPELYKPIFRVESQKRDSFHRSSFRKCETQLSQGS

SEQ ID NO: 26 - DNA, 3597 bp, synthetic construct
  source 1..3597: mol_type = other DNA; note = 939 CfaC-CF-DOCK8 - second half (3')
atggtgaagatcatcagcagaaagagcctgggcacccagaacgtgtacgacatcggcgtggaaaaggaccacaactttctgctcaagaacggcctggtggccagcaactgctttagcggcagctctgatgcccctagctctccagctgctcctagacctgccagcaagaagcactttcacgaggaactggccctgcagatggtcgtgtctaccggcatggttcgagagacagtgtttaagtacgcctggttcttcttcgaactgctggtcaagagcatggcccagcacgtgcacaacatggacaagcgggacagcttccggcggaccagattcagcgacagattcatggacgacatcaccaccatcgtgaacgtggtcaccagcgagattgctgccctgctggttaagccccagaaagagaacgagcaggccgaaaagatgaatatctctctggccttcttcctctacgacctgctgagcctgatggaccggggcttcgtgttcaacctgatccggcactactgctcccagctgtctgccaagctgtccaacctgcctacactgatctctatgagactcgagttcctgaggatcctgtgcagccacgagcactacctgaacctgaatctgttcttcatgaacgccgacacagcccctacaagcccctgtccttctatcagcagccagaacagcagcagctgtagcagctttcaggatcagaaaatcgccagcatgttcgatctgaccagcgagtaccggcagcagcactttctgaccggcctgctgtttaccgaactggcagccgctcttgatgccgaaggcgagggaatcagcaaggtgcagcggaaagccgtgtctgccattcacagcctgctgtctagccacgacctggatcctagatgcgtgaagcctgaagtgaaagtgaaaatcgctgccctgtacctgcctctcgtgggcatcattctggatgccctgcctcagctgtgtgatttcaccgtggccgacaccagacggtacagaacctctggctccgacgaggaacaagaaggcgctggcgccatcaaccagaatgtggcccttgccattgccgggaacaacttcaatctgaaaaccagcggcatcgtgctgagcagcctgccttacaagcagtacaacatgctgaatgccgacactacccgcaacctgatgatctgcttcctgtggattatgaagaacgccgatcagagcctgattcggaagtggattgccgacctgccaagcactcagctgaaccggattctggacctcctgttcatctgcgtgctgtgcttcgagtacaagggcaagcagtccagcgacaaggtgtccactcaggtgctgcagaaaagccgggatgtgaaggccaggctggaagaggcacttctgagaggcgaaggcgccagaggcgagatgatgaggcgtagagcccctggcaacgatagattccccggcctgaatgagaacctgcggtggaagaaagagcagacccattggagacaggccaacgagaaactggacaagaccaaggccgaactggaccaagaggccctgatctctggcaacctggccacagaagcccacctgattatcctggacatgcaagagaacatcatccaggccagtagcgccctggattgcaaggattctctgctcggaggcgtgctgagggtgctcgtgaatagcctgaattgcgaccagagcaccacctacctgacacactgcttcgccacactgagggccctgattgccaagtttggggacctgctgttcgaggaagaggtggaacagtgcttcgacctgtgtcaccaagtgctgcaccactgcagcagctccatggacgtgacaagatcccaggcctgcgccactctgtatctgctgatgcggtttagcttcggcgccaccagcaatttcgccagagtgaagatgcaagtgaccatgagcctggccagcctcgttggaagggcccctgacttcaatgaggaacacctcagaagaagcctgcggaccatcctggcctacagcgaagaggataccgccatgcagatgaccccatttccaacacaagtggaagaactgctgtgcaacctgaattccatcctgtacgataccgtgaagatgagagagttccaagaggaccccgagatgctgatggatctgatgtaccggatcgccaagagctaccaggcctctcctgatctgagactgacctggctgcagaacatggccgagaagcacaccaagaagaagtgctacaccgaggccgccatgtgcctggttcatgctgctgcactcgtggccgagtacctgagcatgctggaagatcactcctacctgccagtgggcagcgtgtccttccagaacatcagctccaacgtcctggaagagtccgtggtgtccgaggacacactgagcccagatgaggatggcgtgtgtgccggacagtactttaccgagtctggcctcgtgggccttctggaacaggccgccgaactgttttctaccggcggactgtacgagacagtgaacgaagtgtacaagctggtcatccccatcctggaagcccacagagagttcagaaagctgacactgacccactccaagctgcagcgggccttcgacagcattgtgaacaaggaccacaagcggatgttcggcacctacttcagagtgggctttttcggcagcaaattcggcgacctggacgagcaagagttcgtgtacaaagagcccgccatcaccaagctgcctgagatctcccatcggctggaagccttctacggccagtgttttggcgccgagttcgtggaagtgatcaaggacagcacccctgtggataagaccaaactggaccctaacaaggcctacatccagatcaccttcgtggaaccctacttcgacgagtacgagatgaaggatagagtgacctactttgagaagaacttcaacctgcggcggttcatgtacaccacaccttttacactggaaggcagacccaggggcgagctgcacgagcagtatagaagaaacaccgtgctgaccaccatgcacgcattcccttacatcaagacccggatcagcgtgatccagaaagaagagtttgtcctgacaccaatcgaggtggccatcgaggacatgaagaagaaaaccctccagctggccgtggccatcaatcaagaacctccagacgccaagatgctccagatggtgctccagggatctgtgggcgccacagtgaatcagggacctctggaagtggcccaagtgttcctggctgagatccccgccgatcctaagctgtacagacaccataacaagctgcggctctgcttcaaagaattcatcatgagatgtggcgaggccgtggaaaagaacaagagactgatcaccgccgaccagagagagtaccagcaagagctgaagaaaaactacaacaagctcaaagaaaatctgcggcccatgatcgagagaaagatccccgagctgtacaagcccatcttccgcgtggaaagccagaagcgggactcctttcacagatccagctttaggaagtgcgagacacagctgtcccagggctcttga

SEQ ID NO: 27 - protein, 1198 aa, synthetic construct
  source 1..1198: note = 939 CfaC-CF-DOCK8 - second half (C-terminal half)
MVKIISRKSLGTQNVYDIGVEKDHNFLLKNGLVASNCFSGSSDAPSSPAAPRPASKKHFHEELALQMVVSTGMVRETVFKYAWFFFELLVKSMAQHVHNMDKRDSFRRTRFSDRFMDDITTIVNVVTSEIAALLVKPQKENEQAEKMNISLAFFLYDLLSLMDRGFVFNLIRHYCSQLSAKLSNLPTLISMRLEFLRILCSHEHYLNLNLFFMNADTAPTSPCPSISSQNSSSCSSFQDQKIASMFDLTSEYRQQHFLTGLLFTELAAALDAEGEGISKVQRKAVSAIHSLLSSHDLDPRCVKPEVKVKIAALYLPLVGIILDALPQLCDFTVADTRRYRTSGSDEEQEGAGAINQNVALAIAGNNFNLKTSGIVLSSLPYKQYNMLNADTTRNLMICFLWIMKNADQSLIRKWIADLPSTQLNRILDLLFICVLCFEYKGKQSSDKVSTQVLQKSRDVKARLEEALLRGEGARGEMMRRRAPGNDRFPGLNENLRWKKEQTHWRQANEKLDKTKAELDQEALISGNLATEAHLIILDMQENIIQASSALDCKDSLLGGVLRVLVNSLNCDQSTTYLTHCFATLRALIAKFGDLLFEEEVEQCFDLCHQVLHHCSSSMDVTRSQACATLYLLMRFSFGATSNFARVKMQVTMSLASLVGRAPDFNEEHLRRSLRTILAYSEEDTAMQMTPFPTQVEELLCNLNSILYDTVKMREFQEDPEMLMDLMYRIAKSYQASPDLRLTWLQNMAEKHTKKKCYTEAAMCLVHAAALVAEYLSMLEDHSYLPVGSVSFQNISSNVLEESVVSEDTLSPDEDGVCAGQYFTESGLVGLLEQAAELFSTGGLYETVNEVYKLVIPILEAHREFRKLTLTHSKLQRAFDSIVNKDHKRMFGTYFRVGFFGSKFGDLDEQEFVYKEPAITKLPEISHRLEAFYGQCFGAEFVEVIKDSTPVDKTKLDPNKAYIQITFVEPYFDEYEMKDRVTYFEKNFNLRRFMYTTPFTLEGRPRGELHEQYRRNTVLTTMHAFPYIKTRISVIQKEEFVLTPIEVAIEDMKKKTLQLAVAINQEPPDAKMLQMVLQGSVGATVNQGPLEVAQVFLAEIPADPKLYRHHNKLRLCFKEFIMRCGEAVEKNKRLITADQREYQQELKKNYNKLKENLRPMIERKIPELYKPIFRVESQKRDSFHRSSFRKCETQLSQGS

SEQ ID NO: 28 - DNA, 9959 bp, synthetic construct; identical to SEQ ID NO: 6
  source 1..9959: mol_type = other DNA; note = EFS-939 DOCK8-CfaN-WPRE first half (5' half)

SEQ ID NO: 29 - DNA, 10439 bp, synthetic construct
  source 1..10439: mol_type = other DNA; note = EFS-939 DOCK8-CfaC-CFN-WPRE second half (3' half)
acgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatagcacctagatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacgggggggtcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgaaaataagagagaaaagaagagtaagaagaaatataagagccaccatggtgaagatcatcagcagaaagagcctgggcacccagaacgtgtacgacatcggcgtggaaaaggaccacaactttctgctcaagaacggcctggtggccagcaactgctttaatagcggcagctctgatgcccctagctctccagctgctcctagacctgccagcaagaagcactttcacgaggaactggccctgcagatggtcgtgtctaccggcatggttcgagagacagtgtttaagtacgcctggttcttcttcgaactgctggtcaagagcatggcccagcacgtgcacaacatggacaagcgggacagcttccggcggaccagattcagcgacagattcatggacgacatcaccaccatcgtgaacgtggtcaccagcgagattgctgccctgctggttaagccccagaaagagaacgagcaggccgaaaagatgaatatctctctggccttcttcctctacgacctgctgagcctgatggaccggggcttcgtgttcaacctgatccggcactactgctcccagctgtctgccaagctgtccaacctgcctacactgatctctatgagactcgagttcctgaggatcctgtgcagccacgagcactacctgaacctgaatctgttcttcatgaacgccgacacagcccctacaagcccctgtccttctatcagcagccagaacagcagcagctgtagcagctttcaggatcagaaaatcgccagcatgttcgatctgaccagcgagtaccggcagcagcactttctgaccggcctgctgtttaccgaactggcagccgctcttgatgccgaaggcgagggaatcagcaaggtgcagcggaaagccgtgtctgccattcacagcctgctgtctagccacgacctggatcctagatgcgtgaagcctgaagtgaaagtgaaaatcgctgccctgtacctgcctctcgtgggcatcattctggatgccctgcctcagctgtgtgatttcaccgtggccgacaccagacggtacagaacctctggctccgacgaggaacaagaaggcgctggcgccatcaaccagaatgtggcccttgccattgccgggaacaacttcaatctgaaaaccagcggcatcgtgctgagcagcctgccttacaagcagtacaacatgctgaatgccgacactacccgcaacctgatgatctgcttcctgtggattatgaagaacgccgatcagagcctgattcggaagtggattgccgacctgccaagcactcagctgaaccggattctggacctcctgttcatctgcgtgctgtgcttcgagtacaagggcaagcagtccagcgacaaggtgtccactcaggtgctgcagaaaagccgggatgtgaaggccaggctggaagaggcacttctgagaggcgaaggcgccagaggcgagatgatgaggcgtagagcccctggcaacgatagattccccggcctgaatgagaacctgcggtggaagaaagagcagacccattggagacaggccaacgagaaactggacaagaccaaggccgaactggaccaagaggccctgatctctggcaacctggccacagaagcccacctgattatcctggacatgcaagagaacatcatccaggccagtagcgccctggattgcaaggattctctgctcggaggcgtgctgagggtgctcgtgaatagcctgaattgcgaccagagcaccacctacctgacacactgcttcgccacactgagggccctgattgccaagtttggggacctgctgttcgaggaagaggtggaacagtgcttcgacctgtgtcaccaagtgctgcaccactgcagcagctccatggacgtgacaagatcccaggcctgcgccactctgtatctgctgatgcggtttagcttcggcgccaccagcaatttcgccagagtgaagatgcaagtgaccatgagcctggccagcctcgttggaagggcccctgacttcaatgaggaacacctcagaagaagcctgcggaccatcctggcctacagcgaagaggataccgccatgcagatgaccccatttccaacacaagtggaagaactgctgtgcaacctgaattccatcctgtacgataccgtgaagatgagagagttccaagaggaccccgagatgctgatggatctgatgtaccggatcgccaagagctaccaggcctctcctgatctgagactgacctggctgcagaacatggccgagaagcacaccaagaagaagtgctacaccgaggccgccatgtgcctggttcatgctgctgcactcgtggccgagtacctgagcatgctggaagatcactcctacctgccagtgggcagcgtgtccttccagaacatcagctccaacgtcctggaagagtccgtggtgtccgaggacacactgagcccagatgaggatggcgtgtgtgccggacagtactttaccgagtctggcctcgtgggccttctggaacaggccgccgaactgttttctaccggcggactgtacgagacagtgaacgaagtgtacaagctggtcatccccatcctggaagcccacagagagttcagaaagctgacactgacccactccaagctgcagcgggccttcgacagcattgtgaacaaggaccacaagcggatgttcggcacctacttcagagtgggctttttcggcagcaaattcggcgacctggacgagcaagagttcgtgtacaaagagcccgccatcaccaagctgcctgagatctcccatcggctggaagccttctacggccagtgttttggcgccgagttcgtggaagtgatcaaggacagcacccctgtggataagaccaaactggaccctaacaaggcctacatccagatcaccttcgtggaaccctacttcgacgagtacgagatgaaggatagagtgacctactttgagaagaacttcaacctgcggcggttcatgtacaccacaccttttacactggaaggcagacccaggggcgagctgcacgagcagtatagaagaaacaccgtgctgaccaccatgcacgcattcccttacatcaagacccggatcagcgtgatccagaaagaagagtttgtcctgacaccaatcgaggtggccatcgaggacatgaagaagaaaaccctccagctggccgtggccatcaatcaagaacctccagacgccaagatgctccagatggtgctccagggatctgtgggcgccacagtgaatcagggacctctggaagtggcccaagtgttcctggctgagatccccgccgatcctaagctgtacagacaccataacaagctgcggctctgcttcaaagaattcatcatgagatgtggcgaggccgtggaaaagaacaagagactgatcaccgccgaccagagagagtaccagcaagagctgaagaaaaactacaacaagctcaaagaaaatctgcggcccatgatcgagagaaagatccccgagctgtacaagcccatcttccgcgtggaaagccagaagcgggactcctttcacagatccagctttaggaagtgcgagacacagctgtcccagggctcttgattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgctaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgttggttttttgtgtgtgcattgtctgagtaggtgtcattctattctggggggtggggtggggcagcacagcaagggggaggattgggaagacaatagcaggcatgctggggatgcggtgggctctatggagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctctcactacttctggaatagctcagaggccgaggcggcctcggcctctgcataaataaaaaaaattagtcgaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggg

SEQ ID NO: 30 - DNA, 10436 bp, synthetic construct; identical to SEQ ID NO: 8
  source 1..10436: mol_type = other DNA; note = EFS-939 DOCK8-CfaC-CF-WPRE second half (3' half)

SEQ ID NO: 31 - DNA, 4107 bp, synthetic construct
  source 1..4107: mol_type = other DNA; note = 1267 DOCK8-CfaN - first half (5' half)
atggccacactgcctagcgctgagagaagggccttcgctctgaagatcaaccggtacagcagcgccgagatccggaagcagtttaccctgcctcctaacctgggccagtaccacagacagagcatcagcaccagcggcttcccatctctgcagctgccccagttctacgaccctgtggaacccgtggatttcgagggcctgctgatgacccacctgaacagcctggatgtgcagctggcccaagagctgggcgacttcaccgacgatgatctggacgtggtgttcacccctaaagagtgcagaaccctgcagcctagcctgcctgaagaaggcgtggaactggatcctcacgtgcgggattgtgtgcagacctacatccgcgagtggctgatcgtgaaccggaagaatcagggcagcccagagatctgcggcttcaagaaaaccggcagccggaaggacttccacaagaccctgcctaagcagaccttcgagagcgagacactggaatgctctgagcctgctgctcaggccggacctagacacctgaatgtgctgtgtgacgtgtccggcaagggacctgtgaccgcctgcgatttcgacctgagaagtctgcagcccgacaagcggctggaaaacctgctccagcaagtgtccgccgaggacttcgagaagcagaacgaggaagccagacggaccaacagacaggccgagctgtttgctctgtaccccagcgtggacgaggaagatgccgtcgagattagacccgtgcctgagtgcccaaaagagcacctgggcaacagaatcctggtcaagctgctgaccctgaagttcgagatcgagattgagcccctgttcgcctctatcgccctgtacgacgtgaaagagcggaagaagatcagcgagaacttccactgcgacctgaactccgaccagttcaagggcttcctgagagcccacacaccatctgtggccgctagctctcaggccagatctgccgtgttcagcgtgacataccccagcagcgacatctacctggtggtcaagatcgagaaggtcctgcagcagggcgagatcggcgattgtgccgagccttacaccgtgatcaaagagagcgacggcggcaagagcaaagagaagattgagaagctgaagctgcaggccgagagcttctgtcagcggctgggcaagtacagaatgccctttgcctgggctcctatcagcctgtccagcttcttcaacgtgtccacactggaacgggaagtgaccgacgtggactctgtcgtgggcagatctagcgtgggcgagagaaggacactggctcagtctagacggctgagcgagagagccctgagcctggaagaaaatggcgtgggcagcaacttcaagaccagcactctgtccgtgtctagcttctttaagcaagagggcgacagactgagcgacgaggacctgttcaagttcctggccgactacaagcggagcagctccctgcagagaagagtgaagtctatccctggcctgctgcgcctggaaatctctacagcccctgagatcatcaactgctgtctgacccctgaaatgctgcccgtgaagccctttccagagaaccggaccagacctcacaaagagatcctggaattccccaccagagaagtgtacgtgccccacaccgtgtaccggaacctgctgtatgtgtacccacagcggctgaacttcgtgaacaagctggcctccgccagaaacatcaccatcaagatccagtttatgtgcggcgaggacgccagcaacgccatgcctgtgatcttcggcaagtctagcggccctgagttcctgcaagaggtgtacacagccgtgacctaccacaacaagagccccgacttctacgaggaagtgaagattaagctgcccgccaagctgaccgtgaatcaccatctgctgttcaccttctaccacatcagctgccagcagaaacagggcgcctctgtggaaacactgctgggctatagctggctgcccatcctgctgaacgagagactgcagaccggcagctactgtctgcctgtggctctggaaaagctgccacctaactacagcatgcactccgccgagaaggtgcccctgcagaatcctcctattaagtgggccgagggccacaagggcgtgttcaatatcgaggtgcaggccgtgtcctccgtgcacacccaggataaccacctggaaaagttcttcaccctgtgccacagcctcgagagccaagtgacattccccatccgcgtgctggaccagaaaatctccgagatggccctggaacacgagctgaaactgagcatcatctgcctgaatagcagcagactggaacccctggtgctgttcctgcatctggtgctggacaagctgttccagctgagcgtgcagcccatggttatcgccggacagaccgccaacttcagccagttcgcctttgagagcgtggtggccattgccaacagcctgcacaacagcaaggacctgagcaaggatcagcacggcagaaactgcctgctggcctcttacgtgcactacgtgttcagactgcctgaggtgcagagggacgtgccaaaatctggcgctcctaccgctctgctggaccccagaagctatcacacctacggcagaacatctgccgccgctgtgtcctctaaactcctgcaggctagagtgatgtccagcagcaaccctgatctggccggaacacacagcgccgctgatgaagaagtgaaaaacatcatgagcagcaagatcgccgaccggaactgcagccggatgagctactactgtagcggcagctctgatgcccctagctctccagctgctcctagacctgccagcaagaagcactttcacgaggaactggccctgcagatggtcgtgtctaccggcatggttcgagagacagtgtttaagtacgcctggttcttcttcgaactgctggtcaagagcatggcccagcacgtgcacaacatggacaagcgggacagcttccggcggaccagattcagcgacagattcatggacgacatcaccaccatcgtgaacgtggtcaccagcgagattgctgccctgctggttaagccccagaaagagaacgagcaggccgaaaagatgaatatctctctggccttcttcctctacgacctgctgagcctgatggaccggggcttcgtgttcaacctgatccggcactactgctcccagctgtctgccaagctgtccaacctgcctacactgatctctatgagactcgagttcctgaggatcctgtgcagccacgagcactacctgaacctgaatctgttcttcatgaacgccgacacagcccctacaagcccctgtccttctatcagcagccagaacagcagcagctgtagcagctttcaggatcagaaaatcgccagcatgttcgatctgaccagcgagtaccggcagcagcactttctgaccggcctgctgtttaccgaactggcagccgctcttgatgccgaaggcgagggaatcagcaaggtgcagcggaaagccgtgtctgccattcacagcctgctgtctagccacgacctggatcctagatgcgtgaagcctgaagtgaaagtgaaaatcgctgccctgtacctgcctctcgtgggcatcattctggatgccctgcctcagctgtgtgatttcaccgtggccgacaccagacggtacagaacctctggctccgacgaggaacaagaaggcgctggcgccatcaaccagaatgtggcccttgccattgccgggaacaactgcctgagctacgacaccgagatcctgaccgtggaatacggcttcctgcctatcggcaagatcgtggaagaacggatcgagtgcaccgtgtacaccgtggacaagaacggcttcgtgtacacacagcctatcgctcagtggcacaaccggggagagcaagaggtgttcgagtactgcctggaagatggcagcatcatccgggccaccaaggaccacaagttcatgaccaccgacggccagatgctgcccatcgacgagatctttgagagaggcctggacctgaaacaggtggacggacttccttga

SEQ ID NO: 32 - DNA, 3801 bp, synthetic construct
  source 1..3801: mol_type = other DNA; note = 1267 DOCK8 - first half (5' half)
atggccacactgcctagcgctgagagaagggccttcgctctgaagatcaaccggtacagcagcgccgagatccggaagcagtttaccctgcctcctaacctgggccagtaccacagacagagcatcagcaccagcggcttcccatctctgcagctgccccagttctacgaccctgtggaacccgtggatttcgagggcctgctgatgacccacctgaacagcctggatgtgcagctggcccaagagctgggcgacttcaccgacgatgatctggacgtggtgttcacccctaaagagtgcagaaccctgcagcctagcctgcctgaagaaggcgtggaactggatcctcacgtgcgggattgtgtgcagacctacatccgcgagtggctgatcgtgaaccggaagaatcagggcagcccagagatctgcggcttcaagaaaaccggcagccggaaggacttccacaagaccctgcctaagcagaccttcgagagcgagacactggaatgctctgagcctgctgctcaggccggacctagacacctgaatgtgctgtgtgacgtgtccggcaagggacctgtgaccgcctgcgatttcgacctgagaagtctgcagcccgacaagcggctggaaaacctgctccagcaagtgtccgccgaggacttcgagaagcagaacgaggaagccagacggaccaacagacaggccgagctgtttgctctgtaccccagcgtggacgaggaagatgccgtcgagattagacccgtgcctgagtgcccaaaagagcacctgggcaacagaatcctggtcaagctgctgaccctgaagttcgagatcgagattgagcccctgttcgcctctatcgccctgtacgacgtgaaagagcggaagaagatcagcgagaacttccactgcgacctgaactccgaccagttcaagggcttcctgagagcccacacaccatctgtggccgctagctctcaggccagatctgccgtgttcagcgtgacataccccagcagcgacatctacctggtggtcaagatcgagaaggtcctgcagcagggcgagatcggcgattgtgccgagccttacaccgtgatcaaagagagcgacggcggcaagagcaaagagaagattgagaagctgaagctgcaggccgagagcttctgtcagcggctgggcaagtacagaatgccctttgcctgggctcctatcagcctgtccagcttcttcaacgtgtccacactggaacgggaagtgaccgacgtggactctgtcgtgggcagatctagcgtgggcgagagaaggacactggctcagtctagacggctgagcgagagagccctgagcctggaagaaaatggcgtgggcagcaacttcaagaccagcactctgtccgtgtctagcttctttaagcaagagggcgacagactgagcgacgaggacctgttcaagttcctggccgactacaagcggagcagctccctgcagagaagagtgaagtctatccctggcctgctgcgcctggaaatctctacagcccctgagatcatcaactgctgtctgacccctgaaatgctgcccgtgaagccctttccagagaaccggaccagacctcacaaagagatcctggaattccccaccagagaagtgtacgtgccccacaccgtgtaccggaacctgctgtatgtgtacccacagcggctgaacttcgtgaacaagctggcctccgccagaaacatcaccatcaagatccagtttatgtgcggcgaggacgccagcaacgccatgcctgtgatcttcggcaagtctagcggccctgagttcctgcaagaggtgtacacagccgtgacctaccacaacaagagccccgacttctacgaggaagtgaagattaagctgcccgccaagctgaccgtgaatcaccatctgctgttcaccttctaccacatcagctgccagcagaaacagggcgcctctgtggaaacactgctgggctatagctggctgcccatcctgctgaacgagagactgcagaccggcagctactgtctgcctgtggctctggaaaagctgccacctaactacagcatgcactccgccgagaaggtgcccctgcagaatcctcctattaagtgggccgagggccacaagggcgtgttcaatatcgaggtgcaggccgtgtcctccgtgcacacccaggataaccacctggaaaagttcttcaccctgtgccacagcctcgagagccaagtgacattccccatccgcgtgctggaccagaaaatctccgagatggccctggaacacgagctgaaactgagcatcatctgcctgaatagcagcagactggaacccctggtgctgttcctgcatctggtgctggacaagctgttccagctgagcgtgcagcccatggttatcgccggacagaccgccaacttcagccagttcgcctttgagagcgtggtggccattgccaacagcctgcacaacagcaaggacctgagcaaggatcagcacggcagaaactgcctgctggcctcttacgtgcactacgtgttcagactgcctgaggtgcagagggacgtgccaaaatctggcgctcctaccgctctgctggaccccagaagctatcacacctacggcagaacatctgccgccgctgtgtcctctaaactcctgcaggctagagtgatgtccagcagcaaccctgatctggccggaacacacagcgccgctgatgaagaagtgaaaaacatcatgagcagcaagatcgccgaccggaactgcagccggatgagctactactgtagcggcagctctgatgcccctagctctccagctgctcctagacctgccagcaagaagcactttcacgaggaactggccctgcagatggtcgtgtctaccggcatggttcgagagacagtgtttaagtacgcctggttcttcttcgaactgctggtcaagagcatggcccagcacgtgcacaacatggacaagcgggacagcttccggcggaccagattcagcgacagattcatggacgacatcaccaccatcgtgaacgtggtcaccagcgagattgctgccctgctggttaagccccagaaagagaacgagcaggccgaaaagatgaatatctctctggccttcttcctctacgacctgctgagcctgatggaccggggcttcgtgttcaacctgatccggcactactgctcccagctgtctgccaagctgtccaacctgcctacactgatctctatgagactcgagttcctgaggatcctgtgcagccacgagcactacctgaacctgaatctgttcttcatgaacgccgacacagcccctacaagcccctgtccttctatcagcagccagaacagcagcagctgtagcagctttcaggatcagaaaatcgccagcatgttcgatctgaccagcgagtaccggcagcagcactttctgaccggcctgctgtttaccgaactggcagccgctcttgatgccgaaggcgagggaatcagcaaggtgcagcggaaagccgtgtctgccattcacagcctgctgtctagccacgacctggatcctagatgcgtgaagcctgaagtgaaagtgaaaatcgctgccctgtacctgcctctcgtgggcatcattctggatgccctgcctcagctgtgtgatttcaccgtggccgacaccagacggtacagaacctctggctccgacgaggaacaagaaggcgctggcgccatcaaccagaatgtggcccttgccattgccgggaacaac

SEQ ID NO: 33 - protein, 1368 aa, synthetic construct
  source 1..1368: note = 1267 DOCK8-CfaN - first half (N-terminal half)
MATLPSAERRAFALKINRYSSAEIRKQFTLPPNLGQYHRQSISTSGFPSLQLPQFYDPVEPVDFEGLLMTHLNSLDVQLAQELGDFTDDDLDVVFTPKECRTLQPSLPEEGVELDPHVRDCVQTYIREWLIVNRKNQGSPEICGFKKTGSRKDFHKTLPKQTFESETLECSEPAAQAGPRHLNVLCDVSGKGPVTACDFDLRSLQPDKRLENLLQQVSAEDFEKQNEEARRTNRQAELFALYPSVDEEDAVEIRPVPECPKEHLGNRILVKLLTLKFEIEIEPLFASIALYDVKERKKISENFHCDLNSDQFKGFLRAHTPSVAASSQARSAVFSVTYPSSDIYLVVKIEKVLQQGEIGDCAEPYTVIKESDGGKSKEKIEKLKLQAESFCQRLGKYRMPFAWAPISLSSFFNVSTLEREVTDVDSVVGRSSVGERRTLAQSRRLSERALSLEENGVGSNFKTSTLSVSSFFKQEGDRLSDEDLFKFLADYKRSSSLQRRVKSIPGLLRLEISTAPEIINCCLTPEMLPVKPFPENRTRPHKEILEFPTREVYVPHTVYRNLLYVYPQRLNFVNKLASARNITIKIQFMCGEDASNAMPVIFGKSSGPEFLQEVYTAVTYHNKSPDFYEEVKIKLPAKLTVNHHLLFTFYHISCQQKQGASVETLLGYSWLPILLNERLQTGSYCLPVALEKLPPNYSMHSAEKVPLQNPPIKWAEGHKGVFNIEVQAVSSVHTQDNHLEKFFTLCHSLESQVTFPIRVLDQKISEMALEHELKLSIICLNSSRLEPLVLFLHLVLDKLFQLSVQPMVIAGQTANFSQFAFESVVAIANSLHNSKDLSKDQHGRNCLLASYVHYVFRLPEVQRDVPKSGAPTALLDPRSYHTYGRTSAAAVSSKLLQARVMSSSNPDLAGTHSAADEEVKNIMSSKIADRNCSRMSYYCSGSSDAPSSPAAPRPASKKHFHEELALQMVVSTGMVRETVFKYAWFFFELLVKSMAQHVHNMDKRDSFRRTRFSDRFMDDITTIVNVVTSEIAALLVKPQKENEQAEKMNISLAFFLYDLLSLMDRGFVFNLIRHYCSQLSAKLSNLPTLISMRLEFLRILCSHEHYLNLNLFFMNADTAPTSPCPSISSQNSSSCSSFQDQKIASMFDLTSEYRQQHFLTGLLFTELAAALDAEGEGISKVQRKAVSAIHSLLSSHDLDPRCVKPEVKVKIAALYLPLVGIILDALPQLCDFTVADTRRYRTSGSDEEQEGAGAINQNVALAIAGNNCLSYDTEILTVEYGFLPIGKIVEERIECTVYTVDKNGFVYTQPIAQWHNRGEQEVFEYCLEDGSIIRATKDHKFMTTDGQMLPIDEIFERGLDLKQVDGLP

SEQ ID NO: 34 - protein, 1267 aa, synthetic construct
  source 1..1267: note = 1267 DOCK8- first half (N-terminal half)
MATLPSAERRAFALKINRYSSAEIRKQFTLPPNLGQYHRQSISTSGFPSLQLPQFYDPVEPVDFEGLLMTHLNSLDVQLAQELGDFTDDDLDVVFTPKECRTLQPSLPEEGVELDPHVRDCVQTYIREWLIVNRKNQGSPEICGFKKTGSRKDFHKTLPKQTFESETLECSEPAAQAGPRHLNVLCDVSGKGPVTACDFDLRSLQPDKRLENLLQQVSAEDFEKQNEEARRTNRQAELFALYPSVDEEDAVEIRPVPECPKEHLGNRILVKLLTLKFEIEIEPLFASIALYDVKERKKISENFHCDLNSDQFKGFLRAHTPSVAASSQARSAVFSVTYPSSDIYLVVKIEKVLQQGEIGDCAEPYTVIKESDGGKSKEKIEKLKLQAESFCQRLGKYRMPFAWAPISLSSFFNVSTLEREVTDVDSVVGRSSVGERRTLAQSRRLSERALSLEENGVGSNFKTSTLSVSSFFKQEGDRLSDEDLFKFLADYKRSSSLQRRVKSIPGLLRLEISTAPEIINCCLTPEMLPVKPFPENRTRPHKEILEFPTREVYVPHTVYRNLLYVYPQRLNFVNKLASARNITIKIQFMCGEDASNAMPVIFGKSSGPEFLQEVYTAVTYHNKSPDFYEEVKIKLPAKLTVNHHLLFTFYHISCQQKQGASVETLLGYSWLPILLNERLQTGSYCLPVALEKLPPNYSMHSAEKVPLQNPPIKWAEGHKGVFNIEVQAVSSVHTQDNHLEKFFTLCHSLESQVTFPIRVLDQKISEMALEHELKLSIICLNSSRLEPLVLFLHLVLDKLFQLSVQPMVIAGQTANFSQFAFESVVAIANSLHNSKDLSKDQHGRNCLLASYVHYVFRLPEVQRDVPKSGAPTALLDPRSYHTYGRTSAAAVSSKLLQARVMSSSNPDLAGTHSAADEEVKNIMSSKIADRNCSRMSYYCSGSSDAPSSPAAPRPASKKHFHEELALQMVVSTGMVRETVFKYAWFFFELLVKSMAQHVHNMDKRDSFRRTRFSDRFMDDITTIVNVVTSEIAALLVKPQKENEQAEKMNISLAFFLYDLLSLMDRGFVFNLIRHYCSQLSAKLSNLPTLISMRLEFLRILCSHEHYLNLNLFFMNADTAPTSPCPSISSQNSSSCSSFQDQKIASMFDLTSEYRQQHFLTGLLFTELAAALDAEGEGISKVQRKAVSAIHSLLSSHDLDPRCVKPEVKVKIAALYLPLVGIILDALPQLCDFTVADTRRYRTSGSDEEQEGAGAINQNVALAIAGNN

SEQ ID NO: 35 - DNA, 2610 bp, synthetic construct
  source 1..2610: mol_type = other DNA; note = 1267 CfaC-CFN-DOCK8 second half (3' half)
atggtgaagatcatcagcagaaagagcctgggcacccagaacgtgtacgacatcggcgtggaaaaggaccacaactttctgctcaagaacggcctggtggccagcaactgcttcaatctgaaaaccagcggcatcgtgctgagcagcctgccttacaagcagtacaacatgctgaatgccgacactacccgcaacctgatgatctgcttcctgtggattatgaagaacgccgatcagagcctgattcggaagtggattgccgacctgccaagcactcagctgaaccggattctggacctcctgttcatctgcgtgctgtgcttcgagtacaagggcaagcagtccagcgacaaggtgtccactcaggtgctgcagaaaagccgggatgtgaaggccaggctggaagaggcacttctgagaggcgaaggcgccagaggcgagatgatgaggcgtagagcccctggcaacgatagattccccggcctgaatgagaacctgcggtggaagaaagagcagacccattggagacaggccaacgagaaactggacaagaccaaggccgaactggaccaagaggccctgatctctggcaacctggccacagaagcccacctgattatcctggacatgcaagagaacatcatccaggccagtagcgccctggattgcaaggattctctgctcggaggcgtgctgagggtgctcgtgaatagcctgaattgcgaccagagcaccacctacctgacacactgcttcgccacactgagggccctgattgccaagtttggggacctgctgttcgaggaagaggtggaacagtgcttcgacctgtgtcaccaagtgctgcaccactgcagcagctccatggacgtgacaagatcccaggcctgcgccactctgtatctgctgatgcggtttagcttcggcgccaccagcaatttcgccagagtgaagatgcaagtgaccatgagcctggccagcctcgttggaagggcccctgacttcaatgaggaacacctcagaagaagcctgcggaccatcctggcctacagcgaagaggataccgccatgcagatgaccccatttccaacacaagtggaagaactgctgtgcaacctgaattccatcctgtacgataccgtgaagatgagagagttccaagaggaccccgagatgctgatggatctgatgtaccggatcgccaagagctaccaggcctctcctgatctgagactgacctggctgcagaacatggccgagaagcacaccaagaagaagtgctacaccgaggccgccatgtgcctggttcatgctgctgcactcgtggccgagtacctgagcatgctggaagatcactcctacctgccagtgggcagcgtgtccttccagaacatcagctccaacgtcctggaagagtccgtggtgtccgaggacacactgagcccagatgaggatggcgtgtgtgccggacagtactttaccgagtctggcctcgtgggccttctggaacaggccgccgaactgttttctaccggcggactgtacgagacagtgaacgaagtgtacaagctggtcatccccatcctggaagcccacagagagttcagaaagctgacactgacccactccaagctgcagcgggccttcgacagcattgtgaacaaggaccacaagcggatgttcggcacctacttcagagtgggctttttcggcagcaaattcggcgacctggacgagcaagagttcgtgtacaaagagcccgccatcaccaagctgcctgagatctcccatcggctggaagccttctacggccagtgttttggcgccgagttcgtggaagtgatcaaggacagcacccctgtggataagaccaaactggaccctaacaaggcctacatccagatcaccttcgtggaaccctacttcgacgagtacgagatgaaggatagagtgacctactttgagaagaacttcaacctgcggcggttcatgtacaccacaccttttacactggaaggcagacccaggggcgagctgcacgagcagtatagaagaaacaccgtgctgaccaccatgcacgcattcccttacatcaagacccggatcagcgtgatccagaaagaagagtttgtcctgacaccaatcgaggtggccatcgaggacatgaagaagaaaaccctccagctggccgtggccatcaatcaagaacctccagacgccaagatgctccagatggtgctccagggatctgtgggcgccacagtgaatcagggacctctggaagtggcccaagtgttcctggctgagatccccgccgatcctaagctgtacagacaccataacaagctgcggctctgcttcaaagaattcatcatgagatgtggcgaggccgtggaaaagaacaagagactgatcaccgccgaccagagagagtaccagcaagagctgaagaaaaactacaacaagctcaaagaaaatctgcggcccatgatcgagagaaagatccccgagctgtacaagcccatcttccgcgtggaaagccagaagcgggactcctttcacagatccagctttaggaagtgcgagacacagctgtcccagggctcttga

SEQ ID NO: 36 - DNA, 2490 bp, synthetic construct
  source 1..2490: mol_type = other DNA; note = 1267 DOCK8 second half (3' half)
ctgaaaaccagcggcatcgtgctgagcagcctgccttacaagcagtacaacatgctgaatgccgacactacccgcaacctgatgatctgcttcctgtggattatgaagaacgccgatcagagcctgattcggaagtggattgccgacctgccaagcactcagctgaaccggattctggacctcctgttcatctgcgtgctgtgcttcgagtacaagggcaagcagtccagcgacaaggtgtccactcaggtgctgcagaaaagccgggatgtgaaggccaggctggaagaggcacttctgagaggcgaaggcgccagaggcgagatgatgaggcgtagagcccctggcaacgatagattccccggcctgaatgagaacctgcggtggaagaaagagcagacccattggagacaggccaacgagaaactggacaagaccaaggccgaactggaccaagaggccctgatctctggcaacctggccacagaagcccacctgattatcctggacatgcaagagaacatcatccaggccagtagcgccctggattgcaaggattctctgctcggaggcgtgctgagggtgctcgtgaatagcctgaattgcgaccagagcaccacctacctgacacactgcttcgccacactgagggccctgattgccaagtttggggacctgctgttcgaggaagaggtggaacagtgcttcgacctgtgtcaccaagtgctgcaccactgcagcagctccatggacgtgacaagatcccaggcctgcgccactctgtatctgctgatgcggtttagcttcggcgccaccagcaatttcgccagagtgaagatgcaagtgaccatgagcctggccagcctcgttggaagggcccctgacttcaatgaggaacacctcagaagaagcctgcggaccatcctggcctacagcgaagaggataccgccatgcagatgaccccatttccaacacaagtggaagaactgctgtgcaacctgaattccatcctgtacgataccgtgaagatgagagagttccaagaggaccccgagatgctgatggatctgatgtaccggatcgccaagagctaccaggcctctcctgatctgagactgacctggctgcagaacatggccgagaagcacaccaagaagaagtgctacaccgaggccgccatgtgcctggttcatgctgctgcactcgtggccgagtacctgagcatgctggaagatcactcctacctgccagtgggcagcgtgtccttccagaacatcagctccaacgtcctggaagagtccgtggtgtccgaggacacactgagcccagatgaggatggcgtgtgtgccggacagtactttaccgagtctggcctcgtgggccttctggaacaggccgccgaactgttttctaccggcggactgtacgagacagtgaacgaagtgtacaagctggtcatccccatcctggaagcccacagagagttcagaaagctgacactgacccactccaagctgcagcgggccttcgacagcattgtgaacaaggaccacaagcggatgttcggcacctacttcagagtgggctttttcggcagcaaattcggcgacctggacgagcaagagttcgtgtacaaagagcccgccatcaccaagctgcctgagatctcccatcggctggaagccttctacggccagtgttttggcgccgagttcgtggaagtgatcaaggacagcacccctgtggataagaccaaactggaccctaacaaggcctacatccagatcaccttcgtggaaccctacttcgacgagtacgagatgaaggatagagtgacctactttgagaagaacttcaacctgcggcggttcatgtacaccacaccttttacactggaaggcagacccaggggcgagctgcacgagcagtatagaagaaacaccgtgctgaccaccatgcacgcattcccttacatcaagacccggatcagcgtgatccagaaagaagagtttgtcctgacaccaatcgaggtggccatcgaggacatgaagaagaaaaccctccagctggccgtggccatcaatcaagaacctccagacgccaagatgctccagatggtgctccagggatctgtgggcgccacagtgaatcagggacctctggaagtggcccaagtgttcctggctgagatccccgccgatcctaagctgtacagacaccataacaagctgcggctctgcttcaaagaattcatcatgagatgtggcgaggccgtggaaaagaacaagagactgatcaccgccgaccagagagagtaccagcaagagctgaagaaaaactacaacaagctcaaagaaaatctgcggcccatgatcgagagaaagatccccgagctgtacaagcccatcttccgcgtggaaagccagaagcgggactcctttcacagatccagctttaggaagtgcgagacacagctgtcccagggctct

SEQ ID NO: 37 - protein, 830 aa, synthetic construct
  source 1..830: note = 1267 DOCK8 second half (C-terminal half)
LKTSGIVLSSLPYKQYNMLNADTTRNLMICFLWIMKNADQSLIRKWIADLPSTQLNRILDLLFICVLCFEYKGKQSSDKVSTQVLQKSRDVKARLEEALLRGEGARGEMMRRRAPGNDRFPGLNENLRWKKEQTHWRQANEKLDKTKAELDQEALISGNLATEAHLIILDMQENIIQASSALDCKDSLLGGVLRVLVNSLNCDQSTTYLTHCFATLRALIAKFGDLLFEEEVEQCFDLCHQVLHHCSSSMDVTRSQACATLYLLMRFSFGATSNFARVKMQVTMSLASLVGRAPDFNEEHLRRSLRTILAYSEEDTAMQMTPFPTQVEELLCNLNSILYDTVKMREFQEDPEMLMDLMYRIAKSYQASPDLRLTWLQNMAEKHTKKKCYTEAAMCLVHAAALVAEYLSMLEDHSYLPVGSVSFQNISSNVLEESVVSEDTLSPDEDGVCAGQYFTESGLVGLLEQAAELFSTGGLYETVNEVYKLVIPILEAHREFRKLTLTHSKLQRAFDSIVNKDHKRMFGTYFRVGFFGSKFGDLDEQEFVYKEPAITKLPEISHRLEAFYGQCFGAEFVEVIKDSTPVDKTKLDPNKAYIQITFVEPYFDEYEMKDRVTYFEKNFNLRRFMYTTPFTLEGRPRGELHEQYRRNTVLTTMHAFPYIKTRISVIQKEEFVLTPIEVAIEDMKKKTLQLAVAINQEPPDAKMLQMVLQGSVGATVNQGPLEVAQVFLAEIPADPKLYRHHNKLRLCFKEFIMRCGEAVEKNKRLITADQREYQQELKKNYNKLKENLRPMIERKIPELYKPIFRVESQKRDSFHRSSFRKCETQLSQGS

SEQ ID NO: 38 - DNA, 10946 bp, synthetic construct; identical to SEQ ID NO: 11
  source 1..10946: mol_type = other DNA; note = EFS-1267-DOCK8-CfaN-WPRE first half (5' half)

SEQ ID NO: 39 - DNA, 9449 bp, synthetic construct; identical to SEQ ID NO: 13
  source 1..9449: mol_type = other DNA; note = EFS-1267-DOCK8-CfaC-CFN-WPRE second half (3' half)

SEQ ID NO: 40 - DNA, 240 bp, synthetic construct
  source 1..240: mol_type = other DNA; note = EFS
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtga

SEQ ID NO: 41 - DNA, 596 bp, synthetic construct
  source 1..596: mol_type = other DNA; note = WPRE
ttccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 42 - protein, 869 aa, synthetic construct
  source 1..869: note = 1267 CfaC-CFN-DOCK8 2nd half (C-terminal half)
MVKIISRKSLGTQNVYDIGVEKDHNFLLKNGLVASNCFNLKTSGIVLSSLPYKQYNMLNADTTRNLMICFLWIMKNADQSLIRKWIADLPSTQLNRILDLLFICVLCFEYKGKQSSDKVSTQVLQKSRDVKARLEEALLRGEGARGEMMRRRAPGNDRFPGLNENLRWKKEQTHWRQANEKLDKTKAELDQEALISGNLATEAHLIILDMQENIIQASSALDCKDSLLGGVLRVLVNSLNCDQSTTYLTHCFATLRALIAKFGDLLFEEEVEQCFDLCHQVLHHCSSSMDVTRSQACATLYLLMRFSFGATSNFARVKMQVTMSLASLVGRAPDFNEEHLRRSLRTILAYSEEDTAMQMTPFPTQVEELLCNLNSILYDTVKMREFQEDPEMLMDLMYRIAKSYQASPDLRLTWLQNMAEKHTKKKCYTEAAMCLVHAAALVAEYLSMLEDHSYLPVGSVSFQNISSNVLEESVVSEDTLSPDEDGVCAGQYFTESGLVGLLEQAAELFSTGGLYETVNEVYKLVIPILEAHREFRKLTLTHSKLQRAFDSIVNKDHKRMFGTYFRVGFFGSKFGDLDEQEFVYKEPAITKLPEISHRLEAFYGQCFGAEFVEVIKDSTPVDKTKLDPNKAYIQITFVEPYFDEYEMKDRVTYFEKNFNLRRFMYTTPFTLEGRPRGELHEQYRRNTVLTTMHAFPYIKTRISVIQKEEFVLTPIEVAIEDMKKKTLQLAVAINQEPPDAKMLQMVLQGSVGATVNQGPLEVAQVFLAEIPADPKLYRHHNKLRLCFKEFIMRCGEAVEKNKRLITADQREYQQELKKNYNKLKENLRPMIERKIPELYKPIFRVESQKRDSFHRSSFRKCETQLSQGS

(19) United States
(12) Patent Application Publication
(10) Pub. No.: US 2026/0317788 A1
(43) Pub. Date: Oct. 15, 2026

(54) GENE THERAPY FOR DOCK8 DEFICIENCY

(71) Applicant: The Regents of the University of California, Oakland, CA (US)

(72) Inventor: Caroline Y. KUO, Beverly Hills, CA (US)

(73) Assignee: The Regents of the University of California, Oakland, CA (US)

(21) Appl. No.: 19/485,039

(22) PCT Filed: May 17, 2024
(86) PCT No.: PCT/US2024/029875

§ 371 Date: Nov. 17, 2025

Related U.S. Application Data
(60) Provisional application No. 63/467,552, filed on May 18, 2023.

(51) Int. Cl. C12N 15/867; A61K 35/12; A61K 35/28; A61K 48/00; C07K 14/47; C12N 5/0775; C12N 5/0789; C12N 15/90

(52) U.S. Cl. CPC C12N 15/867; A61K 35/28; A61K 48/0025; A61K 48/005; C07K 14/4702; C12N 5/0647; C12N 5/0663; C12N 5/0665; C12N 15/90; A61K 2035/128; C07K 2319/92; C12N 2510/00; C12N 2740/15043; C12N 2800/40; C12N 2830/34

(57) ABSTRACT

Described herein are systems including nucleic acid constructs for DOCK8 protein expression in cells and uses thereof. Further described are Lentiviral vectors containing these expression systems, and methods of use thereof for treating a DOCK8 deficiency in a subject in need.

### CROSS REFERENCE TO RELATED APPLICATIONS

This application claims the benefit of U.S. Provisional Application Ser. No. 63/467,552, filed on May 18, 2023, and entitled GENE THERAPY FOR DOCK8 DEFICIENCY, which is incorporated herein in its entirety by reference.

### SEQUENCE LISTING STATEMENT

The instant application contains a Sequence Listing which has been submitted electronically in XML format and is hereby incorporated by reference in its entirety. The XML format copy, created on May 14, 2024, is named P-636966-PC_05_14_2024__SL.xml and is 241.2 kilobytes in size.

### BACKGROUND

DOCK8 deficiency results from mutations in DOCK8, a large gene on chromosome 9 comprised of 48 exons that encodes a protein about 190 kDa in size. The DOCK8 protein C is constitutively expressed in cells of hematopoietic origin and is highly expressed by healthy lymphocytes. Affected patients present within the first months to early years of life and are severely affected by the disease. The median survival is 10-20 years and is complicated by multiple disease-related comorbidities, including recalcitrant infections, autoimmunity, and predisposition to the development of malignancies. Similar to many other inborn errors of immunity, allogeneic HSCT can be curative for DOCK8 deficiency but is frequently complicated by acute and chronic GvHD, sinusoidal obstruction syndrome, and worsening of pre-existing disease. Despite the associated risks of allogeneic bone marrow transplantation, the therapeutic benefits of allogeneic HSCT suggests that ex vivo gene modification of autologous HSPC may be an effective treatment for the disease with fewer side effects.

Autologous bone marrow transplantation would not require an HLA-matched donor and would allow affected individuals to be transplanted when they are younger, which is associated with improved overall survival.

### SUMMARY

Disclosed here are systems for expressing DOCK8 protein and uses thereof.

In certain aspects, a system for expressing human DOCK8 protein in a cell, comprises a first nucleic acid encoding a first fragment of a human DOCK8 protein fused at the C terminal to an N-intein; and a second nucleic acid encoding a second fragment of the human DOCK8 protein fused at the N terminal with a C-intein;
wherein the first fragment of the DOCK8 protein ligated to the second fragment of the DOCK8 protein forms a full-length human DOCK8 protein,
wherein the first fragment of the DOCK8 protein and the second fragment of the DOCK8 protein are selected to provide a split between said first fragment and said second fragment in an external loop that does not contribute to intra-protein interactions, wherein said first fragment comprises the DOCK homology region 1 (DHR1) and said second fragment comprises the DOCK homology region 2 (DHR2), and
wherein said first nucleic acid further comprises a promoter located 5′ of said nucleic acid encoding the first fragment of DOCK8 and said second nucleic acid further comprises a promoter located 5′ of said nucleic acid encoding the C-intein, wherein optionally said promoter comprises EFS. In a related aspect, the amino acid sequence of said first fragment of DOCK8 is set forth in SEQ ID NO: 18 and the amino acid sequence of said second fragment of DOCK8 is set forth in SEQ ID NO: 25, or the amino acid sequence of said first fragment of DOCK8 is set forth in SEQ ID NO: 34 and the amino acid sequence of said second fragment of DOCK8 is set forth in SEQ ID NO: 37.

In another related aspect, the first nucleic acid encoding a first fragment of a human DOCK8 is codon-optimized and wherein said second nucleic acid encoding a second fragment of the human DOCK8 protein is codon-optimized.

In another related aspect, the second fragment of the DOCK8 protein comprise an extein linker where said linker comprises C-F or C-F-N. In a further related aspect, the N-intein and C-intein comprise a Cfa split intein, wherein the N-intein comprises a CfaN sequence and the C-intein comprise a CfaC sequence, wherein optionally said nucleotide sequence encoding said CfaN amino acid sequence and said nucleotide sequence encoding said CfaC amino acid sequence are codon optimized for expression in a human cell. In yet another further related aspect, the amino acid sequence of said first fragment of the human DOCK8 protein fused at the C terminal to an N-intein is set forth in SEQ ID NO: 17 and the amino acid sequence of said second fragment of the human DOCK8 protein fused at the N terminal with a C-intein is set forth in SEQ ID NO: 23 or SEQ ID NO: 27; or the amino acid sequence of said first fragment of the human DOCK8 protein fused at the C terminal to an N-intein is set forth in SEQ ID NO: 33 and the amino acid sequence of said second fragment of the human DOCK8 protein fused at the N terminal with a C-intein is set forth in SEQ ID NO: 42.

In another related aspect of the system, the first nucleic acid is comprised in a first lentiviral vector (first LV) and the second nucleic acid is comprised in a second lentiviral vector (second LV). In a further related aspect, the first LV and said second LV are each TAT-independent and self-inactivating (SIN) lentiviral vectors. Still another further related aspect, the first nucleic acid further comprises a WPRE element located 3′ of said nucleic acid encoding the N-intein and said second nucleic acid further comprises a WPRE element located 3′ of said nucleic acid encoding the second fragment of DOCK8 protein.

In another related aspect of the system, the nucleic acid sequence of said first nucleic acid is set forth in SEQ ID NO: 28 and the nucleic acid sequence of said second nucleic acid is set forth in SEQ ID NO: 29 or SEQ ID NO: 30.

In yet another related aspect of the system, the nucleic acid sequence of said first nucleic acid is set forth in SEQ ID NO: 38 and the nucleic acid sequence of said second nucleic acid is set forth in SEQ ID NO: 39.

Disclosed herein in one aspect is a pair of recombinant lentiviral vectors (LVs) comprising:
a first LV comprising a first nucleic acid encoding a first fragment of a human DOCK8 protein fused at the C termina to an N-intein; and
a second LV comprising a second nucleic acid encoding a second fragment of the human DOCK8 protein fused at the N terminal with a C-intein;
wherein the first fragment of the DOCK8 protein ligated to the second fragment of the DOCK8 protein forms a full-length DOCK8 protein,
wherein the first fragment of the DOCK8 protein and the second fragment of the DOCK8 protein are selected to provide a split between said first fragment and said second fragment in an external loop that does not contribute to intra-protein interactions,
wherein said first fragment comprises the DOCK homology region 1 (DHR1) and said second fragment comprises the DOCK homology region 2 (DHR2), and
wherein said first nucleic acid further comprises a promoter located 5′ of said nucleic acid encoding the first fragment of DOCK8 and said second nucleic acid further comprises a promoter located 5′ of said nucleic acid encoding the C-intein.

In a related aspect to the pair of LVs, the amino acid sequence of said first fragment of DOCK8 is set forth in SEQ ID NO: 18 and the amino acid sequence of said second fragment of DOCK8 is set forth in SEQ ID NO: 25, or the amino acid sequence of said first fragment of DOCK8 is set forth in SEQ ID NO: 34 and the amino acid sequence of said second fragment of DOCK8 is set forth in SEQ ID NO: 37. In another related aspect to the pair of LVs, the first nucleic acid encoding a first fragment of a human DOCK8 is codon-optimized and wherein said second nucleic acid encoding a second fragment of the human DOCK8 protein is codon-optimized.

In a related aspect to the pair of LVs, the second fragment of the DOCK8 protein comprise an extein linker where said linker comprises C-F or C-F-N. In a further related aspect, the N-intein and C-intein comprise a Cfa split intein, wherein the N-intein comprises a CfaN sequence and the C-intein comprise a CfaC sequence, wherein optionally said nucleotide sequence encoding said CfaN amino acid sequence and said nucleotide sequence encoding said CfaC amino acid sequence are codon optimized for expression in a human cell. In yet another further related aspect, the amino acid sequence of said first fragment of the human DOCK8 protein fused at the C terminal to an N-intein is set forth in SEQ ID NO: 17 and the amino acid sequence of said second fragment of the human DOCK8 protein fused at the N terminal with a C-intein is set forth in SEQ ID NO: 23 or SEQ ID NO: 27; or the amino acid sequence of said first fragment of the human DOCK8 protein fused at the C terminal to an N-intein is set forth in SEQ ID NO: 33 and the amino acid sequence of said second fragment of the human DOCK8 protein fused at the N terminal with a C-intein is set forth in SEQ ID NO: 42.

In another related aspect of the pair of LVs, the first nucleic acid further comprises a WPRE element located 3′ of said nucleic acid encoding the N-intein and said second nucleic acid further comprises a WPRE element located 3′ of said nucleic acid encoding the second fragment of DOCK8 protein.

In still another related aspect of the pair of LVs, the nucleic acid sequence of said first nucleic acid is set forth in SEQ ID NO: 28 and the nucleic acid sequence of said second nucleic acid is set forth in SEQ ID NO: 29 or SEQ ID NO: 30. In yet another related aspect of the pair of LVs, the nucleic acid sequence of said first nucleic acid is set forth in SEQ ID NO: 38 and the nucleic acid sequence of said second nucleic acid is set forth in SEQ ID NO: 39. In a further related aspect of the pair of LVs, the first LV and said second LV are each TAT-independent and self-inactivating (SIN) lentiviral vectors.

In one aspect, disclosed herein is a host cell transduced with the pair of lentiviral vectors according to another of the aspects disclosed herein. In a related aspect, the cell is a stem cell derived from bone marrow, umbilical cord blood, or from peripheral blood, or any combination thereof, or is a hematopoietic progenitor cell. In another related aspect, the human hematopoietic progenitor cell is a CD34+ cell.

In one aspect, disclosed herein is method of treating a DOCK8 deficiency in a human subject, said method comprising:
transducing a stem cell and/or progenitor cell from said subject with a pair of lentiviral vectors according to claim 12 to produce transduced cells expressing functional DOCK8 protein; and
transplanting said transduced cell or cells derived therefrom into said subject, where said cells therefrom express functional DOCK8 protein. In a related aspect, the DOCK8 protein is constitutively expressed. In another related aspect, the cell is a stem cell or human hematopoietic progenitor cell or a combination thereof. In a further related aspect, the stem cell is derived from bone marrow. In yet another further related aspect, the human hematopoietic progenitor cell is a CD34+ cell.

### BRIEF DESCRIPTION OF THE DRAWINGS

The subject matter regarded as the system for expressing DOCK8 protein in a cell and methods of use thereof, is particularly pointed out and distinctly claimed in the concluding portion of the specification. The system for expressing DOCK8 protein in a cell and methods of use thereof, however, both as to organization and method of operation, together with objects, features, and advantages thereof, may best be understood by reference to the following detailed description when read with the accompanying drawings in which:

FIGS. 1A-1D. FIG. 1A shows a schematic of the two half-DOCK8 cDNAs integrating into the genome of a peripheral blood stem cell (PBSC). P/P indicates Primer/Probe sites. E/P indicates an upstream enhancer/promotor. pA is a polyA sequence. FIG. 1B shows percent (%) integration as measured by the different probes. FIG. 1C shows viability of the transduced PBSC. FIG. 1D shows proliferation (Fold Expansion) of the transduced PBSC.

FIG. 2. DOCK8 lentiviral vectors include an internal EFS promoter driving expression of two codon-optimized versions of the therapeutic gene cassette with and without WPRE. Codon optimization was performed using either GeneArt or GenScript methods. The nucleotide sequence of the vector comprising EFS-GeneArtDOCK8-WPRE is set forth in SEQ ID NO: 4.

FIGS. 3A-3B. DOCK8 lentiviral vectors were tested in Jurkat T cells. FIG. 3A. GeneArt codon optimization of the DOCK8 cDNA resulted in restoration of DOCK8 expression. Y axis represents Count. X-axis represent Dock8. FIG. 3B. Inclusion of the WPRE sequence improved DOCK8 expression at similar vector copy number (VCN) when measured by flow cytometry.

FIGS. 4A and 4B. The VCN of PBSC transduced with EFS-GeneArtDOCK8-WPRE at 1e7 TU/ml (FIG. 4A) or the VCN of PBSC transduced with EFS-GeneArtDOCK8-WPRE at various TU/mL (FIG. 4B), alone or in combination with various transduction enhancers. Cells are typically transduced after one day pre-stimulation, except samples labeled as day 2, which were transduced after 2 days pre-stimulation. 2 hit samples refer to those which were transduced twice, after one and two days pre-stimulation. Results are from 5 different PBSC donors mixed together.

FIGS. 5A-5B. The beta-can shape of the GFP protein is shown in FIG. 5A. The split site (red line) for GFP was chosen at one of the short helical segments at the ends of the beta-sheet cylinder. FIG. 5B. Schematic of GFP-CfaN and CfaC-GFP (C-F) expression plasmids.

FIG. 6. K562 cells electroporated with the CfaN or CfaC plasmids alone do not express GFP. (top row) Cells electroporated with both plasmids express high levels of GFP. Both CFN and CF adjacent extein sequences appear to allow equal levels of protein splicing.

FIG. 7. DOCK8 protein structure (top) and two DHR domains important for protein function (bottom) from Biggs et al. (2017) DOCK8 deficiency: Insights into pathophysiology, clinical features and management. Clin Immunol. 181:75-82.

FIG. 8. Two different split sites in the DOCK8 protein were tested at amino acid positions 939 and 1267. The split site is just before the 939^{th} amino acid of DOCK8 (939 plasmid vectors) or split site for this cassette is just after the 1267th amino acid of DOCK8 (1267 plasmid vectors).

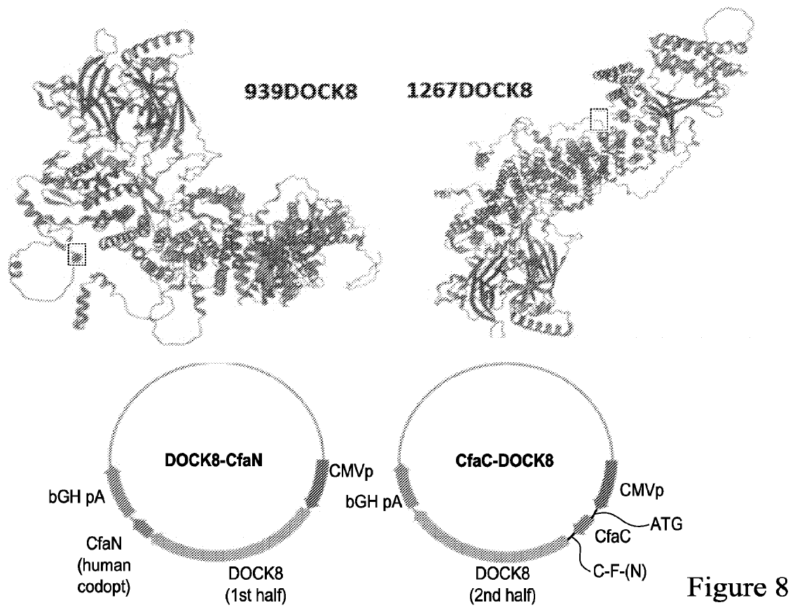

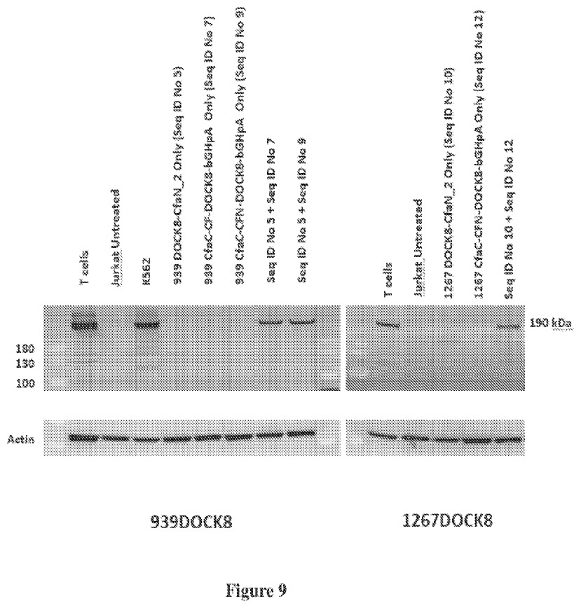

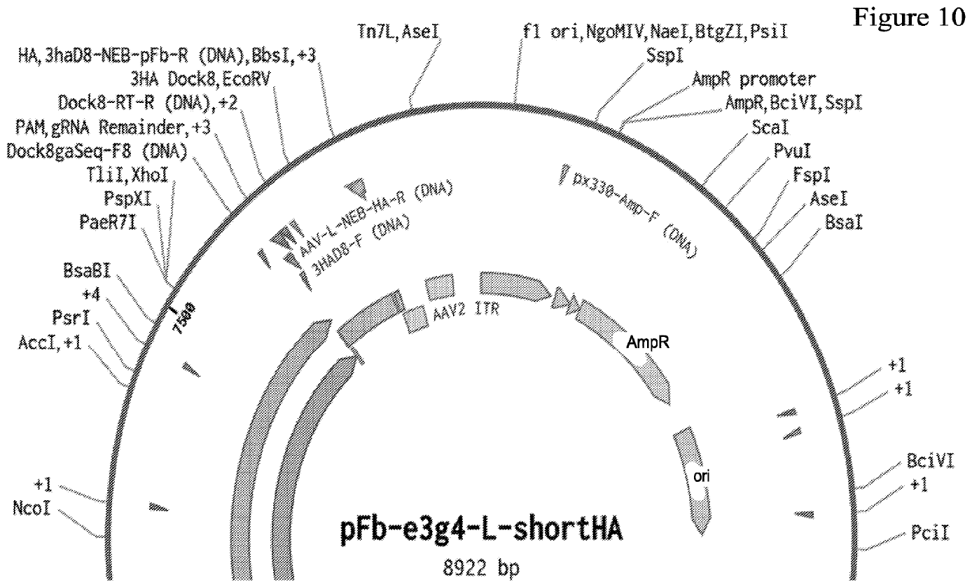

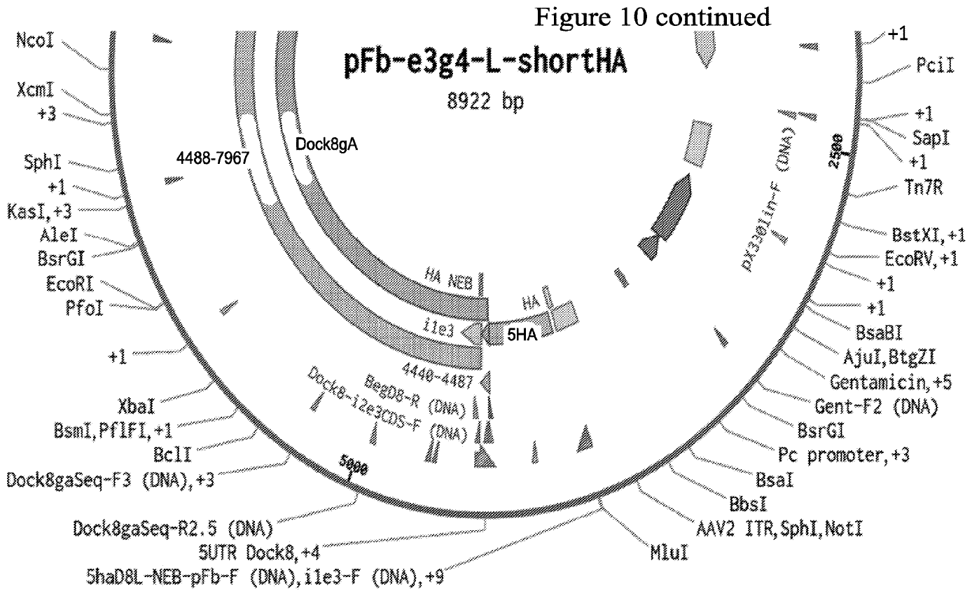

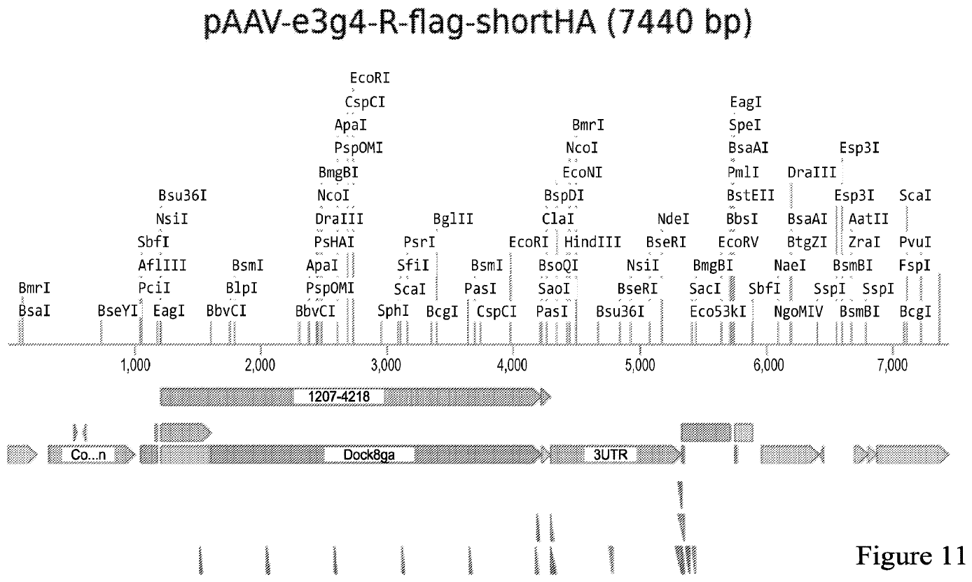

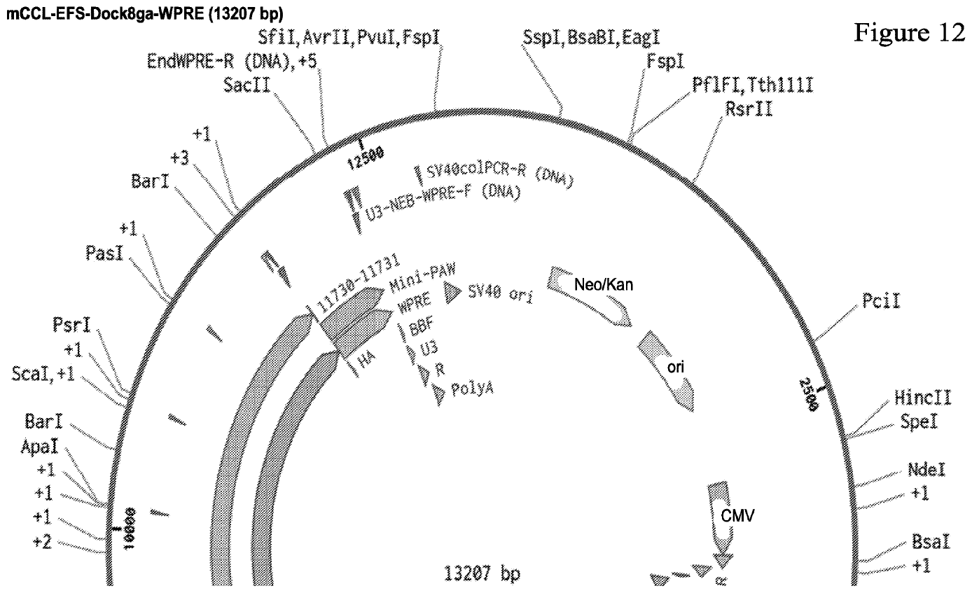

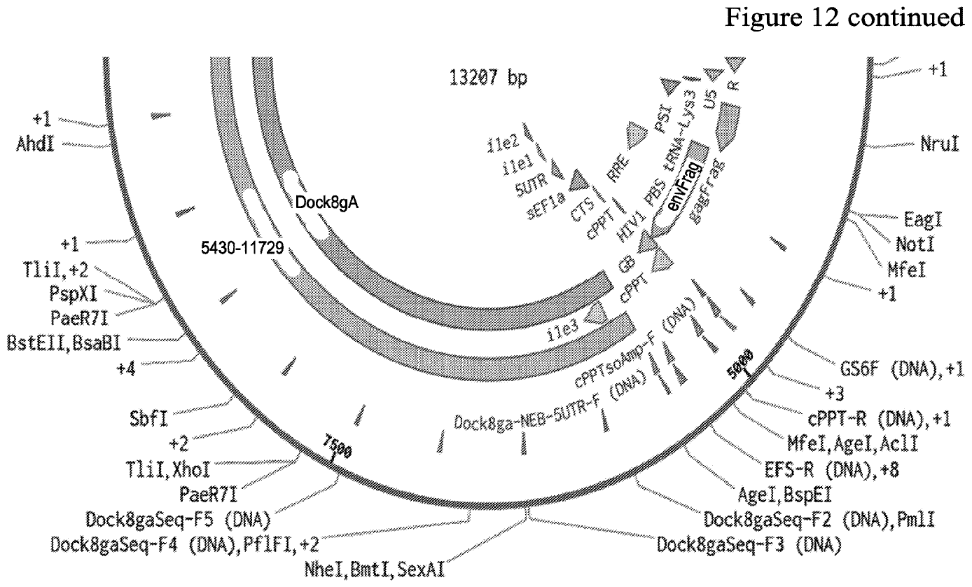

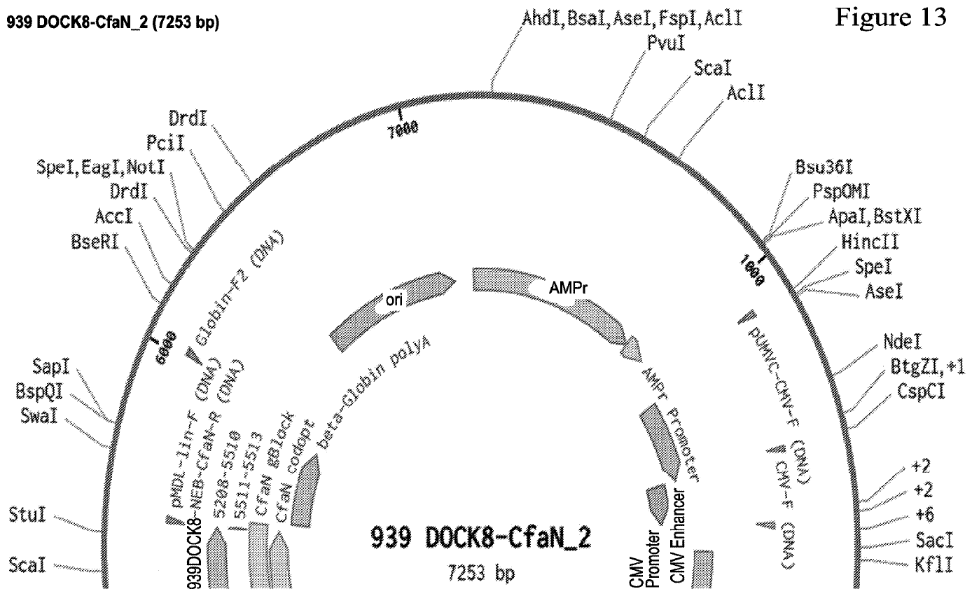

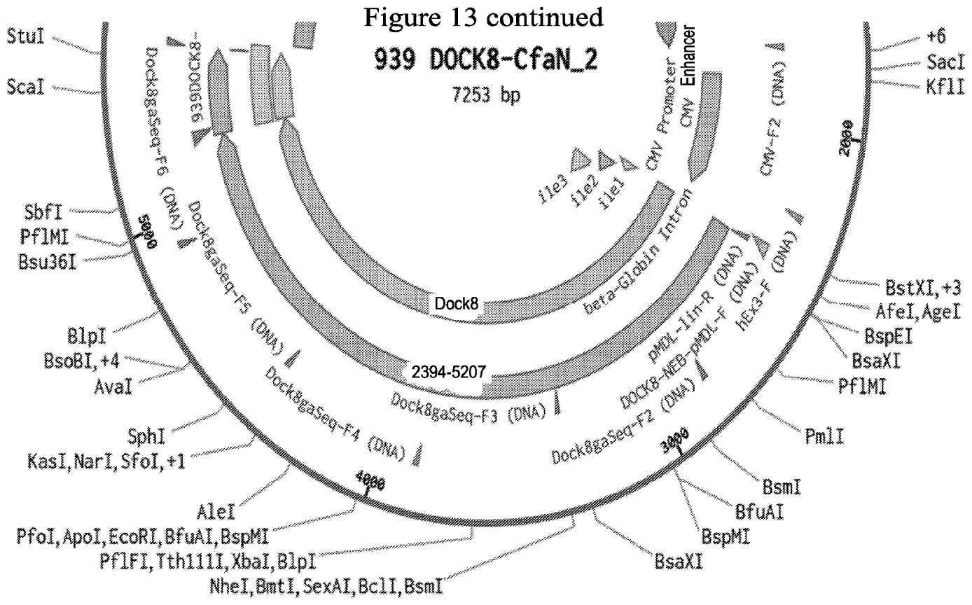

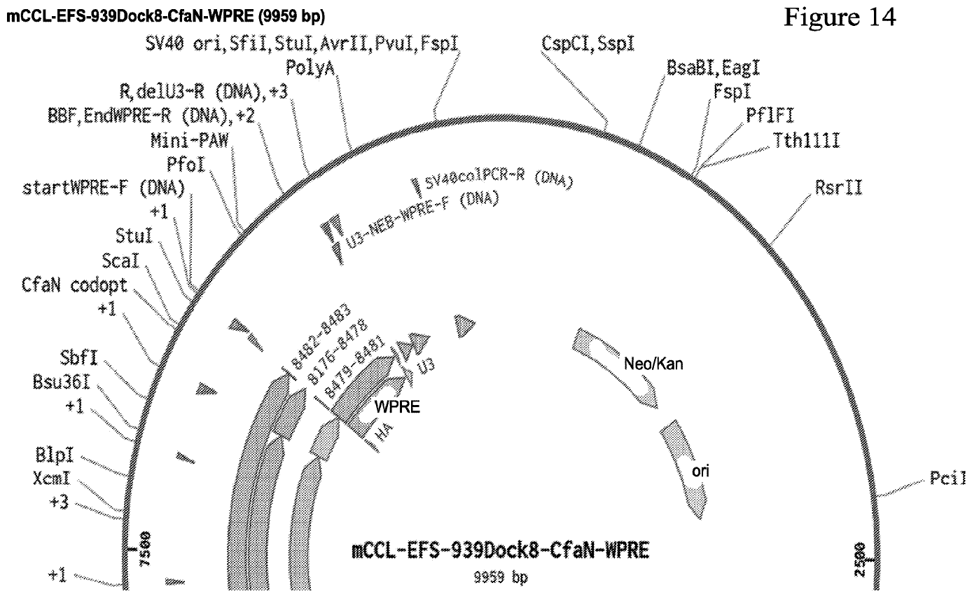

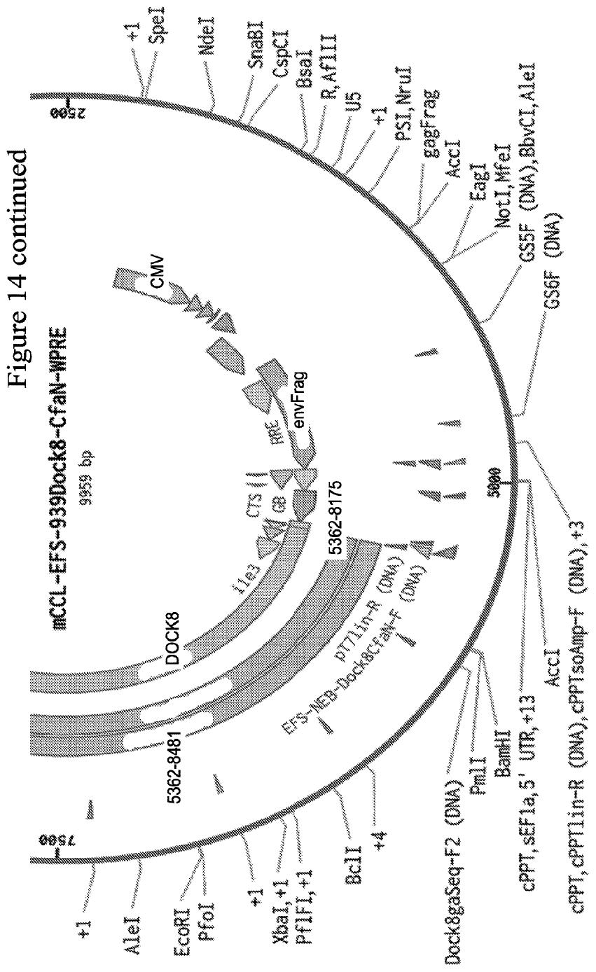

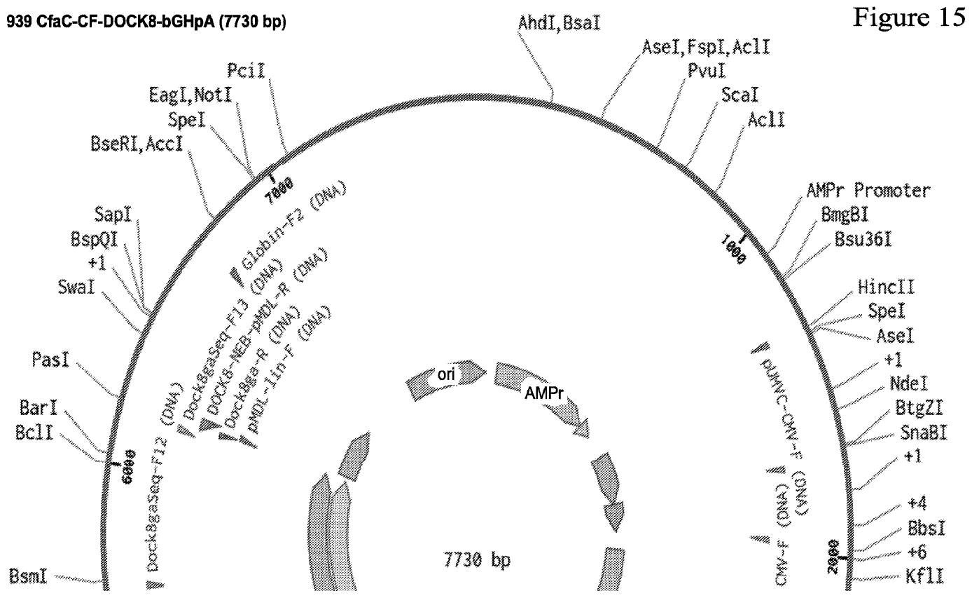

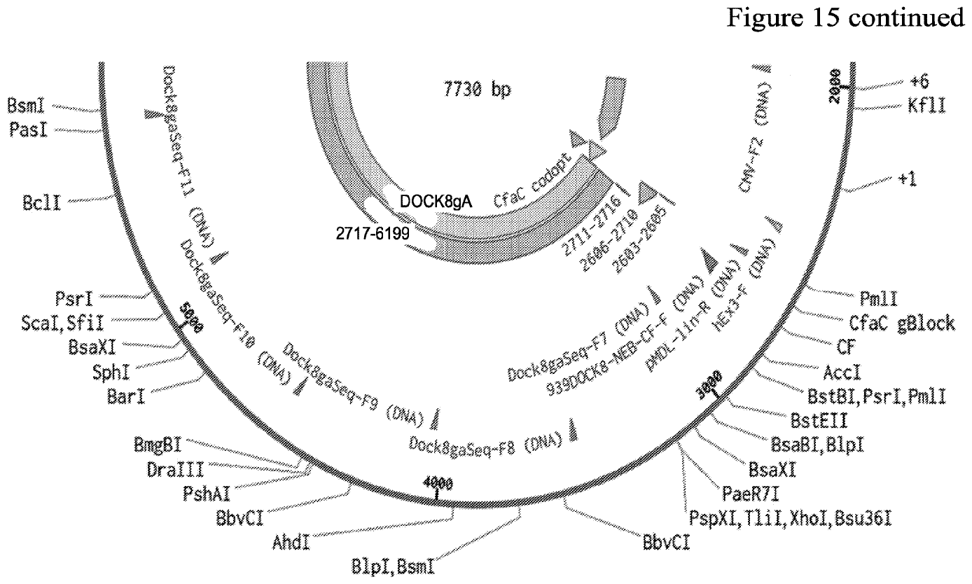

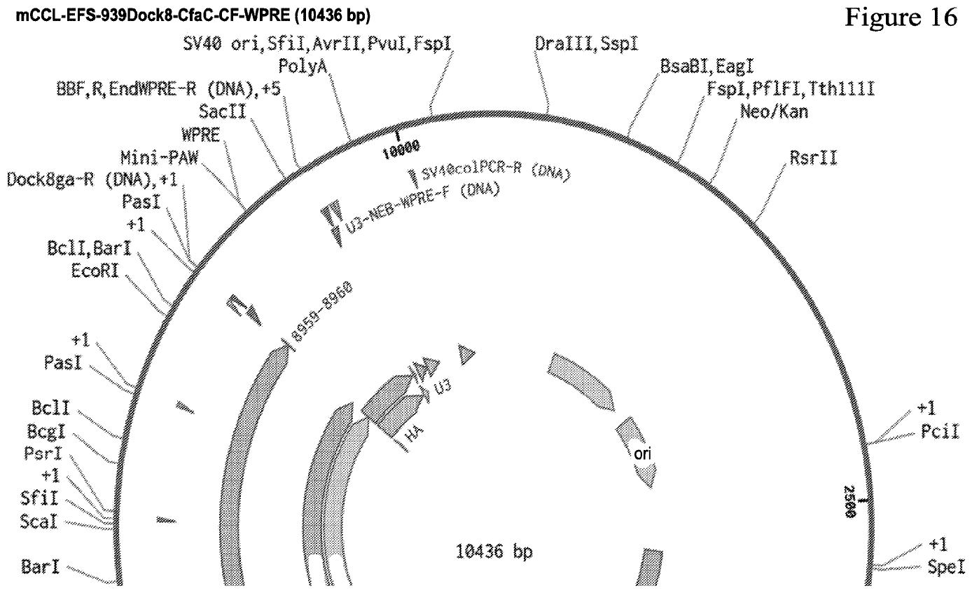

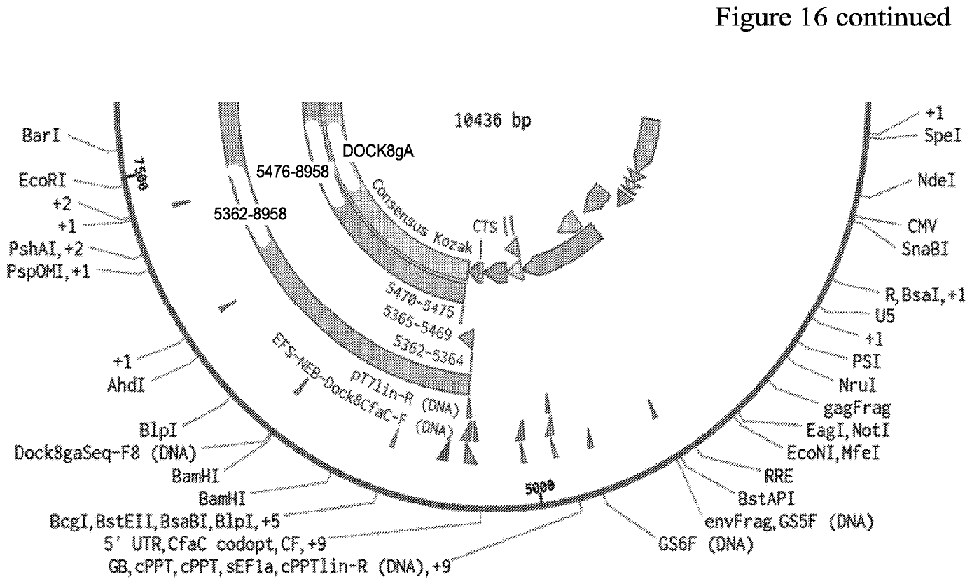

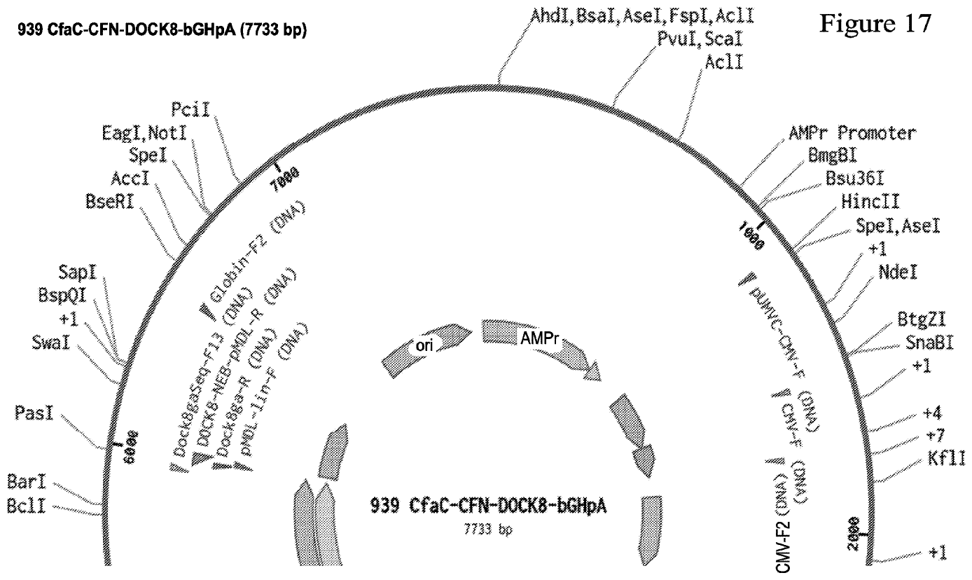

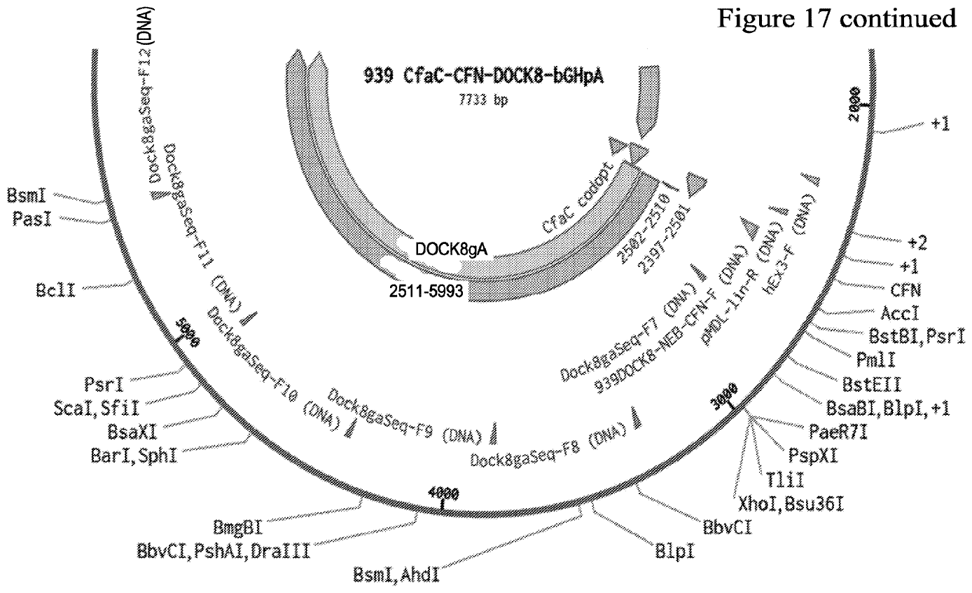

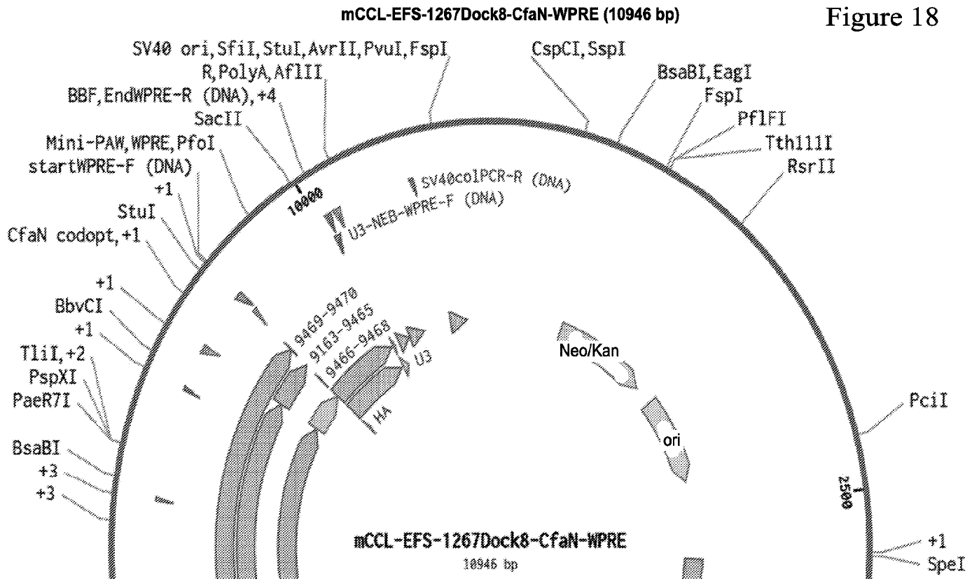

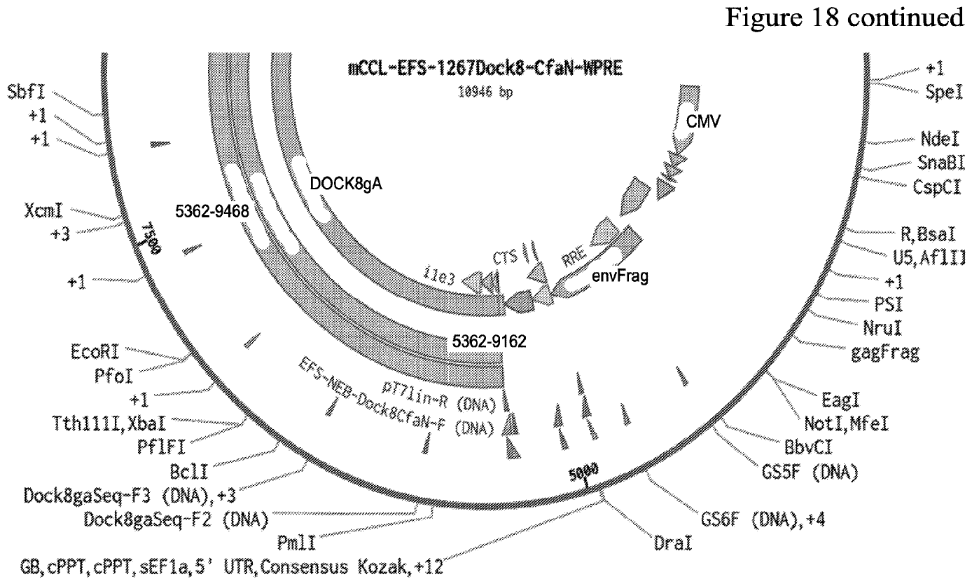

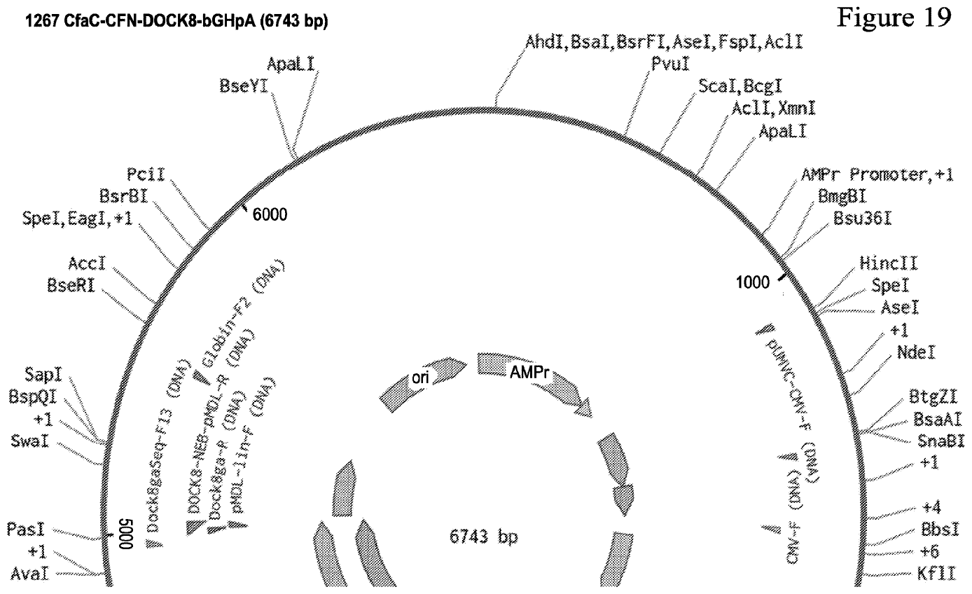

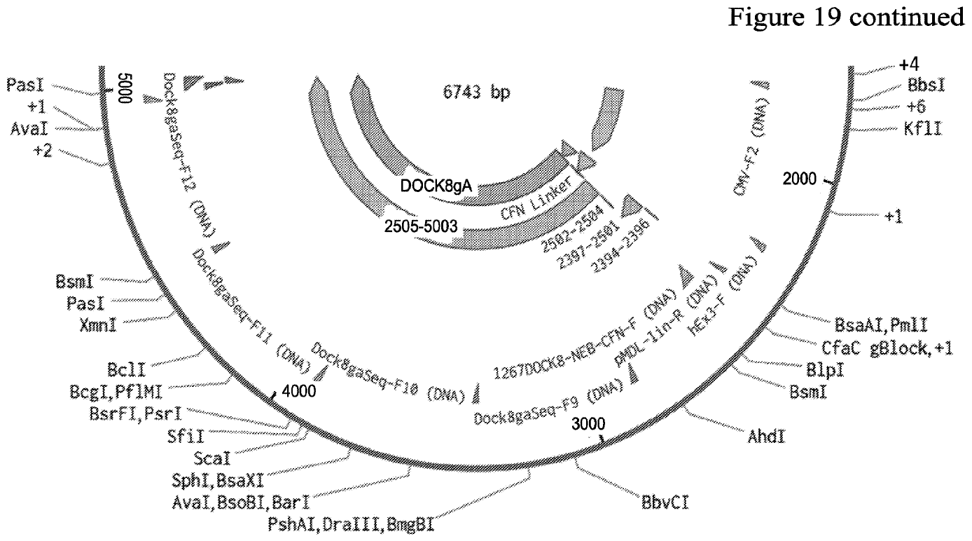

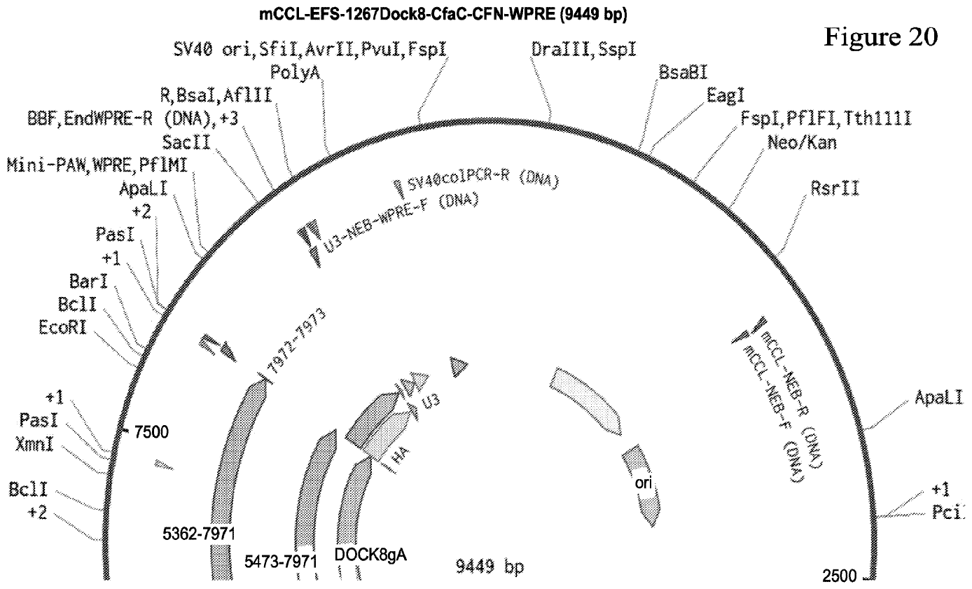

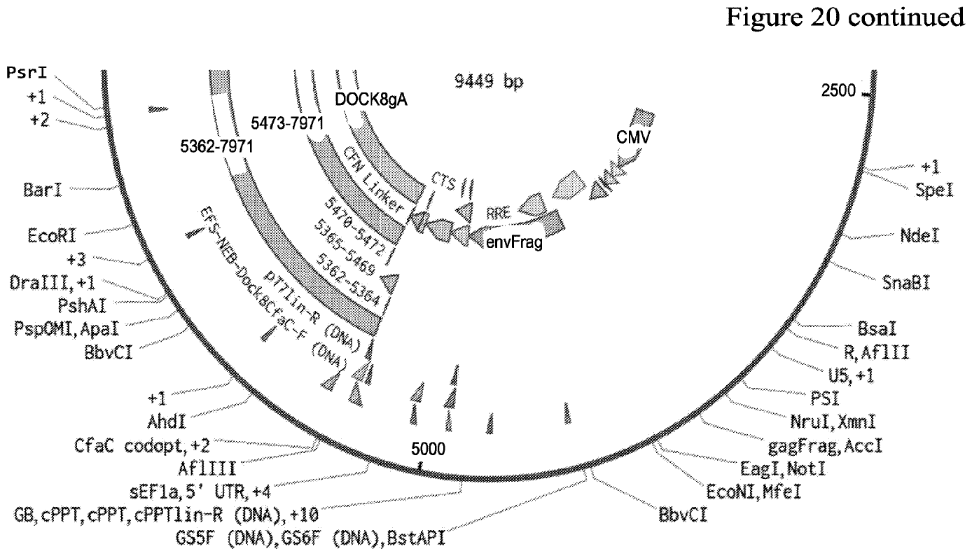

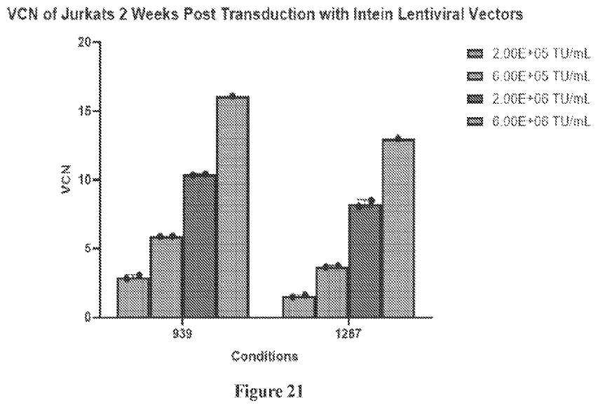

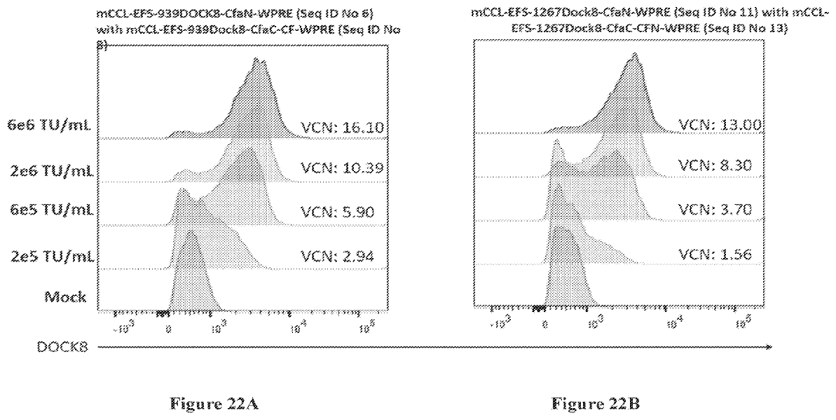

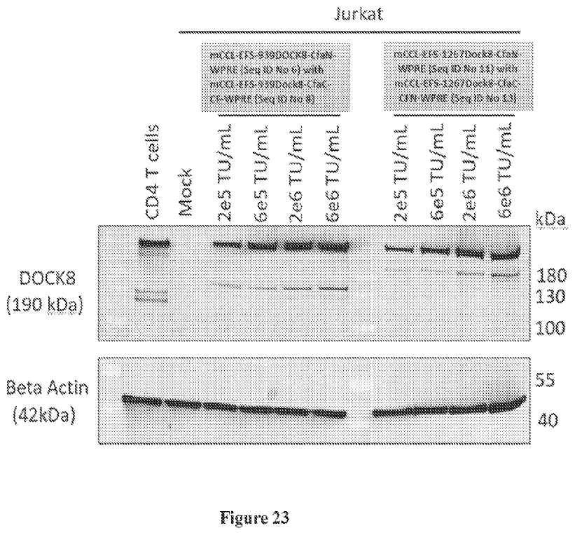

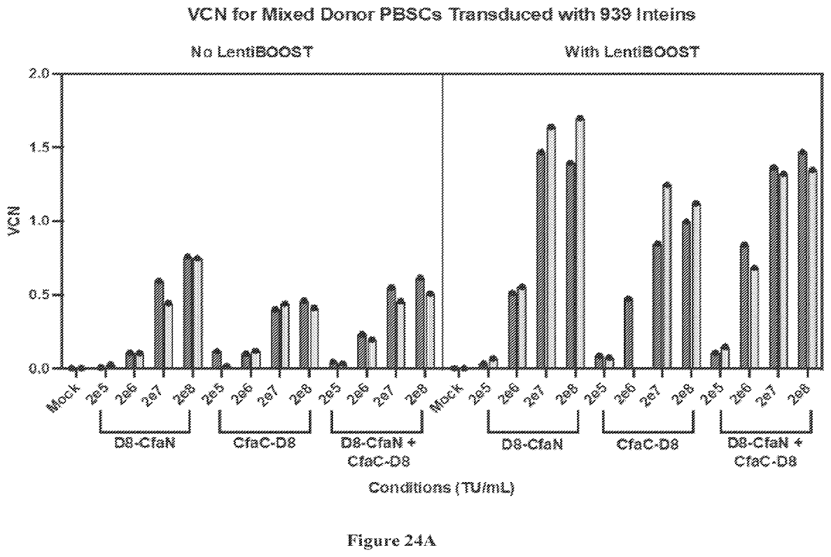

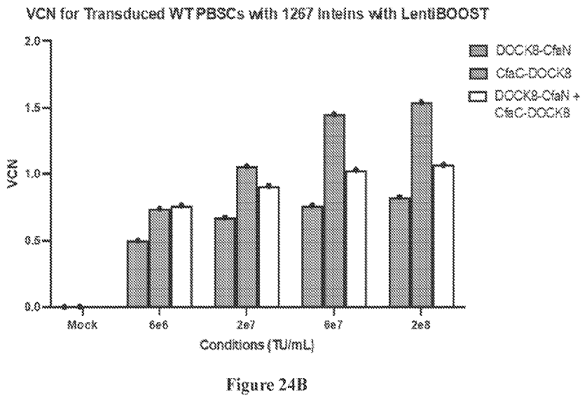

Each plasmid vector of paired plasmid vectors (See for example FIGS. 12-20) comprises nucleic acid sequences encoding either the N-terminal or C-terminal half of DOCK8. A skilled artisan would recognize that the term “half” is used to denote either a N-terminal (5′) half of DOCK8 protein (nucleic acid) or a C-terminal (3′) half of DOCK8 protein (nucleic acid). The term half is not meant to infer or define a quantity of the DOCK8 protein or nucleotide sequence as being exactly half (50%). While the two halves when spliced together form a full-length DOCK8, the term “half” is employed to denote a portion of the protein or nucleotide sequence encoding the protein. The split sites in these 939 or 1267 vectors are as described here for FIG. 8.

FIG. 9. Immunoblots demonstrating restoration of DOCK8 protein expression in Jurkat T cells treated with a combination of the CfaN and CfaC intein/DOCK8 plasmids. Untreated Jurkat T cells do not express DOCK8 at baseline, and cells treated with only one of the intein plasmids does not express detectable DOCK8 protein. Positive controls include primary T cells from healthy individuals and K562 cell lines.

FIG. 10. Map of AAV donor sequences for sequential editing approach left (5′) side. pFb-e3g4-L-shortHA (8922 bp).

FIG. 11. Map of AAV donor sequences for sequential editing approach right (3′) side pAAV-e3g4-R-flag-shortHA (7440 bp).

FIG. 12. Map of lentiviral plasmid sequence for gADOCK8-WPRE Lentiviral approach. mCCL-EFS-Dock8ga-WPRE (13207 bp).

FIG. 13. Map of DOCK8 939 CfaN Intein plasmid sequence. 939 DOCK8-CfaN_2 (7253 bp).

FIG. 14. Map of DOCK8 939 CfaN Intein Lentiviral plasmid sequence. mCCL-EFS-939Dock8-CfaN-WPRE (9959 bp).

FIG. 15. Map of DOCK8 939 CfaC-CF Intein plasmid sequence. 939 CfaC-CF-DOCK8-bGHpA (7730 bp).

FIG. 16. Map of DOCK8 939 CfaC-CF Intein Lentiviral plasmid sequence. mCCL-EFS-939Dock8-CfaC-CF-WPRE (10436 bp).

FIG. 17. Map of DOCK8 939 CfaC-CFN Intein plasmid sequence. 939 CfaC-CFN-DOCK8-bGHpA (7733 bp).

FIG. 18. Map of DOCK8 1267 CfaN Intein Lentiviral plasmid sequence. mCCL-EFS-1267Dock8-CfaN-WPRE (10946 bp).

FIG. 19. Map of DOCK8 1267 CfaC-CFN plasmid sequence. 1267 CfaC-CFN-DOCK8-bGHpA (6743 bp).

FIG. 20. Map of DOCK8 1267 CfaC-CFN Lentiviral plasmid sequence. mCCL-EFS-1267Dock8-CfaC-CFN-WPRE (9449 bp).

FIG. 21. Vector Copy Number (VCN) of Jurkat cells 2 weeks post transduction with intein Lentiviral vectors.

FIGS. 22A and 22B. VCN and Expression of DOCK8 was analyzed in Jurkat cells transduced with different doses of intein Lentivirus 2 weeks post transduction.

FIG. 23. Western Blot of transduced Jurkat cells demonstrates DOCK8 expression.

FIGS. 24A and 24B. Intein Lentiviral vectors in human Peripheral Blood Stem Cells (PBSC).

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION

In the following detailed description, numerous specific details are set forth in order to provide a thorough understanding of the system for expressing DOCK8 protein in a stem and or progenitor cell and methods of use thereof. However, it will be understood by those skilled in the art that the systems and vectors comprising the DOCK8 protein and the methods of use thereof may be practiced without these specific details. In other instances, well-known methods, procedures, and components have not been described in detail so as not to obscure the DOCK8 system and vectors.

### DOCK8 Protein and Systems for Expressing a Human DOCK8 Protein

In humans, the Dedicator Of Cytokinesis 8 (DOCK8) gene encodes a DOCK8 protein that is a guanine nucleotide releasing factor involved in intracellular signaling networks. DOCK8 protein (2099 AA; ~190 kDa) is exceptionally large, which can lead to challenges for heterologous expression as part of a gene therapy regime.

In some embodiments, disclosed herein is a system for expressing human DOCK8 protein in a cell, wherein the system comprises:
a first nucleic acid encoding a first fragment of a human DOCK8 protein fused at the C terminal to an N-intein; and
a second nucleic acid encoding a second fragment of the human DOCK8 protein fused at the N terminal with a C-intein;
wherein the first fragment of the DOCK8 protein ligated to the second fragment of the DOCK8 protein forms a full-length human DOCK8 protein,
wherein the first fragment of the DOCK8 protein and the second fragment of the DOCK8 protein are selected to provide a split between said first fragment and said second fragment in an external loop that does not contribute to intra-protein interactions, wherein said first fragment comprises the DOCK homology region 1 (DHR1) and said second fragment comprises the DOCK homology region 2 (DHR2), and
wherein said first nucleic acid further comprises a promoter located 5′ of said nucleic acid encoding the first fragment of DOCK8 and said second nucleic acid further comprises a promoter located 5′ of said nucleic acid encoding the C-intein.

One skilled in the art would appreciate that it is important to choose split sites that do not appear to contribute to intra-protein interactions because the DOCK8 extein that is fused to the CfaC intein requires a CF or CFN linker that will be incorporated into the resulting DOCK8 protein. The split sites of 939 and 1267 meet this criteria. The 939 split site is just before the 939^{th} amino acid of DOCK8 (939 plasmid vectors). The 1267 split site for cassette is just after the 1267^{th} amino acid of DOCK8 (1267 plasmid vectors).

In some embodiments, the promoter comprises a constitutive promoter. In some embodiments, said constitutive promoter comprises an EFS promoter, a PGK (phosphoglycerate kinase 1) promoter, a CMV (cytomegalovirus immediate) promoter, a MND promoter (synthetic promoter containing U3 region of modified Maloney murine leukemia virus and enhancer from myeloproliferative sarcoma virus), an endogenous DOCK8 promoter, or a EF-1alpha promoter (human eukaryotic translation elongation factor 1 alpha 1). In some embodiments, said constitutive promoter comprises an EFS promoter. While in some embodiments, the shortened EF-1alpha promoter is used in the Examples below, a skilled artisan would recognize that the full length version of the EF-1alpha promoter could also be used.

In some embodiments, a system comprises a first nucleic acid and a second nucleic acid, each encoding a fragment of human DOCK8 protein, wherein the two fragments when ligated together form a full-length human DOCK8 protein. In some embodiments of a system, each nucleic acid is comprised in a vector.

In some embodiments, the first fragment comprises the DOCK homology region 1 (DHR1) and the second fragment comprises the DOCK homology region 2 (DHR2).

In some embodiments of a system, the first and second nucleic acids may integrate into a host cell genome, and once integrated express the full-length DOCK8 protein. In some embodiments, expression is constitutive. In some embodiments, the expressed full-length DOCK8 protein promotes STAT3 phosphorylation and translocation to the nucleus.

The term “first fragment of DOCK8” and grammatical variations thereof, may be used interchangeably with the term “first half” or “first half of DOCK8” and grammatical variations thereof, having all the same meanings and qualities. Similarly, the term “second fragment of DOCK8” and grammatical variations thereof, many be used interchangeably with the term “second half” or “second half of DOCK8” and grammatical variations thereof, having all the same meanings and qualities.

A skilled artisan would recognize that the term “half” as used herein, refers to a portion or a part of DOCK8, wherein a first and second half of DOCK8 together form a full-length DOCK8 protein or encode a full-length DOCK8 protein, although each half is not defined as being 50% of DOCK8. For example, in certain embodiments, a first half of a DOCK8 protein comprises the protein up until the amino acid before position 939. The split site is just before the 939^{th} amino acid of DOCK8 so the DOCK8 cDNA for this sequence ends at the codon encoding amino acid 938. In that same embodiment, the second half includes amino acid 939 of DOCK8 until the end of the DOCK8 protein sequence. These halves are termed “939DOCK8” first half and second half. Similarly, in certain embodiments, a first half of a DOCK8 protein comprises the protein up until and including amino acid 1267. The split site for this cassette is just after the 1267th amino acid of DOCK8. In that same embodiment, the second half includes amino acid 1268 of DOCK8 through to the end of the DOCK8 protein sequence. These halves are termed “1267DOCK8” first half and second half.

In some embodiments, a first nucleic acid and a second nucleic acid each comprise a promoter to drive the expression of the encoded DOCK8 fragment. In certain embodiments, the promoter comprises a constitutive human promoter. In certain embodiments, the promoter comprises an elongation factor 1α (EF-1α) promoter. In some embodiments, the promoter comprises an EF-1α Short (EFS) intron-less promoter.

In some embodiments, a first nucleic acid encodes a first fragment of a human DOCK8 protein fused at the C terminal to an N-intein. In some embodiments, the N-intein and C-intein comprise a Cfa split intein, wherein the N-intein comprises a CfaN sequence and the C-intein comprise a CfaC sequence, wherein optionally said nucleotide sequence encoding said CfaN amino acid sequence and said nucleotide sequence encoding said CfaC amino acid sequence are codon optimized for expression in a human cell. In some embodiments, the N-intein and C-intein comprise a Cfa split intein, wherein the N-intein comprises a CfaN sequence and the C-intein comprise a CfaC sequence. In some embodiments, the nucleotide sequence encoding the CfaN amino acid sequence and the nucleotide sequence encoding said CfaC amino acid sequence are codon optimized for expression in a human cell. In some embodiments, a codon-optimized CfaN nucleotide sequence is set forth in SEQ ID NO: 16. In some embodiments, a CfaN amino acid sequence is set forth in SEQ ID NO: 19. In some embodiments, a codon-optimized CfaC nucleotide sequence is set forth in SEQ ID NO:21. In some embodiments, a CfaC amino acid sequence is set forth in SEQ ID NO: 24.

In some embodiments, a first nucleic acid encodes a first half of DOCK8 fused at the C terminal to an CfaN. In some embodiments, a first nucleic acid encodes a first half of 939DOCK8 fused at the C terminal to an CfaN. In some embodiments, a first nucleic acid encodes a first half of 1267DOCK8 fused at the C terminal to an CfaN.

In some embodiments, a first nucleic acid encodes a first fragment of a human DOCK8 protein fused at the C terminal to an N-intein. In some embodiments, a first nucleic acid encodes a first half of DOCK8 fused at the C terminal to an CfaN. In some embodiments, a first nucleic acid encodes a first half of 939DOCK8 fused at the C terminal to an CfaN. In some embodiments, a first nucleic acid encodes a first half of 1267DOCK8 fused at the C terminal to an CfaN.

In some embodiments, a second nucleic acid encodes a second fragment of the human DOCK8 protein fused at the N terminal with a C-intein. In some embodiments, a second nucleic acid encodes a second half of DOCK8 fused at the N terminal with CfaC. In some embodiments, a second half of DOCK8 protein comprises an extein linker. In some embodiments, the linker comprises amino acids Cysteine-Phenylalanine (C-F) or Cysteine-Phenylalanine-Asparagine (C-F-N). In some embodiments, a second nucleic acid encodes a second half of 939DOCK8 fused at the N terminal to C-F-CfaC. In some embodiments, a second nucleic acid encodes a second half of 939DOCK8 fused at the N terminal to C-F-N-CfaC. In some embodiments, a second nucleic acid encodes a second half of 1267DOCK8 fused at the N terminal to C-F-N-CfaC, wherein the N residue is part of the 1267DOCK8 second half amino acid sequence.

In some embodiments, the amino acid sequence of said first fragment of DOCK8 is set forth in SEQ ID NO: 18 and the amino acid sequence of said second fragment of DOCK8 is set forth in SEQ ID NO: 25, or the amino acid sequence of said first fragment of DOCK8 is set forth in SEQ ID NO: 34 and the amino acid sequence of said second fragment of DOCK8 is set forth in SEQ ID NO: 37. In some embodiments, the amino acid sequence of said first fragment of DOCK8 is set forth in SEQ ID NO: 18 and the amino acid sequence of said second fragment of DOCK8 is set forth in SEQ ID NO: 25. In some embodiments, the amino acid sequence of said first fragment of DOCK8 is set forth in SEQ ID NO: 34 and the amino acid sequence of said second fragment of DOCK8 is set forth in SEQ ID NO: 37.

In some embodiments, the nucleotide sequence encoding the DOCK8 protein is codon-optimized for translation in humans. In some embodiments, the nucleotide sequence encoding a first fragment of DOCK8 is codon-optimized. In some embodiments, the nucleotide sequence encoding a second fragment of DOCK8 is codon-optimized. In some embodiments, the first nucleic acid encoding a first fragment of a human DOCK8 is codon-optimized for translation in humans and the second nucleic acid encoding a second fragment of the human DOCK8 protein is codon-optimized for translation in humans.

In some embodiments, the optimized nucleic acid sequence encoding said first fragment of DOCK8 is set forth in SEQ ID NO: 15 and the optimized nucleic acid sequence encoding said second fragment of DOCK8 is set forth in SEQ ID NO: 22, or the optimized nucleic acid sequence encoding first fragment of DOCK8 is set forth in SEQ ID NO: 32 and the optimized nucleic acid sequence encoding said second fragment of DOCK8 is set forth in SEQ ID NO: 36. In some embodiments, the optimized nucleic acid sequence encoding said first fragment of DOCK8 is set forth in SEQ ID NO: 15 and the optimized nucleic acid sequence encoding second fragment of DOCK8 is set forth in SEQ ID NO: 22. In some embodiments, the optimized nucleic acid sequence encoding said first fragment of DOCK8 is set forth in SEQ ID NO:32 and the optimized nucleic acid sequence encoding said second fragment of DOCK8 is set forth in SEQ ID NO: 36.

In some embodiments, the amino acid sequence of said first fragment of the human DOCK8 protein fused at the C terminal to an N-intein is set forth in SEQ ID NO: 17 and the amino acid sequence of said second fragment of the human DOCK8 protein fused at the N terminal with a C-intein is set forth in SEQ ID NO: 23 or SEQ ID NO: 27; or the amino acid sequence of said first fragment of the human DOCK8 protein fused at the C terminal to an N-intein is set forth in SEQ ID NO: 33 and the amino acid sequence of said second fragment of the human DOCK8 protein fused at the N terminal with a C-intein is set forth in SEQ ID NO: 42. In some embodiments, the amino acid sequence of said first fragment of the human DOCK8 protein fused at the C terminal to an N-intein is set forth in SEQ ID NO: 17 and the amino acid sequence of said second fragment of the human DOCK8 protein fused at the N terminal with a C-intein is set forth in SEQ ID NO: 23. In some embodiments, the amino acid sequence of said first fragment of the human DOCK8 protein fused at the C terminal to an N-intein is set forth in SEQ ID NO: 17 and the amino acid sequence of said second fragment of the human DOCK8 protein fused at the N terminal with a C-intein is set forth in SEQ ID NO: 27. In some embodiments, the amino acid sequence of said first fragment of the human DOCK8 protein fused at the C terminal to an N-intein is set forth in SEQ ID NO: 33 and the amino acid sequence of said second fragment of the human DOCK8 protein fused at the N terminal with a C-intein is set forth in SEQ ID NO: 42.

In some embodiments, the nucleic acid sequence encoding said first fragment of the human DOCK8 protein fused at the C terminal to an N-intein is set forth in SEQ ID NO: 14 and the nucleic acid sequence encoding said second fragment of the human DOCK8 protein fused at the N terminal with a C-intein is set forth in SEQ ID NO: 20 or SEQ ID NO: 26; or the nucleic acid sequence encoding said first fragment of the human DOCK8 protein fused at the C terminal to an N-intein is set forth in SEQ ID NO: 31 and the nucleic acid sequence encoding said second fragment of the human DOCK8 protein fused at the N terminal with a C-intein is set forth in SEQ ID NO: 35. In some embodiments, the nucleic acid sequence encoding said first fragment of the human DOCK8 protein fused at the C terminal to an N-intein is set forth in SEQ ID NO: 14 and the nucleic acid sequence encoding said second fragment of the human DOCK8 protein fused at the N terminal with a C-intein is set forth in SEQ ID NO: 20. In some embodiments, the nucleic acid sequence encoding said first fragment of the human DOCK8 protein fused at the C terminal to an N-intein is set forth in SEQ ID NO: 14 and the nucleic acid sequence encoding said second fragment of the human DOCK8 protein fused at the N terminal with a C-intein is set forth in SEQ ID NO:26. In some embodiments, the nucleic acid sequence encoding said first fragment of the human DOCK8 protein fused at the C terminal to an N-intein is set forth in SEQ ID NO: 31 and the nucleic acid sequence encoding said second fragment of the human DOCK8 protein fused at the N terminal with a C-intein is set forth in SEQ ID NO:35.

In some embodiments, the cell in which the system for expressing DOCK8 is a human host cell. In some embodiments, the human host cell comprises a stem cell. In some embodiments, a human stem cell is derived from bone marrow, umbilical cord, or peripheral blood, or any combination thereof. In some embodiments, a human stem cell is derived from bone marrow. In some embodiments, a human stem cell is derived from umbilical cord. In some embodiments, a human stem cell is derived from peripheral blood. In some embodiments, a human stem cell is derived from bone marrow, umbilical cord, and peripheral blood. In some embodiments, the human host cell comprises a human hematopoietic progenitor cell. In some embodiments, a human hematopoietic progenitor cell comprises a CD34⁺ cell. In some embodiments, a human host cell comprises a combination of stem cells and hematopoietic progenitor cells, which may be referred to as hematopoietic stem and progenitor cells (HSPC).

In certain embodiments, a first nucleic acid is comprised in a first lentiviral vector (first LV) and a second nucleic acid is comprised in a second lentiviral vector (second LV). In some embodiments, the first LV and the second LV are each TAT-independent and self-inactivating (SIN) lentiviral vectors. A skilled artisan would appreciate that TAT-independent and self-inactivating (SIN) lentiviral vectors and their use are well known in the art, for example but not limited to the disclosure present in Kohn et al., Autologous Ex Vivo Lentiviral Gene Therapy for Adenosine Deaminase Deficiency. N Engl J Med. 2021 May 27; 384(21):2002-2013; Cowan et al. Lentiviral Gene Therapy for Artemis-Deficient SCID. N Engl J Med. 2022 Dec. 22; 387(25):2344-2355; and Poletti and Mavilio Designing Lentiviral Vectors for Gene Therapy of Genetic Diseases. Viruses. 2021 Aug. 2; 13(8):1526. Similarly, the skilled artisan would appreciate and know how to comprise nucleic acid sequences, for example a first nucleic acid sequence or a second nucleic acid sequence in a TAT-independent and self-inactivating (SIN) lentiviral vector based on knowledge in the art.

In some embodiments, a first nucleic acid comprised in a first LV further comprises a WPRE element located 3′ of said nucleic acid encoding the N-intein and s second nucleic acid comprised in a second LV further comprises a WPRE element located 3′ of said nucleic acid encoding the second fragment of DOCK8 protein.

In some embodiments of a system, the nucleic acid sequence of a first nucleic acid comprised in a first LV is set forth in SEQ ID NO: 28 and the nucleic acid sequence of said second nucleic acid is set forth in SEQ ID NO: 29 or SEQ ID NO: 30, or the nucleic acid sequence of a first nucleic acid comprised in a first LV is set forth in SEQ ID NO: 38 and the nucleic acid sequence of said second nucleic acid is set forth in SEQ ID NO: 39. In some embodiments of a system, the nucleic acid sequence of a first nucleic acid comprised in a first LV is set forth in SEQ ID NO: 28 and the nucleic acid sequence of said second nucleic acid is set forth in SEQ ID NO: 29 or SEQ ID NO: 30. In some embodiments of a system, the nucleic acid sequence of a first nucleic acid comprised in a first LV is set forth in SEQ ID NO: 28 and the nucleic acid sequence of said second nucleic acid is set forth in SEQ ID NO: 29. In some embodiments of a system, the nucleic acid sequence of a first nucleic acid comprised in a first LV is set forth in SEQ ID NO: 28 and the nucleic acid sequence of said second nucleic acid is set forth in SEQ ID NO: 30. In some embodiments of a system, the nucleic acid sequence of a first nucleic acid comprised in a first LV is set forth in SEQ ID NO:38 and the nucleic acid sequence of said second nucleic acid is set forth in SEQ ID NO: 39.

### Lentiviral Vectors Comprising a System for Expressing Human DOCK8

In some embodiments, disclosed herein is a pair of recombinant lentiviral vectors (LVs) comprising:
a first LV comprising a first nucleic acid encoding a first fragment of a human DOCK8 protein fused at the C termina to an N-intein; and
a second LV comprising a second nucleic acid encoding a second fragment of the human DOCK8 protein fused at the N terminal with a C-intein;
wherein the first fragment of the DOCK8 protein ligated to the second fragment of the DOCK8 protein forms a full-length DOCK8 protein,
wherein the first fragment of the DOCK8 protein and the second fragment of the DOCK8 protein are selected to provide a split between said first fragment and said second fragment in an external loop that does not contribute to intra-protein interactions,
wherein said first fragment comprises the DOCK homology region 1 (DHR1) and said second fragment comprises the DOCK homology region 2 (DHR2), and
wherein said first nucleic acid further comprises a promoter located 5′ of said nucleic acid encoding the first fragment of DOCK8 and said second nucleic acid further comprises a promoter located 5′ of said nucleic acid encoding the C-intein.

In certain embodiments, said first LV and said second LV are each TAT-independent and self-inactivating (SIN) lentiviral vectors. The elements and amino acid and nucleic sequences have been described in detail above (DOCK8 Protein and Systems for Expressing a human DOCK8 protein).

In some embodiments of a pair of recombinant LVs, the nucleic acid sequence of a first LV is set forth in SEQ ID NO: 6 and the nucleic acid sequence of a second LV is set forth in SEQ ID NO: 8. In some embodiments of a pair of recombinant LVs, the nucleic acid sequence of a first LV is set forth in SEQ ID NO: 11 and the nucleic acid sequence of a second LV is set forth in SEQ ID NO: 13. One skilled in the art would appreciate that the LV backbones comprised in the LV vectors set forth in SEQ ID NO:6, 8, 11, and 13, would be interchangeable with other TAT-independent and self-inactivating (SIN) LV vector backbones, and would further understand how to build such LV vectors based on knowledge in the art and the disclosure herein of at least the DOCK8 and Intein components.

### Host Cells

In some embodiments, a host cell may be transduced with the pair of lentiviral vectors (LVs) as described herein. In some embodiments, a host cell is transduced using methods well known in the art.

In some embodiments, a host cell is a human host cell. In some embodiments, a host cell comprises any of the immune cells such as lymphocytes, neutrophils, dendritic cells, monocytes, etc. In some embodiments, the transduction is in vitro. In some embodiments, the transduced host cell expresses a full-length DOCK8 protein. In some embodiments, the transduced host cell expresses a full-length, functional DOCK8 protein. In some embodiments, the transduced host cell expresses a full-length, functional DOCK8 protein constitutively.

In some embodiments, the human host cell comprises a stem cell. In some embodiments, a human stem cell is derived from bone marrow, umbilical cord, or peripheral blood, or any combination thereof. In some embodiments, a human stem cell is derived from bone marrow. In some embodiments, a human stem cell is derived from umbilical cord. In some embodiments, a human stem cell is derived from peripheral blood. In some embodiments, a human stem cell is derived from bone marrow, umbilical cord, and peripheral blood. In some embodiments, the human host cell comprises a human hematopoietic progenitor cell. In some embodiments, a human hematopoietic progenitor cell comprises a CD34⁺ cell. In some embodiments, a human host cell comprises a combination of stem cells and hematopoietic progenitor cells, which may be referred to as hematopoietic stem and progenitor cells (HSPC).

In some embodiments, the pair of LVs comprises a system for expressing human DOCK8 protein in a cell, wherein the system comprises:
a first nucleic acid encoding a first fragment of a human DOCK8 protein fused at the C terminal to an N-intein; and
a second nucleic acid encoding a second fragment of the human DOCK8 protein fused at the N terminal with a C-intein;
wherein the first fragment of the DOCK8 protein ligated to the second fragment of the DOCK8 protein forms a full-length human DOCK8 protein, as described in detail above (DOCK8 Protein and Systems for Expressing a human DOCK8 protein).

### Treating DOCK8 Deficiency

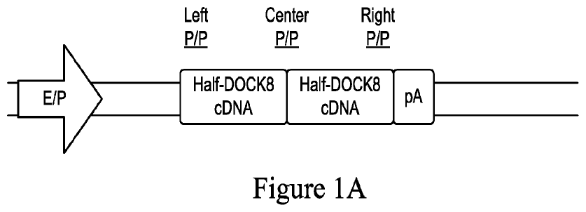

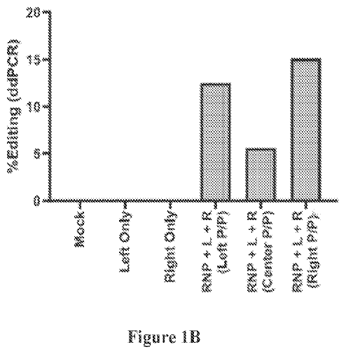

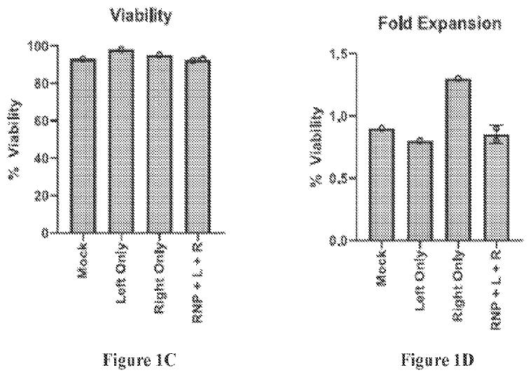

The are multiple known patient mutations in DOCK8 gene that lead to DOCK8 deficiency (See, FIG. 1 in Ravendran et al. CRISPR/Cas-Based Gene Editing Strategies for DOCK8 Immunodeficiency Syndrome. Front Genome Ed. 2022 Mar. 17; 4:793010) and it is known in the art that DOCK8 protein is expressed primarily in hematopoietic tissues.

In some embodiments, disclosed herein is a method of treating a DOCK8 deficiency in a human subject, said method comprising:
transducing a stem cell and/or progenitor cell from said subject with a pair of lentiviral vectors as described herein above (See, Sections Lentiviral Vectors comprising a System for expressing human DOCK8 & DOCK8 Protein and Systems for Expressing a human DOCK8 protein incorporated herein in their entirety) to produce transduced cells expressing functional DOCK8 protein; and
transplanting said transduced cell or cells derived therefrom into said subject, where said cells therefrom express functional DOCK8 protein.

In some embodiments, of a methods for treating a DOCK8 deficiency, wherein the DOCK8 protein is constitutively expressed in said transduced cell. In some embodiments, the constitutive expression of DOCK8 protein treats the DOCK8 deficiency. In some embodiments, the constitutive expression of DOCK8 protein improves the subject's physical wellbeing.

In some embodiments, the stem cell and or progenitor cell is (are) obtained from said subject, and is therefore autologous with the subject. This is an advantage over known therapies in the art for DOCK8 deficiency, for example, allogeneic hematopoietic stem cell transplant (HSCT) has been used to treat DOCK8 deficiency but this method frequently results in acute and or chronic Graft-versus-host disease (GvHD). Gene therapy using autologous cells, including stem cells and hematopoietic progenitor cells that are corrected with normal DOCK8 gene expression, will have a beneficial effect on blood cell production or function without the immunologic complications of HSCT.

In some embodiments, the cell is a combination of stem cells and human hematopoietic progenitor cells. In some embodiments, the stem cell is derived from bone marrow. In some embodiments, the human hematopoietic progenitor cell is a CD34+ cell. In some embodiments, the cell is a stem cell derived from bone marrow. In some embodiments, the cell is a human hematopoietic progenitor cell, and said human hematopoietic progenitor cell is a CD34+ cell.

### Additional Embodiments

Embodiment 1: A system for expressing DOCK8 protein in a cell said system comprising:
a first nucleic acid encoding a first fragment of a DOCK8 protein attached at the C termina to an N-intein (In); and
a second nucleic acid encoding a second fragment of a DOCK8 protein attached at the N terminal with a C-intein (Ic);
where the first fragment of a DOCK8 protein ligated to the second fragment of a DOCK8 protein forms a full-length normal (wildtype) DOCK8 protein.

Embodiment 2: The system of embodiment 1, wherein the N-intein and C-intein comprise a Cfa split intein where the N-intein comprises a CfaN sequence and the C-intein comprise a CfaC sequence.

Embodiment 3: The system according to any one of embodiments 1-2, wherein said the first nucleic acid encoding an N-intein and the second nucleic acid encoding said C-intein are codon optimized for expression in a human cell.

Embodiment 4: The system according to any one of embodiments 1-3, wherein the first fragment of a DOCK8 protein and the second fragment of a DOCK8 protein are selected to provide a split between said first fragment and said second fragment in an external loop that does not contribute to intra-protein interactions.

Embodiment 5: The system according to any one of embodiments 1-3, wherein the first fragment of a DOCK8 protein and the second fragment of a DOCK8 protein are selected to provide the split between said first fragment and said second fragment between DOCK homology region 1 (DHR1) and DOCK homology region 2 (DHR2).

Embodiment 6: The system of embodiment 5, wherein the first fragment of a DOCK8 protein and the second fragment of a DOCK8 protein are selected to provide the split between said first fragment and said second fragment before amino acid 939 or after amino acid 1267.

Embodiment 7: The system according to any one of embodiments 1-6, wherein said second fragment of a DOCK8 protein comprise an extein linker where said linker comprise C-F or C-F-N.

Embodiment 8: The system according to any one of embodiments 1-7, wherein said first nucleic acid is disposed in a first lentiviral vector (first LV) and said second nucleic acid is disposed in a second lentiviral vector (second LV).

Embodiment 9: The system of embodiment 8, wherein said first LV and said second LV are each TAT-independent and self-inactivating (SIN) lentiviral vectors.

Embodiment 10: The system according to any one of embodiments 8-9, wherein said lentiviral vectors each contain a WPRE element.

Embodiment 11: A set of recombinant lentiviral vectors (LVs) for the treatment of DOCK8 deficiency, said set of vectors comprising:
a first LV comprising a first nucleic acid encoding a first fragment of a DOCK8 protein attached at the C termina to an N-intein (In); and
a second LV comprising a second nucleic acid encoding a second fragment of a DOCK8 protein attached at the N terminal with a C-intein (Ic);
where the first fragment of a DOCK8 protein ligated to the second fragment of a DOCK8 protein forms a full-length normal (wildtype) DOCK8 protein.

Embodiment 12: The set of lentiviral vectors of embodiment 11, wherein the N-intein and C-intein comprise a Cfa split intein where the N-intein comprises a CfaN sequence and the C-intein comprise a CfaC sequence.

Embodiment 13: The set of lentiviral vectors according to any one of embodiments 11-12, wherein said the first nucleic acid encoding an N-intein and the second nucleic acid encoding said C-intein are codon optimized for expression in a human cell.

Embodiment 14: The set of lentiviral vectors according to any one of embodiments 11-13, wherein the first fragment of a DOCK8 protein and the second fragment of a DOCK8 protein are selected to provide a split between said first fragment and said second fragment in an external loop that does not contribute to intra-protein interactions.

Embodiment 15: The set of lentiviral vectors according to any one of embodiments 11-13, wherein the first fragment of a DOCK8 protein and the second fragment of a DOCK8 protein are selected to provide the split between said first fragment and said second fragment between DOCK homology region 1 (DHR1) and DOCK homology region 2 (DHR2).

Embodiment 16: The set of lentiviral vectors of embodiment 15, wherein the first fragment of a DOCK8 protein and the second fragment of a DOCK8 protein are selected to provide the split between said first fragment and said second fragment is before amino acid 939 or after amino acid 1267.

Embodiment 17: The set of lentiviral vectors according to any one of embodiments 11-16, wherein said second fragment of a DOCK8 protein comprise an extein linker where said linker comprise C-F or C-F-N.

Embodiment 18: The set of lentiviral vectors according to any one of embodiments 11-17, wherein said first nucleic acid is disposed in a first lentiviral vector (first LV) and said second nucleic acid is disposed in a second lentiviral vector (second LV).

Embodiment 19: The set of lentiviral vectors of embodiment 18, wherein said first LV and said second LV are each TAT-independent and self-inactivating (SIN) lentiviral vectors.

Embodiment 20: The set of lentiviral vectors according to any one of embodiments 18-19, wherein said lentiviral vectors each contain a WPRE element.

Embodiment 21: A host cell transduced with a set of lentiviral vectors according to any one of embodiments 11-20.

Embodiment 22: The host cell of embodiment 21, wherein the cell is a stem cell.

Embodiment 23: The host cell of embodiment 22, wherein said cell is a stem cell derived from bone marrow, and/or from umbilical cord blood, and/or from peripheral blood.

Embodiment 24: The host cell of embodiment 21, wherein the cell is a 293T cell.

Embodiment 25: The host cell of embodiment 21, wherein the cell is a human hematopoietic progenitor cell.

Embodiment 26: The host cell of embodiment 25, wherein the human hematopoietic progenitor cell is a CD34+ cell.

Embodiment 27: A method of treating a DOCK8 deficiency in a subject, said method comprising:
transducing a stem cell and/or progenitor cell from said subject with a system according to any one of embodiments 1-7 to produce transduced cells; and
transplanting said transduced cell or cells derived therefrom into said subject where said cells or derivatives therefrom express functional DOCK8 protein.

Embodiment 28: The method of embodiment 27, wherein the expression of DOCK8 protein recapitulates in vivo the expression pattern of the wildtype DOCK8 protein.

Embodiment 29: The method according to any one of embodiments 27-28, wherein said transducing comprises introducing a set of lentiviral vectors according to any one of embodiments 11-20.

Embodiment 30: The method according to any one of embodiments 27-29, wherein the cell is a stem cell.

Embodiment 31: The method of embodiment 30, wherein said cell is a stem cell derived from bone marrow.

Embodiment 32: The method according to any one of embodiments 27-29, wherein the cell is a human hematopoietic progenitor cell.

Embodiment 33: The method of embodiment 32, wherein the human hematopoietic progenitor cell is a CD34+ cell.

Embodiment 34: A method of introducing a nucleic acid that encodes a normal (wildtype) full length DOC8 protein into a cell, said method comprising:
using a programmable nuclease to perform targeted genome editing to introduce a first corrective nucleic acid that encodes a first fragment of a normal (non-mutated) DOCK8 protein; and
using a programmable nuclease to perform targeted genome editing to introduce a second corrective nucleic acid that encodes a second fragment of a normal (non-mutated) DOCK8 protein;
wherein said second corrective nucleic acid is inserted adjacent to said first corrective nucleic acid and said first corrective nucleic acid combined with said second corrective nucleic acid encode a full length normal DOCK8 protein.

Embodiment 35: The method of embodiment 34, wherein said programmable nuclease comprise a nuclease selected from the group consisting of a zinc finger nuclease (ZFN), a transcription activator like effector nuclease (TALEN), and a Clustered Regularly Interspaced Short Palindromic Repeats (CRISPR)/Cas9 nuclease.

Embodiment 36: The method of embodiment 35, wherein said programmable nuclease comprises a CRISPR/Cas9 nuclease.

Embodiment 37: The method according to any one of embodiments 34-36, wherein introduction of said first corrective nucleic acid into said cell is by the use of a first AAV vector and introduction of said second corrective nucleic acid into said cell is by the use of a second AAV vector.

Embodiment 38: The method of embodiment 37, wherein said first AAV vector and said second AAV vector are independently selected from the group consisting of AAV1, AAV2, AAV4, AAV5, AAV6, AAV8, and AAV9.

Embodiment 39: The method of embodiment 38 wherein said first AAV vector and said second AAV vector are AAV6 vectors.

Embodiment 40: The method according to any one of embodiments 34-39, wherein said first corrective nucleic acid and said second corrective nucleic acid are codon optimized.

Embodiment 41: The method according to any one of embodiments 34-40, wherein said cell is a cell derived from a subject that has a DOCK8 mutation.

Embodiment 42: The method of embodiment 41, wherein said cell is a hematopoietic stem cell.

Embodiment 43: The method of embodiment 41, wherein said cell is a hematopoietic cell precursor.

Embodiment 44: The method according to any one of embodiments 34-43, wherein said first corrective nucleic acid and said second corrective nucleic acid are inserted downstream of the endogenous DOCK8 promoter in said cell.

Embodiment 45: The method of embodiment 44, wherein said first corrective nucleic acid and said second corrective nucleic acid are inserted between the promoter and exon1 of the endogenous DOCK8 gene in said cell.

Embodiment 46: A method of treating a subject that has one or more DOCK8 mutations, said method comprising:
using a method according to any one of embodiments 34-45 transducing a stem cell and/or progenitor cell obtained from said subject with a nucleic acid that encodes a normal (wildtype) full length DOC8 protein to produce transduced cells; and
transplanting said transduced cell or cells derived therefrom into said subject where said cells or derivatives therefrom express a normal (wildtype) full length DOC8 protein.

Embodiment 47: The method of embodiment 46, wherein the expression of said full length DOC8 protein recapitulates in vivo the expression pattern of the wildtype DOCK8 gene.

Embodiment 48: The method according to any one of embodiments 46-47, wherein the cell is a stem cell.

Embodiment 49: The host cell of embodiment 48, wherein said cell is a stem cell derived from bone marrow.

Embodiment 50: The method according to any one of embodiments 46-47, wherein the cell is a human hematopoietic progenitor cell.

Embodiment 51: The method of embodiment 50, wherein the human hematopoietic progenitor cell is a CD34+ cell.

### Examples

### Example 1: Integration of DOCK8 cDNAs in Stem Cells Using CRISPR/Cas9 Editing

Objective: To develop a human stem cell transfer method for the treatment of DOCK8 deficiency using CRISPR/Cas9 editing of human hematopoietic stem cells.

### Methods:

sgRNA and AAV6 donor vector sequences
sgRNA e3g4:
(SEQ ID NO: 1)
uccacagggucauaaaacug
pFb-e3g4-L-shortHA (FIG. 10; GeneART sequence optimized DOCK8 fragment is from nucleotides
4488-7967)
(SEQ ID NO: 2)
gacgcgccctgtagcggcgcattaagcgcggcgggtgtggtggttacgcgcagcgtgaccgctacacttgccagcgccctagc
gcccgctcctttcgctttcttcccttcctttctcgccacgttcgccggctttccccgtcaagctctaaatcgggggctccctttagggttc
cgatttagtgctttacggcacctcgaccccaaaaaacttgattagggtgatggttcacgtagtgggccatcgccctgatagacggtttt
tcgccctttgacgttggagtccacgttctttaatagtggactcttgttccaaactggaacaacactcaaccctatctcggtctattcttttg
atttataagggattttgccgatttcggcctattggttaaaaaatgagctgatttaacaaaaatttaacgcgaattttaacaaaatattaacg
tttacaatttcaggtggcacttttcggggaaatgtgcgcggaacccctatttgtttatttttctaaatacattcaaatatgtatccgctcatg
agacaataaccctgataaatgcttcaataatattgaaaaaggaagagtatgagtattcaacatttccgtgtcgcccttattcccttttttgc
ggcattttgccttcctgtttttgctcacccagaaacgctggtgaaagtaaaagatgctgaagatcagttgggtgcacgagtgggttac
atcgaactggatctcaacagcggtaagatccttgagagttttcgccccgaagaacgttttccaatgatgagcacttttaaagttctgcta
tgtggcgcggtattatcccgtattgacgccgggcaagagcaactcggtcgccgcatacactattctcagaatgacttggttgagtact
caccagtcacagaaaagcatcttacggatggcatgacagtaagagaattatgcagtgctgccataaccatgagtgataacactgcg
gccaacttacttctgacaacgatcggaggaccgaaggagctaaccgcttttttgcacaacatgggggatcatgtaactcgccttgat
cgttgggaaccggagctgaatgaagccataccaaacgacgagcgtgacaccacgatgcctgtagcaatggcaacaacgttgcgc
aaactattaactggcgaactacttactctagcttcccggcaacaattaatagactggatggaggcggataaagttgcaggaccacttc
tgcgctcggcccttccggctggctggtttattgctgataaatctggagccggtgagcgtgggtctcgcggtatcattgcagcactgg
ggccagatggtaagccctcccgtatcgtagttatctacacgacggggagtcaggcaactatggatgaacgaaatagacagatcgct
gagataggtgcctcactgattaagcattggtaactgtcagaccaagtttactcatatatactttagattgatttaaaacttcatttttaattta
aaaggatctaggtgaagatcctttttgataatctcatgaccaaaatcccttaacgtgagttttcgttccactgagcgtcagaccccgtag
aaaagatcaaaggatcttcttgagatcctttttttctgcgcgtaatctgctgcttgcaaacaaaaaaaccaccgctaccagcggtggttt
gtttgccggatcaagagctaccaactctttttccgaaggtaactggcttcagcagagcgcagataccaaatactgtccttctagtgtag
ccgtagttaggccaccacttcaagaactctgtagcaccgcctacatacctcgctctgctaatcctgttaccagtggctgctgccagtg
gcgataagtcgtgtcttaccgggttggactcaagacgatagttaccggataaggcgcagcggtcgggctgaacggggggttcgtg
cacacagcccagcttggagcgaacgacctacaccgaactgagatacctacagcgtgagcattgagaaagcgccacgcttcccga
agggagaaaggcggacaggtatccggtaagcggcagggtcggaacaggagagcgcacgagggagcttccagggggaaacg
cctggtatctttatagtcctgtcgggtttcgccacctctgacttgagcgtcgatttttgtgatgctcgtcaggggggcggagcctatgga
aaaacgccagcaacgcggcctttttacggttcctggccttttgctggccttttgctcacatgttctttcctgcgttatcccctgattctgtg
gataaccgtattaccgcctttgagtgagctgataccgctcgccgcagccgaacgaccgagcgcagcgagtcagtgagcgaggaa
gcggaagagcgcctgatgcggtattttctccttacgcatctgtgcggtatttcacaccgcagaccagccgcgtaacctggcaaaatc
ggttacggttgagtaataaatggatgccctgcgtaagcgggtgtgggcggacaataaagtcttaaactgaacaaaatagatctaaac
tatgacaataaagtcttaaactagacagaatagttgtaaactgaaatcagtccagttatgctgtgaaaaagcatactggacttttgttatg
gctaaagcaaactcttcattttctgaagtgcaaattgcccgtcgtattaaagaggggcgtggccaagggcatggtaaagactatattc
gcggcgttgtgacaatttaccgaacaactccgcggccgggaagccgatctcggcttgaacgaattgttaggtggcggtacttgggt
cgatatcaaagtgcatcacttcttcccgtatgcccaactttgtatagagagccactgcgggatcgtcaccgtaatctgcttgcacgtag
atcacataagcaccaagcgcgttggcctcatgcttgaggagattgatgagcgcggtggcaatgccctgcctccggtgctcgccgg
agactgcgagatcatagatatagatctcactacgcggctgctcaaacctgggcagaacgtaagccgcgagagcgccaacaaccg
cttcttggtcgaaggcagcaagcgcgatgaatgtcttactacggagcaagttcccgaggtaatcggagtccggctgatgttgggag
taggtggctacgtctccgaactcacgaccgaaaagatcaagagcagcccgcatggatttgacttggtcagggccgagcctacatgt
gcgaatgatgcccatacttgagccacctaactttgttttagggcgactgccctgctgcgtaacatcgttgctgctccataacatcaaac
atcgacccacggcgtaacgcgcttgctgcttggatgcccgaggcatagactgtacaaaaaaacagtcataacaagccatgaaaac
cgccactgcgccgttaccaccgctgcgttcggtcaaggttctggaccagttgcgtgagcgcatacgctacttgcattacagtttacga
accgaacaggcttatgtcaactgggttcgtgccttcatccgtttccacggtgtgcgtcacccggcaacctgggcagcagcgaagtc
gaggcatttctgtcctggctggcgaacgagcgcaaggtttcggtctccacgcatcgtcaggcattggcggccttgctgttcttctacg
gcaaggtgctgtgcacggatctgccctggcttcaggagatcggaagacctcggccgtcgcggcgcttgccggtggtgctgaccc
cggatgaagtggttcgcatcctcggttttctggaaggcgagcatcgtttgttcgcccaggactctagctatagttctagtggttggcta
cagcttgcatgcctgcaggcagctgcgcgctcgctcgctcactgaggccgcccgggcaaagcccgggcgtcgggcgacctttg
gtcgcccggcctcagtgagcgagcgagcgcgcagagagggagtggccaactccatcactaggggttcctgcggccgcacgcg
tactagtTCTAGAggaaaattgtctccgttccctagttcactgtatttgggtaaacaaaacatggcctcggagagcctttgcggg
agaaattataaaaattcagcaaagctttgtgatctcattttggatctcctcaggattcctcatcaagttgcaattaaggagagaaaaattc
cgccttggggtgggattttaattactcatcatgttatgacttgactttctgtgctgcatttttttccatatcactacttctgtgtttgacagct
cctccaaagagtcgctccgttttatgccaggagcgaagtacttactaagtcaaaggaaagcaaggcaaacatctactattgcctttgtgc
ttttaccagaaaactgggtgagaacctccttttccttttccctgcccctccagcctcaAttCtatgaccctgtggagccagtggacttt
gaaggacttctgATGACCCACCTGAACAGCCTGGATGTGCAGCTGGCCCAAGAGCTG
GGCGACTTCACCGACGATGATCTGGACGTGGTGTTCACCCCTAAAGAGTGCAG
AACCCTGCAGCCTAGCCTGCCTGAAGAAGGCGTGGAACTGGATCCTCACGTG
CGGGATTGTGTGCAGACCTACATCCGCGAGTGGCTGATCGTGAACCGGAAGA
ATCAGGGCAGCCCAGAGATCTGCGGCTTCAAGAAAACCGGCAGCCGGAAGGA
CTTCCACAAGACCCTGCCTAAGCAGACCTTCGAGAGCGAGACACTGGAATGC
TCTGAGCCTGCTGCTCAGGCCGGACCTAGACACCTGAATGTGCTGTGTGACGT
GTCCGGCAAGGGACCTGTGACCGCCTGCGATTTCGACCTGAGAAGTCTGCAGC
CCGACAAGCGGCTGGAAAACCTGCTCCAGCAAGTGTCCGCCGAGGACTTCGA
GAAGCAGAACGAGGAAGCCAGACGGACCAACAGACAGGCCGAGCTGTTTGC
TCTGTACCCCAGCGTGGACGAGGAAGATGCCGTCGAGATTAGACCCGTGCCT
GAGTGCCCAAAAGAGCACCTGGGCAACAGAATCCTGGTCAAGCTGCTGACCC
TGAAGTTCGAGATCGAGATTGAGCCCCTGTTCGCCTCTATCGCCCTGTACGAC
GTGAAAGAGCGGAAGAAGATCAGCGAGAACTTCCACTGCGACCTGAACTCCG
ACCAGTTCAAGGGCTTCCTGAGAGCCCACACACCATCTGTGGCCGCTAGCTCT
CAGGCCAGATCTGCCGTGTTCAGCGTGACATACCCCAGCAGCGACATCTACCT
GGTGGTCAAGATCGAGAAGGTCCTGCAGCAGGGCGAGATCGGCGATTGTGCC
GAGCCTTACACCGTGATCAAAGAGAGCGACGGCGGCAAGAGCAAAGAGAAG
ATTGAGAAGCTGAAGCTGCAGGCCGAGAGCTTCTGTCAGCGGCTGGGCAAGT
ACAGAATGCCCTTTGCCTGGGCTCCTATCAGCCTGTCCAGCTTCTTCAACGTGT
CCACACTGGAACGGGAAGTGACCGACGTGGACTCTGTCGTGGGCAGATCTAG
CGTGGGCGAGAGAAGGACACTGGCTCAGTCTAGACGGCTGAGCGAGAGAGCC
CTGAGCCTGGAAGAAAATGGCGTGGGCAGCAACTTCAAGACCAGCACTCTGT
CCGTGTCTAGCTTCTTTAAGCAAGAGGGCGACAGACTGAGCGACGAGGACCT
GTTCAAGTTCCTGGCCGACTACAAGCGGAGCAGCTCCCTGCAGAGAAGAGTG
AAGTCTATCCCTGGCCTGCTGCGCCTGGAAATCTCTACAGCCCCTGAGATCAT
CAACTGCTGTCTGACCCCTGAAATGCTGCCCGTGAAGCCCTTTCCAGAGAACC
GGACCAGACCTCACAAAGAGATCCTGGAATTCCCCACCAGAGAAGTGTACGT
GCCCCACACCGTGTACCGGAACCTGCTGTATGTGTACCCACAGCGGCTGAACT
TCGTGAACAAGCTGGCCTCCGCCAGAAACATCACCATCAAGATCCAGTTTATG
TGCGGCGAGGACGCCAGCAACGCCATGCCTGTGATCTTCGGCAAGTCTAGCG
GCCCTGAGTTCCTGCAAGAGGTGTACACAGCCGTGACCTACCACAACAAGAG
CCCCGACTTCTACGAGGAAGTGAAGATTAAGCTGCCCGCCAAGCTGACCGTG
AATCACCATCTGCTGTTCACCTTCTACCACATCAGCTGCCAGCAGAAACAGGG
CGCCTCTGTGGAAACACTGCTGGGCTATAGCTGGCTGCCCATCCTGCTGAACG
AGAGACTGCAGACCGGCAGCTACTGTCTGCCTGTGGCTCTGGAAAAGCTGCC
ACCTAACTACAGCATGCACTCCGCCGAGAAGGTGCCCCTGCAGAATCCTCCTA
TTAAGTGGGCCGAGGGCCACAAGGGCGTGTTCAATATCGAGGTGCAGGCCGT
GTCCTCCGTGCACACCCAGGATAACCACCTGGAAAAGTTCTTCACCCTGTGCC
ACAGCCTCGAGAGCCAAGTGACATTCCCCATCCGCGTGCTGGACCAGAAAAT
CTCCGAGATGGCCCTGGAACACGAGCTGAAACTGAGCATCATCTGCCTGAAT
AGCAGCAGACTGGAACCCCTGGTGCTGTTCCTGCATCTGGTGCTGGACAAGCT
GTTCCAGCTGAGCGTGCAGCCCATGGTTATCGCCGGACAGACCGCCAACTTCA
GCCAGTTCGCCTTTGAGAGCGTGGTGGCCATTGCCAACAGCCTGCACAACAGC
AAGGACCTGAGCAAGGATCAGCACGGCAGAAACTGCCTGCTGGCCTCTTACG
TGCACTACGTGTTCAGACTGCCTGAGGTGCAGAGGGACGTGCCAAAATCTGG
CGCTCCTACCGCTCTGCTGGACCCCAGAAGCTATCACACCTACGGCAGAACAT
CTGCCGCCGCTGTGTCCTCTAAACTCCTGCAGGCTAGAGTGATGTCCAGCAGC
AACCCTGATCTGGCCGGAACACACAGCGCCGCTGATGAAGAAGTGAAAAACA
TCATGAGCAGCAAGATCGCCGACCGGAACTGCAGCCGGATGAGCTACTACTG
TAGCGGCAGCTCTGATGCCCCTAGCTCTCCAGCTGCTCCTAGACCTGCCAGCA
AGAAGCACTTTCACGAGGAACTGGCCCTGCAGATGGTCGTGTCTACCGGCATG
GTTCGAGAGACAGTGTTTAAGTACGCCTGGTTCTTCTTCGAACTGCTGGTCAA
GAGCATGGCCCAGCACGTGCACAACATGGACAAGCGGGACAGCTTCCGGCGG
ACCAGATTCAGCGACAGATTCATGGACGACATCACCACCATCGTGAACGTGG
TCACCAGCGAGATTGCTGCCCTGCTGGTTAAGCCCCAGAAAGAGAACGAGCA
GGCCGAAAAGATGAATATCTCTCTGGCCTTCTTCCTCTACGACCTGCTGAGCC
TGATGGACCGGGGCTTCGTGTTCAACCTGATCCGGCACTACTGCTCCCAGCTG
TCTGCCAAGCTGTCCAACCTGCCTACACTGATCTCTATGAGACTCGAGTTCCT
GAGGATCCTGTGCAGCCACGAGCACTACCTGAACCTGAATCTGTTCTTCATGA
ACGCCGACACAGCCCCTACAAGCCCCTGTCCTTCTATCAGCAGCCAGAACAGC
AGCAGCTGTAGCAGCTTTCAGGATCAGAAAATCGCCAGCATGTTCGATCTGAC
CAGCGAGTACCGGCAGCAGCACTTTCTGACCGGCCTGCTGTTTACCGAACTGG
CAGCCGCTCTTGATGCCGAAGGCGAGGGAATCAGCAAGGTGCAGCGGAAAGC
CGTGTCTGCCATTCACAGCCTGCTGTCTAGCCACGACCTGGATCCTAGATGCG
TGAAGCCTGAAGTGAAAGTGAAAATCGCTGCCCTGTACCTGCCTCTCGTGGGC
ATCATTCTGGATGCCCTGCCTCAGttttatgaccctgtggagccagtggactttgaaggacttctgatgacaca
cctgaacagcctggatgtgcagcttgcccaggagctcggggacttcactgatgacgacttggacgtggtgttcacgccaaaggaat
gtaggactttgcagccctctttgccggaggaagggtaaatagttttctaaaatgtagatgtgattgggattgtcatgattgttttcaataa
gtgggtaggggagatgccttcaatctgaacttaaaaataaaataaaattactcaatccattcaaatgtgtgggacagctatatgatatc
atcatgtaagtatagatagtttttaaattagtttggccagagattttgaaaagctatgtgaagactaaggGTAACCACGTGC
GGACCGAGCGGCCGCaggaacccctagtgatggagttggccactccctctctgcgcgctcgctcgctcactgaggc
cgggcgaccaaaggtcgcccgacgcccgggctttgcccgggcggcctcagtgagcgagcgagcgcgcagctgcctgcaggt
ctgagacaataaccctgataaatgcttcaataatgtaagctctaggagatccgaaccagataagtgaaatctagttccaaactattttgt
catttttaattttcgtattagcttacgacgctacacccagttcccatctattttgtcactcttccctaaataatccttaaaaactccatttcca
cccccccagttcccaactattttgtccgcccacagcggggcatttttcttcctgttatgtttttaatcaaacatcctgccaactccatgtga
caaaccgtcatcttcggctactttttctctgtcacagaatgaaaatttttctgtcatctcttcgttattaatgtttgtaattgactgaatatca
acgcttatttgcagcctgaatggcgaatg.
The pFb-3eg4-L-short HA plasmid includes a plasmid backbone (named pFB) is used for
packaging the AAV. pFb-3eg4-L-short HA includes DOCK8 amino acids 1-1160.
pFB-e3g4-Rflag-shortHA (FIG. 11; GeneART sequence optimized DOCK8 fragment is from nucleotides
1207-4218):
(SEQ ID NO: 3)
gacgcgccctgtagcggcgcattaagcgcggcgggtgtggtggttacgcgcagcgtgaccgctacacttgccagcgccctagc
gcccgctcctttcgctttcttcccttcctttctcgccacgttcgccggctttccccgtcaagctctaaatcgggggctccctttagggttc
cgatttagtgctttacggcacctcgaccccaaaaaacttgattagggtgatggttcacgtagtgggccatcgccctgatagacggtttt
tcgccctttgacgttggagtccacgttctttaatagtggactcttgttccaaactggaacaacactcaaccctatctcggtctattcttttg
atttataagggattttgccgatttcggcctattggttaaaaaatgagctgatttaacaaaaatttaacgcgaattttaacaaaatattaacg
tttacaatttcaggtggcacttttcggggaaatgtgcgcggaacccctatttgtttatttttctaaatacattcaaatatgtatccgctcatg
agacaataaccctgataaatgcttcaataatattgaaaaaggaagagtatgagtattcaacatttccgtgtcgcccttattcccttttttgc
ggcattttgccttcctgtttttgctcacccagaaacgctggtgaaagtaaaagatgctgaagatcagttgggtgcacgagtgggttac
atcgaactggatctcaacagcggtaagatccttgagagttttcgccccgaagaacgttttccaatgatgagcacttttaaagttctgcta
tgtggcgcggtattatcccgtattgacgccgggcaagagcaactcggtcgccgcatacactattctcagaatgacttggttgagtact
caccagtcacagaaaagcatcttacggatggcatgacagtaagagaattatgcagtgctgccataaccatgagtgataacactgcg
gccaacttacttctgacaacgatcggaggaccgaaggagctaaccgcttttttgcacaacatgggggatcatgtaactcgccttgat
cgttgggaaccggagctgaatgaagccataccaaacgacgagcgtgacaccacgatgcctgtagcaatggcaacaacgttgcgc
aaactattaactggcgaactacttactctagcttcccggcaacaattaatagactggatggaggcggataaagttgcaggaccacttc
tgcgctcggcccttccggctggctggtttattgctgataaatctggagccggtgagcgtgggtctcgcggtatcattgcagcactgg
ggccagatggtaagccccccgtatcgtagttatctacacgacggggagtcaggcaactatggatgaacgaaatagacagatcgct
gagataggtgcctcactgattaagcattggtaactgtcagaccaagtttactcatatatactttagattgatttaaaacttcatttttaattta
aaaggatctaggtgaagatcctttttgataatctcatgaccaaaatcccttaacgtgagttttcgttccactgagcgtcagaccccgtag
aaaagatcaaaggatcttcttgagatcctttttttctgcgcgtaatctgctgcttgcaaacaaaaaaaccaccgctaccagcggtggttt
gtttgccggatcaagagctaccaactctttttccgaaggtaactggcttcagcagagcgcagataccaaatactgtccttctagtgtag
ccgtagttaggccaccacttcaagaactctgtagcaccgcctacatacctcgctctgctaatcctgttaccagtggctgctgccagtg
gcgataagtcgtgtcttaccgggttggactcaagacgatagttaccggataaggcgcagcggtcgggctgaacggggggttcgtg
cacacagcccagcttggagcgaacgacctacaccgaactgagatacctacagcgtgagcattgagaaagcgccacgcttcccga
agggagaaaggcggacaggtatccggtaagcggcagggtcggaacaggagagcgcacgagggagcttccagggggaaacg
cctggtatctttatagtcctgtcgggtttcgccacctctgacttgagcgtcgatttttgtgatgctcgtcaggggggcggagcctatgga
aaaacgccagcaacgcggcctttttacggttcctggccttttgctggccttttgctcacatgttctttcctgcgttatcccctgattctgtg
gataaccgtattaccgcctttgagtgagctgataccgctcgccgcagccgaacgaccgagcgcagcgagtcagtgagcgaggaa
gcggaagagcgcctgatgcggtattttctccttacgcatctgtgcggtatttcacaccgcagaccagccgcgtaacctggcaaaatc
ggttacggttgagtaataaatggatgccctgcgtaagcgggtgtgggcggacaataaagtcttaaactgaacaaaatagatctaaac
tatgacaataaagtcttaaactagacagaatagttgtaaactgaaatcagtccagttatgctgtgaaaaagcatactggacttttgttatg
gctaaagcaaactcttcattttctgaagtgcaaattgcccgtcgtattaaagaggggcgtggccaagggcatggtaaagactatattc
gcggcgttgtgacaatttaccgaacaactccgcggccgggaagccgatctcggcttgaacgaattgttaggtggcggtacttgggt
cgatatcaaagtgcatcacttcttcccgtatgcccaactttgtatagagagccactgcgggatcgtcaccgtaatctgcttgcacgtag
atcacataagcaccaagcgcgttggcctcatgcttgaggagattgatgagcgcggtggcaatgccctgcctccggtgctcgccgg
agactgcgagatcatagatatagatctcactacgcggctgctcaaacctgggcagaacgtaagccgcgagagcgccaacaaccg
cttcttggtcgaaggcagcaagcgcgatgaatgtcttactacggagcaagttcccgaggtaatcggagtccggctgatgttgggag
taggtggctacgtctccgaactcacgaccgaaaagatcaagagcagcccgcatggatttgacttggtcagggccgagcctacatgt
gcgaatgatgcccatacttgagccacctaactttgttttagggcgactgccctgctgcgtaacatcgttgctgctccataacatcaaac
atcgacccacggcgtaacgcgcttgctgcttggatgcccgaggcatagactgtacaaaaaaacagtcataacaagccatgaaaac
cgccactgcgccgttaccaccgctgcgttcggtcaaggttctggaccagttgcgtgagcgcatacgctacttgcattacagtttacga
accgaacaggcttatgtcaactgggttcgtgccttcatccgtttccacggtgtgcgtcacccggcaaccttgggcagcagcgaagtc
gaggcatttctgtcctggctggcgaacgagcgcaaggtttcggtctccacgcatcgtcaggcattggcggccttgctgttcttctacg
gcaaggtgctgtgcacggatctgccctggcttcaggagatcggaagacctcggccgtcgcggcgcttgccggtggtgctgaccc
cggatgaagtggttcgcatcctcggttttctggaaggcgagcatcgtttgttcgcccaggactctagctatagttctagtggttggcta
cagcttgcatgcctgcaggcagctgcgcgctcgctcgctcactgaggccgcccgggcaaagcccgggcgtcgggcgacctttg
gtcgcccggcctcagtgagcgagcgagcgcgcagagagggagtggccaactccatcactaggggttcctgcggccgcacgcg
tactagtTCTAGAGTTCCTGAGGATCCTGTGCAGCCACGAGCACTACCTGAACCTG
AATCTGTTCTTCATGAACGCCGACACAGCCCCTACAAGCCCCTGTCCTTCTATC
AGCAGCCAGAACAGCAGCAGCTGTAGCAGCTTTCAGGATCAGAAAATCGCCA
GCATGTTCGATCTGACCAGCGAGTACCGGCAGCAGCACTTTCTGACCGGCCTG
CTGTTTACCGAACTGGCAGCCGCTCTTGATGCCGAAGGCGAGGGAATCAGCA
AGGTGCAGCGGAAAGCCGTGTCTGCCATTCACAGCCTGCTGTCTAGCCACGAC
CTGGATCCTAGATGCGTGAAGCCTGAAGTGAAAGTGAAAATCGCTGCCCTGT
ACCTGCCTCTCGTGGGCATCATTCTGGATGCCCTGCCTCAGCTGTGTGATTTCA
CCGTGGCCGACACCAGACGGTACAGAACCTCTGGCTCCGACGAGGAACAAGA
AGGCGCTGGCGCCATCAACCAGAATGTGGCCCTTGCCATTGCCGGGAACAAC
TTCAATCTGAAAACCAGCGGCATCGTGCTGAGCAGCCTGCCTTACAAGCAGTA
CAACATGCTGAATGCCGACACTACCCGCAACCTGATGATCTGCTTCCTGTGGA
TTATGAAGAACGCCGATCAGAGCCTGATTCGGAAGTGGATTGCCGACCTGCC
AAGCACTCAGCTGAACCGGATTCTGGACCTCCTGTTCATCTGCGTGCTGTGCT
TCGAGTACAAGGGCAAGCAGTCCAGCGACAAGGTGTCCACTCAGGTGCTGCA
GAAAAGCCGGGATGTGAAGGCCAGGCTGGAAGAGGCACTTCTGAGAGGCGA
AGGCGCCAGAGGCGAGATGATGAGGCGTAGAGCCCCTGGCAACGATAGATTC
CCCGGCCTGAATGAGAACCTGCGGTGGAAGAAAGAGCAGACCCATTGGAGAC
AGGCCAACGAGAAACTGGACAAGACCAAGGCCGAACTGGACCAAGAGGCCC
TGATCTCTGGCAACCTGGCCACAGAAGCCCACCTGATTATCCTGGACATGCAA
GAGAACATCATCCAGGCCAGTAGCGCCCTGGATTGCAAGGATTCTCTGCTCGG
AGGCGTGCTGAGGGTGCTCGTGAATAGCCTGAATTGCGACCAGAGCACCACC
TACCTGACACACTGCTTCGCCACACTGAGGGCCCTGATTGCCAAGTTTGGGGA
CCTGCTGTTCGAGGAAGAGGTGGAACAGTGCTTCGACCTGTGTCACCAAGTGC
TGCACCACTGCAGCAGCTCCATGGACGTGACAAGATCCCAGGCCTGCGCCACT
CTGTATCTGCTGATGCGGTTTAGCTTCGGCGCCACCAGCAATTTCGCCAGAGT
GAAGATGCAAGTGACCATGAGCCTGGCCAGCCTCGTTGGAAGGGCCCCTGAC
TTCAATGAGGAACACCTCAGAAGAAGCCTGCGGACCATCCTGGCCTACAGCG
AAGAGGATACCGCCATGCAGATGACCCCATTTCCAACACAAGTGGAAGAACT
GCTGTGCAACCTGAATTCCATCCTGTACGATACCGTGAAGATGAGAGAGTTCC
AAGAGGACCCCGAGATGCTGATGGATCTGATGTACCGGATCGCCAAGAGCTA
CCAGGCCTCTCCTGATCTGAGACTGACCTGGCTGCAGAACATGGCCGAGAAG
CACACCAAGAAGAAGTGCTACACCGAGGCCGCCATGTGCCTGGTTCATGCTG
CTGCACTCGTGGCCGAGTACCTGAGCATGCTGGAAGATCACTCCTACCTGCCA
GTGGGCAGCGTGTCCTTCCAGAACATCAGCTCCAACGTCCTGGAAGAGTCCGT
GGTGTCCGAGGACACACTGAGCCCAGATGAGGATGGCGTGTGTGCCGGACAG
TACTTTACCGAGTCTGGCCTCGTGGGCCTTCTGGAACAGGCCGCCGAACTGTT
TTCTACCGGCGGACTGTACGAGACAGTGAACGAAGTGTACAAGCTGGTCATC
CCCATCCTGGAAGCCCACAGAGAGTTCAGAAAGCTGACACTGACCCACTCCA
AGCTGCAGCGGGCCTTCGACAGCATTGTGAACAAGGACCACAAGCGGATGTT
CGGCACCTACTTCAGAGTGGGCTTTTTCGGCAGCAAATTCGGCGACCTGGACG
AGCAAGAGTTCGTGTACAAAGAGCCCGCCATCACCAAGCTGCCTGAGATCTC
CCATCGGCTGGAAGCCTTCTACGGCCAGTGTTTTGGCGCCGAGTTCGTGGAAG
TGATCAAGGACAGCACCCCTGTGGATAAGACCAAACTGGACCCTAACAAGGC
CTACATCCAGATCACCTTCGTGGAACCCTACTTCGACGAGTACGAGATGAAGG
ATAGAGTGACCTACTTTGAGAAGAACTTCAACCTGCGGCGGTTCATGTACACC
ACACCTTTTACACTGGAAGGCAGACCCAGGGGCGAGCTGCACGAGCAGTATA
GAAGAAACACCGTGCTGACCACCATGCACGCATTCCCTTACATCAAGACCCG
GATCAGCGTGATCCAGAAAGAAGAGTTTGTCCTGACACCAATCGAGGTGGCC
ATCGAGGACATGAAGAAGAAAACCCTCCAGCTGGCCGTGGCCATCAATCAAG
AACCTCCAGACGCCAAGATGCTCCAGATGGTGCTCCAGGGATCTGTGGGCGC
CACAGTGAATCAGGGACCTCTGGAAGTGGCCCAAGTGTTCCTGGCTGAGATCC
CCGCCGATCCTAAGCTGTACAGACACCATAACAAGCTGCGGCTCTGCTTCAAA
GAATTCATCATGAGATGTGGCGAGGCCGTGGAAAAGAACAAGAGACTGATCA
CCGCCGACCAGAGAGAGTACCAGCAAGAGCTGAAGAAAAACTACAACAAGC
TCAAAGAAAATCTGCGGCCCATGATCGAGAGAAAGATCCCCGAGCTGTACAA
GCCCATCTTCCGCGTGGAAAGCCAGAAGCGGGACTCCTTTCACAGATCCAGCT
TTAGGAAGTGCGAGACACAGCTGTCCCAGGGCTCTtcgagcgactacaaagaccatgacggtg
attataaagatcatgacatcgattacaaggatgacgatgacaagTGAGAAAAGCCATCTTCATTCGTGGA
GACTGTGGCCCTGCAACCCTGGAGAAGGACTTGCTGGTACTTAAAAAATGGG
ACATTTGCCACCCAGGACTGACTGTACACTCCCTGATCAGCCAGCACTCTGGA
AGCTTTGGGATCCCAGGAACCATGGAATTATTCCCAAATGGACTCTGACCAGA
TTTTTGCCATACTGGGGGGTGGCGGGATGGAGGATGGGTACTCAGGCATGACT
GCGTATTTATTAAAGTGTGTTTTTCCACAATGTACCAAACAAGGCATAAGCAG
CTTCTCCTGCTGACTGGCCAATCACTGCCCATCTGAGAGATGATTTCCTCTGGC
CCATATTTGAATTTATTGGAGTAACTCAAATTGCCTGAGGAAAAATGGAAAAA
TTATCCACCAGTCGATTCAAACTGAATTTCACTCTTTATAGGAAGGCAGGGCA
AACTTGTAGGAGTACGAAACATTTTCAATAAATCTACAAAGGGAAGCCTTACT
ACAATTCCAAAAATCATCATGGTTGGAAATTTGGGAGGAGATTATTTGTGAAC
TTGTTACCCTTTTGGTAATGGTGGACTAATTGCTGTATAGTTATTTTTGTTTTAT
TATTACTGTTACATTAATTTAACATGCATTTATAGAAGAATACATTCAAAGCA
CTGATGTAGGAGATACACGGTACTTGGAGCAGTCAGCCAGAAATCACAGATA
CTGCTTTCACTTAAATGGAAACAATTCTCCGATAATGCTTTGCTTTTTTTCTTA
TGTCACTCTTGTGTACTATCTATTTTTCTCCTCTCTGGGACCAAGTTTCTTTTTA
TAAAGCAATAATATCTCTGTTTTCATTTCAGAACATTGTGCTGTCTGTCAGCAT
ATGTATATCAGCTACAAAATATATTCAACTTTGACTTCTTTTGACAAAGGACTT
TAGGAAAAAGAGGAACAAAGACATTATTTGAGAATTAAATTATATATTTTTAA
TATGACTGTGACCTTGACTGATAATAAAGATGTAATAAGAATTGCAAGCTAAA
ttttatgaccctgtggagccagtggactttgaaggacttctgatgacacacctgaacagcctggatgtgcagcttgcccaggagctc
ggggacttcactgatgacgacttggacgtggtgttcacgccaaaggaatgtaggactttgcagccctctttgccggaggaagggta
aatagttttctaaaatgtagatgtgattgggattgtcatgattgttttcaataagtgggtaggggagatgccttcaatctgaacttaaaaat
aaaataaaattactcaatccattcaaatgtgtgggacagctatatgatatcatcatgtaagtatagatagtttttaaattagtttggccaga
gattttgaaaagctatgtgaagactaaggGTAACCACGTGCGGACCGAGCGGCCGCaggaacccctagt
gatggagttggccactccctctctgcgcgctcgctcgctcactgaggccgggcgaccaaaggtcgcccgacgcccgggctttgc
ccgggcggcctcagtgagcgagcgagcgcgcagctgcctgcaggtctgagacaataaccctgataaatgcttcaataatgtaagc
tctaggagatccgaaccagataagtgaaatctagttccaaactattttgtcatttttaattttcgtattagcttacgacgctacacccagttc
ccatctattttgtcactcttccctaaataatccttaaaaactccatttccacccctcccagttcccaactattttgtccgcccacagcgggg
catttttcttcctgttatgtttttaatcaaacatcctgccaactccatgtgacaaaccgtcatcttcggctactttttctctgtcacagaatgaa
aatttttctgtcatctcttcgttattaatgtttgtaattgactgaatatcaacgcttatttgcagcctgaatggcgaatg.

The pFB-e3g4-Rflag-shortHA plasmid includes a plasmid backbone (named pFB) is used for packaging the AAV. pFB-e3g4-Rflag-shortHA includes DOCK8 amino acids 1161-2099.

The pFb-e3g4-L-shortHA and pFB-e3g4-Rflag-shortHA comprising the 5′ and 3′ DOCK8 halves were designed based on the CRISPR editing approach.

Healthy donor derived peripheral blood stem cells (PBSC) were pre-stimulated in X-VIVO15 Serum-free Hematopoietic Cell Medium (Lonza Bioscience) with 50 ng/mL Human Stem Cell Factor (hSCF; Peprotech), 50 ng/mL human fins like tyrosine kinase 3 (hFlt-3; Peprotech) ligand, and 50 ng/mL human thrombopoietin (hTPO; Peprotech). After two days in culture, DOCK8 sgRNA e3g4 (SEQ ID NO: 1) pre-complexed to Cas9 protein were delivered to PBSC as ribonucleoprotein complexes using the BTX square wave electroporator (250 V, 5 mS; Harvard Apparatus).

PBSC cells used herein comprise a mix of stem and progenitor cells, also called HSPC—Hematopoietic Stem and Progenitor Cells.

Immediately following electroporation, cells were transduced with the two AAV6 donors, each containing half of the DOCK8 cDNA with their respective homology arms (SEQ ID NO: 2 and SEQ ID NO: 3) for 16-24 hours to allow for site specific integration.

Rates of site specific integration were quantified by droplet digital PCR with three different sets of probes, one binding in the 5′ end of the cDNA donor, one in the 3′ end, and one where the two halves of the DOCK8 cDNA come together (FIGS. 1A and 1B).

Viability and fold expansion were measured by trypan blue exclusion 24 hours after treatment (FIGS. 1C and 1D).

### Results:

Gene integration occurred in 12.5% of cells when measured with the Left Primer/Probe set, while 15.1% gene integration was quantified using the Right Primer/Probe set. The Center Primer/Probe set measured 5.6% gene integration (FIG. 1B). The center P/P is measuring integration of both halves of the DOCK8 cDNA by the two AAV donors, while the Left and Right P/P are also detecting events in which only the pFb-e3g4-L-shortHA (SEQ ID NO: 2) or pFB-e3g4-Rflag-shortHA (SEQ ID NO: 3) has integrated independent of the other. Use of the dual AAV system does not significantly affect cell viability and fold expansion as measured by trypan blue exclusion (FIGS. 1C and 1D).

These results demonstrated the sequential integration of DOCK8 cDNA (e3g4-L-short HA donor was integrated first followed by integration of the e3g4-Rflag-short HA) is enabled by two AAV6 donors in peripheral blood stem cell (PBSC).

### Example 2: Integration of Full-Length DOCK8 cDNAs in Stem Cells Using Lentiviral Vectors

Objective: To develop a human stem cell transfer method for the treatment of DOCK8 deficiency using Lentiviral vector transduction of human hematopoietic stem cells approach.

### Materials

### Vector and Component Sequences

### Elongation Factor 1 Alpha Short (EFS):

(SEQ ID NO: 40)
ggctccggtgcccgtcagtgggcagagcgcacatcgcccacagtccccg
agaagttggggggaggggtcggcaattgaaccggtgcctagagaaggtg
gcgcggggtaaactgggaaagtgatgtcgtgtactggctccgccttttt
cccgagggtgggggagaaccgtatataagtgcagtagtcgccgtgaacg
ttctttttcgcaacgggtttgccgccagaacacaggtgtcgtga.

### Woodchuck Hepatitis Virus Post-Transcriptional Regulatory Element (WPRE):

(SEQ ID NO: 41)
ttccgataatcaacctctggattacaaaatttgtgaaagattgactggt
attcttaactatgttgctccttttacgctatgtggatacgctgctttaa
tgcctttgtatcatgctattgcttcccgtatggctttcattttctcctc
cttgtataaatcctggttgctgtctctttatgaggagttgtggcccgtt
gtcaggcaacgtggcgtggtgtgcactgtgtttgctgacgcaaccccca
ctggttggggcattgccaccacctgtcagctcctttccgggactttcgc
tttccccctccctattgccacggcggaactcatcgccgcctgccttgcc
cgctgctggacaggggctcggctgttgggcactgacaattccgtggtgt
tgtcggggaaATCATcgtcctttccTtggctgctcgcctgtgttgccac
ctggattctgcgcgggacgtccttctgctacgtcccttcggccctcaat
ccagcggaccttccttcccgcggcctgctgccggctctgcggcctcttc
cgcgtcttcgccttcgccctcagacgagtcggatctccctttgggccgc
ctccccgc

mCCL-EFS-DOCK8ga-WPRE (FIG. 12):

(SEQ ID NO: 4)
acgcgccctgtagcggcgcattaagcgcggcgggtgtggtggttacgcgcagcgtgaccgctacacttgccagcgccctagcgc
ccgctcctttcgctttcttcccttcctttctcgccacgttcgccggctttccccgtcaagctctaaatcgggggctccctttagggttccg
atttagtgctttacggcacctcgaccccaaaaaacttgattagggtgatggttcacgtagtgggccatcgccctgatagacggtttttc
gccctttgacgttggagtccacgttctttaatagtggactcttgttccaaactggaacaacactcaaccctatctcggtctattcttttgatt
tataagggattttgccgatttcggcctattggttaaaaaatgagctgatttaacaaaaatttaacgcgaattttaacaaaatattaacgctt
acaatttaggtggcacttttcggggaaatgtgcgcggaacccctatttgtttatttttctaaatacattcaaatatgtatccgctcatgaga
caataaccctgataaatgcttcaataatagcacctagatcaagagacaggatgaggatcgtttcgcatgattgaacaagatggattgc
acgcaggttctccggccgcttgggtggagaggctattcggctatgactgggcacaacagacaatcggctgctctgatgccgccgt
gttccggctgtcagcgcaggggcgcccggttctttttgtcaagaccgacctgtccggtgccctgaatgaactgcaagacgaggca
gcgcggctatcgtggctggccacgacgggcgttccttgcgcagctgtgctcgacgttgtcactgaagcgggaagggactggctg
ctattgggcgaagtgccggggcaggatctcctgtcatctcaccttgctcctgccgagaaagtatccatcatggctgatgcaatgcgg
cggctgcatacgcttgatccggctacctgcccattcgaccaccaagcgaaacatcgcatcgagcgagcacgtactcggatggaag
ccggtcttgtcgatcaggatgatctggacgaagagcatcaggggctcgcgccagccgaactgttcgccaggctcaaggcgagca
tgcccgacggcgaggatctcgtcgtgacccatggcgatgcctgcttgccgaatatcatggtggaaaatggccgcttttctggattcat
cgactgtggccggctgggtgtggcggaccgctatcaggacatagcgttggctacccgtgatattgctgaagagcttggcggcgaa
tgggctgaccgcttcctcgtgctttacggtatcgccgctcccgattcgcagcgcatcgccttctatcgccttcttgacgagttcttctga
attattaacgcttacaatttcctgatgcggtattttctccttacgcatctgtgcggtatttcacaccgcatcaggtggcacttttcggggaa
atgtgcgcggaacccctatttgtttatttttctaaatacattcaaatatgtatccgctcatgaccaaaatcccttaacgtgagttttcgttcc
actgagcgtcagaccccgtagaaaagatcaaaggatcttcttgagatcctttttttctgcgcgtaatctgctgcttgcaaacaaaaaaa
ccaccgctaccagcggtggtttgtttgccggatcaagagctaccaactctttttccgaaggtaactggcttcagcagagcgcagata
ccaaatactgttcttctagtgtagccgtagttaggccaccacttcaagaactctgtagcaccgcctacatacctcgctctgctaatcctg
ttaccagtggctgctgccagtggcgataagtcgtgtcttaccgggttggactcaagacgatagttaccggataaggcgcagcggtc
gggctgaacgggggggtcgtgcacacagcccagcttggagcgaacgacctacaccgaactgagatacctacagcgtgagctat
gagaaagcgccacgcttcccgaagggagaaaggcggacaggtatccggtaagcggcagggtcggaacaggagagcgcacg
agggagcttccagggggaaacgcctggtatctttatagtcctgtcgggtttcgccacctctgacttgagcgtcgatttttgtgatgctc
gtcaggggggcggagcctatggaaaaacgccagcaacgcggcctttttacggttcctggccttttgctggccttttgctcacatgttc
tttcctgcgttatcccctgattctgtggataaccgtattaccgcctttgagtgagctgataccgctcgccgcagccgaacgaccgagc
gcagcgagtcagtgagcgaggaagcggaagagcgcccaatacgcaaaccgcctctccccgcgcgttggccgattcattaatgc
agctggcacgacaggtttcccgactggaaagcgggcagtgagcgcaacgcaattaatgtgagttagctcactcattaggcacccc
aggctttacactttatgcttccggctcgtatgttgtgtggaattgtgagcggataacaatttcacacaggaaacagctatgaccatgatt
acgccaagcgcgcaattaaccctcactaaagggaacaaaagctggagctgcaagcttggccattgcatacgttgtatccatatcata
atatgtacatttatattggctcatgtccaacattaccgccatgttgacattgattattgactagttattaatagtaatcaattacggggtcatt
agttcatagcccatatatggagttccgcgttacataacttacggtaaatggcccgcctggctgaccgcccaacgacccccgcccatt
gacgtcaataatgacgtatgttcccatagtaacgccaatagggactttccattgacgtcaatgggggagtatttacggtaaactgccc
acttggcagtacatcaagtgtatcatatgccaagtacgccccctattgacgtcaatgacggtaaatggcccgcctggcattatgccca
gtacatgaccttatgggactttcctacttggcagtacatctacgtattagtcatcgctattaccatggtgatgcggttttggcagtacatc
aatgggcgtggatagcggtttgactcacggggatttccaagtctccaccccattgacgtcaatgggagtttgttttggcaccaaaatc
aacgggactttccaaaatgtcgtaacaactccgccccattgacgcaaatgggcggtaggcgtgtacggtgggaggtctatataagc
agagctcgtttagtgaaccgGGGTCTCTCTGGTTAGACCAGATCTGAGCCTGGGAGCTCTC
TGGCTAACTAGGGAACCCACTGCTTAAGCCTCAATAAAGCTTGCCTTGAGTGC
TTCAAGTAGTGTGTGCCCGTCTGTTGTGTGACTCTGGTAACTAGAGATCCCTC
AGACCCTTTTAGTCAGTGTGGAAAATCTCTAGCagtggcgcccgaacagggacttgaaagcgaa
agggaaaccagaggagctctcTCGACGCAGGACTCGGCTTGCTGAAGCGCGCACGGCAAG
AGGCGAGGGGCGGCGACTGGTGAGTACGCCAAAAATTTTGACTAGCGGAGGC
TAGAAGGAGAGAGATGGGTGCGAGAGCGTCAGTATTAAGCGGGGGAGaattagat
cgcgatgggaaaaaattcggttaaggccagggggaaagaaaaaatataaattaaaacatatagtatgggcaagcagggagctag
aacgattcgcagttaatcctggcctgttagaaacatcagaaggctgtagacaaatactgggacagctacaaccatcccttcagacag
gatcagaagaacttagatcattatataatacagtagcaaccctctattgtgtgcatcaaaggatagagataaaagacaccaaggaag
ctttagacaagatagaggaagagcaaaacaaaagtaagaccaccgcacagcaagcGGCCGCTgatcttcagacctggag
gaggagatatgagggacaattggagaagtgaattatataaatataaagtagtaaaaattgaaccattaggagtagcacccaccaag
gcaaagagaagagtggtgcagagagaaaaaagaGCAGTGGGAATaggagctttgttccttgggttcttgggagcagca
ggaagcactatgggcgcagcgtcaatgacgctgacggtacaggccagacaattattgtctggtatagtgcagcagcagaacaattt
gctgagggctattgaggcgcaacagcatctgttgcaactcacagtctggggcatcaagcagctccaggcaagaatcctggctgtg
gaaagatacctaaaggatcaacagctcctggggatttggggttgctctggaaaactcatttgcaccactgctgtgccttggaatgcta
gttggagtaataaatctctggaacagatttggaatcacacgacctggatggagtgggacagagaaattaacaattacacaagcttaa
tacactccttaattgaagaatcgcaaaaccagcaagaaaagaatgaacaagaattattggaattagataaatgggcaagtttgtgga
attggtttaacataacaaattggctgtggtatataaaattattcataatgatagtaggaggcttggtaggtttaagaatagtttttgctgtac
tttctatagtgaatagagttaggcagggatattcaccattatcgtttcagacccacctcccaaccccgaggggacccgacaggcccg
aaggaatagaagaagaaggtggagagagagacagagacagatccattcgattagtgaacggatCacaaatggcagtattcatcc
acaattttaaaagaaaaggggggattggggggtacagtgcaggggaaagaatagtagacataatagcaacagacatacaaacta
aagaattacaaaaacaaattacaaaaattcaaaattttcgggtttattacagggacagcagaggctccggtgcccgtcagtgggcag
agcgcacatcgcccacagtccccgagaagttggggggaggggtcggcaattgaaccggtgcctagagaaggtggcgcggggt
aaactgggaaagtgatgtcgtgtactggctccgcctttttcccgagggtgggggagaaccgtatataagtgcagtagtcgccgtga
acgttctttttcgcaacgggtttgccgccagaacacaggtgtcgtgaGACAGACGAGGTTTGCGCTTGGCTGG
GCATGTTCCGCGGCTACTCTGCGGCGCGCCAGGCCCCCGCTTTCCGCACCCCGC
GACCCTAGAAGCCACCGAACCGCCGGCGGGCCATGGCCACACTGCCTAGCGCTG
AGAGAAGGGCCTTCGCTCTGAAGATCAACCGGTACAGCAGCGCCGAGATCCGGAA
GCAGTTTACCCTGCCTCCTAACCTGGGCCAGTACCACAGACAGAGCATCAGCACCA
GCGGCTTCCCATCTCTGCAGCTGCCCCAGTTCTACGACCCTGTGGAACCCGTGGAT
TTCGAGGGCCTGCTGATGACCCACCTGAACAGCCTGGATGTGCAGCTGGCCCAAG
AGCTGGGCGACTTCACCGACGATGATCTGGACGTGGTGTTCACCCCTAAAGAGTGC
AGAACCCTGCAGCCTAGCCTGCCTGAAGAAGGCGTGGAACTGGATCCTCACGTGC
GGGATTGTGTGCAGACCTACATCCGCGAGTGGCTGATCGTGAACCGGAAGAATCA
GGGCAGCCCAGAGATCTGCGGCTTCAAGAAAACCGGCAGCCGGAAGGACTTCCAC
AAGACCCTGCCTAAGCAGACCTTCGAGAGCGAGACACTGGAATGCTCTGAGCCTG
CTGCTCAGGCCGGACCTAGACACCTGAATGTGCTGTGTGACGTGTCCGGCAAGGG
ACCTGTGACCGCCTGCGATTTCGACCTGAGAAGTCTGCAGCCCGACAAGCGGCTG
GAAAACCTGCTCCAGCAAGTGTCCGCCGAGGACTTCGAGAAGCAGAACGAGGAAG
CCAGACGGACCAACAGACAGGCCGAGCTGTTTGCTCTGTACCCCAGCGTGGACGA
GGAAGATGCCGTCGAGATTAGACCCGTGCCTGAGTGCCCAAAAGAGCACCTGGGC
AACAGAATCCTGGTCAAGCTGCTGACCCTGAAGTTCGAGATCGAGATTGAGCCCCT
GTTCGCCTCTATCGCCCTGTACGACGTGAAAGAGCGGAAGAAGATCAGCGAGAACT
TCCACTGCGACCTGAACTCCGACCAGTTCAAGGGCTTCCTGAGAGCCCACACACCA
TCTGTGGCCGCTAGCTCTCAGGCCAGATCTGCCGTGTTCAGCGTGACATACCCCAG
CAGCGACATCTACCTGGTGGTCAAGATCGAGAAGGTCCTGCAGCAGGGCGAGATC
GGCGATTGTGCCGAGCCTTACACCGTGATCAAAGAGAGCGACGGCGGCAAGAGCA
AAGAGAAGATTGAGAAGCTGAAGCTGCAGGCCGAGAGCTTCTGTCAGCGGCTGGG
CAAGTACAGAATGCCCTTTGCCTGGGCTCCTATCAGCCTGTCCAGCTTCTTCAACGT
GTCCACACTGGAACGGGAAGTGACCGACGTGGACTCTGTCGTGGGCAGATCTAGC
GTGGGCGAGAGAAGGACACTGGCTCAGTCTAGACGGCTGAGCGAGAGAGCCCTG
AGCCTGGAAGAAAATGGCGTGGGCAGCAACTTCAAGACCAGCACTCTGTCCGTGTC
TAGCTTCTTTAAGCAAGAGGGCGACAGACTGAGCGACGAGGACCTGTTCAAGTTCC
TGGCCGACTACAAGCGGAGCAGCTCCCTGCAGAGAAGAGTGAAGTCTATCCCTGG
CCTGCTGCGCCTGGAAATCTCTACAGCCCCTGAGATCATCAACTGCTGTCTGACCC
CTGAAATGCTGCCCGTGAAGCCCTTTCCAGAGAACCGGACCAGACCTCACAAAGAG
ATCCTGGAATTCCCCACCAGAGAAGTGTACGTGCCCCACACCGTGTACCGGAACCT
GCTGTATGTGTACCCACAGCGGCTGAACTTCGTGAACAAGCTGGCCTCCGCCAGAA
ACATCACCATCAAGATCCAGTTTATGTGCGGCGAGGACGCCAGCAACGCCATGCCT
GTGATCTTCGGCAAGTCTAGCGGCCCTGAGTTCCTGCAAGAGGTGTACACAGCCGT
GACCTACCACAACAAGAGCCCCGACTTCTACGAGGAAGTGAAGATTAAGCTGCCCG
CCAAGCTGACCGTGAATCACCATCTGCTGTTCACCTTCTACCACATCAGCTGCCAG
CAGAAACAGGGCGCCTCTGTGGAAACACTGCTGGGCTATAGCTGGCTGCCCATCC
TGCTGAACGAGAGACTGCAGACCGGCAGCTACTGTCTGCCTGTGGCTCTGGAAAA
GCTGCCACCTAACTACAGCATGCACTCCGCCGAGAAGGTGCCCCTGCAGAATCCTC
CTATTAAGTGGGCCGAGGGCCACAAGGGCGTGTTCAATATCGAGGTGCAGGCCGT
GTCCTCCGTGCACACCCAGGATAACCACCTGGAAAAGTTCTTCACCCTGTGCCACA
GCCTCGAGAGCCAAGTGACATTCCCCATCCGCGTGCTGGACCAGAAAATCTCCGA
GATGGCCCTGGAACACGAGCTGAAACTGAGCATCATCTGCCTGAATAGCAGCAGAC
TGGAACCCCTGGTGCTGTTCCTGCATCTGGTGCTGGACAAGCTGTTCCAGCTGAGC
GTGCAGCCCATGGTTATCGCCGGACAGACCGCCAACTTCAGCCAGTTCGCCTTTGA
GAGCGTGGTGGCCATTGCCAACAGCCTGCACAACAGCAAGGACCTGAGCAAGGAT
CAGCACGGCAGAAACTGCCTGCTGGCCTCTTACGTGCACTACGTGTTCAGACTGCC
TGAGGTGCAGAGGGACGTGCCAAAATCTGGCGCTCCTACCGCTCTGCTGGACCCC
AGAAGCTATCACACCTACGGCAGAACATCTGCCGCCGCTGTGTCCTCTAAACTCCT
GCAGGCTAGAGTGATGTCCAGCAGCAACCCTGATCTGGCCGGAACACACAGCGCC
GCTGATGAAGAAGTGAAAAACATCATGAGCAGCAAGATCGCCGACCGGAACTGCAG
CCGGATGAGCTACTACTGTAGCGGCAGCTCTGATGCCCCTAGCTCTCCAGCTGCTC
CTAGACCTGCCAGCAAGAAGCACTTTCACGAGGAACTGGCCCTGCAGATGGTCGT
GTCTACCGGCATGGTTCGAGAGACAGTGTTTAAGTACGCCTGGTTCTTCTTCGAACT
GCTGGTCAAGAGCATGGCCCAGCACGTGCACAACATGGACAAGCGGGACAGCTTC
CGGCGGACCAGATTCAGCGACAGATTCATGGACGACATCACCACCATCGTGAACGT
GGTCACCAGCGAGATTGCTGCCCTGCTGGTTAAGCCCCAGAAAGAGAACGAGCAG
GCCGAAAAGATGAATATCTCTCTGGCCTTCTTCCTCTACGACCTGCTGAGCCTGATG
GACCGGGGCTTCGTGTTCAACCTGATCCGGCACTACTGCTCCCAGCTGTCTGCCAA
GCTGTCCAACCTGCCTACACTGATCTCTATGAGACTCGAGTTCCTGAGGATCCTGT
GCAGCCACGAGCACTACCTGAACCTGAATCTGTTCTTCATGAACGCCGACACAGCC
CCTACAAGCCCCTGTCCTTCTATCAGCAGCCAGAACAGCAGCAGCTGTAGCAGCTT
TCAGGATCAGAAAATCGCCAGCATGTTCGATCTGACCAGCGAGTACCGGCAGCAGC
ACTTTCTGACCGGCCTGCTGTTTACCGAACTGGCAGCCGCTCTTGATGCCGAAGGC
GAGGGAATCAGCAAGGTGCAGCGGAAAGCCGTGTCTGCCATTCACAGCCTGCTGT
CTAGCCACGACCTGGATCCTAGATGCGTGAAGCCTGAAGTGAAAGTGAAAATCGCT
GCCCTGTACCTGCCTCTCGTGGGCATCATTCTGGATGCCCTGCCTCAGCTGTGTGA
TTTCACCGTGGCCGACACCAGACGGTACAGAACCTCTGGCTCCGACGAGGAACAA
GAAGGCGCTGGCGCCATCAACCAGAATGTGGCCCTTGCCATTGCCGGGAACAACT
TCAATCTGAAAACCAGCGGCATCGTGCTGAGCAGCCTGCCTTACAAGCAGTACAAC
ATGCTGAATGCCGACACTACCCGCAACCTGATGATCTGCTTCCTGTGGATTATGAAG
AACGCCGATCAGAGCCTGATTCGGAAGTGGATTGCCGACCTGCCAAGCACTCAGCT
GAACCGGATTCTGGACCTCCTGTTCATCTGCGTGCTGTGCTTCGAGTACAAGGGCA
AGCAGTCCAGCGACAAGGTGTCCACTCAGGTGCTGCAGAAAAGCCGGGATGTGAA
GGCCAGGCTGGAAGAGGCACTTCTGAGAGGCGAAGGCGCCAGAGGCGAGATGAT
GAGGCGTAGAGCCCCTGGCAACGATAGATTCCCCGGCCTGAATGAGAACCTGCGG
TGGAAGAAAGAGCAGACCCATTGGAGACAGGCCAACGAGAAACTGGACAAGACCA
AGGCCGAACTGGACCAAGAGGCCCTGATCTCTGGCAACCTGGCCACAGAAGCCCA
CCTGATTATCCTGGACATGCAAGAGAACATCATCCAGGCCAGTAGCGCCCTGGATT
GCAAGGATTCTCTGCTCGGAGGCGTGCTGAGGGTGCTCGTGAATAGCCTGAATTG
CGACCAGAGCACCACCTACCTGACACACTGCTTCGCCACACTGAGGGCCCTGATTG
CCAAGTTTGGGGACCTGCTGTTCGAGGAAGAGGTGGAACAGTGCTTCGACCTGTGT
CACCAAGTGCTGCACCACTGCAGCAGCTCCATGGACGTGACAAGATCCCAGGCCT
GCGCCACTCTGTATCTGCTGATGCGGTTTAGCTTCGGCGCCACCAGCAATTTCGCC
AGAGTGAAGATGCAAGTGACCATGAGCCTGGCCAGCCTCGTTGGAAGGGCCCCTG
ACTTCAATGAGGAACACCTCAGAAGAAGCCTGCGGACCATCCTGGCCTACAGCGAA
GAGGATACCGCCATGCAGATGACCCCATTTCCAACACAAGTGGAAGAACTGCTGTG
CAACCTGAATTCCATCCTGTACGATACCGTGAAGATGAGAGAGTTCCAAGAGGACC
CCGAGATGCTGATGGATCTGATGTACCGGATCGCCAAGAGCTACCAGGCCTCTCCT
GATCTGAGACTGACCTGGCTGCAGAACATGGCCGAGAAGCACACCAAGAAGAAGT
GCTACACCGAGGCCGCCATGTGCCTGGTTCATGCTGCTGCACTCGTGGCCGAGTA
CCTGAGCATGCTGGAAGATCACTCCTACCTGCCAGTGGGCAGCGTGTCCTTCCAGA
ACATCAGCTCCAACGTCCTGGAAGAGTCCGTGGTGTCCGAGGACACACTGAGCCC
AGATGAGGATGGCGTGTGTGCCGGACAGTACTTTACCGAGTCTGGCCTCGTGGGC
CTTCTGGAACAGGCCGCCGAACTGTTTTCTACCGGCGGACTGTACGAGACAGTGAA
CGAAGTGTACAAGCTGGTCATCCCCATCCTGGAAGCCCACAGAGAGTTCAGAAAGC
TGACACTGACCCACTCCAAGCTGCAGCGGGCCTTCGACAGCATTGTGAACAAGGAC
CACAAGCGGATGTTCGGCACCTACTTCAGAGTGGGCTTTTTCGGCAGCAAATTCGG
CGACCTGGACGAGCAAGAGTTCGTGTACAAAGAGCCCGCCATCACCAAGCTGCCT
GAGATCTCCCATCGGCTGGAAGCCTTCTACGGCCAGTGTTTTGGCGCCGAGTTCGT
GGAAGTGATCAAGGACAGCACCCCTGTGGATAAGACCAAACTGGACCCTAACAAGG
CCTACATCCAGATCACCTTCGTGGAACCCTACTTCGACGAGTACGAGATGAAGGAT
AGAGTGACCTACTTTGAGAAGAACTTCAACCTGCGGCGGTTCATGTACACCACACC
TTTTACACTGGAAGGCAGACCCAGGGGCGAGCTGCACGAGCAGTATAGAAGAAAC
ACCGTGCTGACCACCATGCACGCATTCCCTTACATCAAGACCCGGATCAGCGTGAT
CCAGAAAGAAGAGTTTGTCCTGACACCAATCGAGGTGGCCATCGAGGACATGAAGA
AGAAAACCCTCCAGCTGGCCGTGGCCATCAATCAAGAACCTCCAGACGCCAAGATG
CTCCAGATGGTGCTCCAGGGATCTGTGGGCGCCACAGTGAATCAGGGACCTCTGG
AAGTGGCCCAAGTGTTCCTGGCTGAGATCCCCGCCGATCCTAAGCTGTACAGACAC
CATAACAAGCTGCGGCTCTGCTTCAAAGAATTCATCATGAGATGTGGCGAGGCCGT
GGAAAAGAACAAGAGACTGATCACCGCCGACCAGAGAGAGTACCAGCAAGAGCTG
AAGAAAAACTACAACAAGCTCAAAGAAAATCTGCGGCCCATGATCGAGAGAAAGATC
CCCGAGCTGTACAAGCCCATCTTCCGCGTGGAAAGCCAGAAGCGGGACTCCTTTCA
CAGATCCAGCTTTAGGAAGTGCGAGACACAGCTGTCCCAGGGCTCTTGAttccgataatc
aacctctggattacaaaatttgtgaaagattgactggtattcttaactatgttgctccttttacgctatgtggatacgctgctttaatgccttt
gtatcatgctattgcttcccgtatggctttcattttctcctccttgtataaatcctggttgctgtctctttatgaggagttgtggcccgttgtca
ggcaacgtggcgtggtgtgcactgtgtttgctgacgcaacccccactggttggggcattgccaccacctgtcagctcctttccggga
ctttcgctttccccctccctattgccacggcggaactcatcgccgcctgccttgcccgctgctggacaggggctcggctgttgggca
ctgacaattccgtggtgttgtcggggaaATCATcgtcctttccTtggctgctcgcctgtgttgccacctggattctgcgcgggac
gtccttctgctacgtcccttcggccctcaatccagcggaccttccttcccgcggcctgctgccggctctgcggcctcttccgcgtcttc
gccttcgccctcagacgagtcggatctccctttgggccgcctccccgcTaaaagaaaaggggggactggaagggctaattcact
cccaacgaagacaagatctgctttttgcttgtactGGGTCTCTCTGGTTAGACCAGATCTGAGCCTGG
GAGCTCTCTGGCTAACTAGGGAACCCACTGCTTAAGCCTCAATAAAGCTTGCC
TTGAGTGCTTCAAGTAGTGTGTTGGTTTTTTGTGTGTGCATTGTCTGAGTAGGT
GTCATTCTATTCTGGGGGGTGGGGTGGGGCAGCACAGCAAGGGGGAGGATTG
GGAAGACAATAGCAGGCATGCTGGGGATGCGGTGGGCTCTATGGagtagtagttcatg
tcatcttattattcagtatttataacttgcaaagaaatgaatatcagagagtgagaggaacttgtttattgcagcttataatggttacaaata
aagcaatagcatcacaaatttcacaaataaagcatttttttcactgcattctagttgtggtttgtccaaactcatcaatgtatcttatcatgtc
tggctctagctatcccgcccctaactccgcccATCCCGCCCCTAACTCCGCCCAGTTCCGCCCATT
CTCCGCCCCATGGCTCTCACTACTTCTGGAATAGCTCAGAGGCCGAGGCGGCC
TCGGCCTCTGCATAAATAAAAAAAATTAGTCGAGGCTTTTTTGGAGGCCTAGG
gacgtacccaattcgccctatagtgagtcgtattacgcgcgctcactggccgtcgttttacaacgtcgtgactgggaaaaccctggc
gttacccaacttaatcgccttgcagcacatccccctttcgccagctggcgtaatagcgaagaggcccgcaccgatcgcccttccca
acagttgcgcagcctgaatggcgaatggg.

The nucleic acid sequence encoding the GeneArt optimized DOCK8 protein is Italicized and represents nucleotide positions 5430-11729 of the mCCL-EFS-DOCK8ga-WPRE vector. A description of the lentiviral transfer vector construct containing the CMV-HIV 5′LTR for the mCCL vectors disclosed (including SEQ ID NOs: 4, 6, 8, 11, and 13) herein can be found in Dull et al., A third-generation lentivirus vector with a conditional packaging system. J Virol. 1998 November; 72(11):8463-71.

### Methods and Results:

PBSC cells used herein comprise a mix of stem and progenitor cells, also called HSPC—Hematopoietic Stem and Progenitor Cells.

Given the constitutive nature of DOCK8 expression in cells of hematopoietic lineage, as a first step, the feasibility of gene addition through lentiviral vectors was evaluated. HIV-1 based lentiviral vectors in which the LTR is deleted and the transgene is driven by an internal promoter were used in this approach as insertional mutagenesis is a concern with gamma-retroviral vectors. Self-inactivating lentiviral vectors not only possess a safer vector integration profile but an improved ability to transduce quiescent, engrafting HSC. (Zufferey et al., Self-inactivating lentivirus vector for safe and efficient in vivo gene delivery. J Virol. 1998 December; 72(12):9873-80.)

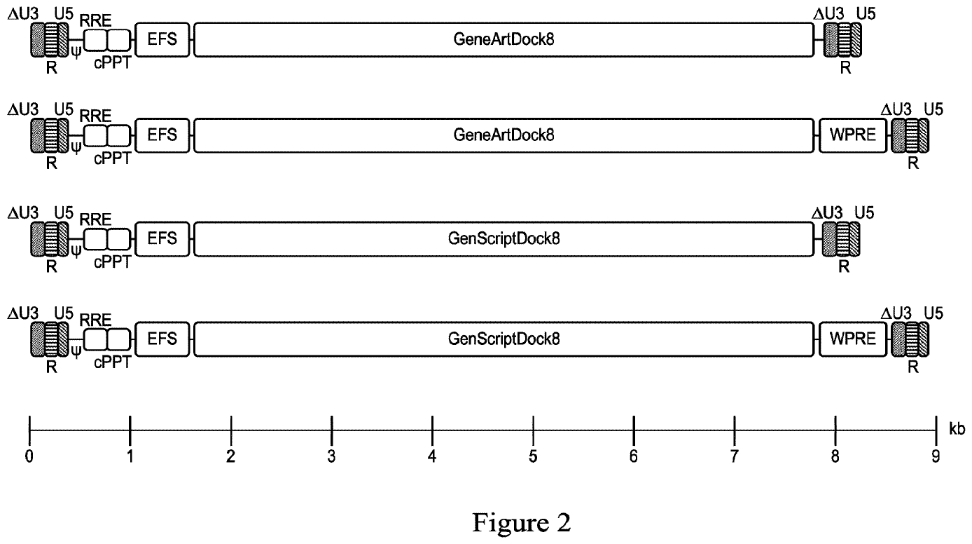

Two codon-optimization algorithms (GeneArt (ga) versus GenScript) were used to generate the DOCK8 cDNA in forward orientation with expression driven by the elongation factor 1alpha short (EFS; SEQ ID NO: 40) as the internal promoter. (FIG. 2). There has been significant experience with the EFS promoter in current clinical trials of gene therapy given its ability to maintain high expression levels of therapeutic transgenes in hematopoietic cells while exhibiting low rates of transactivation of neighboring genomic loci. In addition, the Woodchuck Hepatitis Virus Post-transcriptional Regulatory Element (WPRE; SEQ ID NO: 41), which is widely used in lentiviral therapies to stabilize the viral RNA and mRNA of the expressed transgene, was included in two versions of the DOCK8 donors.

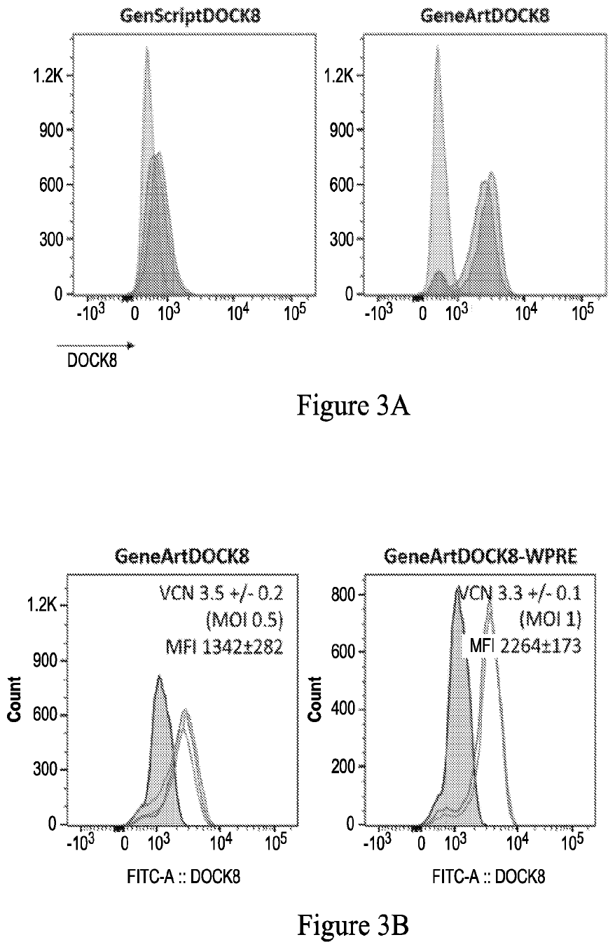

DOCK8 lentiviral vectors were first tested in Jurkat cell lines, which do not express DOCK8 at baseline (any detectable DOCK8 expression after viral transduction is from the transgene.) DOCK8 expression was restored only in cells treated with the EFS-GeneArtDOCK8 vector, with minimal DOCK8 expression when using the GenScript codon optimization. (FIG. 3A) Inclusion of the WPRE sequence improved DOCK8 expression at similar vector copy number (VCN) when measured by flow cytometry. (FIG. 3B).

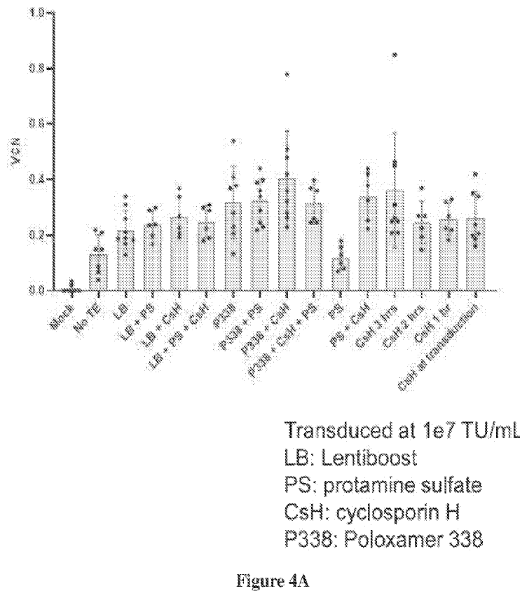

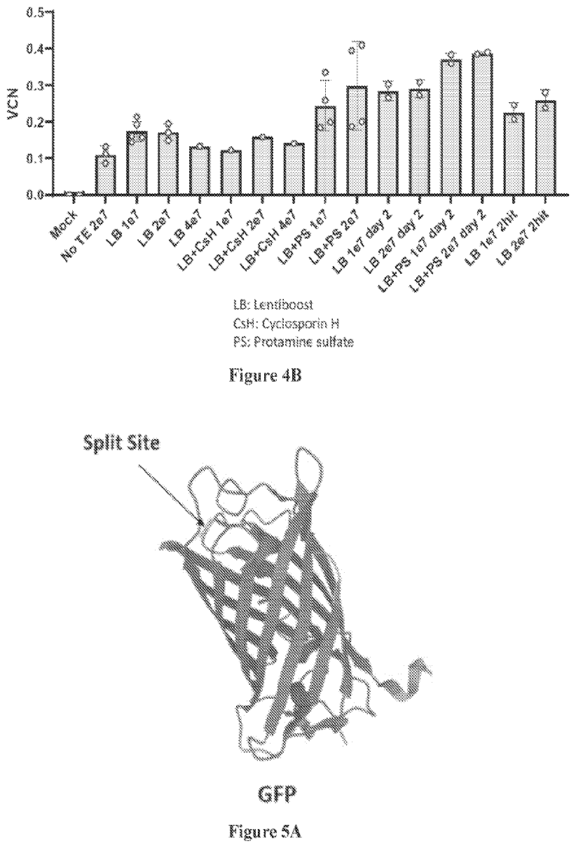

Moving forward with the EFS-GeneArtDOCK8-WPRE lentiviral vector (FIG. 2; SEQ ID NO: 4), peripheral blood stem cells (PBSC) from healthy donors were transduced at 1e7 TU/mL with a combination of various small molecules previously shown to enhance transduction efficiency, including Lentiboost (Sirion Biotech)., cyclosporin H, and protamine sulfate. VCN 12-14 days after transduction is shown in FIG. 4. Increasing amounts of virus beyond 1e7 TU/mL did not appear to increase VCN. Addition of protamine sulfate in addition to LentiBoost increased transduction efficiencies, with improvement when transduction was performed after two day pre-stimulation in XVIVO15 media (Lonza Biosciences) with 50 ng/mL of hSCF, hFlt3L, and hTPO. There was no additional benefit from transducing cells both on day 1 and day 2 of pre-stimulation.

While VCNs of 0.4 (reflecting that 40% of cells contain the therapeutic transgene) are likely to significantly ameliorate the disease phenotype, improved methods to deliver large transgenes more efficiently were considered worth exploring.

### Example 3: Integration of GFP Split Inteins

Objective: To assess integration of GFP split inteins in human cells using protein splicing.

### Methods and Results:

Investigations using inteins were begun in an effort to identify improved methods for the delivery of large transgenes, such as DOCK8, into cells. Inteins are protein segments capable of ligating flanking exteins (external proteins) into a new protein in a process known as protein splicing. Specifically, split inteins divided into two fragments called N-intein and C-intein may be capable of restoring DOCK8 expression using two lentiviral vectors. An example of a split intein is the Cfa split intein (Stevens A J, et al. (2016) Design of a Split Intein with Exceptional Protein Splicing Activity. J. Am. Chem. Soc. 138: 2162-2165).

As the Cfa split intein has been evaluated largely in non-mammalian cells, the published sequences of CfaN and CfaC were codon-optimized using ThermoFisher's GeneOptimizer software to account for human codon usage bias.

### Codon-Optimized CfaN Nucleotide Sequence:

(SEQ ID NO: 16)
TGCCTGAGCTACGACACCGAGATCCTGACCGTGGAATACGGCTTCCTGC
CTATCGGCAAGATCGTGGAAGAACGGATCGAGTGCACCGTGTACACCGT
GGACAAGAACGGCTTCGTGTACACACAGCCTATCGCTCAGTGGCACAAC
CGGGGAGAGCAAGAGGTGTTCGAGTACTGCCTGGAAGATGGCAGCATCA
TCCGGGCCACCAAGGACCACAAGTTCATGACCACCGACGGCCAGATGCT
GCCCATCGACGAGATCTTTGAGAGAGGCCTGGACCTGAAACAGGTGGAC
GGACTTCCT.

### CfaN Amino Acid Sequence:

(SEQ ID NO: 19)
CLSYDTEILTVEYGFLPIGKIVEERIECTVYTVDKNGFVYTQPIAQWHN
RGEQEVFEYCLEDGSIIRATKDHKFMTTDGQMLPIDEIFERGLDLKQVD
GLP

### Codon-Optimized CfaC Nucleotide Sequence:

(SEQ ID NO: 21)
GTGAAGATCATCAGCAGAAAGAGCCTGGGCACCCAGAACGTGTACGACA
TCGGCGTGGAAAAGGACCACAACTTTCTGCTCAAGAACGGCCTGGTGGC
CAGCAAC

### CfaC Amino Acid Sequence:

(SEQ ID NO: 24)
VKIISRKSLGTQNVYDIGVEKDHNFLLKNGLVASN

First, the ability of the human codon-optimized Cfa split intein to splice together two halves of the green fluorescent protein (GFP) was evaluated.

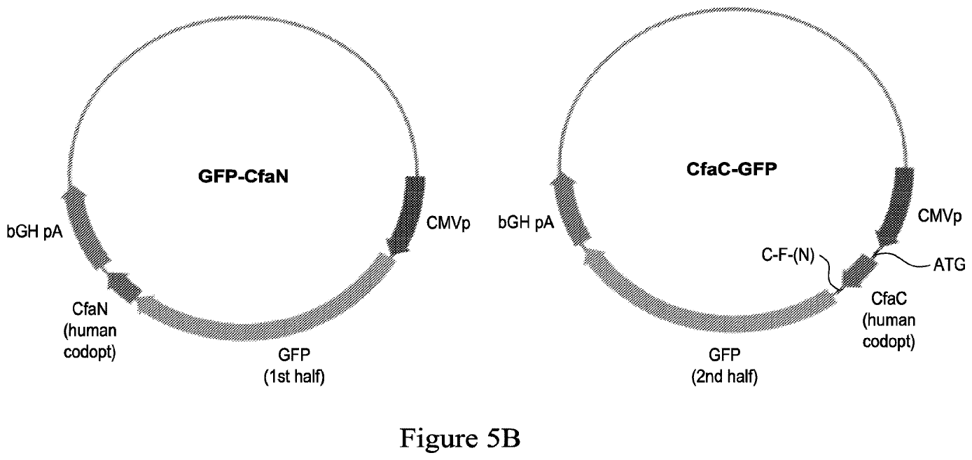

Two separate expression plasmids driven by the CMV promoter were cloned to contain either the CfaN sequence (SEQ ID NO: 16) fused to the first half of GFP or the second half of GFP fused to the CfaC sequence (SEQ ID NO: 21) (FIG. 5B). Two versions of the CfaC plasmids were created to test the required extein amino acids adjacent to the intein, which can be C-F-N (cysteine-phenylalanine-asparagine) or C-F (the plasmid with the C-F-N is not shown but is identical to the CfaC-GFP plasmid except at the site of the C-F). The split site for the GFP protein was chosen at one of the short helical segments on the ends of the beta-sheet cylinder to avoid disruption of the beta-can that is important for GFP expression. (FIG. 5A).

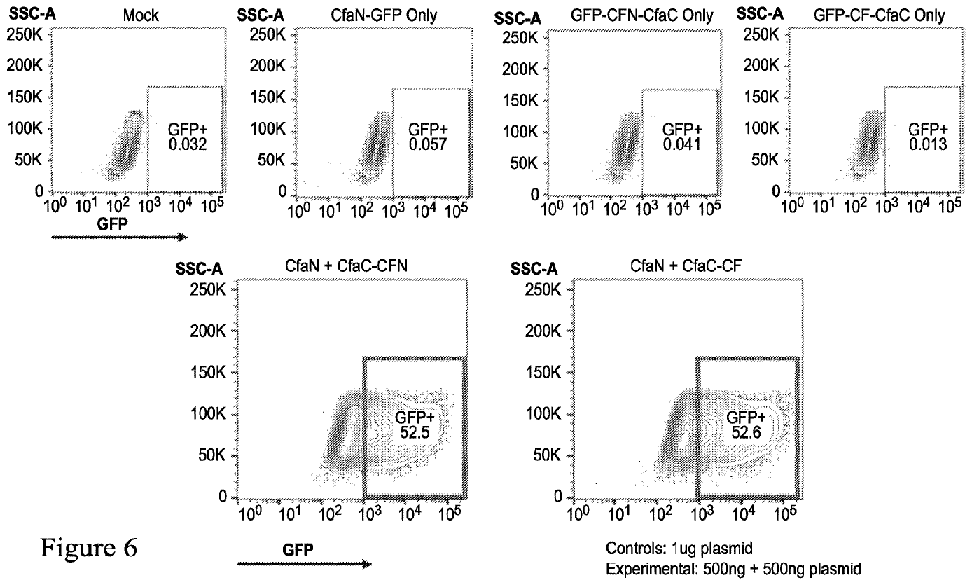

These sequences were then tested in human cell lines, K562 cells, derived from the bone marrow of a female with chronic myelogenous leukemia. Briefly, K562 cells were electroporated on the Lonza 4D platform (Lonza Biosciences) with a total of 1 ug of plasmid. Cells that received the CfaN or CfaC plasmids alone did not express GFP, while those that received both expressed high levels of GFP as measured by flow cytometry. (FIG. 6). The C-F linker for the CfaC intein resulted in similarly efficient protein splicing compared to the C-F-N linker.

### Example 4: Integration of DOCK8 cDNA Split Inteins

Objectives: To assess if splicing of the DOCK8 protein would be feasible by inteins.

### Materials

### Vector and Component Sequences

The plasmid vector constructs provided in Table 1 below were used in Example 4 and Example 5. In these experiments, the nucleotide sequences for both DOCK8 fragments (halves) and the intein halves were codon-optimized using GeneArt algorithms.

TABLE 1
Plasmid Vector Constructs
Construct Sequences

DOCK8 939 CfaN Intein plasmid sequence
939 DOCK8-CfaN_2 (7253 bp)
(see e.g. FIG. 13)
ctgtctatttcgttcatccatagttgcctgactccccgtcgtgtagataactacgatacgggagg
gcttaccatctggccccagtgctgcaatgataccgcgagacccacgctcaccggctccagattta
tcagcaataaaccagccagccggaagggccgagcgcagaagtggtcctgcaactttatccgcctc
catccagtctattaattgttgccgggaagctagagtaagtagttcgccagttaatagtttgcgca
acgttgttgccattgctacaggcatcgtggtgtcacgctcgtcgtttggtatggcttcattcagc
tccggttcccaacgatcaaggcgagttacatgatcccccatgttgtgcaaaaaagcggttagctc
cttcggtcctccgatcgttgtcagaagtaagttggccgcagtgttatcactcatggttatggcag
cactgcataattctcttactgtcatgccatccgtaagatgcttttctgtgactggtgagtactca
accaagtcattctgagaatagtgtatgcggcgaccgagttgctcttgcccggcgtcaatacggga
taataccgcgccacatagcagaactttaaaagtgctcatcattggaaaacgttcttcggggcgaa
aactctcaaggatcttaccgctgttgagatccagttcgatgtaacccactcgtgcacccaactga
tcttcagcatcttttactttcaccagcgtttctgggtgagcaaaaacaggaaggcaaaatgccgc
aaaaaagggaataagggcgacacggaaatgttgaatactcatactcttcctttttcaatattatt
gaagcatttatcagggttattgtctcatgagcggatacatatttgaatgtatttagaaaaataaa
caaataggggttccgcgcacatttccccgaaaagtgccacctgacgtggatcccctgagggggcc
cccatgggctagaggatccggcctcggcctctgcataaataaaaaaaattagtcagccatgagct
tggcccattgcatacgttgtatccatatcataatatgtacatttatattggctcatgtccaacat
taccgccatgttgacattgattattgactagttattaatagtaatcaattacggggtcattagtt
catagcccatatatggagttccgcgttacataacttacggtaaatggcccgcctggctgaccgcc
caacgacccccgcccattgacgtcaataatgacgtatgttcccatagtaacgccaatagggactt
tccattgacgtcaatgggggagtatttacggtaaactgcccacttggcagtacatcaagtgtatc
atatgccaagtacgccccctattgacgtcaatgacggtaaatggcccgcctggcattatgcccag
tacatgaccttatgggactttcctacttggcagtacatctacgtattagtcatcgctattaccat
ggtgatgcggttttggcagtacatcaatgggcgtggatagcggtttgactcacggggatttccaa
gtctccaccccattgacgtcaatgggagtttgttttggcaccaaaatcaacgggactttccaaaa
tgtcgtaacaactccgccccattgacgcaaatgggcggtaggcgtgtacggtgggaggtctatat
aagcagagctcgtttagtgaaccgtcagatcgcctggagacgccatccacgctgttttgacctcc
atagaagacaccgggaccgatccagcctcccctcgaagcttacatgtggtaccgagctcggatcc
tgagaacttcagggtgagtctatgggacccttgatgttttctttccccttcttttctatggttaa
gttcatgtcataggaaggggagaagtaacagggtacacatattgaccaaatcagggtaattttgc
atttgtaattttaaaaaatgctttcttcttttaatatacttttttgtttatcttatttctaatac
tttccctaatctctttctttcagggcaataatgatacaatgtatcatgcctctttgcaccattct
aaagaataacagtgataatttctgggttaaggcaatagcaatatttctgcatataaatatttctg
catataaattgtaactgatgtaagaggtttcatattgctaatagcagctacaatccagctaccat
tctgcttttattttatggttgggataaggctggattattctgagtccaagctaggcccttttgct
aatcatgttcatacctcttatcttcctcccacagctcctgggcaacgtgctggtctgtgtgctgg
cccatcactttggcaaagcacgtgagatctgaattcgagatctgccgccgccATGGCCACACTGC
CTAGCGCTGAGAGAAGGGCCTTCGCTCTGAAGATCAACCGGTACAGCAGCGCCGAGATCCGGAAG
CAGTTTACCCTGCCTCCTAACCTGGGCCAGTACCACAGACAGAGCATCAGCACCAGCGGCTTCCC
ATCTCTGCAGCTGCCCCAGTTCTACGACCCTGTGGAACCCGTGGATTTCGAGGGCCTGCTGATGA
CCCACCTGAACAGCCTGGATGTGCAGCTGGCCCAAGAGCTGGGCGACTTCACCGACGATGATCTG
GACGTGGTGTTCACCCCTAAAGAGTGCAGAACCCTGCAGCCTAGCCTGCCTGAAGAAGGCGTGGA
ACTGGATCCTCACGTGCGGGATTGTGTGCAGACCTACATCCGCGAGTGGCTGATCGTGAACCGGA
AGAATCAGGGCAGCCCAGAGATCTGCGGCTTCAAGAAAACCGGCAGCCGGAAGGACTTCCACAAG
ACCCTGCCTAAGCAGACCTTCGAGAGCGAGACACTGGAATGCTCTGAGCCTGCTGCTCAGGCCGG
ACCTAGACACCTGAATGTGCTGTGTGACGTGTCCGGCAAGGGACCTGTGACCGCCTGCGATTTCG
ACCTGAGAAGTCTGCAGCCCGACAAGCGGCTGGAAAACCTGCTCCAGCAAGTGTCCGCCGAGGAC
TTCGAGAAGCAGAACGAGGAAGCCAGACGGACCAACAGACAGGCCGAGCTGTTTGCTCTGTACCC
CAGCGTGGACGAGGAAGATGCCGTCGAGATTAGACCCGTGCCTGAGTGCCCAAAAGAGCACCTGG
GCAACAGAATCCTGGTCAAGCTGCTGACCCTGAAGTTCGAGATCGAGATTGAGCCCCTGTTCGCC
TCTATCGCCCTGTACGACGTGAAAGAGCGGAAGAAGATCAGCGAGAACTTCCACTGCGACCTGAA
CTCCGACCAGTTCAAGGGCTTCCTGAGAGCCCACACACCATCTGTGGCCGCTAGCTCTCAGGCCA
GATCTGCCGTGTTCAGCGTGACATACCCCAGCAGCGACATCTACCTGGTGGTCAAGATCGAGAAG
GTCCTGCAGCAGGGCGAGATCGGCGATTGTGCCGAGCCTTACACCGTGATCAAAGAGAGCGACGG
CGGCAAGAGCAAAGAGAAGATTGAGAAGCTGAAGCTGCAGGCCGAGAGCTTCTGTCAGCGGCTGG
GCAAGTACAGAATGCCCTTTGCCTGGGCTCCTATCAGCCTGTCCAGCTTCTTCAACGTGTCCACA
CTGGAACGGGAAGTGACCGACGTGGACTCTGTCGTGGGCAGATCTAGCGTGGGCGAGAGAAGGAC
ACTGGCTCAGTCTAGACGGCTGAGCGAGAGAGCCCTGAGCCTGGAAGAAAATGGCGTGGGCAGCA
ACTTCAAGACCAGCACTCTGTCCGTGTCTAGCTTCTTTAAGCAAGAGGGCGACAGACTGAGCGAC
GAGGACCTGTTCAAGTTCCTGGCCGACTACAAGCGGAGCAGCTCCCTGCAGAGAAGAGTGAAGTC
TATCCCTGGCCTGCTGCGCCTGGAAATCTCTACAGCCCCTGAGATCATCAACTGCTGTCTGACCC
CTGAAATGCTGCCCGTGAAGCCCTTTCCAGAGAACCGGACCAGACCTCACAAAGAGATCCTGGAA
TTCCCCACCAGAGAAGTGTACGTGCCCCACACCGTGTACCGGAACCTGCTGTATGTGTACCCACA
GCGGCTGAACTTCGTGAACAAGCTGGCCTCCGCCAGAAACATCACCATCAAGATCCAGTTTATGT
GCGGCGAGGACGCCAGCAACGCCATGCCTGTGATCTTCGGCAAGTCTAGCGGCCCTGAGTTCCTG
CAAGAGGTGTACACAGCCGTGACCTACCACAACAAGAGCCCCGACTTCTACGAGGAAGTGAAGAT
TAAGCTGCCCGCCAAGCTGACCGTGAATCACCATCTGCTGTTCACCTTCTACCACATCAGCTGCC
AGCAGAAACAGGGCGCCTCTGTGGAAACACTGCTGGGCTATAGCTGGCTGCCCATCCTGCTGAAC
GAGAGACTGCAGACCGGCAGCTACTGTCTGCCTGTGGCTCTGGAAAAGCTGCCACCTAACTACAG
CATGCACTCCGCCGAGAAGGTGCCCCTGCAGAATCCTCCTATTAAGTGGGCCGAGGGCCACAAGG
GCGTGTTCAATATCGAGGTGCAGGCCGTGTCCTCCGTGCACACCCAGGATAACCACCTGGAAAAG
TTCTTCACCCTGTGCCACAGCCTCGAGAGCCAAGTGACATTCCCCATCCGCGTGCTGGACCAGAA
AATCTCCGAGATGGCCCTGGAACACGAGCTGAAACTGAGCATCATCTGCCTGAATAGCAGCAGAC
TGGAACCCCTGGTGCTGTTCCTGCATCTGGTGCTGGACAAGCTGTTCCAGCTGAGCGTGCAGCCC
ATGGTTATCGCCGGACAGACCGCCAACTTCAGCCAGTTCGCCTTTGAGAGCGTGGTGGCCATTGC
CAACAGCCTGCACAACAGCAAGGACCTGAGCAAGGATCAGCACGGCAGAAACTGCCTGCTGGCCT
CTTACGTGCACTACGTGTTCAGACTGCCTGAGGTGCAGAGGGACGTGCCAAAATCTGGCGCTCCT
ACCGCTCTGCTGGACCCCAGAAGCTATCACACCTACGGCAGAACATCTGCCGCCGCTGTGTCCTC
TAAACTCCTGCAGGCTAGAGTGATGTCCAGCAGCAACCCTGATCTGGCCGGAACACACAGCGCCG
CTGATGAAGAAGTGAAAAACATCATGAGCAGCAAGATCGCCGACCGGAACTGCAGCCGGATGAGC
TACTACTGCCTGAGCTACGACACCGAGATCCTGACCGTGGAATACGGCTTCCTGCCTATCGGCAA
GATCGTGGAAGAACGGATCGAGTGCACCGTGTACACCGTGGACAAGAACGGCTTCGTGTACACAC
AGCCTATCGCTCAGTGGCACAACCGGGGAGAGCAAGAGGTGTTCGAGTACTGCCTGGAAGATGGC
AGCATCATCCGGGCCACCAAGGACCACAAGTTCATGACCACCGACGGCCAGATGCTGCCCATCGA
CGAGATCTTTGAGAGAGGCCTGGACCTGAAACAGGTGGACGGACTTCCTtgactggtgtggctaa
tgccctggcccacaagtAtcactaagctcgcttTcttgctgtccaatttctattaaaggttcctt
Tgttccctaagtccaactactaaactgggggatattatgaagggccttgagcatctggattctgc
ctaataaaaaacatttattttcattgcaatgatgtatttaaattatttctgaatattttactaaa
aagggaatgtgggaggtcagtgcatttaaaacataaagaaatgaagagctagttcaaaccttggg
aaaatacactatatcttaaactccatgaaagaaggtgaggctgcaaacagctaatgcacattggc
aacagCccctgatgcctatgccttattcatccctcagaaaaggattcaagtagaggcttgatttg
gaggttaaagtttTgctatgctgtattttacattacttattgttttagctgtcctcatgaatgtc
ttttcactacccatttgcttatcctgcatctctcagccttgactccactcagttctcttgcttag
agataccacctttcccctgaagtgttccttccatgttttacggcgagatggtttctcctcgcctg
gccactcagccttagttgtctctgttgtcttatagaggtctacttgaagaaggaaaaacaggggg
catggtttgactgtcctgtgagcccttcttccctgcctcccccactcacagtgacccggaatccc
tcgacatggcagtctagcactagtgcggccgcagatctgcttcctcgctcactgactcgctgcgc
tcggtcgttcggctgcggcgagcggtatcagctcactcaaaggcggtaatacggttatccacaga
atcaggggataacgcaggaaagaacatgtgagcaaaaggccagcaaaaggccaggaaccgtaaaa
aggccgcgttgctggcgtttttccataggctccgcccccctgacgagcatcacaaaaatcgacgc
tcaagtcagaggtggcgaaacccgacaggactataaagataccaggcgtttccccctggaagctc
cctcgtgcgctctcctgttccgaccctgccgcttaccggatacctgtccgcctttctcccttcgg
gaagcgtggcgctttctcaTAgctcacgctgtaggtatctcagttcggtgtaggtcgttcgctcc
aagctgggctgtgtgcacgaaccccccgttcagcccgaccgctgcgccttatccggtaactatcg
tcttgagtccaacccggtaagacacgacttatcgccactggcagcagccactggtaacaggatta
gcagagcgaggtatgtaggcggtgctacagagttcttgaagtggtggcctaactacggctacact
agaagAacagtatttggtatctgcgctctgctgaagccagttaccttcggaaaaagagttggtag
ctcttgatccggcaaacaaaccaccgctggtagcggtggtttttttgtttgcaagcagcagatta
cgcgcagaaaaaaaggatctcaagaagatcctttgatcttttctacggggtctgacgctcagtgg
aacgaaaactcacgttaagggattttggtcatgagattatcaaaaaggatcttcacctagatcct
tttaaattaaaaatgaagttttaaatcaatctaaagtatatatgagtaaacttggtctgacagtt
accaatgcttaatcagtgaggcacctatctcagcgat
(SEQ ID NO: 5)
DOCK8 939 CfaN Intein Lentiviral plasmid sequence
mCCL-EFS-939Dock8-CfaN-WPRE (9959 bp)
(see e.g. FIG. 14)
acgcgccctgtagcggcgcattaagcgcggcgggtgtggtggttacgcgcagcgtgaccgctaca
cttgccagcgccctagcgcccgctcctttcgctttcttcccttcctttctcgccacgttcgccgg
ctttccccgtcaagctctaaatcgggggctccctttagggttccgatttagtgctttacggcacc
tcgaccccaaaaaacttgattagggtgatggttcacgtagtgggccatcgccctgatagacggtt
tttcgccctttgacgttggagtccacgttctttaatagtggactcttgttccaaactggaacaac
actcaaccctatctcggtctattcttttgatttataagggattttgccgatttcggcctattggt
taaaaaatgagctgatttaacaaaaatttaacgcgaattttaacaaaatattaacgcttacaatt
taggtggcacttttcggggaaatgtgcgcggaacccctatttgtttatttttctaaatacattca
aatatgtatccgctcatgagacaataaccctgataaatgcttcaataatagcacctagatcaaga
gacaggatgaggatcgtttcgcatgattgaacaagatggattgcacgcaggttctccggccgctt
gggtggagaggctattcggctatgactgggcacaacagacaatcggctgctctgatgccgccgtg
ttccggctgtcagcgcaggggcgcccggttctttttgtcaagaccgacctgtccggtgccctgaa
tgaactgcaagacgaggcagcgcggctatcgtggctggccacgacgggcgttccttgcgcagctg
tgctcgacgttgtcactgaagcgggaagggactggctgctattgggcgaagtgccggggcaggat
ctcctgtcatctcaccttgctcctgccgagaaagtatccatcatggctgatgcaatgcggcggct
gcatacgcttgatccggctacctgcccattcgaccaccaagcgaaacatcgcatcgagcgagcac
gtactcggatggaagccggtcttgtcgatcaggatgatctggacgaagagcatcaggggctcgcg
ccagccgaactgttcgccaggctcaaggcgagcatgcccgacggcgaggatctcgtcgtgaccca
tggcgatgcctgcttgccgaatatcatggtggaaaatggccgcttttctggattcatcgactgtg
gccggctgggtgtggcggaccgctatcaggacatagcgttggctacccgtgatattgctgaagag
cttggcggcgaatgggctgaccgcttcctcgtgctttacggtatcgccgctcccgattcgcagcg
catcgccttctatcgccttcttgacgagttcttctgaattattaacgcttacaatttcctgatgc
ggtattttctccttacgcatctgtgcggtatttcacaccgcatcaggtggcacttttcggggaaa
tgtgcgcggaacccctatttgtttatttttctaaatacattcaaatatgtatccgctcatgacca
aaatcccttaacgtgagttttcgttccactgagcgtcagaccccgtagaaaagatcaaaggatct
tcttgagatcctttttttctgcgcgtaatctgctgcttgcaaacaaaaaaaccaccgctaccagc
ggtggittgtttgccggatcaagagctaccaactctttttccgaaggtaactggcttcagcagag
cgcagataccaaatactgttcttctagtgtagccgtagttaggccaccacttcaagaactctgta
gcaccgcctacatacctcgctctgctaatcctgttaccagtggctgctgccagtggcgataagtc
gtgtcttaccgggttggactcaagacgatagttaccggataaggcgcagcggtcgggctgaacgg
gggggtcgtgcacacagcccagcttggagcgaacgacctacaccgaactgagatacctacagcgt
gagctatgagaaagcgccacgcttcccgaagggagaaaggcggacaggtatccggtaagcggcag
ggtcggaacaggagagcgcacgagggagcttccagggggaaacgcctggtatctttatagtcctg
tcgggtttcgccacctctgacttgagcgtcgatttttgtgatgctcgtcaggggggcggagccta
tggaaaaacgccagcaacgcggcctttttacggttcctggccttttgctggccttttgctcacat
gttctttcctgcgttatcccctgattctgtggataaccgtattaccgcctttgagtgagctgata
ccgctcgccgcagccgaacgaccgagcgcagcgagtcagtgagcgaggaagcggaagagcgccca
atacgcaaaccgcctctccccgcgcgttggccgattcattaatgcagctggcacgacaggtttcc
cgactggaaagcgggcagtgagcgcaacgcaattaatgtgagttagctcactcattaggcacccc
aggctttacactttatgcttccggctcgtatgttgtgtggaattgtgagcggataacaatttcac
acaggaaacagctatgaccatgattacgccaagcgcgcaattaaccctcactaaagggaacaaaa
gctggagctgcaagcttggccattgcatacgttgtatccatatcataatatgtacatttatattg
gctcatgtccaacattaccgccatgttgacattgattattgactagttattaatagtaatcaatt
acggggtcattagttcatagcccatatatggagttccgcgttacataacttacggtaaatggccc
gcctggctgaccgcccaacgacccccgcccattgacgtcaataatgacgtatgttcccatagtaa
cgccaatagggactttccattgacgtcaatgggtggagtatttacggtaaactgcccacttggca
gtacatcaagtgtatcatatgccaagtacgccccctattgacgtcaatgacggtaaatggcccgc
ctggcattatgcccagtacatgaccttatgggactttcctacttggcagtacatctacgtattag
tcatcgctattaccatggtgatgcggttttggcagtacatcaatgggcgtggatagcggtttgac
tcacggggatttccaagtctccaccccattgacgtcaatgggagtttgttttggcaccaaaatca
acgggactttccaaaatgtcgtaacaactccgccccattgacgcaaatgggcggtaggcgtgtac
ggtgggaggtctatataagcagagctcgtttagtgaaccgGGGTCTCTCTGGTTAGACCAGATCT
GAGCCTGGGAGCTCTCTGGCTAACTAGGGAACCCACTGCTTAAGCCTCAATAAAGCTTGCCTTGA
GTGCTTCAAGTAGTGTGTGCCCGTCTGTTGTGTGACTCTGGTAACTAGAGATCCCTCAGACCCTT
TTAGTCAGTGTGGAAAATCTCTAGCagtggcgcccgaacagggacttgaaagcgaaagggaaacc
agaggagctctcTCGACGCAGGACTCGGCTTGCTGAAGCGCGCACGGCAAGAGGCGAGGGGCGGC
GACTGGTGAGTACGCCAAAAATTTTGACTAGCGGAGGCTAGAAGGAGAGAGATGGGTGCGAGAGC
GTCAGTATTAAGCGGGGGAGaattagatcgcgatgggaaaaaattcggttaaggccagggggaaa
gaaaaaatataaattaaaacatatagtatgggcaagcagggagctagaacgattcgcagttaatc
ctggcctgttagaaacatcagaaggctgtagacaaatactgggacagctacaaccatcccttcag
acaggatcagaagaacttagatcattatataatacagtagcaaccctctattgtgtgcatcaaag
gatagagataaaagacaccaaggaagctttagacaagatagaggaagagcaaaaaaaagtaagac
caccgcacagcaagcGGCCGCTgatcttcagacctggaggaggagatatgagggacaattggaga
agtgaattatataaatataaagtagtaaaaattgaaccattaggagtagcacccaccaaggcaaa
gagaagagtggtgcagagagaaaaaagaGCAGTGGGAATaggagctttgttccttgggttcttgg
gagcagcaggaagcactatgggcgcagcgtcaatgacgctgacggtacaggccagacaattattg
tctggtatagtgcagcagcagaacaatttgctgagggctattgaggcgcaacagcatctgttgca
actcacagtctggggcatcaagcagctccaggcaagaatcctggctgtggaaagatacctaaagg
atcaacagctcctggggatttggggttgctctggaaaactcatttgcaccactgctgtgccttgg
aatgctagttggagtaataaatctctggaacagatttggaatcacacgacctggatggagtggga
cagagaaattaacaattacacaagcttaatacactccttaattgaagaatcgcaaaaccagcaag
aaaagaatgaacaagaattattggaattagataaatgggcaagtttgtggaattggtttaacata
acaaattggctgtggtatataaaattattcataatgatagtaggaggcttggtaggtttaagaat
agtttttgctgtactttctatagtgaatagagttaggcagggatattcaccattatcgtttcaga
cccacctcccaaccccgaggggacccgacaggcccgaaggaatagaagaagaaggtggagagaga
gacagagacagatccattcgattagtgaacggatCacaaatggcagtattcatccacaattttaa
aagaaaaggggggattggggggtacagtgcaggggaaagaatagtagacataatagcaacagaca
tacaaactaaagaattacaaaaacaaattacaaaaattcaaaattttcgggtttattacagggac
agcagaggctccggtgcccgtcagtgggcagagcgcacatcgcccacagtccccgagaagttggg
gggaggggtcggcaattgaaccggtgcctagagaaggtggcgcggggtaaactgggaaagtgatg
tcgtgtactggctccgcctttttcccgaggggggggagaaccgtatataagtgcagtagtcgccg
tgaacgttctttttcgcaacgggtttgccgccagaacacaggtgtcgtgaAAATAAGAGAGAAAA
GAAGAGTAAGAAGAAATATAAGAGCCACCATGGCCACACTGCCTAGCGCTGAGAGAAGGGCCTTC
GCTCTGAAGATCAACCGGTACAGCAGCGCCGAGATCCGGAAGCAGTTTACCCTGCCTCCTAACCT
GGGCCAGTACCACAGACAGAGCATCAGCACCAGCGGCTTCCCATCTCTGCAGCTGCCCCAGTTCT
ACGACCCTGTGGAACCCGTGGATTTCGAGGGCCTGCTGATGACCCACCTGAACAGCCTGGATGTG
CAGCTGGCCCAAGAGCTGGGCGACTTCACCGACGATGATCTGGACGTGGTGTTCACCCCTAAAGA
GTGCAGAACCCTGCAGCCTAGCCTGCCTGAAGAAGGCGTGGAACTGGATCCTCACGTGCGGGATT
GTGTGCAGACCTACATCCGCGAGTGGCTGATCGTGAACCGGAAGAATCAGGGCAGCCCAGAGATC
TGCGGCTTCAAGAAAACCGGCAGCCGGAAGGACTTCCACAAGACCCTGCCTAAGCAGACCTTCGA
GAGCGAGACACTGGAATGCTCTGAGCCTGCTGCTCAGGCCGGACCTAGACACCTGAATGTGCTGT
GTGACGTGTCCGGCAAGGGACCTGTGACCGCCTGCGATTTCGACCTGAGAAGTCTGCAGCCCGAC
AAGCGGCTGGAAAACCTGCTCCAGCAAGTGTCCGCCGAGGACTTCGAGAAGCAGAACGAGGAAGC
CAGACGGACCAACAGACAGGCCGAGCTGTTTGCTCTGTACCCCAGCGTGGACGAGGAAGATGCCG
TCGAGATTAGACCCGTGCCTGAGTGCCCAAAAGAGCACCTGGGCAACAGAATCCTGGTCAAGCTG
CTGACCCTGAAGTTCGAGATCGAGATTGAGCCCCTGTTCGCCTCTATCGCCCTGTACGACGTGAA
AGAGCGGAAGAAGATCAGCGAGAACTTCCACTGCGACCTGAACTCCGACCAGTTCAAGGGCTTCC
TGAGAGCCCACACACCATCTGTGGCCGCTAGCTCTCAGGCCAGATCTGCCGTGTTCAGCGTGACA
TACCCCAGCAGCGACATCTACCTGGTGGTCAAGATCGAGAAGGTCCTGCAGCAGGGCGAGATCGG
CGATTGTGCCGAGCCTTACACCGTGATCAAAGAGAGCGACGGCGGCAAGAGCAAAGAGAAGATTG
AGAAGCTGAAGCTGCAGGCCGAGAGCTTCTGTCAGCGGCTGGGCAAGTACAGAATGCCCTTTGCC
TGGGCTCCTATCAGCCTGTCCAGCTTCTTCAACGTGTCCACACTGGAACGGGAAGTGACCGACGT
GGACTCTGTCGTGGGCAGATCTAGCGTGGGCGAGAGAAGGACACTGGCTCAGTCTAGACGGCTGA
GCGAGAGAGCCCTGAGCCTGGAAGAAAATGGCGTGGGCAGCAACTTCAAGACCAGCACTCTGTCC
GTGTCTAGCTTCTTTAAGCAAGAGGGCGACAGACTGAGCGACGAGGACCTGTTCAAGTTCCTGGC
CGACTACAAGCGGAGCAGCTCCCTGCAGAGAAGAGTGAAGTCTATCCCTGGCCTGCTGCGCCTGG
AAATCTCTACAGCCCCTGAGATCATCAACTGCTGTCTGACCCCTGAAATGCTGCCCGTGAAGCCC
TTTCCAGAGAACCGGACCAGACCTCACAAAGAGATCCTGGAATTCCCCACCAGAGAAGTGTACGT
GCCCCACACCGTGTACCGGAACCTGCTGTATGTGTACCCACAGCGGCTGAACTTCGTGAACAAGC
TGGCCTCCGCCAGAAACATCACCATCAAGATCCAGTTTATGTGCGGCGAGGACGCCAGCAACGCC
ATGCCTGTGATCTTCGGCAAGTCTAGCGGCCCTGAGTTCCTGCAAGAGGTGTACACAGCCGTGAC
CTACCACAACAAGAGCCCCGACTTCTACGAGGAAGTGAAGATTAAGCTGCCCGCCAAGCTGACCG
TGAATCACCATCTGCTGTTCACCTTCTACCACATCAGCTGCCAGCAGAAACAGGGCGCCTCTGTG
GAAACACTGCTGGGCTATAGCTGGCTGCCCATCCTGCTGAACGAGAGACTGCAGACCGGCAGCTA
CTGTCTGCCTGTGGCTCTGGAAAAGCTGCCACCTAACTACAGCATGCACTCCGCCGAGAAGGTGC
CCCTGCAGAATCCTCCTATTAAGTGGGCCGAGGGCCACAAGGGCGTGTTCAATATCGAGGTGCAG
GCCGTGTCCTCCGTGCACACCCAGGATAACCACCTGGAAAAGTTCTTCACCCTGTGCCACAGCCT
CGAGAGCCAAGTGACATTCCCCATCCGCGTGCTGGACCAGAAAATCTCCGAGATGGCCCTGGAAC
ACGAGCTGAAACTGAGCATCATCTGCCTGAATAGCAGCAGACTGGAACCCCTGGTGCTGTTCCTG
CATCTGGTGCTGGACAAGCTGTTCCAGCTGAGCGTGCAGCCCATGGTTATCGCCGGACAGACCGC
CAACTTCAGCCAGTTCGCCTTTGAGAGCGTGGTGGCCATTGCCAACAGCCTGCACAACAGCAAGG
ACCTGAGCAAGGATCAGCACGGCAGAAACTGCCTGCTGGCCTCTTACGTGCACTACGTGTTCAGA
CTGCCTGAGGTGCAGAGGGACGTGCCAAAATCTGGCGCTCCTACCGCTCTGCTGGACCCCAGAAG
CTATCACACCTACGGCAGAACATCTGCCGCCGCTGTGTCCTCTAAACTCCTGCAGGCTAGAGTGA
TGTCCAGCAGCAACCCTGATCTGGCCGGAACACACAGCGCCGCTGATGAAGAAGTGAAAAACATC
ATGAGCAGCAAGATCGCCGACCGGAACTGCAGCCGGATGAGCTACTACTGCCTGAGCTACGACAC
CGAGATCCTGACCGTGGAATACGGCTTCCTGCCTATCGGCAAGATCGTGGAAGAACGGATCGAGT
GCACCGTGTACACCGTGGACAAGAACGGCTTCGTGTACACACAGCCTATCGCTCAGTGGCACAAC
CGGGGAGAGCAAGAGGTGTTCGAGTACTGCCTGGAAGATGGCAGCATCATCCGGGCCACCAAGGA
CCACAAGTTCATGACCACCGACGGCCAGATGCTGCCCATCGACGAGATCTTTGAGAGAGGCCTGG
ACCTGAAACAGGTGGACGGACTTCCTtgattccgataatcaacctctggattacaaaatttgtga
aagattgactggtattcttaactatgttgctccttttacgctatgtggatacgctgctttaatgc
ctttgtatcatgctattgcttcccgtatggctttcattttctcctccttgtataaatcctggttg
ctgtctctttatgaggagttgtggcccgttgtcaggcaacgtggcgtggtgtgcactgtgtttgc
tgacgcaacccccactggttggggcattgccaccacctgtcagctcctttccgggactttcgctt
tccccctccctattgccacggcggaactcatcgccgcctgccttgcccgctgctggacaggggct
cggctgttgggcactgacaattccgtggtgttgtcggggaaATCATcgtcctttccTtggctgct
cgcctgtgttgccacctggattctgcgcgggacgtccttctgctacgtcccttcggccctcaatc
cagcggaccttccttcccgcggcctgctgccggctctgcggcctcttccgcgtcttcgccttcgc
cctcagacgagtcggatctc20cctttgggccgcctccccgcTaaaagaaaaggggggactggaa
gggctaattcactcccaacgaagacaagatctgctttttgcttgtactGGGTCTCTCTGGTTAGA
CCAGATCTGAGCCTGGGAGCTCTCTGGCTAACTAGGGAACCCACTGCTTAAGCCTCAATAAAGCT
TGCCTTGAGTGCTTCAAGTAGTGTGTTGGTTTTTTGTGTGTGCATTGTCTGAGTAGGTGTCATTC
TATTCTGGGGGGTGGGGTGGGGCAGCACAGCAAGGGGGAGGATTGGGAAGACAATAGCAGGCATG
CTGGGGATGCGGTGGGCTCTATGGagtagtagttcatgtcatcttattattcagtatttataact
tgcaaagaaatgaatatcagagagtgagaggaacttgtttattgcagcttataatggttacaaat
aaagcaatagcatcacaaatttcacaaataaagcatttttttcactgcattctagttgtggtttg
tccaaactcatcaatgtatcttatcatgtctggctctagctatcccgcccctaactccgcccATC
CCGCCCCTAACTCCGCCCAGTTCCGCCCATTCTCCGCCCCATGGCTCTCACTACTTCTGGAATAG
CTCAGAGGCCGAGGCGGCCTCGGCCTCTGCATAAATAAAAAAAATTAGTCGAGGCTTTTTTGGAG
GCCTAGGgacgtacccaattcgccctatagtgagtcgtattacgcgcgctcactggccgtcgttt
tacaacgtcgtgactgggaaaaccctggcgttacccaacttaatcgccttgcagcacatccccct
ttcgccagctggcgtaatagcgaagaggcccgcaccgatcgcccttcccaacagttgcgcagcct
gaatggcgaatggg
(SEQ ID NO: 6)
DOCK8 939 CfaC-CF Intein plasmid sequence
939 ClaC-CF-DOCK8-bGHpA (7730 bp)
(see e.g. FIG. 15)
gaagatcctttgatcttttctacggggtctgacgctcagtggaacgaaaactcacgttaagggat
tttggtcatgagattatcaaaaaggatcttcacctagatccttttaaattaaaaatgaagtttta
aatcaatctaaagtatatatgagtaaacttggtctgacagttaccaatgcttaatcagtgaggca
cctatctcagcgatctgtctatttcgttcatccatagttgcctgactccccgtcgtgtagataac
tacgatacgggagggcttaccatctggccccagtgctgcaatgataccgcgagacccacgctcac
cggctccagatttatcagcaataaaccagccagccggaagggccgagcgcagaagtggtcctgca
actttatccgcctccatccagtctattaattgttgccgggaagctagagtaagtagttcgccagt
taatagtttgcgcaacgttgttgccattgctacaggcatcgtggtgtcacgctcgtcgtttggta
tggcttcattcagctccggttcccaacgatcaaggcgagttacatgatcccccatgttgtgcaaa
aaagcggttagctccticggtcctccgatcgitgtcagaagtaagtiggccgcagtgttatcact
catggttatggcagcactgcataattctcttactgtcatgccatccgtaagatgcttttctgtga
ctggtgagtactcaaccaagtcattctgagaatagtgtatgcggcgaccgagttgctcttgcccg
gcgtcaatacgggataataccgcgccacatagcagaactttaaaagtgctcatcattggaaaacg
ttcttcggggcgaaaactctcaaggatcttaccgctgttgagatccagttcgatgtaacccactc
gtgcacccaactgatcttcagcatcttttactttcaccagcgtttctgggtgagcaaaaacagga
aggcaaaatgccgcaaaaaagggaataagggcgacacggaaatgttgaatactcatactcttcct
ttttcaatattattgaagcatttatcagggttattgtctcatgagcggatacatatttgaatgta
tttagaaaaataaacaaataggggttccgcgcacatttccccgaaaagtgccacctgacgtggat
cccctgagggggcccccatgggctagaggatccggcctcggcctctgcataaataaaaaaaatta
gtcagccatgagcttggcccattgcatacgttgtatccatatcataatatgtacatttatattgg
ctcatgtccaacattaccgccatgttgacattgattattgactagttattaatagtaatcaatta
cggggtcattagttcatagcccatatatggagttccgcgttacataacttacggtaaatggcccg
cctggctgaccgcccaacgacccccgcccattgacgtcaataatgacgtatgttcccatagtaac
gccaatagggactttccattgacgtcaatgggtggagtatttacggtaaactgcccacttggcag
tacatcaagtgtatcatatgccaagtacgccccctattgacgtcaatgacggtaaatggcccgcc
tggcattatgcccagtacatgaccttatgggactttcctacttggcagtacatctacgtattagt
catcgctattaccatggtgatgcggttttggcagtacatcaatgggcgtggatagcggtttgact
cacggggatttccaagtctccaccccattgacgtcaatgggagtttgttttggcaccaaaatcaa
cgggactttccaaaatgtcgtaacaactccgccccattgacgcaaatgggcggtaggcgtgtacg
gtgggaggtctatataagcagagctcgtttagtgaaccgtcagatcgcctggagacgccatccac
gctgttttgacctccatagaagacaccgggaccgatccagcctcccctcgaagcttacatgtggt
accgagctcggatcctgagaacttcagggtgagtctatgggacccttgatgttttctttcccctt
cttttctatggttaagttcatgtcataggaaggggagaagtaacagggtacacatattgaccaaa
tcagggtaattttgcatttgtaattttaaaaaatgctttcttcttttaatatacttttttgttta
tcttatttctaatactttccctaatctctttctttcagggcaataatgatacaatgtatcatgcc
tctttgcaccattctaaagaataacagtgataatttctgggttaaggcaatagcaatatttctgc
atataaatatttctgcatataaattgtaactgatgtaagaggtttcatattgctaatagcagcta
caatccagctaccattctgcttttattttatggttgggataaggctggattattctgagtccaag
ctaggcccttttgctaatcatgttcatacctcttatcttcctcccacagctcctgggcaacgtgc
tggtctgtgtgctggcccatcactttggcaaagcacgtgagatctgaattcgagatctgccgccg
ccatgGTGAAGATCATCAGCAGAAAGAGCCTGGGCACCCAGAACGTGTACGACATCGGCGTGGAA
AAGGACCACAACTTTCTGCTCAAGAACGGCCTGGTGGCCAGCAACTGCTTTAGCGGCAGCTCTGA
TGCCCCTAGCTCTCCAGCTGCTCCTAGACCTGCCAGCAAGAAGCACTTTCACGAGGAACTGGCCC
TGCAGATGGTCGTGTCTACCGGCATGGTTCGAGAGACAGTGTTTAAGTACGCCTGGTTCTTCTTC
GAACTGCTGGTCAAGAGCATGGCCCAGCACGTGCACAACATGGACAAGCGGGACAGCTTCCGGCG
GACCAGATTCAGCGACAGATTCATGGACGACATCACCACCATCGTGAACGTGGTCACCAGCGAGA
TTGCTGCCCTGCTGGTTAAGCCCCAGAAAGAGAACGAGCAGGCCGAAAAGATGAATATCTCTCTG
GCCTTCTTCCTCTACGACCTGCTGAGCCTGATGGACCGGGGCTTCGTGTTCAACCTGATCCGGCA
CTACTGCTCCCAGCTGTCTGCCAAGCTGTCCAACCTGCCTACACTGATCTCTATGAGACTCGAGT
TCCTGAGGATCCTGTGCAGCCACGAGCACTACCTGAACCTGAATCTGTTCTTCATGAACGCCGAC
ACAGCCCCTACAAGCCCCTGTCCTTCTATCAGCAGCCAGAACAGCAGCAGCTGTAGCAGCTTTCA
GGATCAGAAAATCGCCAGCATGTTCGATCTGACCAGCGAGTACCGGCAGCAGCACTTTCTGACCG
GCCTGCTGTTTACCGAACTGGCAGCCGCTCTTGATGCCGAAGGCGAGGGAATCAGCAAGGTGCAG
CGGAAAGCCGTGTCTGCCATTCACAGCCTGCTGTCTAGCCACGACCTGGATCCTAGATGCGTGAA
GCCTGAAGTGAAAGTGAAAATCGCTGCCCTGTACCTGCCTCTCGTGGGCATCATTCTGGATGCCC
TGCCTCAGCTGTGTGATTTCACCGTGGCCGACACCAGACGGTACAGAACCTCTGGCTCCGACGAG
GAACAAGAAGGCGCTGGCGCCATCAACCAGAATGTGGCCCTTGCCATTGCCGGGAACAACTTCAA
TCTGAAAACCAGCGGCATCGTGCTGAGCAGCCTGCCTTACAAGCAGTACAACATGCTGAATGCCG
ACACTACCCGCAACCTGATGATCTGCTTCCTGTGGATTATGAAGAACGCCGATCAGAGCCTGATT
CGGAAGTGGATTGCCGACCTGCCAAGCACTCAGCTGAACCGGATTCTGGACCTCCTGTTCATCTG
CGTGCTGTGCTTCGAGTACAAGGGCAAGCAGTCCAGCGACAAGGTGTCCACTCAGGTGCTGCAGA
AAAGCCGGGATGTGAAGGCCAGGCTGGAAGAGGCACTTCTGAGAGGCGAAGGCGCCAGAGGCGAG
ATGATGAGGCGTAGAGCCCCTGGCAACGATAGATTCCCCGGCCTGAATGAGAACCTGCGGTGGAA
GAAAGAGCAGACCCATTGGAGACAGGCCAACGAGAAACTGGACAAGACCAAGGCCGAACTGGACC
AAGAGGCCCTGATCTCTGGCAACCTGGCCACAGAAGCCCACCTGATTATCCTGGACATGCAAGAG
AACATCATCCAGGCCAGTAGCGCCCTGGATTGCAAGGATTCTCTGCTCGGAGGCGTGCTGAGGGT
GCTCGTGAATAGCCTGAATTGCGACCAGAGCACCACCTACCTGACACACTGCTTCGCCACACTGA
GGGCCCTGATTGCCAAGTTTGGGGACCTGCTGTTCGAGGAAGAGGTGGAACAGTGCTTCGACCTG
TGTCACCAAGTGCTGCACCACTGCAGCAGCTCCATGGACGTGACAAGATCCCAGGCCTGCGCCAC
TCTGTATCTGCTGATGCGGTTTAGCTTCGGCGCCACCAGCAATTTCGCCAGAGTGAAGATGCAAG
TGACCATGAGCCTGGCCAGCCTCGTTGGAAGGGCCCCTGACTTCAATGAGGAACACCTCAGAAGA
AGCCTGCGGACCATCCTGGCCTACAGCGAAGAGGATACCGCCATGCAGATGACCCCATTTCCAAC
ACAAGTGGAAGAACTGCTGTGCAACCTGAATTCCATCCTGTACGATACCGTGAAGATGAGAGAGT
TCCAAGAGGACCCCGAGATGCTGATGGATCTGATGTACCGGATCGCCAAGAGCTACCAGGCCTCT
CCTGATCTGAGACTGACCTGGCTGCAGAACATGGCCGAGAAGCACACCAAGAAGAAGTGCTACAC
CGAGGCCGCCATGTGCCTGGTTCATGCTGCTGCACTCGTGGCCGAGTACCTGAGCATGCTGGAAG
ATCACTCCTACCTGCCAGTGGGCAGCGTGTCCTTCCAGAACATCAGCTCCAACGTCCTGGAAGAG
TCCGTGGTGTCCGAGGACACACTGAGCCCAGATGAGGATGGCGTGTGTGCCGGACAGTACTTTAC
CGAGTCTGGCCTCGTGGGCCTTCTGGAACAGGCCGCCGAACTGTTTTCTACCGGCGGACTGTACG
AGACAGTGAACGAAGTGTACAAGCTGGTCATCCCCATCCTGGAAGCCCACAGAGAGTTCAGAAAG
CTGACACTGACCCACTCCAAGCTGCAGCGGGCCTTCGACAGCATTGTGAACAAGGACCACAAGCG
GATGTTCGGCACCTACTTCAGAGTGGGCTTTTTCGGCAGCAAATTCGGCGACCTGGACGAGCAAG
AGTTCGTGTACAAAGAGCCCGCCATCACCAAGCTGCCTGAGATCTCCCATCGGCTGGAAGCCTTC
TACGGCCAGTGTTTTGGCGCCGAGTTCGTGGAAGTGATCAAGGACAGCACCCCTGTGGATAAGAC
CAAACTGGACCCTAACAAGGCCTACATCCAGATCACCTTCGTGGAACCCTACTTCGACGAGTACG
AGATGAAGGATAGAGTGACCTACTTTGAGAAGAACTTCAACCTGCGGCGGTTCATGTACACCACA
CCTTTTACACTGGAAGGCAGACCCAGGGGCGAGCTGCACGAGCAGTATAGAAGAAACACCGTGCT
GACCACCATGCACGCATTCCCTTACATCAAGACCCGGATCAGCGTGATCCAGAAAGAAGAGTTTG
TCCTGACACCAATCGAGGTGGCCATCGAGGACATGAAGAAGAAAACCCTCCAGCTGGCCGTGGCC
ATCAATCAAGAACCTCCAGACGCCAAGATGCTCCAGATGGTGCTCCAGGGATCTGTGGGCGCCAC
AGTGAATCAGGGACCTCTGGAAGTGGCCCAAGTGTTCCTGGCTGAGATCCCCGCCGATCCTAAGC
TGTACAGACACCATAACAAGCTGCGGCTCTGCTTCAAAGAATTCATCATGAGATGTGGCGAGGCC
GTGGAAAAGAACAAGAGACTGATCACCGCCGACCAGAGAGAGTACCAGCAAGAGCTGAAGAAAAA
CTACAACAAGCTCAAAGAAAATCTGCGGCCCATGATCGAGAGAAAGATCCCCGAGCTGTACAAGC
CCATCTTCCGCGTGGAAAGCCAGAAGCGGGACTCCTTTCACAGATCCAGCTTTAGGAAGTGCGAG
ACACAGCTGTCCCAGGGCTCTTGActggtgtggctaatgccctggcccacaagtAtcactaagct
cgcttTcttgctgtccaatttctattaaaggttccttTgttccctaagtccaactactaaactgg
gggatattatgaagggccttgagcatctggattctgcctaataaaaaacatttattttcattgca
atgatgtatttaaattatttctgaatattttactaaaaagggaatgtgggaggtcagtgcattta
aaacataaagaaatgaagagctagttcaaaccttgggaaaatacactatatcttaaactccatga
aagaaggtgaggctgcaaacagctaatgcacattggcaacagCccctgatgcctatgccttattc
atccctcagaaaaggattcaagtagaggcttgatttggaggttaaagtttTgctatgctgtattt
tacattacttattgttttagctgtcctcatgaatgtcttttcactacccatttgcttatcctgca
tctctcagccttgactccactcagttctcttgcttagagataccacctttcccctgaagtgttcc
ttccatgttttacggcgagatggtttctcctcgcctggccactcagccttagttgtctctgttgt
cttatagaggtctacttgaagaaggaaaaacagggggcatggtttgactgtcctgtgagcccttc
ttccctgcctcccccactcacagtgacccggaatccctcgacatggcagtctagcactagtgcgg
ccgcagatctgcttcctcgctcactgactcgctgcgctcggtcgttcggctgcggcgagcggtat
cagctcactcaaaggcggtaatacggttatccacagaatcaggggataacgcaggaaagaacatg
tgagcaaaaggccagcaaaaggccaggaaccgtaaaaaggccgcgttgctggcgtttttccatag
gctccgcccccctgacgagcatcacaaaaatcgacgctcaagtcagaggtggcgaaacccgacag
gactataaagataccaggcgtttccccctggaagctccctcgtgcgctctcctgttccgaccctg
ccgcttaccggatacctgtccgcctttctcccttcgggaagcgtggcgctttctcaTAgctcacg
ctgtaggtatctcagttcggtgtaggtcgttcgctccaagctgggctgtgtgcacgaaccccccg
ttcagcccgaccgctgcgccttatccggtaactatcgtcttgagtccaacccggtaagacacgac
ttatcgccactggcagcagccactggtaacaggattagcagagcgaggtatgtaggcggtgctac
agagttcttgaagtggtggcctaactacggctacactagaagAacagtatttggtatctgcgctc
tgctgaagccagttaccttcggaaaaagagttggtagctcttgatccggcaaacaaaccaccgct
ggtagcggtggtttttttgtttgcaagcagcagattacgcgcagaaaaaaaggatctcaa
(SEQ ID NO: 7)
DOCK8 939 CfaC-CF Intein Lentiviral plasmid sequence
mCCL-EFS-939Dock8-CfaC-CF-WPRE (10436 bp)
(see e.g. FIG. 16)
acgcgccctgtagcggcgcattaagcgcggcgggtgtggtggttacgcgcagcgtgaccgctaca
cttgccagcgccctagcgcccgctcctttcgctttcttcccttcctttctcgccacgttcgccgg
ctttccccgtcaagctctaaatcgggggctccctttagggttccgatttagtgctttacggcacc
tcgaccccaaaaaacttgattagggtgatggttcacgtagtgggccatcgccctgatagacggtt
tttcgccctttgacgttggagtccacgttctttaatagtggactcttgttccaaactggaacaac
actcaaccctatctcggtctattcttttgatttataagggattttgccgatttcggcctattggt
taaaaaatgagctgatttaacaaaaatttaacgcgaattttaacaaaatattaacgcttacaatt
taggtggcacttttcggggaaatgtgcgcggaacccctatttgtttatttttctaaatacattca
aatatgtatccgctcatgagacaataaccctgataaatgcttcaataatagcacctagatcaaga
gacaggatgaggatcgtttcgcatgattgaacaagatggattgcacgcaggttctccggccgctt
gggtggagaggctattcggctatgactgggcacaacagacaatcggctgctctgatgccgccgtg
ttccggctgtcagcgcaggggcgcccggttctttttgtcaagaccgacctgtccggtgccctgaa
tgaactgcaagacgaggcagcgcggctatcgtggctggccacgacgggcgttccttgcgcagctg
tgctcgacgttgtcactgaagcgggaagggactggctgctattgggcgaagtgccggggcaggat
ctcctgtcatctcaccttgctcctgccgagaaagtatccatcatggctgatgcaatgcggcggct
gcatacgcttgatccggctacctgcccattcgaccaccaagcgaaacatcgcatcgagcgagcac
gtactcggatggaagccggtcttgtcgatcaggatgatctggacgaagagcatcaggggctcgcg
ccagccgaactgttcgccaggctcaaggcgagcatgcccgacggcgaggatctcgtcgtgaccca
tggcgatgcctgcttgccgaatatcatggtggaaaatggccgcttttctggattcatcgactgtg
gccggctgggtgtggcggaccgctatcaggacatagcgttggctacccgtgatattgctgaagag
cttggcggcgaatgggctgaccgcttcctcgtgctttacggtatcgccgctcccgattcgcagcg
catcgccttctatcgccttcttgacgagttcttctgaattattaacgcttacaatttcctgatgc
ggtattttctccttacgcatctgtgcggtatttcacaccgcatcaggtggcacttttcggggaaa
tgtgcgcggaacccctatttgtttatttttctaaatacattcaaatatgtatccgctcatgacca
aaatcccttaacgtgagttttcgttccactgagcgtcagaccccgtagaaaagatcaaaggatct
tcttgagatcctttttttctgcgcgtaatctgctgcttgcaaacaaaaaaaccaccgctaccagc
ggtggtttgtttgccggatcaagagctaccaactctttttccgaaggtaactggcttcagcagag
cgcagataccaaatactgttcttctagtgtagccgtagttaggccaccacttcaagaactctgta
gcaccgcctacatacctcgctctgctaatcctgttaccagtggctgctgccagtggcgataagtc
gtgtcttaccgggttggactcaagacgatagttaccggataaggcgcagcggtcgggctgaacgg
gggggtcgtgcacacagcccagcttggagcgaacgacctacaccgaactgagatacctacagcgt
gagctatgagaaagcgccacgcttcccgaagggagaaaggcggacaggtatccgglaagcggcag
ggtcggaacaggagagcgcacgagggagcttccagggggaaacgcciggtatctttatagtcctg
tcgggtttcgccacctctgacttgagcgtcgatttttgtgatgctcgtcaggggggcggagccta
tggaaaaacgccagcaacgcggcctttttacggttcctggccttttgctggccttttgctcacat
gttctttcctgcgttatcccctgattctgtggataaccgtattaccgcctttgagtgagctgata
ccgctcgccgcagccgaacgaccgagcgcagcgagtcagtgagcgaggaagcggaagagcgccca
atacgcaaaccgcctctccccgcgcgttggccgattcattaatgcagctggcacgacaggtttcc
cgactggaaagcgggcagtgagcgcaacgcaattaatgtgagttagctcactcattaggcacccc
aggctttacactttatgcttccggctcgtatgttgtgtggaattgtgagcggataacaatttcac
acaggaaacagctatgaccatgattacgccaagcgcgcaattaaccctcactaaagggaacaaaa
gctggagctgcaagcttggccattgcatacgttgtatccatatcataatatgtacatttatattg
gctcatgtccaacattaccgccatgttgacattgattattgactagttattaatagtaatcaatt
acggggtcattagttcatagcccatatatggagttccgcgttacataacttacggtaaatggccc
gcctggctgaccgcccaacgacccccgcccattgacgtcaataatgacgtatgttcccatagtaa
cgccaatagggactttccattgacgtcaatgggtggagtatttacggtaaactgcccacttggca
gtacatcaagtgtatcatatgccaagtacgccccctattgacgtcaatgacggtaaatggcccgc
ctggcattatgcccagtacatgaccttatgggactttcctacttggcagtacatctacgtattag
tcatcgctattaccatggtgatgcggttttggcagtacatcaatgggcgtggatagcggtttgac
tcacggggatttccaagtctccaccccattgacgtcaatgggagtttgttttggcaccaaaatca
acgggactttccaaaatgtcgtaacaactccgccccattgacgcaaatgggcggtaggcgtgtac
ggtgggaggtctatataagcagagctcgtttagtgaaccgGGGTCTCTCTGGTTAGACCAGATCT
GAGCCTGGGAGCTCTCTGGCTAACTAGGGAACCCACTGCTTAAGCCTCAATAAAGCTTGCCTTGA
GTGCTTCAAGTAGTGTGTGCCCGTCTGTTGTGTGACTCTGGTAACTAGAGATCCCTCAGACCCTT
TTAGTCAGTGTGGAAAATCTCTAGCagtggcgcccgaacagggacttgaaagcgaaagggaaacc
agaggagctctcTCGACGCAGGACTCGGCTTGCTGAAGCGCGCACGGCAAGAGGCGAGGGGCGGC
GACTGGTGAGTACGCCAAAAATTTTGACTAGCGGAGGCTAGAAGGAGAGAGATGGGTGCGAGAGC
GTCAGTATTAAGCGGGGGAGaattagatcgcgatgggaaaaaattcggttaaggccagggggaaa
gaaaaaatataaattaaaacatatagtatgggcaagcagggagctagaacgattcgcagttaatc
ctggcctgttagaaacatcagaaggctgtagacaaatactgggacagctacaaccatcccttcag
acaggatcagaagaacttagatcattatataatacagtagcaaccctctattgtgtgcatcaaag
gatagagataaaagacaccaaggaagctttagacaagatagaggaagagcaaaacaaaagtaaga
ccaccgcacagcaagcGGCCGCTgatcttcagacctggaggaggagatatgagggacaattggag
aagtgaattatataaatataaagtagtaaaaattgaaccattaggagtagcacccaccaaggcaa
agagaagagtggtgcagagagaaaaaagaGCAGTGGGAATaggagctttgttccttgggttcttg
ggagcagcaggaagcactatgggcgcagcgtcaatgacgctgacggtacaggccagacaattatt
gtctggtatagtgcagcagcagaacaatttgctgagggctattgaggcgcaacagcatctgttgc
aactcacagtctggggcatcaagcagctccaggcaagaatcctggctgtggaaagatacctaaag
gatcaacagctcctggggatttggggttgctctggaaaactcatttgcaccactgctgtgccttg
gaatgctagttggagtaataaatctctggaacagatttggaatcacacgacctggatggagtggg
acagagaaattaacaattacacaagcttaatacactccttaattgaagaatcgcaaaaccagcaa
gaaaagaatgaacaagaattattggaattagataaatgggcaagtttgtggaattggtttaacat
aacaaattggctgtggtatataaaattattcataatgatagtaggaggcttggtaggtttaagaa
tagtttttgctgtactttctatagtgaatagagttaggcagggatattcaccattatcgtttcag
acccacctcccaaccccgaggggacccgacaggcccgaaggaatagaagaagaaggtggagagag
agacagagacagatccattcgattagtgaacggatCacaaatggcagtattcatccacaatttta
aaagaaaaggggggattggggggtacagtgcaggggaaagaatagtagacataatagcaacagac
atacaaactaaagaattacaaaaacaaattacaaaaattcaaaattttcgggtttattacaggga
cagcagaggctccggtgcccgtcagtgggcagagcgcacatcgcccacagtccccgagaagttgg
ggggaggggtcggcaattgaaccggtgcctagagaaggtggcgcggggtaaactgggaaagtgat
gtcgtgtactggctccgcctttttcccgaggggggggagaaccgtatataagtgcagtagtcgcc
gtgaacgttctttttcgcaacgggtttgccgccagaacacaggtgtcgtgaAAATAAGAGAGAAA
AGAAGAGTAAGAAGAAATATAAGAGCCACCatgGTGAAGATCATCAGCAGAAAGAGCCTGGGCAC
CCAGAACGTGTACGACATCGGCGTGGAAAAGGACCACAACTTTCTGCTCAAGAACGGCCTGGTGG
CCAGCAACTGCTTTAGCGGCAGCTCTGATGCCCCTAGCTCTCCAGCTGCTCCTAGACCTGCCAGC
AAGAAGCACTTTCACGAGGAACTGGCCCTGCAGATGGTCGTGTCTACCGGCATGGTTCGAGAGAC
AGTGTTTAAGTACGCCTGGTTCTTCTTCGAACTGCTGGTCAAGAGCATGGCCCAGCACGTGCACA
ACATGGACAAGCGGGACAGCTTCCGGCGGACCAGATTCAGCGACAGATTCATGGACGACATCACC
ACCATCGTGAACGTGGTCACCAGCGAGATTGCTGCCCTGCTGGTTAAGCCCCAGAAAGAGAACGA
GCAGGCCGAAAAGATGAATATCTCTCTGGCCTTCTTCCTCTACGACCTGCTGAGCCTGATGGACC
GGGGCTTCGTGTTCAACCTGATCCGGCACTACTGCTCCCAGCTGTCTGCCAAGCTGTCCAACCTG
CCTACACTGATCTCTATGAGACTCGAGTTCCTGAGGATCCTGTGCAGCCACGAGCACTACCTGAA
CCTGAATCTGTTCTTCATGAACGCCGACACAGCCCCTACAAGCCCCTGTCCTTCTATCAGCAGCC
AGAACAGCAGCAGCTGTAGCAGCTTTCAGGATCAGAAAATCGCCAGCATGTTCGATCTGACCAGC
GAGTACCGGCAGCAGCACTTTCTGACCGGCCTGCTGTTTACCGAACTGGCAGCCGCTCTTGATGC
CGAAGGCGAGGGAATCAGCAAGGTGCAGCGGAAAGCCGTGTCTGCCATTCACAGCCTGCTGTCTA
GCCACGACCTGGATCCTAGATGCGTGAAGCCTGAAGTGAAAGTGAAAATCGCTGCCCTGTACCTG
CCTCTCGTGGGCATCATTCTGGATGCCCTGCCTCAGCTGTGTGATTTCACCGTGGCCGACACCAG
ACGGTACAGAACCTCTGGCTCCGACGAGGAACAAGAAGGCGCTGGCGCCATCAACCAGAATGTGG
CCCTTGCCATTGCCGGGAACAACTTCAATCTGAAAACCAGCGGCATCGTGCTGAGCAGCCTGCCT
TACAAGCAGTACAACATGCTGAATGCCGACACTACCCGCAACCTGATGATCTGCTTCCTGTGGAT
TATGAAGAACGCCGATCAGAGCCTGATTCGGAAGTGGATTGCCGACCTGCCAAGCACTCAGCTGA
ACCGGATTCTGGACCTCCTGTTCATCTGCGTGCTGTGCTTCGAGTACAAGGGCAAGCAGTCCAGC
GACAAGGTGTCCACTCAGGTGCTGCAGAAAAGCCGGGATGTGAAGGCCAGGCTGGAAGAGGCACT
TCTGAGAGGCGAAGGCGCCAGAGGCGAGATGATGAGGCGTAGAGCCCCTGGCAACGATAGATTCC
CCGGCCTGAATGAGAACCTGCGGTGGAAGAAAGAGCAGACCCATTGGAGACAGGCCAACGAGAAA
CTGGACAAGACCAAGGCCGAACTGGACCAAGAGGCCCTGATCTCTGGCAACCTGGCCACAGAAGC
CCACCTGATTATCCTGGACATGCAAGAGAACATCATCCAGGCCAGTAGCGCCCTGGATTGCAAGG
ATTCTCTGCTCGGAGGCGTGCTGAGGGTGCTCGTGAATAGCCTGAATTGCGACCAGAGCACCACC
TACCTGACACACTGCTTCGCCACACTGAGGGCCCTGATTGCCAAGTTTGGGGACCTGCTGTTCGA
GGAAGAGGTGGAACAGTGCTTCGACCTGTGTCACCAAGTGCTGCACCACTGCAGCAGCTCCATGG
ACGTGACAAGATCCCAGGCCTGCGCCACTCTGTATCTGCTGATGCGGTTTAGCTTCGGCGCCACC
AGCAATTTCGCCAGAGTGAAGATGCAAGTGACCATGAGCCTGGCCAGCCTCGTTGGAAGGGCCCC
TGACTTCAATGAGGAACACCTCAGAAGAAGCCTGCGGACCATCCTGGCCTACAGCGAAGAGGATA
CCGCCATGCAGATGACCCCATTTCCAACACAAGTGGAAGAACTGCTGTGCAACCTGAATTCCATC
CTGTACGATACCGTGAAGATGAGAGAGTTCCAAGAGGACCCCGAGATGCTGATGGATCTGATGTA
CCGGATCGCCAAGAGCTACCAGGCCTCTCCTGATCTGAGACTGACCTGGCTGCAGAACATGGCCG
AGAAGCACACCAAGAAGAAGTGCTACACCGAGGCCGCCATGTGCCTGGTTCATGCTGCTGCACTC
GTGGCCGAGTACCTGAGCATGCTGGAAGATCACTCCTACCTGCCAGTGGGCAGCGTGTCCTTCCA
GAACATCAGCTCCAACGTCCTGGAAGAGTCCGTGGTGTCCGAGGACACACTGAGCCCAGATGAGG
ATGGCGTGTGTGCCGGACAGTACTTTACCGAGTCTGGCCTCGTGGGCCTTCTGGAACAGGCCGCC
GAACTGTTTTCTACCGGCGGACTGTACGAGACAGTGAACGAAGTGTACAAGCTGGTCATCCCCAT
CCTGGAAGCCCACAGAGAGTTCAGAAAGCTGACACTGACCCACTCCAAGCTGCAGCGGGCCTTCG
ACAGCATTGTGAACAAGGACCACAAGCGGATGTTCGGCACCTACTTCAGAGTGGGCTTTTTCGGC
AGCAAATTCGGCGACCTGGACGAGCAAGAGTTCGTGTACAAAGAGCCCGCCATCACCAAGCTGCC
TGAGATCTCCCATCGGCTGGAAGCCTTCTACGGCCAGTGTTTTGGCGCCGAGTTCGTGGAAGTGA
TCAAGGACAGCACCCCTGTGGATAAGACCAAACTGGACCCTAACAAGGCCTACATCCAGATCACC
TTCGTGGAACCCTACTTCGACGAGTACGAGATGAAGGATAGAGTGACCTACTTTGAGAAGAACTT
CAACCTGCGGCGGTTCATGTACACCACACCTTTTACACTGGAAGGCAGACCCAGGGGCGAGCTGC
ACGAGCAGTATAGAAGAAACACCGTGCTGACCACCATGCACGCATTCCCTTACATCAAGACCCGG
ATCAGCGTGATCCAGAAAGAAGAGTTTGTCCTGACACCAATCGAGGTGGCCATCGAGGACATGAA
GAAGAAAACCCTCCAGCTGGCCGTGGCCATCAATCAAGAACCTCCAGACGCCAAGATGCTCCAGA
TGGTGCTCCAGGGATCTGTGGGCGCCACAGTGAATCAGGGACCTCTGGAAGTGGCCCAAGTGTTC
CTGGCTGAGATCCCCGCCGATCCTAAGCTGTACAGACACCATAACAAGCTGCGGCTCTGCTTCAA
AGAATTCATCATGAGATGTGGCGAGGCCGTGGAAAAGAACAAGAGACTGATCACCGCCGACCAGA
GAGAGTACCAGCAAGAGCTGAAGAAAAACTACAACAAGCTCAAAGAAAATCTGCGGCCCATGATC
GAGAGAAAGATCCCCGAGCTGTACAAGCCCATCTTCCGCGTGGAAAGCCAGAAGCGGGACTCCTT
TCACAGATCCAGCTTTAGGAAGTGCGAGACACAGCTGTCCCAGGGCTCTTGAttccgataatcaa
cctctggattacaaaatttgtgaaagattgactggtattcttaactatgttgctccttttacgct
atgtggatacgctgctttaatgcctttgtatcatgctattgcttcccgtatggctttcattttct
cctccttgtataaatcctggttgctgtctctttatgaggagttgtggcccgttgtcaggcaacgt
ggcgtggtgtgcactgtgtttgctgacgcaacccccactggttggggcattgccaccacctgtca
gctcctttccgggactttcgctttccccctccctattgccacggcggaactcatcgccgcctgcc
ttgcccgctgctggacaggggctcggctgttgggcactgacaattccgtggtgttgtcggggaaA
TCATcgtcctttccTtggctgctcgcctgtgttgccacctggattctgcgcgggacgtccttctg
ctacgtcccttcggccctcaatccagcggaccttccttcccgcggcctgctgccggctctgcggc
ctcttccgcgtcttcgccttcgccctcagacgagtcggatctccctttgggccgcctccccgcTa
aaagaaaaggggggactggaagggctaattcactcccaacgaagacaagatctgctttttgcttg
tactGGGTCTCTCTGGTTAGACCAGATCTGAGCCTGGGAGCTCTCTGGCTAACTAGGGAACCCAC
TGCTTAAGCCTCAATAAAGCTTGCCTTGAGTGCTTCAAGTAGTGTGTTGGTTTTTTGTGTGTGCA
TTGTCTGAGTAGGTGTCATTCTATTCTGGGGGGTGGGGTGGGGCAGCACAGCAAGGGGGAGGATT
GGGAAGACAATAGCAGGCATGCTGGGGATGCGGTGGGCTCTATGGagtagtagttcatgtcatct
tattattcagtatttataacttgcaaagaaatgaatatcagagagtgagaggaacttgtttattg
cagcttataatggttacaaataaagcaatagcatcacaaatttcacaaataaagcatttttttca
ctgcattctagttgtggtttgtccaaactcatcaatgtatcttatcatgtctggctctagctatc
ccgcccctaactccgcccATCCCGCCCCTAACTCCGCCCAGTTCCGCCCATTCTCCGCCCCATGG
CTCTCACTACTTCTGGAATAGCTCAGAGGCCGAGGCGGCCTCGGCCTCTGCATAAATAAAAAAAA
TTAGTCGAGGCTTTTTTGGAGGCCTAGGgacgtacccaattcgccctatagtgagtcgtattacg
cgcgctcactggccgtcgttttacaacgtcgtgactgggaaaaccctggcgttacccaacttaat
cgccttgcagcacatccccctttcgccagctggcgtaatagcgaagaggcccgcaccgatcgccc
ttcccaacagttgcgcagcctgaatggcgaatggg
(SEQ ID NO: 8)
DOCK8 939 CfaC-CFN Intein plasmid sequence
939 CfaC-CFN-DOCK8-bGHpA (7733 bp)
(see e.g. FIG. 17)
ctgtctatttcgttcatccatagttgcctgactccccgtcgtgtagataactacgatacgggagg
gcttaccatctggccccagtgctgcaatgataccgcgagacccacgctcaccggctccagattta
tcagcaataaaccagccagccggaagggccgagcgcagaagtggtcctgcaactttatccgcctc
catccagtctattaattgttgccgggaagctagagtaagtagttcgccagttaatagtuigcgca
acgitgtigccatigctacaggcatcgtggtgtcacgctcgicgittggtatggcttcattcagc
tccggttcccaacgatcaaggcgagttacatgatcccccatgttgtgcaaaaaagcggttagctc
cttcggtcctccgatcgttgtcagaagtaagttggccgcagtgttatcactcatggttatggcag
cactgcataattctcttactgtcatgccatccgtaagatgcttttctgtgactggtgagtactca
accaagtcattctgagaatagtgtatgcggcgaccgagttgctcttgcccggcgtcaatacggga
taataccgcgccacatagcagaactttaaaagtgctcatcattggaaaacgttcttcggggcgaa
aactctcaaggatcttaccgctgttgagatccagttcgatgtaacccactcgtgcacccaactga
tcttcagcatcttttactttcaccagcgtttctgggtgagcaaaaacaggaaggcaaaatgccgc
aaaaaagggaataagggcgacacggaaatgttgaatactcatactcttcctttttcaatattatt
gaagcatttatcagggttattgtctcatgagcggatacatatttgaatgtatttagaaaaataaa
caaataggggttccgcgcacatttccccgaaaagtgccacctgacgtggatcccctgagggggcc
cccatgggctagaggatccggcctcggcctctgcataaataaaaaaaattagtcagccatgagct
tggcccattgcatacgttgtatccatatcataatatgtacatttatattggctcatgtccaacat
taccgccatgttgacattgattattgactagttattaatagtaatcaattacggggtcattagtt
catagcccatatatggagttccgcgttacataacttacggtaaatggcccgcctggctgaccgcc
caacgacccccgcccattgacgtcaataatgacgtatgttcccatagtaacgccaatagggactt
tccattgacgtcaatgggtggagtatttacggtaaactgcccacttggcagtacatcaagtgtat
catatgccaagtacgccccctattgacgtcaatgacggtaaatggcccgcctggcattatgccca
gtacatgaccttatgggactttcctacttggcagtacatctacgtattagtcatcgctattacca
tggtgatgcggttttggcagtacatcaatgggcgtggatagcggtttgactcacggggatttcca
agtctccaccccattgacgtcaatgggagtttgttttggcaccaaaatcaacgggactttccaaa
atgtcgtaacaactccgccccattgacgcaaatgggcggtaggcgtgtacggtgggaggtctata
taagcagagctcgtttagtgaaccgtcagatcgcctggagacgccatccacgctgttttgacctc
catagaagacaccgggaccgatccagcctcccctcgaagcttacatgtggtaccgagctcggatc
ctgagaacttcagggtgagtctatgggacccttgatgttttctttccccttcttttctatggtta
agttcatgtcataggaaggggagaagtaacagggtacacatattgaccaaatcagggtaattttg
catttgtaattttaaaaaatgctttcttcttttaatatacttttttgtttatcttatttctaata
ctttccctaatctctttctttcagggcaataatgatacaatgtatcatgcctctttgcaccattc
taaagaataacagtgataatttctgggttaaggcaatagcaatatttctgcatataaatatttct
gcatataaattgtaactgatgtaagaggtttcatattgctaatagcagctacaatccagctacca
ttctgcttttattttatggttgggataaggctggattattctgagtccaagctaggcccttttgc
taatcatgttcatacctcttatcttcctcccacagctcctgggcaacgtgctggtctgtgtgctg
gcccatcactttggcaaagcacgtgagatctgaattcgagatctgccgccgccatgGTGAAGATC
ATCAGCAGAAAGAGCCTGGGCACCCAGAACGTGTACGACATCGGCGTGGAAAAGGACCACAACTT
TCTGCTCAAGAACGGCCTGGTGGCCAGCAACTGCTTTAATAGCGGCAGCTCTGATGCCCCTAGCT
CTCCAGCTGCTCCTAGACCTGCCAGCAAGAAGCACTTTCACGAGGAACTGGCCCTGCAGATGGTC
GTGTCTACCGGCATGGTTCGAGAGACAGTGTTTAAGTACGCCTGGTTCTTCTTCGAACTGCTGGT
CAAGAGCATGGCCCAGCACGTGCACAACATGGACAAGCGGGACAGCTTCCGGCGGACCAGATTCA
GCGACAGATTCATGGACGACATCACCACCATCGTGAACGTGGTCACCAGCGAGATTGCTGCCCTG
CTGGTTAAGCCCCAGAAAGAGAACGAGCAGGCCGAAAAGATGAATATCTCTCTGGCCTTCTTCCT
CTACGACCTGCTGAGCCTGATGGACCGGGGCTTCGTGTTCAACCTGATCCGGCACTACTGCTCCC
AGCTGTCTGCCAAGCTGTCCAACCTGCCTACACTGATCTCTATGAGACTCGAGTTCCTGAGGATC
CTGTGCAGCCACGAGCACTACCTGAACCTGAATCTGTTCTTCATGAACGCCGACACAGCCCCTAC
AAGCCCCTGTCCTTCTATCAGCAGCCAGAACAGCAGCAGCTGTAGCAGCTTTCAGGATCAGAAAA
TCGCCAGCATGTTCGATCTGACCAGCGAGTACCGGCAGCAGCACTTTCTGACCGGCCTGCTGTTT
ACCGAACTGGCAGCCGCTCTTGATGCCGAAGGCGAGGGAATCAGCAAGGTGCAGCGGAAAGCCGT
GTCTGCCATTCACAGCCTGCTGTCTAGCCACGACCTGGATCCTAGATGCGTGAAGCCTGAAGTGA
AAGTGAAAATCGCTGCCCTGTACCTGCCTCTCGTGGGCATCATTCTGGATGCCCTGCCTCAGCTG
TGTGATTTCACCGTGGCCGACACCAGACGGTACAGAACCTCTGGCTCCGACGAGGAACAAGAAGG
CGCTGGCGCCATCAACCAGAATGTGGCCCTTGCCATTGCCGGGAACAACTTCAATCTGAAAACCA
GCGGCATCGTGCTGAGCAGCCTGCCTTACAAGCAGTACAACATGCTGAATGCCGACACTACCCGC
AACCTGATGATCTGCTTCCTGTGGATTATGAAGAACGCCGATCAGAGCCTGATTCGGAAGTGGAT
TGCCGACCTGCCAAGCACTCAGCTGAACCGGATTCTGGACCTCCTGTTCATCTGCGTGCTGTGCT
TCGAGTACAAGGGCAAGCAGTCCAGCGACAAGGTGTCCACTCAGGTGCTGCAGAAAAGCCGGGAT
GTGAAGGCCAGGCTGGAAGAGGCACTTCTGAGAGGCGAAGGCGCCAGAGGCGAGATGATGAGGCG
TAGAGCCCCTGGCAACGATAGATTCCCCGGCCTGAATGAGAACCTGCGGTGGAAGAAAGAGCAGA
CCCATTGGAGACAGGCCAACGAGAAACTGGACAAGACCAAGGCCGAACTGGACCAAGAGGCCCTG
ATCTCTGGCAACCTGGCCACAGAAGCCCACCTGATTATCCTGGACATGCAAGAGAACATCATCCA
GGCCAGTAGCGCCCTGGATTGCAAGGATTCTCTGCTCGGAGGCGTGCTGAGGGTGCTCGTGAATA
GCCTGAATTGCGACCAGAGCACCACCTACCTGACACACTGCTTCGCCACACTGAGGGCCCTGATT
GCCAAGTTTGGGGACCTGCTGTTCGAGGAAGAGGTGGAACAGTGCTTCGACCTGTGTCACCAAGT
GCTGCACCACTGCAGCAGCTCCATGGACGTGACAAGATCCCAGGCCTGCGCCACTCTGTATCTGC
TGATGCGGTTTAGCTTCGGCGCCACCAGCAATTTCGCCAGAGTGAAGATGCAAGTGACCATGAGC
CTGGCCAGCCTCGTTGGAAGGGCCCCTGACTTCAATGAGGAACACCTCAGAAGAAGCCTGCGGAC
CATCCTGGCCTACAGCGAAGAGGATACCGCCATGCAGATGACCCCATTTCCAACACAAGTGGAAG
AACTGCTGTGCAACCTGAATTCCATCCTGTACGATACCGTGAAGATGAGAGAGTTCCAAGAGGAC
CCCGAGATGCTGATGGATCTGATGTACCGGATCGCCAAGAGCTACCAGGCCTCTCCTGATCTGAG
ACTGACCTGGCTGCAGAACATGGCCGAGAAGCACACCAAGAAGAAGTGCTACACCGAGGCCGCCA
TGTGCCTGGTTCATGCTGCTGCACTCGTGGCCGAGTACCTGAGCATGCTGGAAGATCACTCCTAC
CTGCCAGTGGGCAGCGTGTCCTTCCAGAACATCAGCTCCAACGTCCTGGAAGAGTCCGTGGTGTC
CGAGGACACACTGAGCCCAGATGAGGATGGCGTGTGTGCCGGACAGTACTTTACCGAGTCTGGCC
TCGTGGGCCTTCTGGAACAGGCCGCCGAACTGTTTTCTACCGGCGGACTGTACGAGACAGTGAAC
GAAGTGTACAAGCTGGTCATCCCCATCCTGGAAGCCCACAGAGAGTTCAGAAAGCTGACACTGAC
CCACTCCAAGCTGCAGCGGGCCTTCGACAGCATTGTGAACAAGGACCACAAGCGGATGTTCGGCA
CCTACTTCAGAGTGGGCTTTTTCGGCAGCAAATTCGGCGACCTGGACGAGCAAGAGTTCGTGTAC
AAAGAGCCCGCCATCACCAAGCTGCCTGAGATCTCCCATCGGCTGGAAGCCTTCTACGGCCAGTG
TTTTGGCGCCGAGTTCGTGGAAGTGATCAAGGACAGCACCCCTGTGGATAAGACCAAACTGGACC
CTAACAAGGCCTACATCCAGATCACCTTCGTGGAACCCTACTTCGACGAGTACGAGATGAAGGAT
AGAGTGACCTACTTTGAGAAGAACTTCAACCTGCGGCGGTTCATGTACACCACACCTTTTACACT
GGAAGGCAGACCCAGGGGCGAGCTGCACGAGCAGTATAGAAGAAACACCGTGCTGACCACCATGC
ACGCATTCCCTTACATCAAGACCCGGATCAGCGTGATCCAGAAAGAAGAGTTTGTCCTGACACCA
ATCGAGGTGGCCATCGAGGACATGAAGAAGAAAACCCTCCAGCTGGCCGTGGCCATCAATCAAGA
ACCTCCAGACGCCAAGATGCTCCAGATGGTGCTCCAGGGATCTGTGGGCGCCACAGTGAATCAGG
GACCTCTGGAAGTGGCCCAAGTGTTCCTGGCTGAGATCCCCGCCGATCCTAAGCTGTACAGACAC
CATAACAAGCTGCGGCTCTGCTTCAAAGAATTCATCATGAGATGTGGCGAGGCCGTGGAAAAGAA
CAAGAGACTGATCACCGCCGACCAGAGAGAGTACCAGCAAGAGCTGAAGAAAAACTACAACAAGC
TCAAAGAAAATCTGCGGCCCATGATCGAGAGAAAGATCCCCGAGCTGTACAAGCCCATCTTCCGC
GTGGAAAGCCAGAAGCGGGACTCCTTTCACAGATCCAGCTTTAGGAAGTGCGAGACACAGCTGTC
CCAGGGCTCTTGActggtgtggctaatgccctggcccacaagtAtcactaagctcgcttTcttgc
tgtccaatttctattaaaggttccttTgttccctaagtccaactactaaactgggggatattatg
aagggccttgagcatctggattctgcctaataaaaaacatttattttcattgcaatgatgtattt
aaattatttctgaatattttactaaaaagggaatgtgggaggtcagtgcatttaaaacataaaga
aatgaagagctagttcaaaccttgggaaaatacactatatcttaaactccatgaaagaaggtgag
gctgcaaacagctaatgcacattggcaacagCccctgatgcctatgccttattcatccctcagaa
aaggattcaagtagaggcttgatttggaggttaaagtttTgctatgctgtattttacattactta
ttgttttagctgtcctcatgaatgtcttttcactacccatttgcttatcctgcatctctcagcct
tgactccactcagttctcttgcttagagataccacctttcccctgaagtgttccttccatgtttt
acggcgagatggtttctcctcgcctggccactcagccttagttgtctctgttgtcttatagaggt
ctacttgaagaaggaaaaacagggggcatggtttgactgtcctgtgagcccttcttccctgcctc
ccccactcacagtgacccggaatccctcgacatggcagtctagcactagtgcggccgcagatctg
cttcctcgctcactgactcgctgcgctcggtcgttcggctgcggcgagcggtatcagctcactca
aaggcggtaatacggttatccacagaatcaggggataacgcaggaaagaacatgtgagcaaaagg
ccagcaaaaggccaggaaccgtaaaaaggccgcgttgctggcgtttttccataggctccgccccc
ctgacgagcatcacaaaaatcgacgctcaagtcagaggtggcgaaacccgacaggactataaaga
taccaggcgtttccccctggaagctccctcgtgcgctctcctgttccgaccctgccgcttaccgg
atacctgtccgcctttctcccttcgggaagcgtggcgctttctcaTAgctcacgctgtaggtatc
tcagttcggtgtaggtcgttcgctccaagctgggctgtgtgcacgaaccccccgttcagcccgac
cgctgcgccttatccggtaactatcgtcttgagtccaacccggtaagacacgacttatcgccact
ggcagcagccactggtaacaggattagcagagcgaggtatgtaggcggtgctacagagttcttga
agtggtggcctaactacggctacactagaagAacagtatttggtatctgcgctctgctgaagcca
gttaccttcggaaaaagagttggtagctcttgatccggcaaacaaaccaccgctggtagcggtgg
tttttttgtttgcaagcagcagattacgcgcagaaaaaaaggatctcaagaagatcctttgatct
tttctacggggtctgacgctcagtggaacgaaaactcacgttaagggattttggtcatgagatta
tcaaaaaggatcttcacctagatccttttaaattaaaaatgaagttttaaatcaatctaaagtat
atatgagtaaacttggtctgacagttaccaatgcttaatcagtgaggcacctatctcagcgat
(SEQ ID NO: 9)
DOCK8 1267 DOCK8-CfaN-2 Intein plasmid sequence
1267 DOCK8-CfaN_2 (8240 bp)
ctgtctatttcgttcatccatagttgcctgactccccgtcgtgtagataactacgatacgggagg
gcttaccatctggccccagtgctgcaatgataccgcgagacccacgctcaccggctccagattta
tcagcaataaaccagccagccggaagggccgagcgcagaagtggtcctgcaactttatccgcctc
catccagtctattaattgttgccgggaagctagagtaagtagttcgccagttaatagtttgcgca
acgttgttgccattgctacaggcatcgtggtgtcacgctcgtcgtttggtatggcttcattcagc
tccggttcccaacgatcaaggcgagttacatgatcccccatgttgtgcaaaaaagcggttagctc
cttcggtcctccgatcgttgtcagaagtaagttggccgcagtgttatcactcatggttatggcag
cactgcataattctcttactgtcatgccatccgtaagatgcttttctgtgactggtgagtactca
accaagtcattctgagaatagtgtatgcggcgaccgagttgctcttgcccggcgtcaatacggga
taataccgcgccacatagcagaactttaaaagtgctcatcattggaaaacgttcttcggggcgaa
aactctcaaggatcttaccgctgttgagatccagttcgatgtaacccactcgtgcacccaactga
tcttcagcatcttttactttcaccagcgtttctgggtgagcaaaaacaggaaggcaaaatgccgc
aaaaaagggaataagggcgacacggaaatgttgaatactcatactcttcctttttcaatattatt
gaagcatttatcagggttattgtctcatgagcggatacatatttgaatgtatttagaaaaataaa
caaataggggttccgcgcacatttccccgaaaagtgccacctgacgtggatcccctgagggggcc
cccatgggctagaggatccggcctcggcctctgcataaataaaaaaaattagtcagccatgagct
tggcccattgcatacgttgtatccatatcataatatgtacatttatattggctcatgtccaacat
taccgccatgttgacattgattattgactagttattaatagtaatcaattacggggtcattagtt
catagcccatatatggagttccgcgttacataacttacggtaaatggcccgcctggctgaccgcc
caacgacccccgcccattgacgtcaataatgacgtatgttcccatagtaacgccaatagggactt
tccattgacgtcaatgggtggagtatttacggtaaactgcccacttggcagtacatcaagtgtat
catatgccaagtacgccccctattgacgtcaatgacggtaaatggcccgcctggcattatgccca
gtacatgaccttatgggactttcctacttggcagtacatctacgtattagtcatcgctattacca
tggtgatgcggttttggcagtacatcaatgggcgtggatagcggtttgactcacggggatttcca
agtctccaccccattgacgtcaatgggagtttgttttggcaccaaaatcaacgggactttccaaa
atgtcgtaacaactccgccccattgacgcaaatgggcggtaggcgtgtacggtgggaggtctata
taagcagagctcgtttagtgaaccgtcagatcgcctggagacgccatccacgctgttttgacctc
catagaagacaccgggaccgatccagcctcccctcgaagcttacatgtggtaccgagctcggatc
ctgagaacttcagggtgagtctatgggacccttgatgttttctttccccttcttttctatggtta
agttcatgtcataggaaggggagaagtaacagggtacacatattgaccaaatcagggtaattttg
catttgtaattttaaaaaatgctttcttcttttaatatacttttttgtttatcttatttctaata
ctttccctaatctctttctttcagggcaataatgatacaatgtatcatgcctctttgcaccattc
taaagaataacagtgataatttctgggttaaggcaatagcaatatttctgcatataaatatttct
gcatataaattgtaactgatgtaagaggtttcatattgctaatagcagctacaatccagctacca
ttctgcttttattttatggttgggataaggctggattattctgagtccaagctaggcccttttgc
taatcatgttcatacctcttatcttcctcccacagctcctgggcaacgtgctggtctgtgtgctg
gcccatcactttggcaaagcacgtgagatctgaattcgagatctgccgccgccATGGCCACACTG
CCTAGCGCTGAGAGAAGGGCCTTCGCTCTGAAGATCAACCGGTACAGCAGCGCCGAGATCCGGAA
GCAGTTTACCCTGCCTCCTAACCTGGGCCAGTACCACAGACAGAGCATCAGCACCAGCGGCTTCC
CATCTCTGCAGCTGCCCCAGTTCTACGACCCTGTGGAACCCGTGGATTTCGAGGGCCTGCTGATG
ACCCACCTGAACAGCCTGGATGTGCAGCTGGCCCAAGAGCTGGGCGACTTCACCGACGATGATCT
GGACGTGGTGTTCACCCCTAAAGAGTGCAGAACCCTGCAGCCTAGCCTGCCTGAAGAAGGCGTGG
AACTGGATCCTCACGTGCGGGATTGTGTGCAGACCTACATCCGCGAGTGGCTGATCGTGAACCGG
AAGAATCAGGGCAGCCCAGAGATCTGCGGCTTCAAGAAAACCGGCAGCCGGAAGGACTTCCACAA
GACCCTGCCTAAGCAGACCTTCGAGAGCGAGACACTGGAATGCTCTGAGCCTGCTGCTCAGGCCG
GACCTAGACACCTGAATGTGCTGTGTGACGTGTCCGGCAAGGGACCTGTGACCGCCTGCGATTTC
GACCTGAGAAGTCTGCAGCCCGACAAGCGGCTGGAAAACCTGCTCCAGCAAGTGTCCGCCGAGGA
CTTCGAGAAGCAGAACGAGGAAGCCAGACGGACCAACAGACAGGCCGAGCTGTTTGCTCTGTACC
CCAGCGTGGACGAGGAAGATGCCGTCGAGATTAGACCCGTGCCTGAGTGCCCAAAAGAGCACCTG
GGCAACAGAATCCTGGTCAAGCTGCTGACCCTGAAGTTCGAGATCGAGATTGAGCCCCTGTTCGC
CTCTATCGCCCTGTACGACGTGAAAGAGCGGAAGAAGATCAGCGAGAACTTCCACTGCGACCTGA
ACTCCGACCAGTTCAAGGGCTTCCTGAGAGCCCACACACCATCTGTGGCCGCTAGCTCTCAGGCC
AGATCTGCCGTGTTCAGCGTGACATACCCCAGCAGCGACATCTACCTGGTGGTCAAGATCGAGAA
GGTCCTGCAGCAGGGCGAGATCGGCGATTGTGCCGAGCCTTACACCGTGATCAAAGAGAGCGACG
GCGGCAAGAGCAAAGAGAAGATTGAGAAGCTGAAGCTGCAGGCCGAGAGCTTCTGTCAGCGGCTG
GGCAAGTACAGAATGCCCTTTGCCTGGGCTCCTATCAGCCTGTCCAGCTTCTTCAACGTGTCCAC
ACTGGAACGGGAAGTGACCGACGTGGACTCTGTCGTGGGCAGATCTAGCGTGGGCGAGAGAAGGA
CACTGGCTCAGTCTAGACGGCTGAGCGAGAGAGCCCTGAGCCTGGAAGAAAATGGCGTGGGCAGC
AACTTCAAGACCAGCACTCTGTCCGTGTCTAGCTTCTTTAAGCAAGAGGGCGACAGACTGAGCGA
CGAGGACCTGTTCAAGTTCCTGGCCGACTACAAGCGGAGCAGCTCCCTGCAGAGAAGAGTGAAGT
CTATCCCTGGCCTGCTGCGCCTGGAAATCTCTACAGCCCCTGAGATCATCAACTGCTGTCTGACC
CCTGAAATGCTGCCCGTGAAGCCCTTTCCAGAGAACCGGACCAGACCTCACAAAGAGATCCTGGA
ATTCCCCACCAGAGAAGTGTACGTGCCCCACACCGTGTACCGGAACCTGCTGTATGTGTACCCAC
AGCGGCTGAACTTCGTGAACAAGCTGGCCTCCGCCAGAAACATCACCATCAAGATCCAGTTTATG
TGCGGCGAGGACGCCAGCAACGCCATGCCTGTGATCTTCGGCAAGTCTAGCGGCCCTGAGTTCCT
GCAAGAGGTGTACACAGCCGTGACCTACCACAACAAGAGCCCCGACTTCTACGAGGAAGTGAAGA
TTAAGCTGCCCGCCAAGCTGACCGTGAATCACCATCTGCTGTTCACCTTCTACCACATCAGCTGC
CAGCAGAAACAGGGCGCCTCTGTGGAAACACTGCTGGGCTATAGCTGGCTGCCCATCCTGCTGAA
CGAGAGACTGCAGACCGGCAGCTACTGTCTGCCTGTGGCTCTGGAAAAGCTGCCACCTAACTACA
GCATGCACTCCGCCGAGAAGGTGCCCCTGCAGAATCCTCCTATTAAGTGGGCCGAGGGCCACAAG
GGCGTGTTCAATATCGAGGTGCAGGCCGTGTCCTCCGTGCACACCCAGGATAACCACCTGGAAAA
GTTCTTCACCCTGTGCCACAGCCTCGAGAGCCAAGTGACATTCCCCATCCGCGTGCTGGACCAGA
AAATCTCCGAGATGGCCCTGGAACACGAGCTGAAACTGAGCATCATCTGCCTGAATAGCAGCAGA
CTGGAACCCCTGGTGCTGTTCCTGCATCTGGTGCTGGACAAGCTGTTCCAGCTGAGCGTGCAGCC
CATGGTTATCGCCGGACAGACCGCCAACTTCAGCCAGTTCGCCTTTGAGAGCGTGGTGGCCATTG
CCAACAGCCTGCACAACAGCAAGGACCTGAGCAAGGATCAGCACGGCAGAAACTGCCTGCTGGCC
TCTTACGTGCACTACGTGTTCAGACTGCCTGAGGTGCAGAGGGACGTGCCAAAATCTGGCGCTCC
TACCGCTCTGCTGGACCCCAGAAGCTATCACACCTACGGCAGAACATCTGCCGCCGCTGTGTCCT
CTAAACTCCTGCAGGCTAGAGTGATGTCCAGCAGCAACCCTGATCTGGCCGGAACACACAGCGCC
GCTGATGAAGAAGTGAAAAACATCATGAGCAGCAAGATCGCCGACCGGAACTGCAGCCGGATGAG
CTACTACTGTAGCGGCAGCTCTGATGCCCCTAGCTCTCCAGCTGCTCCTAGACCTGCCAGCAAGA
AGCACTTTCACGAGGAACTGGCCCTGCAGATGGTCGTGTCTACCGGCATGGTTCGAGAGACAGTG
TTTAAGTACGCCTGGTTCTTCTTCGAACTGCTGGTCAAGAGCATGGCCCAGCACGTGCACAACAT
GGACAAGCGGGACAGCTTCCGGCGGACCAGATTCAGCGACAGATTCATGGACGACATCACCACCA
TCGTGAACGTGGTCACCAGCGAGATTGCTGCCCTGCTGGTTAAGCCCCAGAAAGAGAACGAGCAG
GCCGAAAAGATGAATATCTCTCTGGCCTTCTTCCTCTACGACCTGCTGAGCCTGATGGACCGGGG
CTTCGTGTTCAACCTGATCCGGCACTACTGCTCCCAGCTGTCTGCCAAGCTGTCCAACCTGCCTA
CACTGATCTCTATGAGACTCGAGTTCCTGAGGATCCTGTGCAGCCACGAGCACTACCTGAACCTG
AATCTGTTCTTCATGAACGCCGACACAGCCCCTACAAGCCCCTGTCCTTCTATCAGCAGCCAGAA
CAGCAGCAGCTGTAGCAGCTTTCAGGATCAGAAAATCGCCAGCATGTTCGATCTGACCAGCGAGT
ACCGGCAGCAGCACTTTCTGACCGGCCTGCTGTTTACCGAACTGGCAGCCGCTCTTGATGCCGAA
GGCGAGGGAATCAGCAAGGTGCAGCGGAAAGCCGTGTCTGCCATTCACAGCCTGCTGTCTAGCCA
CGACCTGGATCCTAGATGCGTGAAGCCTGAAGTGAAAGTGAAAATCGCTGCCCTGTACCTGCCTC
TCGTGGGCATCATTCTGGATGCCCTGCCTCAGCTGTGTGATTTCACCGTGGCCGACACCAGACGG
TACAGAACCTCTGGCTCCGACGAGGAACAAGAAGGCGCTGGCGCCATCAACCAGAATGTGGCCCT
TGCCATTGCCGGGAACAACTGCCTGAGCTACGACACCGAGATCCTGACCGTGGAATACGGCTTCC
TGCCTATCGGCAAGATCGTGGAAGAACGGATCGAGTGCACCGTGTACACCGTGGACAAGAACGGC
TTCGTGTACACACAGCCTATCGCTCAGTGGCACAACCGGGGAGAGCAAGAGGTGTTCGAGTACTG
CCTGGAAGATGGCAGCATCATCCGGGCCACCAAGGACCACAAGTTCATGACCACCGACGGCCAGA
TGCTGCCCATCGACGAGATCTTTGAGAGAGGCCTGGACCTGAAACAGGTGGACGGACTTCCTtga
ctggtgtggctaatgccctggcccacaagtAtcactaagctcgcttTcttgctgtccaatttcta
ttaaaggttccttTgttccctaagtccaactactaaactgggggatattatgaagggccttgagc
atctggattctgcctaataaaaaacatttattttcattgcaatgatgtatttaaattatttctga
atattttactaaaaagggaatgtgggaggtcagtgcatttaaaacataaagaaatgaagagctag
ttcaaaccttgggaaaatacactatatcttaaactccatgaaagaaggtgaggctgcaaacagct
aatgcacattggcaacagCccctgatgcctatgccttattcatccctcagaaaaggattcaagta
gaggcttgatttggaggttaaagtttTgctatgctgtattttacattacttattgttttagctgt
cctcatgaatgtcttttcactacccatttgcttatcctgcatctctcagccttgactccactcag
ttctcttgcttagagataccacctttcccctgaagtgttccttccatgttttacggcgagatggt
ttctcctcgcctggccactcagccttagttgtctctgttgtcttatagaggtctacttgaagaag
gaaaaacagggggcatggtttgactgtcctgtgagcccttcttccctgcctcccccactcacagt
gacccggaatccctcgacatggcagtctagcactagtgcggccgcagatctgcttcctcgctcac
tgactcgctgcgctcggtcgttcggctgcggcgagcggtatcagctcactcaaaggcggtaatac
ggttatccacagaatcaggggataacgcaggaaagaacatgtgagcaaaaggccagcaaaaggcc
aggaaccgtaaaaaggccgcgttgctggcgtttttccataggctccgcccccctgacgagcatca
caaaaatcgacgctcaagtcagaggtggcgaaacccgacaggactataaagataccaggcgtttc
cccctggaagctccctcgtgcgctctcctgttccgaccctgccgcttaccggatacctgtccgcc
tttctcccttcgggaagcgtggcgctttctcaTAgctcacgctgtaggtatctcagttcggtgta
ggtcgttcgctccaagctgggctgtgtgcacgaaccccccgttcagcccgaccgctgcgccttat
ccggtaactatcgtcttgagtccaacccggtaagacacgacttatcgccactggcagcagccact
ggtaacaggattagcagagcgaggtatgtaggcggtgctacagagttcttgaagtggtggcctaa
ctacggctacactagaagAacagtatttggtatctgcgctctgctgaagccagttaccttcggaa
aaagagttggtagctcttgatccggcaaacaaaccaccgctggtagcggtggtttttttgtttgc
aagcagcagattacgcgcagaaaaaaaggatctcaagaagatcctttgatcttttctacggggtc
tgacgctcagtggaacgaaaactcacgttaagggattttggtcatgagattatcaaaaaggatct
tcacctagatccttttaaattaaaaatgaagttttaaatcaatctaaagtatatatgagtaaact
tggtctgacagttaccaatgcttaatcagtgaggcacctatctcagcgat
(SEQ ID NO:10)
DOCK8 1267 CfaN Intein Lentiviral plasmid sequence
mCCL-EFS-1267Dock8-CfaN-WPRE (10946 bp)
(see e.g. FIG. 18)
acgcgccctgtagcggcgcattaagcgcggcgggtgtggtggttacgcgcagcgtgaccgctaca
cttgccagcgccctagcgcccgctcctttcgctttcttcccttcctttctcgccacgttcgccgg
ctttccccgtcaagctctaaatcgggggctccctttagggttccgatttagtgctttacggcacc
tcgaccccaaaaaacttgattagggtgatggttcacgtagtgggccatcgccctgatagacggtt
tttcgccctttgacgttggagtccacgttctttaatagtggactcttgttccaaactggaacaac
actcaaccclatctcggiclaticititgatttataagggattitgccgatttcggcctattggt
taaaaaatgagctgatttaacaaaaatttaacgcgaattttaacaaaatattaacgcttacaatt
taggtggcacttttcggggaaatgtgcgcggaacccctatttgtttatttttclaaatacattca
aatatgtatccgctcatgagacaataaccctgataaatgcttcaataatagcacctagatcaaga
gacaggatgaggatcgtttcgcatgattgaacaagatggattgcacgcaggttctccggccgctt
gggtggagaggctattcggctatgactgggcacaacagacaatcggctgctctgatgccgccgtg
ttccggctgtcagcgcaggggcgcccggttctttttgtcaagaccgacctgtccggtgccctgaa
tgaactgcaagacgaggcagcgcggctatcgtggctggccacgacgggcgttccttgcgcagctg
tgctcgacgttgtcactgaagcgggaagggactggctgctattgggcgaagtgccggggcaggat
ctcctgtcatctcaccttgctcctgccgagaaagtatccatcatggctgatgcaatgcggcggct
gcatacgcttgatccggctacctgcccattcgaccaccaagcgaaacatcgcatcgagcgagcac
gtactcggatggaagccggtcttgtcgatcaggatgatctggacgaagagcatcaggggctcgcg
ccagccgaactgttcgccaggctcaaggcgagcatgcccgacggcgaggatctcgtcgtgaccca
tggcgatgcctgcttgccgaatatcatggtggaaaatggccgcttttctggattcatcgactgtg
gccggctgggtgtggcggaccgctatcaggacatagcgttggctacccgtgatattgctgaagag
cttggcggcgaatgggctgaccgcttcctcgtgctttacggtatcgccgctcccgattcgcagcg
catcgccttctatcgccttcttgacgagttcttctgaattattaacgcttacaatttcctgatgc
ggtattttctccttacgcatctgtgcggtatttcacaccgcatcaggtggcacttttcggggaaa
tgtgcgcggaacccctatttgtttatttttctaaatacattcaaatatgtatccgctcatgacca
aaatcccttaacgtgagttttcgttccactgagcgtcagaccccgtagaaaagatcaaaggatct
tcttgagatcctttttttctgcgcgtaatctgctgcttgcaaacaaaaaaaccaccgctaccagc
ggtggtttgtttgccggatcaagagctaccaactctttttccgaaggtaactggcttcagcagag
cgcagataccaaatactgttcttctagtgtagccgtagttaggccaccacttcaagaactctgta
gcaccgcctacatacctcgctctgctaatcctgttaccagtggctgctgccagtggcgataagtc
gtgtcttaccgggttggactcaagacgatagttaccggataaggcgcagcggtcgggctgaacgg
gggggtcgtgcacacagcccagcttggagcgaacgacctacaccgaactgagatacctacagcgt
gagctatgagaaagcgccacgcttcccgaagggagaaaggcggacaggtatccggtaagcggcag
ggtcggaacaggagagcgcacgagggagcttccagggggaaacgcctggtatctttatagtcctg
tcgggtttcgccacctctgacttgagcgtcgatttttgtgatgctcgtcaggggggcggagccta
tggaaaaacgccagcaacgcggcctttttacggttcctggccttttgctggccttttgctcacat
gttctttcctgcgttatcccctgattctgtggataaccgtattaccgcctttgagtgagctgata
ccgctcgccgcagccgaacgaccgagcgcagcgagtcagtgagcgaggaagcggaagagcgccca
atacgcaaaccgcctctccccgcgcgttggccgattcattaatgcagctggcacgacaggtttcc
cgactggaaagcgggcagtgagcgcaacgcaattaatgtgagttagctcactcattaggcacccc
aggctttacactttatgcttccggctcgtatgttgtgtggaattgtgagcggataacaatttcac
acaggaaacagctatgaccatgattacgccaagcgcgcaattaaccctcactaaagggaacaaaa
gctggagctgcaagcttggccattgcatacgttgtatccatatcataatatgtacatttatattg
gctcatgtccaacattaccgccatgttgacattgattattgactagttattaatagtaatcaatt
acggggtcattagttcatagcccatatatggagttccgcgttacataacttacggtaaatggccc
gcctggctgaccgcccaacgacccccgcccattgacgtcaataatgacgtatgttcccatagtaa
cgccaatagggactttccattgacgtcaatgggtggagtatttacggtaaactgcccacttggca
gtacatcaagtgtatcatatgccaagtacgccccctattgacgtcaatgacggtaaatggcccgc
ctggcattatgcccagtacatgaccttatgggactttcctacttggcagtacatctacgtattag
tcatcgctattaccatggtgatgcggttttggcagtacatcaatgggcgtggatagcggtttgac
tcacggggatttccaagtctccaccccattgacgtcaatgggagtttgttttggcaccaaaatca
acgggactttccaaaatgtcgtaacaactccgccccattgacgcaaatgggcggtaggcgtgtac
ggtgggaggtctatataagcagagctcgtttagtgaaccgGGGTCTCTCTGGTTAGACCAGATCT
GAGCCTGGGAGCTCTCTGGCTAACTAGGGAACCCACTGCTTAAGCCTCAATAAAGCTTGCCTTGA
GTGCTTCAAGTAGTGTGTGCCCGTCTGTTGTGTGACTCTGGTAACTAGAGATCCCTCAGACCCTT
TTAGTCAGTGTGGAAAATCTCTAGCagtggcgcccgaacagggacttgaaagcgaaagggaaacc
agaggagctctcTCGACGCAGGACTCGGCTTGCTGAAGCGCGCACGGCAAGAGGCGAGGGGCGGC
GACTGGTGAGTACGCCAAAAATTTTGACTAGCGGAGGCTAGAAGGAGAGAGATGGGTGCGAGAGC
GTCAGTATTAAGCGGGGGAGaattagatcgcgatgggaaaaaattcggttaaggccagggggaaa
gaaaaaatataaattaaaacatatagtatgggcaagcagggagctagaacgattcgcagttaatc
ctggcctgttagaaacatcagaaggctgtagacaaatactgggacagctacaaccatcccttcag
acaggatcagaagaacttagatcattatataatacagtagcaaccctctattgtgtgcatcaaag
gatagagataaaagacaccaaggaagctttagacaagatagaggaagagcaaaacaaaagtaaga
ccaccgcacagcaagcGGCCGCTgatcttcagacctggaggaggagatatgagggacaattggag
aagtgaattatataaatataaagtagtaaaaattgaaccattaggagtagcacccaccaaggcaa
agagaagagtggtgcagagagaaaaaagaGCAGTGGGAATaggagctttgttccttgggttcttg
ggagcagcaggaagcactatgggcgcagcgtcaatgacgctgacggtacaggccagacaattatt
gtctggtatagtgcagcagcagaacaatttgctgagggctattgaggcgcaacagcatctgttgc
aactcacagtctggggcatcaagcagctccaggcaagaatcctggctgtggaaagatacctaaag
gatcaacagctcctggggatttggggttgctctggaaaactcatttgcaccactgctgtgccttg
gaatgctagttggagtaataaatctctggaacagatttggaatcacacgacctggatggagtggg
acagagaaattaacaattacacaagcttaatacactccttaattgaagaatcgcaaaaccagcaa
gaaaagaatgaacaagaattattggaattagataaatgggcaagtttgtggaattggtttaacat
aacaaattggctgtggtatataaaattattcataatgatagtaggaggcttggtaggtttaagaa
tagtttttgctgtactttctatagtgaatagagttaggcagggatattcaccattatcgtttcag
acccacctcccaaccccgaggggacccgacaggcccgaaggaatagaagaagaaggtggagagag
agacagagacagatccattcgattagtgaacggatCacaaatggcagtattcatccacaatttta
aaagaaaaggggggattggggggtacagtgcaggggaaagaatagtagacataatagcaacagac
atacaaactaaagaattacaaaaacaaattacaaaaattcaaaattttcgggtttattacaggga
cagcagaggctccggtgcccgtcagtgggcagagcgcacatcgcccacagtccccgagaagttgg
ggggaggggtcggcaattgaaccggtgcctagagaaggtggcgcggggtaaactgggaaagtgat
gtcgtgtactggctccgcctttttcccgaggggggggagaaccgtatataagtgcagtagtcgcc
gtgaacgttctttttcgcaacgggtttgccgccagaacacaggtgtcgtgaAAATAAGAGAGAAA
AGAAGAGTAAGAAGAAATATAAGAGCCACCATGGCCACACTGCCTAGCGCTGAGAGAAGGGCCTT
CGCTCTGAAGATCAACCGGTACAGCAGCGCCGAGATCCGGAAGCAGTTTACCCTGCCTCCTAACC
TGGGCCAGTACCACAGACAGAGCATCAGCACCAGCGGCTTCCCATCTCTGCAGCTGCCCCAGTTC
TACGACCCTGTGGAACCCGTGGATTTCGAGGGCCTGCTGATGACCCACCTGAACAGCCTGGATGT
GCAGCTGGCCCAAGAGCTGGGCGACTTCACCGACGATGATCTGGACGTGGTGTTCACCCCTAAAG
AGTGCAGAACCCTGCAGCCTAGCCTGCCTGAAGAAGGCGTGGAACTGGATCCTCACGTGCGGGAT
TGTGTGCAGACCTACATCCGCGAGTGGCTGATCGTGAACCGGAAGAATCAGGGCAGCCCAGAGAT
CTGCGGCTTCAAGAAAACCGGCAGCCGGAAGGACTTCCACAAGACCCTGCCTAAGCAGACCTTCG
AGAGCGAGACACTGGAATGCTCTGAGCCTGCTGCTCAGGCCGGACCTAGACACCTGAATGTGCTG
TGTGACGTGTCCGGCAAGGGACCTGTGACCGCCTGCGATTTCGACCTGAGAAGTCTGCAGCCCGA
CAAGCGGCTGGAAAACCTGCTCCAGCAAGTGTCCGCCGAGGACTTCGAGAAGCAGAACGAGGAAG
CCAGACGGACCAACAGACAGGCCGAGCTGTTTGCTCTGTACCCCAGCGTGGACGAGGAAGATGCC
GTCGAGATTAGACCCGTGCCTGAGTGCCCAAAAGAGCACCTGGGCAACAGAATCCTGGTCAAGCT
GCTGACCCTGAAGTTCGAGATCGAGATTGAGCCCCTGTTCGCCTCTATCGCCCTGTACGACGTGA
AAGAGCGGAAGAAGATCAGCGAGAACTTCCACTGCGACCTGAACTCCGACCAGTTCAAGGGCTTC
CTGAGAGCCCACACACCATCTGTGGCCGCTAGCTCTCAGGCCAGATCTGCCGTGTTCAGCGTGAC
ATACCCCAGCAGCGACATCTACCTGGTGGTCAAGATCGAGAAGGTCCTGCAGCAGGGCGAGATCG
GCGATTGTGCCGAGCCTTACACCGTGATCAAAGAGAGCGACGGCGGCAAGAGCAAAGAGAAGATT
GAGAAGCTGAAGCTGCAGGCCGAGAGCTTCTGTCAGCGGCTGGGCAAGTACAGAATGCCCTTTGC
CTGGGCTCCTATCAGCCTGTCCAGCTTCTTCAACGTGTCCACACTGGAACGGGAAGTGACCGACG
TGGACTCTGTCGTGGGCAGATCTAGCGTGGGCGAGAGAAGGACACTGGCTCAGTCTAGACGGCTG
AGCGAGAGAGCCCTGAGCCTGGAAGAAAATGGCGTGGGCAGCAACTTCAAGACCAGCACTCTGTC
CGTGTCTAGCTTCTTTAAGCAAGAGGGCGACAGACTGAGCGACGAGGACCTGTTCAAGTTCCTGG
CCGACTACAAGCGGAGCAGCTCCCTGCAGAGAAGAGTGAAGTCTATCCCTGGCCTGCTGCGCCTG
GAAATCTCTACAGCCCCTGAGATCATCAACTGCTGTCTGACCCCTGAAATGCTGCCCGTGAAGCC
CTTTCCAGAGAACCGGACCAGACCTCACAAAGAGATCCTGGAATTCCCCACCAGAGAAGTGTACG
TGCCCCACACCGTGTACCGGAACCTGCTGTATGTGTACCCACAGCGGCTGAACTTCGTGAACAAG
CTGGCCTCCGCCAGAAACATCACCATCAAGATCCAGTTTATGTGCGGCGAGGACGCCAGCAACGC
CATGCCTGTGATCTTCGGCAAGTCTAGCGGCCCTGAGTTCCTGCAAGAGGTGTACACAGCCGTGA
CCTACCACAACAAGAGCCCCGACTTCTACGAGGAAGTGAAGATTAAGCTGCCCGCCAAGCTGACC
GTGAATCACCATCTGCTGTTCACCTTCTACCACATCAGCTGCCAGCAGAAACAGGGCGCCTCTGT
GGAAACACTGCTGGGCTATAGCTGGCTGCCCATCCTGCTGAACGAGAGACTGCAGACCGGCAGCT
ACTGTCTGCCTGTGGCTCTGGAAAAGCTGCCACCTAACTACAGCATGCACTCCGCCGAGAAGGTG
CCCCTGCAGAATCCTCCTATTAAGTGGGCCGAGGGCCACAAGGGCGTGTTCAATATCGAGGTGCA
GGCCGTGTCCTCCGTGCACACCCAGGATAACCACCTGGAAAAGTTCTTCACCCTGTGCCACAGCC
TCGAGAGCCAAGTGACATTCCCCATCCGCGTGCTGGACCAGAAAATCTCCGAGATGGCCCTGGAA
CACGAGCTGAAACTGAGCATCATCTGCCTGAATAGCAGCAGACTGGAACCCCTGGTGCTGTTCCT
GCATCTGGTGCTGGACAAGCTGTTCCAGCTGAGCGTGCAGCCCATGGTTATCGCCGGACAGACCG
CCAACTTCAGCCAGTTCGCCTTTGAGAGCGTGGTGGCCATTGCCAACAGCCTGCACAACAGCAAG
GACCTGAGCAAGGATCAGCACGGCAGAAACTGCCTGCTGGCCTCTTACGTGCACTACGTGTTCAG
ACTGCCTGAGGTGCAGAGGGACGTGCCAAAATCTGGCGCTCCTACCGCTCTGCTGGACCCCAGAA
GCTATCACACCTACGGCAGAACATCTGCCGCCGCTGTGTCCTCTAAACTCCTGCAGGCTAGAGTG
ATGTCCAGCAGCAACCCTGATCTGGCCGGAACACACAGCGCCGCTGATGAAGAAGTGAAAAACAT
CATGAGCAGCAAGATCGCCGACCGGAACTGCAGCCGGATGAGCTACTACTGTAGCGGCAGCTCTG
ATGCCCCTAGCTCTCCAGCTGCTCCTAGACCTGCCAGCAAGAAGCACTTTCACGAGGAACTGGCC
CTGCAGATGGTCGTGTCTACCGGCATGGTTCGAGAGACAGTGTTTAAGTACGCCTGGTTCTTCTT
CGAACTGCTGGTCAAGAGCATGGCCCAGCACGTGCACAACATGGACAAGCGGGACAGCTTCCGGC
GGACCAGATTCAGCGACAGATTCATGGACGACATCACCACCATCGTGAACGTGGTCACCAGCGAG
ATTGCTGCCCTGCTGGTTAAGCCCCAGAAAGAGAACGAGCAGGCCGAAAAGATGAATATCTCTCT
GGCCTTCTTCCTCTACGACCTGCTGAGCCTGATGGACCGGGGCTTCGTGTTCAACCTGATCCGGC
ACTACTGCTCCCAGCTGTCTGCCAAGCTGTCCAACCTGCCTACACTGATCTCTATGAGACTCGAG
TTCCTGAGGATCCTGTGCAGCCACGAGCACTACCTGAACCTGAATCTGTTCTTCATGAACGCCGA
CACAGCCCCTACAAGCCCCTGTCCTTCTATCAGCAGCCAGAACAGCAGCAGCTGTAGCAGCTTTC
AGGATCAGAAAATCGCCAGCATGTTCGATCTGACCAGCGAGTACCGGCAGCAGCACTTTCTGACC
GGCCTGCTGTTTACCGAACTGGCAGCCGCTCTTGATGCCGAAGGCGAGGGAATCAGCAAGGTGCA
GCGGAAAGCCGTGTCTGCCATTCACAGCCTGCTGTCTAGCCACGACCTGGATCCTAGATGCGTGA
AGCCTGAAGTGAAAGTGAAAATCGCTGCCCTGTACCTGCCTCTCGTGGGCATCATTCTGGATGCC
CTGCCTCAGCTGTGTGATTTCACCGTGGCCGACACCAGACGGTACAGAACCTCTGGCTCCGACGA
GGAACAAGAAGGCGCTGGCGCCATCAACCAGAATGTGGCCCTTGCCATTGCCGGGAACAACTGCC
TGAGCTACGACACCGAGATCCTGACCGTGGAATACGGCTTCCTGCCTATCGGCAAGATCGTGGAA
GAACGGATCGAGTGCACCGTGTACACCGTGGACAAGAACGGCTTCGTGTACACACAGCCTATCGC
TCAGTGGCACAACCGGGGAGAGCAAGAGGTGTTCGAGTACTGCCTGGAAGATGGCAGCATCATCC
GGGCCACCAAGGACCACAAGTTCATGACCACCGACGGCCAGATGCTGCCCATCGACGAGATCTTT
GAGAGAGGCCTGGACCTGAAACAGGTGGACGGACTTCCTtgattccgataatcaacctctggatt
acaaaatttgtgaaagattgactggtattcttaactatgttgctccttttacgctatgtggatac
gctgctttaatgcctttgtatcatgctattgcttcccgtatggctttcattttctcctccttgta
taaatcctggttgctgtctctttatgaggagttgtggcccgttgtcaggcaacgtggcgtggtgt
gcactgtgtttgctgacgcaacccccactggttggggcattgccaccacctgtcagctcctttcc
gggactttcgctttccccctccctattgccacggcggaactcatcgccgcctgccttgcccgctg
ctggacaggggctcggctgttgggcactgacaattccgtggtgttgtcggggaaATCATcgtcct
ttccTtggctgctcgcctgtgttgccacctggattctgcgcgggacgtccttctgctacgtccct
tcggccctcaatccagcggaccttccttcccgcggcctgctgccggctctgcggcctcttccgcg
tcttcgccttcgccctcagacgagtcggatctccctttgggccgcctccccgcTaaaagaaaagg
ggggactggaagggctaattcactcccaacgaagacaagatctgctttttgcttgtactGGGTCT
CTCTGGTTAGACCAGATCTGAGCCTGGGAGCTCTCTGGCTAACTAGGGAACCCACTGCTTAAGCC
TCAATAAAGCTTGCCTTGAGTGCTTCAAGTAGTGTGTTGGTTTTTTGTGTGTGCATTGTCTGAGT
AGGTGTCATTCTATTCTGGGGGGTGGGGTGGGGCAGCACAGCAAGGGGGAGGATTGGGAAGACAA
TAGCAGGCATGCTGGGGATGCGGTGGGCTCTATGGagtagtagttcatgtcatcttattattcag
tatttataacttgcaaagaaatgaatatcagagagtgagaggaacttgtttattgcagcttataa
tggttacaaataaagcaatagcatcacaaatttcacaaataaagcatttttttcactgcattcta
gttgtggtttgtccaaactcatcaatgtatcttatcatgtctggctctagctatcccgcccctaa
ctccgcccATCCCGCCCCTAACTCCGCCCAGTTCCGCCCATTCTCCGCCCCATGGCTCTCACTAC
TTCTGGAATAGCTCAGAGGCCGAGGCGGCCTCGGCCTCTGCATAAATAAAAAAAATTAGTCGAGG
CTTTTTTGGAGGCCTAGGgacgtacccaattcgccctatagtgagtcgtattacgcgcgctcact
ggccgtcgttttacaacgtcgtgactgggaaaaccctggcgttacccaacttaatcgccttgcag
cacatccccctttcgccagctggcgtaatagcgaagaggcccgcaccgatcgcccttcccaacag
ttgcgcagcctgaatggcgaatggg
(SEQ ID NO: 11)
DOCK8 1267 CfaC-CFN plasmid sequence
1267 CfaC-CFN-DOCK8-bGHpA (6743 bp)
(see e.g. FIG. 19)
ctgtctatttcgttcatccatagttgcctgactccccgtcgtgtagataactacgatacgggagg
gcttaccatctggccccagtgctgcaatgataccgcgagacccacgctcaccggctccagattta
tcagcaataaaccagccagccggaagggccgagcgcagaagtggtcctgcaactttatccgcctc
catccagtctattaattgttgccgggaagctagagtaagtagttcgccagttaatagtttgcgca
acgttgttgccattgctacaggcatcgtggtgtcacgctcgtcgtttggtatggcttcattcagc
tccggttcccaacgatcaaggcgagttacatgatcccccatgttgtgcaaaaaagcggttagctc
cttcggtcctccgatcgttgtcagaagtaagttggccgcagtgttatcactcatggttatggcag
cactgcataattctcttactgtcatgccatccgtaagatgcttttctgtgactggtgagtactca
accaagtcattctgagaatagtgtatgcggcgaccgagttgctcttgcccggcgtcaatacggga
taataccgcgccacatagcagaactttaaaagtgctcatcattggaaaacgttcttcggggcgaa
aactctcaaggatcttaccgctgttgagatccagttcgatgtaacccactcgtgcacccaactga
tcttcagcatcttttactttcaccagcgtttctgggtgagcaaaaacaggaaggcaaaatgccgc
aaaaaagggaataagggcgacacggaaatgttgaatactcatactcttcctttttcaatattatt
gaagcatttatcagggttattgtctcatgagcggatacatatttgaatgtatttagaaaaataaa
caaataggggttccgcgcacatttccccgaaaagtgccacctgacgtggatcccctgagggggcc
cccatgggctagaggatccggcctcggcctctgcataaataaaaaaaattagtcagccatgagct
tggcccattgcatacgttgtatccatatcataatatgtacatttatattggctcatgtccaacat
taccgccatgttgacattgattattgactagttattaatagtaatcaattacggggtcattagtt
catagcccatatatggagttccgcgttacataacttacggtaaatggcccgcctggctgaccgcc
caacgacccccgcccattgacgtcaataatgacgtatgttcccatagtaacgccaatagggactt
tccattgacgtcaatgggtggagtatttacggtaaactgcccacttggcagtacatcaagtgtat
catatgccaagtacgccccctattgacgtcaatgacggtaaatggcccgcctggcattatgccca
gtacatgaccttatgggactttcctacttggcagtacatctacgtattagtcatcgctattacca
tggtgatgcggttttggcagtacatcaatgggcgtggatagcggtttgactcacggggatttcca
agtctccaccccattgacgtcaatgggagtttgttttggcaccaaaatcaacgggactttccaaa
atgtcgtaacaactccgccccattgacgcaaatgggcggtaggcgtgtacggtgggaggtctata
taagcagagctcgtttagtgaaccgtcagatcgcctggagacgccatccacgctgttttgacctc
catagaagacaccgggaccgatccagcctcccctcgaagcttacatgtggtaccgagctcggatc
ctgagaacttcagggtgagtctatgggacccttgatgttttctttccccttcttttctatggtta
agttcatgtcataggaaggggagaagtaacagggtacacatattgaccaaatcagggtaattttg
catttgtaattttaaaaaatgctttcttcttttaatatacttttttgtttatcttatttctaata
ctttccctaatctctttctttcagggcaataatgatacaatgtatcatgcctctttgcaccattc
taaagaataacagtgataatttctgggttaaggcaatagcaatatttctgcatataaatatttct
gcatataaattgtaactgatgtaagaggtttcatattgctaatagcagctacaatccagctacca
ttctgcttttattttatggttgggataaggctggattattctgagtccaagctaggcccttttgc
taatcatgttcatacctcttatcttcctcccacagctcctgggcaacgtgctggtctgtgtgctg
gcccatcactttggcaaagcacgtgagatctgaattcgagatctgccgccgccatgGTGAAGATC
ATCAGCAGAAAGAGCCTGGGCACCCAGAACGTGTACGACATCGGCGTGGAAAAGGACCACAACTT
TCTGCTCAAGAACGGCCTGGTGGCCAGCAACTGCTTCAATCTGAAAACCAGCGGCATCGTGCTGA
GCAGCCTGCCTTACAAGCAGTACAACATGCTGAATGCCGACACTACCCGCAACCTGATGATCTGC
TTCCTGTGGATTATGAAGAACGCCGATCAGAGCCTGATTCGGAAGTGGATTGCCGACCTGCCAAG
CACTCAGCTGAACCGGATTCTGGACCTCCTGTTCATCTGCGTGCTGTGCTTCGAGTACAAGGGCA
AGCAGTCCAGCGACAAGGTGTCCACTCAGGTGCTGCAGAAAAGCCGGGATGTGAAGGCCAGGCTG
GAAGAGGCACTTCTGAGAGGCGAAGGCGCCAGAGGCGAGATGATGAGGCGTAGAGCCCCTGGCAA
CGATAGATTCCCCGGCCTGAATGAGAACCTGCGGTGGAAGAAAGAGCAGACCCATTGGAGACAGG
CCAACGAGAAACTGGACAAGACCAAGGCCGAACTGGACCAAGAGGCCCTGATCTCTGGCAACCTG
GCCACAGAAGCCCACCTGATTATCCTGGACATGCAAGAGAACATCATCCAGGCCAGTAGCGCCCT
GGATTGCAAGGATTCTCTGCTCGGAGGCGTGCTGAGGGTGCTCGTGAATAGCCTGAATTGCGACC
AGAGCACCACCTACCTGACACACTGCTTCGCCACACTGAGGGCCCTGATTGCCAAGTTTGGGGAC
CTGCTGTTCGAGGAAGAGGTGGAACAGTGCTTCGACCTGTGTCACCAAGTGCTGCACCACTGCAG
CAGCTCCATGGACGTGACAAGATCCCAGGCCTGCGCCACTCTGTATCTGCTGATGCGGTTTAGCT
TCGGCGCCACCAGCAATTTCGCCAGAGTGAAGATGCAAGTGACCATGAGCCTGGCCAGCCTCGTT
GGAAGGGCCCCTGACTTCAATGAGGAACACCTCAGAAGAAGCCTGCGGACCATCCTGGCCTACAG
CGAAGAGGATACCGCCATGCAGATGACCCCATTTCCAACACAAGTGGAAGAACTGCTGTGCAACC
TGAATTCCATCCTGTACGATACCGTGAAGATGAGAGAGTTCCAAGAGGACCCCGAGATGCTGATG
GATCTGATGTACCGGATCGCCAAGAGCTACCAGGCCTCTCCTGATCTGAGACTGACCTGGCTGCA
GAACATGGCCGAGAAGCACACCAAGAAGAAGTGCTACACCGAGGCCGCCATGTGCCTGGTTCATG
CTGCTGCACTCGTGGCCGAGTACCTGAGCATGCTGGAAGATCACTCCTACCTGCCAGTGGGCAGC
GTGTCCTTCCAGAACATCAGCTCCAACGTCCTGGAAGAGTCCGTGGTGTCCGAGGACACACTGAG
CCCAGATGAGGATGGCGTGTGTGCCGGACAGTACTTTACCGAGTCTGGCCTCGTGGGCCTTCTGG
AACAGGCCGCCGAACTGTTTTCTACCGGCGGACTGTACGAGACAGTGAACGAAGTGTACAAGCTG
GTCATCCCCATCCTGGAAGCCCACAGAGAGTTCAGAAAGCTGACACTGACCCACTCCAAGCTGCA
GCGGGCCTTCGACAGCATTGTGAACAAGGACCACAAGCGGATGTTCGGCACCTACTTCAGAGTGG
GCTTTTTCGGCAGCAAATTCGGCGACCTGGACGAGCAAGAGTTCGTGTACAAAGAGCCCGCCATC
ACCAAGCTGCCTGAGATCTCCCATCGGCTGGAAGCCTTCTACGGCCAGTGTTTTGGCGCCGAGTT
CGTGGAAGTGATCAAGGACAGCACCCCTGTGGATAAGACCAAACTGGACCCTAACAAGGCCTACA
TCCAGATCACCTTCGTGGAACCCTACTTCGACGAGTACGAGATGAAGGATAGAGTGACCTACTTT
GAGAAGAACTTCAACCTGCGGCGGTTCATGTACACCACACCTTTTACACTGGAAGGCAGACCCAG
GGGCGAGCTGCACGAGCAGTATAGAAGAAACACCGTGCTGACCACCATGCACGCATTCCCTTACA
TCAAGACCCGGATCAGCGTGATCCAGAAAGAAGAGTTTGTCCTGACACCAATCGAGGTGGCCATC
GAGGACATGAAGAAGAAAACCCTCCAGCTGGCCGTGGCCATCAATCAAGAACCTCCAGACGCCAA
GATGCTCCAGATGGTGCTCCAGGGATCTGTGGGCGCCACAGTGAATCAGGGACCTCTGGAAGTGG
CCCAAGTGTTCCTGGCTGAGATCCCCGCCGATCCTAAGCTGTACAGACACCATAACAAGCTGCGG
CTCTGCTTCAAAGAATTCATCATGAGATGTGGCGAGGCCGTGGAAAAGAACAAGAGACTGATCAC
CGCCGACCAGAGAGAGTACCAGCAAGAGCTGAAGAAAAACTACAACAAGCTCAAAGAAAATCTGC
GGCCCATGATCGAGAGAAAGATCCCCGAGCTGTACAAGCCCATCTTCCGCGTGGAAAGCCAGAAG
CGGGACTCCTTTCACAGATCCAGCTTTAGGAAGTGCGAGACACAGCTGTCCCAGGGCTCTTGAct
ggtgtggctaatgccctggcccacaagtAtcactaagctcgcttTcttgctgtccaatttctatt
aaaggttccttTgttccctaagtccaactactaaactgggggatattatgaagggccttgagcat
ctggattctgcctaataaaaaacatttattttcattgcaatgatgtatttaaattatttctgaat
attttactaaaaagggaatgtgggaggtcagtgcatttaaaacataaagaaatgaagagctagtt
caaaccttgggaaaatacactatatcttaaactccatgaaagaaggtgaggctgcaaacagctaa
tgcacattggcaacagCccctgatgcctatgccttattcatccctcagaaaaggattcaagtaga
ggcttgatttggaggttaaagtttTgctatgctgtattttacattacttattgttttagctgtcc
tcatgaatgtcttttcactacccatttgcttatcctgcatctctcagccttgactccactcagtt
ctcttgcttagagataccacctttcccctgaagtgttccttccatgttttacggcgagatggttt
ctcctcgcctggccactcagccttagttgtctctgttgtcttatagaggtctacttgaagaagga
aaaacagggggcatggtttgactgtcctgtgagcccttcttccctgcctcccccactcacagtga
cccggaatccctcgacatggcagtctagcactagtgcggccgcagatctgcttcctcgctcactg
actcgctgcgctcggtcgttcggctgcggcgagcggtatcagctcactcaaaggcggtaatacgg
ttatccacagaatcaggggataacgcaggaaagaacatgtgagcaaaaggccagcaaaaggccag
gaaccgtaaaaaggccgcgttgctggcgtttttccataggctccgcccccctgacgagcatcaca
aaaatcgacgctcaagtcagaggtggcgaaacccgacaggactataaagataccaggcgtttccc
cctggaagctccctcgtgcgctctcctgttccgaccctgccgcttaccggatacctgtccgcctt
tctcccttcgggaagcgtggcgctttctcaTAgctcacgctgtaggtatctcagttcggtgtagg
tcgttcgctccaagctgggctgtgtgcacgaaccccccgttcagcccgaccgctgcgccttatcc
ggtaactatcgtcttgagtccaacccggtaagacacgacttatcgccactggcagcagccactgg
taacaggattagcagagcgaggtatgtaggcggtgctacagagttcttgaagtggtggcctaact
acggctacactagaagAacagtatttggtatctgcgctctgctgaagccagttaccttcggaaaa
agagttggtagctcttgatccggcaaacaaaccaccgctggtagcggtggtttttttgtttgcaa
gcagcagattacgcgcagaaaaaaaggatctcaagaagatcctttgatcttttctacggggtctg
acgctcagtggaacgaaaactcacgttaagggattttggtcatgagattatcaaaaaggatcttc
acctagatccttttaaattaaaaatgaagttttaaatcaatctaaagtatatatgagtaaacttg
gtctgacagttaccaatgcttaatcagtgaggcacctatctcagcgat(SEQIDNO:12)
DOCK8 1267 CfaC-CFN Lentiviral plasmid sequence
mCCL-EFS-1267Dock8-CfaC-CFN-WPRE (9449 bp)
(see e.g. FIG. 20)
acgcgccctgtagcggcgcattaagcgcggcgggtgtggtggttacgcgcagcgtgaccgctaca
cttgccagcgccctagcgcccgctcctttcgctttcttcccttcctttctcgccacgttcgccgg
ctttccccgtcaagctctaaatcgggggctccctttagggttccgatttagtgctttacggcacc
tcgaccccaaaaaacttgattagggtgatggttcacgtagtgggccatcgccctgatagacggtt
tttcgccctttgacgttggagtccacgttctttaatagtggactcttgttccaaactggaacaac
actcaaccctatctcggtctattcttttgatttataagggattttgccgatttcggcctattggt
taaaaaatgagctgatttaacaaaaatttaacgcgaattttaacaaaatattaacgcttacaatt
taggtggcacttttcggggaaatgtgcgcggaacccctatttgtttatttttctaaatacattca
aatatgtatccgctcatgagacaataaccctgataaatgcttcaataatagcacctagatcaaga
gacaggatgaggatcgtttcgcatgattgaacaagatggattgcacgcaggttctccggccgctt
gggtggagaggctattcggctatgactgggcacaacagacaatcggctgctctgatgccgccgtg
ttccggctgtcagcgcaggggcgcccggttctttttgtcaagaccgacctgtccggtgccctgaa
tgaactgcaagacgaggcagcgcggctatcgtggctggccacgacgggcgttccttgcgcagctg
tgctcgacgttgtcactgaagcgggaagggactggctgctattgggcgaagtgccggggcaggat
ctcctgtcatctcaccttgctcctgccgagaaagtatccatcatggctgatgcaatgcggcggct
gcatacgcttgatccggctacctgcccattcgaccaccaagcgaaacatcgcatcgagcgagcac
gtactcggatggaagccggtcttgtcgatcaggatgatctggacgaagagcatcaggggctcgcg
ccagccgaactgttcgccaggctcaaggcgagcatgcccgacggcgaggatctcgtcgtgaccca
tggcgatgcctgcttgccgaatatcatggtggaaaatggccgcttttctggattcatcgactgtg
gccggctgggtgtggcggaccgctatcaggacatagcgttggctacccgtgatattgctgaagag
cttggcggcgaatgggctgaccgcttcctcgtgctttacggtatcgccgctcccgattcgcagcg
catcgccttctatcgccttcttgacgagttcttctgaattattaacgcttacaatttcctgatgc
ggtattttctccttacgcatctgtgcggtatttcacaccgcatcaggtggcacttttcggggaaa
tgtgcgcggaacccctatttgtttatttttctaaatacattcaaatatgtatccgctcatgacca
aaatcccttaacgtgagttttcgttccactgagcgtcagaccccgtagaaaagatcaaaggatct
tcttgagatcctttttttctgcgcgtaatctgctgcttgcaaacaaaaaaaccaccgctaccagc
ggtggtttgtttgccggatcaagagctaccaactctttttccgaaggtaactggcttcagcagag
cgcagataccaaatactgttcttctagtgtagccgtagttaggccaccacttcaagaactctgta
gcaccgcctacatacctcgctctgctaatcctgttaccagtggctgctgccagtggcgataagtc
gtgtcttaccgggttggactcaagacgatagttaccggataaggcgcagcggtcgggctgaacgg
gggggtcgtgcacacagcccagcttggagcgaacgacctacaccgaactgagatacctacagcgt
gagctatgagaaagcgccacgcttcccgaagggagaaaggcggacaggtatccggtaagcggcag
ggtcggaacaggagagcgcacgagggagcttccagggggaaacgcctggtatctttatagtcctg
tcgggtttcgccacctctgacttgagcgtcgatttttgtgatgctcgtcaggggggcggagccta
tggaaaaacgccagcaacgcggcctttttacggttcctggccttttgctggccttttgctcacat
gttctttcctgcgttatcccctgattctgtggataaccgtattaccgcctttgagtgagctgata
ccgctcgccgcagccgaacgaccgagcgcagcgagtcagtgagcgaggaagcggaagagcgccca
atacgcaaaccgcctctccccgcgcgttggccgattcattaatgcagctggcacgacaggtttcc
cgactggaaagcgggcagtgagcgcaacgcaattaatgtgagttagctcactcattaggcacccc
aggctttacactttatgcttccggctcgtatgttgtgtggaattgtgagcggataacaatttcac
acaggaaacagctatgaccatgattacgccaagcgcgcaattaaccctcactaaagggaacaaaa
gctggagctgcaagcttggccattgcatacgttgtatccatatcataatatgtacatttatattg
gctcatgtccaacattaccgccatgttgacattgattattgactagttattaatagtaatcaatt
acggggtcattagttcatagcccatatatggagttccgcgttacataacttacggtaaatggccc
gcctggctgaccgcccaacgacccccgcccattgacgtcaataatgacgtatgttcccatagtaa
cgccaatagggactttccattgacgtcaatgggtggagtatttacggtaaactgcccacttggca
gtacatcaagtgtatcatatgccaagtacgccccctattgacgtcaatgacggtaaatggcccgc
ctggcattatgcccagtacatgaccttatgggactttcctacttggcagtacatctacgtattag
tcatcgctattaccatggtgatgcggttttggcagtacatcaatgggcgtggatagcggtttgac
tcacggggatttccaagtctccaccccattgacgtcaatgggagtttgttttggcaccaaaatca
acgggactttccaaaatgtcgtaacaactccgccccattgacgcaaatgggcggtaggcgtgtac
ggtgggaggtctatataagcagagctcgtttagtgaaccgGGGTCTCTCTGGTTAGACCAGATCT
GAGCCTGGGAGCTCTCTGGCTAACTAGGGAACCCACTGCTTAAGCCTCAATAAAGCTTGCCTTGA
GTGCTTCAAGTAGTGTGTGCCCGTCTGTTGTGTGACTCTGGTAACTAGAGATCCCTCAGACCCTT
TTAGTCAGTGTGGAAAATCTCTAGCagtggcgcccgaacagggacttgaaagcgaaagggaaacc
agaggagctctcTCGACGCAGGACTCGGCTTGCTGAAGCGCGCACGGCAAGAGGCGAGGGGCGGC
GACTGGTGAGTACGCCAAAAATTTTGACTAGCGGAGGCTAGAAGGAGAGAGATGGGTGCGAGAGC
GTCAGTATTAAGCGGGGGAGaattagatcgcgatgggaaaaaattcggttaaggccagggggaaa
gaaaaaatataaattaaaacatatagtatgggcaagcagggagctagaacgattcgcagttaatc
ctggcctgttagaaacatcagaaggctgtagacaaatactgggacagctacaaccatcccttcag
acaggatcagaagaacttagatcattatataatacagtagcaaccctctattgtgtgcatcaaag
gatagagataaaagacaccaaggaagctttagacaagatagaggaagagcaaaacaaaagtaaga
ccaccgcacagcaagcGGCCGCTgatcttcagacctggaggaggagatatgagggacaattggag
aagtgaattatataaatataaagtagtaaaaattgaaccattaggagtagcacccaccaaggcaa
agagaagagtggtgcagagagaaaaaagaGCAGTGGGAATaggagctttgttccttgggttcttg
ggagcagcaggaagcactatgggcgcagcgtcaatgacgctgacggtacaggccagacaattatt
gtctggtatagtgcagcagcagaacaatttgctgagggctattgaggcgcaacagcatctgttgc
aactcacagtctggggcatcaagcagctccaggcaagaatcctggctgtggaaagatacctaaag
gatcaacagctcctggggatttggggttgctctggaaaactcatttgcaccactgctgtgccttg
gaatgctagttggagtaataaatctctggaacagatttggaatcacacgacctggatggagtggg
acagagaaattaacaattacacaagcttaatacactccttaattgaagaatcgcaaaaccagcaa
gaaaagaatgaacaagaattattggaattagataaatgggcaagtttgtggaattggtttaacat
aacaaattggctgtggtatataaaattattcataatgatagtaggaggcttggtaggtttaagaa
tagtttttgctgtactttctatagtgaatagagttaggcagggatattcaccattatcgtttcag
acccacctcccaaccccgaggggacccgacaggcccgaaggaatagaagaagaaggtggagagag
agacagagacagatccattcgattagtgaacggatCacaaatggcagtattcatccacaatttta
aaagaaaaggggggattggggggtacagtgcaggggaaagaatagtagacataatagcaacagac
atacaaactaaagaattacaaaaacaaattacaaaaattcaaaattttcgggtttattacaggga
cagcagaggctccggtgcccgtcagtgggcagagcgcacatcgcccacagtccccgagaagttgg
ggggaggggtcggcaattgaaccggtgcctagagaaggtggcgcggggtaaactgggaaagtgat
gtcgtgtactggctccgcctttttcccgaggggggggagaaccgtatataagtgcagtagtcgcc
gtgaacgttctttttcgcaacgggtttgccgccagaacacaggtgtcgtgaAAATAAGAGAGAAA
AGAAGAGTAAGAAGAAATATAAGAGCCACCatgGTGAAGATCATCAGCAGAAAGAGCCTGGGCAC
CCAGAACGTGTACGACATCGGCGTGGAAAAGGACCACAACTTTCTGCTCAAGAACGGCCTGGTGG
CCAGCAACTGCTTCAATCTGAAAACCAGCGGCATCGTGCTGAGCAGCCTGCCTTACAAGCAGTAC
AACATGCTGAATGCCGACACTACCCGCAACCTGATGATCTGCTTCCTGTGGATTATGAAGAACGC
CGATCAGAGCCTGATTCGGAAGTGGATTGCCGACCTGCCAAGCACTCAGCTGAACCGGATTCTGG
ACCTCCTGTTCATCTGCGTGCTGTGCTTCGAGTACAAGGGCAAGCAGTCCAGCGACAAGGTGTCC
ACTCAGGTGCTGCAGAAAAGCCGGGATGTGAAGGCCAGGCTGGAAGAGGCACTTCTGAGAGGCGA
AGGCGCCAGAGGCGAGATGATGAGGCGTAGAGCCCCTGGCAACGATAGATTCCCCGGCCTGAATG
AGAACCTGCGGTGGAAGAAAGAGCAGACCCATTGGAGACAGGCCAACGAGAAACTGGACAAGACC
AAGGCCGAACTGGACCAAGAGGCCCTGATCTCTGGCAACCTGGCCACAGAAGCCCACCTGATTAT
CCTGGACATGCAAGAGAACATCATCCAGGCCAGTAGCGCCCTGGATTGCAAGGATTCTCTGCTCG
GAGGCGTGCTGAGGGTGCTCGTGAATAGCCTGAATTGCGACCAGAGCACCACCTACCTGACACAC
TGCTTCGCCACACTGAGGGCCCTGATTGCCAAGTTTGGGGACCTGCTGTTCGAGGAAGAGGTGGA
ACAGTGCTTCGACCTGTGTCACCAAGTGCTGCACCACTGCAGCAGCTCCATGGACGTGACAAGAT
CCCAGGCCTGCGCCACTCTGTATCTGCTGATGCGGTTTAGCTTCGGCGCCACCAGCAATTTCGCC
AGAGTGAAGATGCAAGTGACCATGAGCCTGGCCAGCCTCGTTGGAAGGGCCCCTGACTTCAATGA
GGAACACCTCAGAAGAAGCCTGCGGACCATCCTGGCCTACAGCGAAGAGGATACCGCCATGCAGA
TGACCCCATTTCCAACACAAGTGGAAGAACTGCTGTGCAACCTGAATTCCATCCTGTACGATACC
GTGAAGATGAGAGAGTTCCAAGAGGACCCCGAGATGCTGATGGATCTGATGTACCGGATCGCCAA
GAGCTACCAGGCCTCTCCTGATCTGAGACTGACCTGGCTGCAGAACATGGCCGAGAAGCACACCA
AGAAGAAGTGCTACACCGAGGCCGCCATGTGCCTGGTTCATGCTGCTGCACTCGTGGCCGAGTAC
CTGAGCATGCTGGAAGATCACTCCTACCTGCCAGTGGGCAGCGTGTCCTTCCAGAACATCAGCTC
CAACGTCCTGGAAGAGTCCGTGGTGTCCGAGGACACACTGAGCCCAGATGAGGATGGCGTGTGTG
CCGGACAGTACTTTACCGAGTCTGGCCTCGTGGGCCTTCTGGAACAGGCCGCCGAACTGTTTTCT
ACCGGCGGACTGTACGAGACAGTGAACGAAGTGTACAAGCTGGTCATCCCCATCCTGGAAGCCCA
CAGAGAGTTCAGAAAGCTGACACTGACCCACTCCAAGCTGCAGCGGGCCTTCGACAGCATTGTGA
ACAAGGACCACAAGCGGATGTTCGGCACCTACTTCAGAGTGGGCTTTTTCGGCAGCAAATTCGGC
GACCTGGACGAGCAAGAGTTCGTGTACAAAGAGCCCGCCATCACCAAGCTGCCTGAGATCTCCCA
TCGGCTGGAAGCCTTCTACGGCCAGTGTTTTGGCGCCGAGTTCGTGGAAGTGATCAAGGACAGCA
CCCCTGTGGATAAGACCAAACTGGACCCTAACAAGGCCTACATCCAGATCACCTTCGTGGAACCC
TACTTCGACGAGTACGAGATGAAGGATAGAGTGACCTACTTTGAGAAGAACTTCAACCTGCGGCG
GTTCATGTACACCACACCTTTTACACTGGAAGGCAGACCCAGGGGCGAGCTGCACGAGCAGTATA
GAAGAAACACCGTGCTGACCACCATGCACGCATTCCCTTACATCAAGACCCGGATCAGCGTGATC
CAGAAAGAAGAGTTTGTCCTGACACCAATCGAGGTGGCCATCGAGGACATGAAGAAGAAAACCCT
CCAGCTGGCCGTGGCCATCAATCAAGAACCTCCAGACGCCAAGATGCTCCAGATGGTGCTCCAGG
GATCTGTGGGCGCCACAGTGAATCAGGGACCTCTGGAAGTGGCCCAAGTGTTCCTGGCTGAGATC
CCCGCCGATCCTAAGCTGTACAGACACCATAACAAGCTGCGGCTCTGCTTCAAAGAATTCATCAT
GAGATGTGGCGAGGCCGTGGAAAAGAACAAGAGACTGATCACCGCCGACCAGAGAGAGTACCAGC
AAGAGCTGAAGAAAAACTACAACAAGCTCAAAGAAAATCTGCGGCCCATGATCGAGAGAAAGATC
CCCGAGCTGTACAAGCCCATCTTCCGCGTGGAAAGCCAGAAGCGGGACTCCTTTCACAGATCCAG
CTTTAGGAAGTGCGAGACACAGCTGTCCCAGGGCTCTTGAttccgataatcaacctctggattac
aaaatttgtgaaagattgactggtattcttaactatgttgctccttttacgctatgtggatacgc
tgctttaatgcctttgtatcatgctattgcttcccgtatggctttcattttctcctccttgtata
aatcctggttgctgtctctttatgaggagttgtggcccgttgtcaggcaacgtggcgtggtgtgc
actgtgtttgctgacgcaacccccactggttggggcattgccaccacctgtcagctcctttccgg
gactttcgctttccccctccctattgccacggcggaactcatcgccgcctgccttgcccgctgct
ggacaggggctcggctgttgggcactgacaattccgtggtgttgtcggggaaATCATcgtccttt
ccTtggctgctcgcctgtgttgccacctggattctgcgcgggacgtccttctgctacgtcccttc
ggccctcaatccagcggaccttccttcccgcggcctgctgccggctctgcggcctcttccgcgtc
ttcgccttcgccctcagacgagtcggatctccctttgggccgcctccccgcTaaaagaaaagggg
ggactggaagggctaattcactcccaacgaagacaagatctgctttttgcttgtactGGGTCTCT
CTGGTTAGACCAGATCTGAGCCTGGGAGCTCTCTGGCTAACTAGGGAACCCACTGCTTAAGCCTC
AATAAAGCTTGCCTTGAGTGCTTCAAGTAGTGTGTTGGTTTTTTGTGTGTGCATTGTCTGAGTAG
GTGTCATTCTATTCTGGGGGGTGGGGTGGGGCAGCACAGCAAGGGGGAGGATTGGGAAGACAATA
GCAGGCATGCTGGGGATGCGGTGGGCTCTATGGagtagtagttcatgtcatcttattattcagta
tttataacttgcaaagaaatgaatatcagagagtgagaggaacttgtttattgcagcttataatg
gttacaaataaagcaatagcatcacaaatttcacaaataaagcatttttttcactgcattctagt
tgtggtttgtccaaactcatcaatgtatcttatcatgtctggctctagctatcccgcccctaact
ccgcccATCCCGCCCCTAACTCCGCCCAGTTCCGCCCATTCTCCGCCCCATGGCTCTCACTACTT
CTGGAATAGCTCAGAGGCCGAGGCGGCCTCGGCCTCTGCATAAATAAAAAAAATTAGTCGAGGCT
TTTTTGGAGGCCTAGGgacgtacccaattcgccctatagtgagtcgtattacgcgcgctcactgg
ccgtcgttttacaacgtcgtgactgggaaaaccctggcgttacccaacttaatcgccttgcagca
catccccctttcgccagctggcgtaatagcgaagaggcccgcaccgatcgcccttcccaacagtt
gcgcagcctgaatggcgaatggg
(SEQ ID NO: 13)

The nucleotide and amino acid sequences of the DOCK8 fragments alone or fused to an intein component are provided in Table 2 below. In addition, the nucleotide sequences of expression cassettes are also provided in Table 2. To identify which portion of DOCK8 is represented in the below sequences, the information presented in the parentheses indicates either the 1^{st} or 2^{nd} fragment, also called the first or second half of DOCK8. One skilled in the art would appreciate that in certain instances a TGA stop codon is present at the 3′ end of some of the nucleic acid sequences below, for example SEQ ID NO: 14.

TABLE 2
Plasmid Vector Components

939 DOCK8-CfaN (1^{st} DOCK8 fragment; 5′ half)
ATGGCCACACTGCCTAGCGCTGAGAGAAGGGCCTTCGCTCTGAAGATCAACCGGTACAGCAGCGC
CGAGATCCGGAAGCAGTTTACCCTGCCTCCTAACCTGGGCCAGTACCACAGACAGAGCATCAGCA
CCAGCGGCTTCCCATCTCTGCAGCTGCCCCAGTTCTACGACCCTGTGGAACCCGTGGATTTCGAG
GGCCTGCTGATGACCCACCTGAACAGCCTGGATGTGCAGCTGGCCCAAGAGCTGGGCGACTTCAC
CGACGATGATCTGGACGTGGTGTTCACCCCTAAAGAGTGCAGAACCCTGCAGCCTAGCCTGCCTG
AAGAAGGCGTGGAACTGGATCCTCACGTGCGGGATTGTGTGCAGACCTACATCCGCGAGTGGCTG
ATCGTGAACCGGAAGAATCAGGGCAGCCCAGAGATCTGCGGCTTCAAGAAAACCGGCAGCCGGAA
GGACTTCCACAAGACCCTGCCTAAGCAGACCTTCGAGAGCGAGACACTGGAATGCTCTGAGCCTG
CTGCTCAGGCCGGACCTAGACACCTGAATGTGCTGTGTGACGTGTCCGGCAAGGGACCTGTGACC
GCCTGCGATTTCGACCTGAGAAGTCTGCAGCCCGACAAGCGGCTGGAAAACCTGCTCCAGCAAGT
GTCCGCCGAGGACTTCGAGAAGCAGAACGAGGAAGCCAGACGGACCAACAGACAGGCCGAGCTGT
TTGCTCTGTACCCCAGCGTGGACGAGGAAGATGCCGTCGAGATTAGACCCGTGCCTGAGTGCCCA
AAAGAGCACCTGGGCAACAGAATCCTGGTCAAGCTGCTGACCCTGAAGTTCGAGATCGAGATTGA
GCCCCTGTTCGCCTCTATCGCCCTGTACGACGTGAAAGAGCGGAAGAAGATCAGCGAGAACTTCC
ACTGCGACCTGAACTCCGACCAGTTCAAGGGCTTCCTGAGAGCCCACACACCATCTGTGGCCGCT
AGCTCTCAGGCCAGATCTGCCGTGTTCAGCGTGACATACCCCAGCAGCGACATCTACCTGGTGGT
CAAGATCGAGAAGGTCCTGCAGCAGGGCGAGATCGGCGATTGTGCCGAGCCTTACACCGTGATCA
AAGAGAGCGACGGCGGCAAGAGCAAAGAGAAGATTGAGAAGCTGAAGCTGCAGGCCGAGAGCTTC
TGTCAGCGGCTGGGCAAGTACAGAATGCCCTTTGCCTGGGCTCCTATCAGCCTGTCCAGCTTCTT
CAACGTGTCCACACTGGAACGGGAAGTGACCGACGTGGACTCTGTCGTGGGCAGATCTAGCGTGG
GCGAGAGAAGGACACTGGCTCAGTCTAGACGGCTGAGCGAGAGAGCCCTGAGCCTGGAAGAAAAT
GGCGTGGGCAGCAACTTCAAGACCAGCACTCTGTCCGTGTCTAGCTTCTTTAAGCAAGAGGGCGA
CAGACTGAGCGACGAGGACCTGTTCAAGTTCCTGGCCGACTACAAGCGGAGCAGCTCCCTGCAGA
GAAGAGTGAAGTCTATCCCTGGCCTGCTGCGCCTGGAAATCTCTACAGCCCCTGAGATCATCAAC
TGCTGTCTGACCCCTGAAATGCTGCCCGTGAAGCCCTTTCCAGAGAACCGGACCAGACCTCACAA
AGAGATCCTGGAATTCCCCACCAGAGAAGTGTACGTGCCCCACACCGTGTACCGGAACCTGCTGT
ATGTGTACCCACAGCGGCTGAACTTCGTGAACAAGCTGGCCTCCGCCAGAAACATCACCATCAAG
ATCCAGTTTATGTGCGGCGAGGACGCCAGCAACGCCATGCCTGTGATCTTCGGCAAGTCTAGCGG
CCCTGAGTTCCTGCAAGAGGTGTACACAGCCGTGACCTACCACAACAAGAGCCCCGACTTCTACG
AGGAAGTGAAGATTAAGCTGCCCGCCAAGCTGACCGTGAATCACCATCTGCTGTTCACCTTCTAC
CACATCAGCTGCCAGCAGAAACAGGGCGCCTCTGTGGAAACACTGCTGGGCTATAGCTGGCTGCC
CATCCTGCTGAACGAGAGACTGCAGACCGGCAGCTACTGTCTGCCTGTGGCTCTGGAAAAGCTGC
CACCTAACTACAGCATGCACTCCGCCGAGAAGGTGCCCCTGCAGAATCCTCCTATTAAGTGGGCC
GAGGGCCACAAGGGCGTGTTCAATATCGAGGTGCAGGCCGTGTCCTCCGTGCACACCCAGGATAA
CCACCTGGAAAAGTTCTTCACCCTGTGCCACAGCCTCGAGAGCCAAGTGACATTCCCCATCCGCG
TGCTGGACCAGAAAATCTCCGAGATGGCCCTGGAACACGAGCTGAAACTGAGCATCATCTGCCTG
AATAGCAGCAGACTGGAACCCCTGGTGCTGTTCCTGCATCTGGTGCTGGACAAGCTGTTCCAGCT
GAGCGTGCAGCCCATGGTTATCGCCGGACAGACCGCCAACTTCAGCCAGTTCGCCTTTGAGAGCG
TGGTGGCCATTGCCAACAGCCTGCACAACAGCAAGGACCTGAGCAAGGATCAGCACGGCAGAAAC
TGCCTGCTGGCCTCTTACGTGCACTACGTGTTCAGACTGCCTGAGGTGCAGAGGGACGTGCCAAA
ATCTGGCGCTCCTACCGCTCTGCTGGACCCCAGAAGCTATCACACCTACGGCAGAACATCTGCCG
CCGCTGTGTCCTCTAAACTCCTGCAGGCTAGAGTGATGTCCAGCAGCAACCCTGATCTGGCCGGA
ACACACAGCGCCGCTGATGAAGAAGTGAAAAACATCATGAGCAGCAAGATCGCCGACCGGAACTG
CAGCCGGATGAGCTACTACTGCCTGAGCTACGACACCGAGATCCTGACCGTGGAATACGGCTTCC
TGCCTATCGGCAAGATCGTGGAAGAACGGATCGAGTGCACCGTGTACACCGTGGACAAGAACGGC
TTCGTGTACACACAGCCTATCGCTCAGTGGCACAACCGGGGAGAGCAAGAGGTGTTCGAGTACTG
CCTGGAAGATGGCAGCATCATCCGGGCCACCAAGGACCACAAGTTCATGACCACCGACGGCCAGA
TGCTGCCCATCGACGAGATCTTTGAGAGAGGCCTGGACCTGAAACAGGTGGACGGACTTCCTTGA
(SEQ ID NO: 14)
939 DOCK8 (1^{st} DOCK8 fragment; 5′ half)
ATGGCCACACTGCCTAGCGCTGAGAGAAGGGCCTTCGCTCTGAAGATCAACCGGTACAGCAGCGC
CGAGATCCGGAAGCAGTTTACCCTGCCTCCTAACCTGGGCCAGTACCACAGACAGAGCATCAGCA
CCAGCGGCTTCCCATCTCTGCAGCTGCCCCAGTTCTACGACCCTGTGGAACCCGTGGATTTCGAG
GGCCTGCTGATGACCCACCTGAACAGCCTGGATGTGCAGCTGGCCCAAGAGCTGGGCGACTTCAC
CGACGATGATCTGGACGTGGTGTTCACCCCTAAAGAGTGCAGAACCCTGCAGCCTAGCCTGCCTG
AAGAAGGCGTGGAACTGGATCCTCACGTGCGGGATTGTGTGCAGACCTACATCCGCGAGTGGCTG
ATCGTGAACCGGAAGAATCAGGGCAGCCCAGAGATCTGCGGCTTCAAGAAAACCGGCAGCCGGAA
GGACTTCCACAAGACCCTGCCTAAGCAGACCTTCGAGAGCGAGACACTGGAATGCTCTGAGCCTG
CTGCTCAGGCCGGACCTAGACACCTGAATGTGCTGTGTGACGTGTCCGGCAAGGGACCTGTGACC
GCCTGCGATTTCGACCTGAGAAGTCTGCAGCCCGACAAGCGGCTGGAAAACCTGCTCCAGCAAGT
GTCCGCCGAGGACTTCGAGAAGCAGAACGAGGAAGCCAGACGGACCAACAGACAGGCCGAGCTGT
TTGCTCTGTACCCCAGCGTGGACGAGGAAGATGCCGTCGAGATTAGACCCGTGCCTGAGTGCCCA
AAAGAGCACCTGGGCAACAGAATCCTGGTCAAGCTGCTGACCCTGAAGTTCGAGATCGAGATTGA
GCCCCTGTTCGCCTCTATCGCCCTGTACGACGTGAAAGAGCGGAAGAAGATCAGCGAGAACTTCC
ACTGCGACCTGAACTCCGACCAGTTCAAGGGCTTCCTGAGAGCCCACACACCATCTGTGGCCGCT
AGCTCTCAGGCCAGATCTGCCGTGTTCAGCGTGACATACCCCAGCAGCGACATCTACCTGGTGGT
CAAGATCGAGAAGGTCCTGCAGCAGGGCGAGATCGGCGATTGTGCCGAGCCTTACACCGTGATCA
AAGAGAGCGACGGCGGCAAGAGCAAAGAGAAGATTGAGAAGCTGAAGCTGCAGGCCGAGAGCTTC
TGTCAGCGGCTGGGCAAGTACAGAATGCCCTTTGCCTGGGCTCCTATCAGCCTGTCCAGCTTCTT
CAACGTGTCCACACTGGAACGGGAAGTGACCGACGTGGACTCTGTCGTGGGCAGATCTAGCGTGG
GCGAGAGAAGGACACTGGCTCAGTCTAGACGGCTGAGCGAGAGAGCCCTGAGCCTGGAAGAAAAT
GGCGTGGGCAGCAACTTCAAGACCAGCACTCTGTCCGTGTCTAGCTTCTTTAAGCAAGAGGGCGA
CAGACTGAGCGACGAGGACCTGTTCAAGTTCCTGGCCGACTACAAGCGGAGCAGCTCCCTGCAGA
GAAGAGTGAAGTCTATCCCTGGCCTGCTGCGCCTGGAAATCTCTACAGCCCCTGAGATCATCAAC
TGCTGTCTGACCCCTGAAATGCTGCCCGTGAAGCCCTTTCCAGAGAACCGGACCAGACCTCACAA
AGAGATCCTGGAATTCCCCACCAGAGAAGTGTACGTGCCCCACACCGTGTACCGGAACCTGCTGT
ATGTGTACCCACAGCGGCTGAACTTCGTGAACAAGCTGGCCTCCGCCAGAAACATCACCATCAAG
ATCCAGTTTATGTGCGGCGAGGACGCCAGCAACGCCATGCCTGTGATCTTCGGCAAGTCTAGCGG
CCCTGAGTTCCTGCAAGAGGTGTACACAGCCGTGACCTACCACAACAAGAGCCCCGACTTCTACG
AGGAAGTGAAGATTAAGCTGCCCGCCAAGCTGACCGTGAATCACCATCTGCTGTTCACCTTCTAC
CACATCAGCTGCCAGCAGAAACAGGGCGCCTCTGTGGAAACACTGCTGGGCTATAGCTGGCTGCC
CATCCTGCTGAACGAGAGACTGCAGACCGGCAGCTACTGTCTGCCTGTGGCTCTGGAAAAGCTGC
CACCTAACTACAGCATGCACTCCGCCGAGAAGGTGCCCCTGCAGAATCCTCCTATTAAGTGGGCC
GAGGGCCACAAGGGCGTGTTCAATATCGAGGTGCAGGCCGTGTCCTCCGTGCACACCCAGGATAA
CCACCTGGAAAAGTTCTTCACCCTGTGCCACAGCCTCGAGAGCCAAGTGACATTCCCCATCCGCG
TGCTGGACCAGAAAATCTCCGAGATGGCCCTGGAACACGAGCTGAAACTGAGCATCATCTGCCTG
AATAGCAGCAGACTGGAACCCCTGGTGCTGTTCCTGCATCTGGTGCTGGACAAGCTGTTCCAGCT
GAGCGTGCAGCCCATGGTTATCGCCGGACAGACCGCCAACTTCAGCCAGTTCGCCTTTGAGAGCG
TGGTGGCCATTGCCAACAGCCTGCACAACAGCAAGGACCTGAGCAAGGATCAGCACGGCAGAAAC
TGCCTGCTGGCCTCTTACGTGCACTACGTGTTCAGACTGCCTGAGGTGCAGAGGGACGTGCCAAA
ATCTGGCGCTCCTACCGCTCTGCTGGACCCCAGAAGCTATCACACCTACGGCAGAACATCTGCCG
CCGCTGTGTCCTCTAAACTCCTGCAGGCTAGAGTGATGTCCAGCAGCAACCCTGATCTGGCCGGA
ACACACAGCGCCGCTGATGAAGAAGTGAAAAACATCATGAGCAGCAAGATCGCCGACCGGAACTG
CAGCCGGATGAGCTACTAC
(SEQ ID NO: 15)
939 DOCK8-CfaN (1^{st} DOCK8 fragment; N-terminal half)
MATLPSAERRAFALKINRYSSAEIRKQFTLPPNLGQYHRQSISTSGFPSLQLPQFYDPVEPVDFE
GLLMTHLNSLDVQLAQELGDFTDDDLDVVFTPKECRTLQPSLPEEGVELDPHVRDCVQTYIREWL
IVNRKNQGSPEICGFKKTGSRKDFHKTLPKQTFESETLECSEPAAQAGPRHLNVLCDVSGKGPVT
ACDFDLRSLQPDKRLENLLQQVSAEDFEKQNEEARRTNRQAELFALYPSVDEEDAVEIRPVPECP
KEHLGNRILVKLLTLKFEIEIEPLFASIALYDVKERKKISENFHCDLNSDQFKGFLRAHTPSVAA
SSQARSAVFSVTYPSSDIYLVVKIEKVLQQGEIGDCAEPYTVIKESDGGKSKEKIEKLKLQAESF
CQRLGKYRMPFAWAPISLSSFFNVSTLEREVTDVDSVVGRSSVGERRTLAQSRRLSERALSLEEN
GVGSNFKTSTLSVSSFFKQEGDRLSDEDLFKFLADYKRSSSLQRRVKSIPGLLRLEISTAPEIIN
CCLTPEMLPVKPFPENRTRPHKEILEFPTREVYVPHTVYRNLLYVYPQRLNFVNKLASARNITIK
IQFMCGEDASNAMPVIFGKSSGPEFLQEVYTAVTYHNKSPDFYEEVKIKLPAKLTVNHHLLFTFY
HISCQQKQGASVETLLGYSWLPILLNERLQTGSYCLPVALEKLPPNYSMHSAEKVPLQNPPIKWA
EGHKGVFNIEVQAVSSVHTQDNHLEKFFTLCHSLESQVTFPIRVLDQKISEMALEHELKLSIICL
NSSRLEPLVLFLHLVLDKLFQLSVQPMVIAGQTANFSQFAFESVVAIANSLHNSKDLSKDQHGRN
CLLASYVHYVFRLPEVQRDVPKSGAPTALLDPRSYHTYGRTSAAAVSSKLLQARVMSSSNPDLAG
THSAADEEVKNIMSSKIADRNCSRMSYYCLSYDTEILTVEYGFLPIGKIVEERIECTVYTVDKNG
FVYTQPIAQWHNRGEQEVFEYCLEDGSIIRATKDHKFMTTDGQMLPIDEIFERGLDLKQVDGLP*
(SEQ ID NO: 17; *- stop codon)
939 DOCK8 (1^{st} DOCK8 fragment; N-terminal half)
MATLPSAERRAFALKINRYSSAEIRKQFTLPPNLGQYHRQSISTSGFPSLQLPQFYDPVEPVDFE
GLLMTHLNSLDVQLAQELGDFTDDDLDVVFTPKECRTLQPSLPEEGVELDPHVRDCVQTYIREWL
IVNRKNQGSPEICGFKKTGSRKDFHKTLPKQTFESETLECSEPAAQAGPRHLNVLCDVSGKGPVT
ACDFDLRSLQPDKRLENLLQQVSAEDFEKQNEEARRTNRQAELFALYPSVDEEDAVEIRPVPECP
KEHLGNRILVKLLTLKFEIEIEPLFASIALYDVKERKKISENFHCDLNSDQFKGFLRAHTPSVAA
SSQARSAVFSVTYPSSDIYLVVKIEKVLQQGEIGDCAEPYTVIKESDGGKSKEKIEKLKLQAESF
CQRLGKYRMPFAWAPISLSSFFNVSTLEREVTDVDSVVGRSSVGERRTLAQSRRLSERALSLEEN
GVGSNFKTSTLSVSSFFKQEGDRLSDEDLFKFLADYKRSSSLQRRVKSIPGLLRLEISTAPEIIN
CCLTPEMLPVKPFPENRTRPHKEILEFPTREVYVPHTVYRNLLYVYPQRLNFVNKLASARNITIK
IQFMCGEDASNAMPVIFGKSSGPEFLQEVYTAVTYHNKSPDFYEEVKIKLPAKLTVNHHLLFTFY
HISCQQKQGASVETLLGYSWLPILLNERLQTGSYCLPVALEKLPPNYSMHSAEKVPLQNPPIKWA
EGHKGVFNIEVQAVSSVHTQDNHLEKFFTLCHSLESQVTFPIRVLDQKISEMALEHELKLSIICL
NSSRLEPLVLFLHLVLDKLFQLSVQPMVIAGQTANFSQFAFESVVAIANSLHNSKDLSKDQHGRN
CLLASYVHYVFRLPEVQRDVPKSGAPTALLDPRSYHTYGRTSAAAVSSKLLQARVMSSSNPDLAG
THSAADEEVKNIMSSKIADRNCSRMSYY
(SEQ ID NO: 18)
939 CfaC-CFN-DOCK8 (2^{nd} DOCK8 fragment; 3′ half)
ATGGTGAAGATCATCAGCAGAAAGAGCCTGGGCACCCAGAACGTGTACGACATCGGCGTGGAAAA
GGACCACAACTTTCTGCTCAAGAACGGCCTGGTGGCCAGCAACTGCTTTAATAGCGGCAGCTCTG
ATGCCCCTAGCTCTCCAGCTGCTCCTAGACCTGCCAGCAAGAAGCACTTTCACGAGGAACTGGCC
CTGCAGATGGTCGTGTCTACCGGCATGGTTCGAGAGACAGTGTTTAAGTACGCCTGGTTCTTCTT
CGAACTGCTGGTCAAGAGCATGGCCCAGCACGTGCACAACATGGACAAGCGGGACAGCTTCCGGC
GGACCAGATTCAGCGACAGATTCATGGACGACATCACCACCATCGTGAACGTGGTCACCAGCGAG
ATTGCTGCCCTGCTGGTTAAGCCCCAGAAAGAGAACGAGCAGGCCGAAAAGATGAATATCTCTCT
GGCCTTCTTCCTCTACGACCTGCTGAGCCTGATGGACCGGGGCTTCGTGTTCAACCTGATCCGGC
ACTACTGCTCCCAGCTGTCTGCCAAGCTGTCCAACCTGCCTACACTGATCTCTATGAGACTCGAG
TTCCTGAGGATCCTGTGCAGCCACGAGCACTACCTGAACCTGAATCTGTTCTTCATGAACGCCGA
CACAGCCCCTACAAGCCCCTGTCCTTCTATCAGCAGCCAGAACAGCAGCAGCTGTAGCAGCTTTC
AGGATCAGAAAATCGCCAGCATGTTCGATCTGACCAGCGAGTACCGGCAGCAGCACTTTCTGACC
GGCCTGCTGTTTACCGAACTGGCAGCCGCTCTTGATGCCGAAGGCGAGGGAATCAGCAAGGTGCA
GCGGAAAGCCGTGTCTGCCATTCACAGCCTGCTGTCTAGCCACGACCTGGATCCTAGATGCGTGA
AGCCTGAAGTGAAAGTGAAAATCGCTGCCCTGTACCTGCCTCTCGTGGGCATCATTCTGGATGCC
CTGCCTCAGCTGTGTGATTTCACCGTGGCCGACACCAGACGGTACAGAACCTCTGGCTCCGACGA
GGAACAAGAAGGCGCTGGCGCCATCAACCAGAATGTGGCCCTTGCCATTGCCGGGAACAACTTCA
ATCTGAAAACCAGCGGCATCGTGCTGAGCAGCCTGCCTTACAAGCAGTACAACATGCTGAATGCC
GACACTACCCGCAACCTGATGATCTGCTTCCTGTGGATTATGAAGAACGCCGATCAGAGCCTGAT
TCGGAAGTGGATTGCCGACCTGCCAAGCACTCAGCTGAACCGGATTCTGGACCTCCTGTTCATCT
GCGTGCTGTGCTTCGAGTACAAGGGCAAGCAGTCCAGCGACAAGGTGTCCACTCAGGTGCTGCAG
AAAAGCCGGGATGTGAAGGCCAGGCTGGAAGAGGCACTTCTGAGAGGCGAAGGCGCCAGAGGCGA
GATGATGAGGCGTAGAGCCCCTGGCAACGATAGATTCCCCGGCCTGAATGAGAACCTGCGGTGGA
AGAAAGAGCAGACCCATTGGAGACAGGCCAACGAGAAACTGGACAAGACCAAGGCCGAACTGGAC
CAAGAGGCCCTGATCTCTGGCAACCTGGCCACAGAAGCCCACCTGATTATCCTGGACATGCAAGA
GAACATCATCCAGGCCAGTAGCGCCCTGGATTGCAAGGATTCTCTGCTCGGAGGCGTGCTGAGGG
TGCTCGTGAATAGCCTGAATTGCGACCAGAGCACCACCTACCTGACACACTGCTTCGCCACACTG
AGGGCCCTGATTGCCAAGTTTGGGGACCTGCTGTTCGAGGAAGAGGTGGAACAGTGCTTCGACCT
GTGTCACCAAGTGCTGCACCACTGCAGCAGCTCCATGGACGTGACAAGATCCCAGGCCTGCGCCA
CTCTGTATCTGCTGATGCGGTTTAGCTTCGGCGCCACCAGCAATTTCGCCAGAGTGAAGATGCAA
GTGACCATGAGCCTGGCCAGCCTCGTTGGAAGGGCCCCTGACTTCAATGAGGAACACCTCAGAAG
AAGCCTGCGGACCATCCTGGCCTACAGCGAAGAGGATACCGCCATGCAGATGACCCCATTTCCAA
CACAAGTGGAAGAACTGCTGTGCAACCTGAATTCCATCCTGTACGATACCGTGAAGATGAGAGAG
TTCCAAGAGGACCCCGAGATGCTGATGGATCTGATGTACCGGATCGCCAAGAGCTACCAGGCCTC
TCCTGATCTGAGACTGACCTGGCTGCAGAACATGGCCGAGAAGCACACCAAGAAGAAGTGCTACA
CCGAGGCCGCCATGTGCCTGGTTCATGCTGCTGCACTCGTGGCCGAGTACCTGAGCATGCTGGAA
GATCACTCCTACCTGCCAGTGGGCAGCGTGTCCTTCCAGAACATCAGCTCCAACGTCCTGGAAGA
GTCCGTGGTGTCCGAGGACACACTGAGCCCAGATGAGGATGGCGTGTGTGCCGGACAGTACTTTA
CCGAGTCTGGCCTCGTGGGCCTTCTGGAACAGGCCGCCGAACTGTTTTCTACCGGCGGACTGTAC
GAGACAGTGAACGAAGTGTACAAGCTGGTCATCCCCATCCTGGAAGCCCACAGAGAGTTCAGAAA
GCTGACACTGACCCACTCCAAGCTGCAGCGGGCCTTCGACAGCATTGTGAACAAGGACCACAAGC
GGATGTTCGGCACCTACTTCAGAGTGGGCTTTTTCGGCAGCAAATTCGGCGACCTGGACGAGCAA
GAGTTCGTGTACAAAGAGCCCGCCATCACCAAGCTGCCTGAGATCTCCCATCGGCTGGAAGCCTT
CTACGGCCAGTGTTTTGGCGCCGAGTTCGTGGAAGTGATCAAGGACAGCACCCCTGTGGATAAGA
CCAAACTGGACCCTAACAAGGCCTACATCCAGATCACCTTCGTGGAACCCTACTTCGACGAGTAC
GAGATGAAGGATAGAGTGACCTACTTTGAGAAGAACTTCAACCTGCGGCGGTTCATGTACACCAC
ACCTTTTACACTGGAAGGCAGACCCAGGGGCGAGCTGCACGAGCAGTATAGAAGAAACACCGTGC
TGACCACCATGCACGCATTCCCTTACATCAAGACCCGGATCAGCGTGATCCAGAAAGAAGAGTTT
GTCCTGACACCAATCGAGGTGGCCATCGAGGACATGAAGAAGAAAACCCTCCAGCTGGCCGTGGC
CATCAATCAAGAACCTCCAGACGCCAAGATGCTCCAGATGGTGCTCCAGGGATCTGTGGGCGCCA
CAGTGAATCAGGGACCTCTGGAAGTGGCCCAAGTGTTCCTGGCTGAGATCCCCGCCGATCCTAAG
CTGTACAGACACCATAACAAGCTGCGGCTCTGCTTCAAAGAATTCATCATGAGATGTGGCGAGGC
CGTGGAAAAGAACAAGAGACTGATCACCGCCGACCAGAGAGAGTACCAGCAAGAGCTGAAGAAAA
ACTACAACAAGCTCAAAGAAAATCTGCGGCCCATGATCGAGAGAAAGATCCCCGAGCTGTACAAG
CCCATCTTCCGCGTGGAAAGCCAGAAGCGGGACTCCTTTCACAGATCCAGCTTTAGGAAGTGCGA
GACACAGCTGTCCCAGGGCTCTTGA
(SEQ ID NO: 20)
939-DOCK8 (2^{nd} DOCK8 fragment; 3′ half)
AGCGGCAGCTCTGATGCCCCTAGCTCTCCAGCTGCTCCTAGACCTGCCAGCAAGAAGCACTTTCA
CGAGGAACTGGCCCTGCAGATGGTCGTGTCTACCGGCATGGTTCGAGAGACAGTGTTTAAGTACG
CCTGGTTCTTCTTCGAACTGCTGGTCAAGAGCATGGCCCAGCACGTGCACAACATGGACAAGCGG
GACAGCTTCCGGCGGACCAGATTCAGCGACAGATTCATGGACGACATCACCACCATCGTGAACGT
GGTCACCAGCGAGATTGCTGCCCTGCTGGTTAAGCCCCAGAAAGAGAACGAGCAGGCCGAAAAGA
TGAATATCTCTCTGGCCTTCTTCCTCTACGACCTGCTGAGCCTGATGGACCGGGGCTTCGTGTTC
AACCTGATCCGGCACTACTGCTCCCAGCTGTCTGCCAAGCTGTCCAACCTGCCTACACTGATCTC
TATGAGACTCGAGTTCCTGAGGATCCTGTGCAGCCACGAGCACTACCTGAACCTGAATCTGTTCT
TCATGAACGCCGACACAGCCCCTACAAGCCCCTGTCCTTCTATCAGCAGCCAGAACAGCAGCAGC
TGTAGCAGCTTTCAGGATCAGAAAATCGCCAGCATGTTCGATCTGACCAGCGAGTACCGGCAGCA
GCACTTTCTGACCGGCCTGCTGTTTACCGAACTGGCAGCCGCTCTTGATGCCGAAGGCGAGGGAA
TCAGCAAGGTGCAGCGGAAAGCCGTGTCTGCCATTCACAGCCTGCTGTCTAGCCACGACCTGGAT
CCTAGATGCGTGAAGCCTGAAGTGAAAGTGAAAATCGCTGCCCTGTACCTGCCTCTCGTGGGCAT
CATTCTGGATGCCCTGCCTCAGCTGTGTGATTTCACCGTGGCCGACACCAGACGGTACAGAACCT
CTGGCTCCGACGAGGAACAAGAAGGCGCTGGCGCCATCAACCAGAATGTGGCCCTTGCCATTGCC
GGGAACAACTTCAATCTGAAAACCAGCGGCATCGTGCTGAGCAGCCTGCCTTACAAGCAGTACAA
CATGCTGAATGCCGACACTACCCGCAACCTGATGATCTGCTTCCTGTGGATTATGAAGAACGCCG
ATCAGAGCCTGATTCGGAAGTGGATTGCCGACCTGCCAAGCACTCAGCTGAACCGGATTCTGGAC
CTCCTGTTCATCTGCGTGCTGTGCTTCGAGTACAAGGGCAAGCAGTCCAGCGACAAGGTGTCCAC
TCAGGTGCTGCAGAAAAGCCGGGATGTGAAGGCCAGGCTGGAAGAGGCACTTCTGAGAGGCGAAG
GCGCCAGAGGCGAGATGATGAGGCGTAGAGCCCCTGGCAACGATAGATTCCCCGGCCTGAATGAG
AACCTGCGGTGGAAGAAAGAGCAGACCCATTGGAGACAGGCCAACGAGAAACTGGACAAGACCAA
GGCCGAACTGGACCAAGAGGCCCTGATCTCTGGCAACCTGGCCACAGAAGCCCACCTGATTATCC
TGGACATGCAAGAGAACATCATCCAGGCCAGTAGCGCCCTGGATTGCAAGGATTCTCTGCTCGGA
GGCGTGCTGAGGGTGCTCGTGAATAGCCTGAATTGCGACCAGAGCACCACCTACCTGACACACTG
CTTCGCCACACTGAGGGCCCTGATTGCCAAGTTTGGGGACCTGCTGTTCGAGGAAGAGGTGGAAC
AGTGCTTCGACCTGTGTCACCAAGTGCTGCACCACTGCAGCAGCTCCATGGACGTGACAAGATCC
CAGGCCTGCGCCACTCTGTATCTGCTGATGCGGTTTAGCTTCGGCGCCACCAGCAATTTCGCCAG
AGTGAAGATGCAAGTGACCATGAGCCTGGCCAGCCTCGTTGGAAGGGCCCCTGACTTCAATGAGG
AACACCTCAGAAGAAGCCTGCGGACCATCCTGGCCTACAGCGAAGAGGATACCGCCATGCAGATG
ACCCCATTTCCAACACAAGTGGAAGAACTGCTGTGCAACCTGAATTCCATCCTGTACGATACCGT
GAAGATGAGAGAGTTCCAAGAGGACCCCGAGATGCTGATGGATCTGATGTACCGGATCGCCAAGA
GCTACCAGGCCTCTCCTGATCTGAGACTGACCTGGCTGCAGAACATGGCCGAGAAGCACACCAAG
AAGAAGTGCTACACCGAGGCCGCCATGTGCCTGGTTCATGCTGCTGCACTCGTGGCCGAGTACCT
GAGCATGCTGGAAGATCACTCCTACCTGCCAGTGGGCAGCGTGTCCTTCCAGAACATCAGCTCCA
ACGTCCTGGAAGAGTCCGTGGTGTCCGAGGACACACTGAGCCCAGATGAGGATGGCGTGTGTGCC
GGACAGTACTTTACCGAGTCTGGCCTCGTGGGCCTTCTGGAACAGGCCGCCGAACTGTTTTCTAC
CGGCGGACTGTACGAGACAGTGAACGAAGTGTACAAGCTGGTCATCCCCATCCTGGAAGCCCACA
GAGAGTTCAGAAAGCTGACACTGACCCACTCCAAGCTGCAGCGGGCCTTCGACAGCATTGTGAAC
AAGGACCACAAGCGGATGTTCGGCACCTACTTCAGAGTGGGCTTTTTCGGCAGCAAATTCGGCGA
CCTGGACGAGCAAGAGTTCGTGTACAAAGAGCCCGCCATCACCAAGCTGCCTGAGATCTCCCATC
GGCTGGAAGCCTTCTACGGCCAGTGTTTTGGCGCCGAGTTCGTGGAAGTGATCAAGGACAGCACC
CCTGTGGATAAGACCAAACTGGACCCTAACAAGGCCTACATCCAGATCACCTTCGTGGAACCCTA
CTTCGACGAGTACGAGATGAAGGATAGAGTGACCTACTTTGAGAAGAACTTCAACCTGCGGCGGT
TCATGTACACCACACCTTTTACACTGGAAGGCAGACCCAGGGGCGAGCTGCACGAGCAGTATAGA
AGAAACACCGTGCTGACCACCATGCACGCATTCCCTTACATCAAGACCCGGATCAGCGTGATCCA
GAAAGAAGAGTTTGTCCTGACACCAATCGAGGTGGCCATCGAGGACATGAAGAAGAAAACCCTCC
AGCTGGCCGTGGCCATCAATCAAGAACCTCCAGACGCCAAGATGCTCCAGATGGTGCTCCAGGGA
TCTGTGGGCGCCACAGTGAATCAGGGACCTCTGGAAGTGGCCCAAGTGTTCCTGGCTGAGATCCC
CGCCGATCCTAAGCTGTACAGACACCATAACAAGCTGCGGCTCTGCTTCAAAGAATTCATCATGA
GATGTGGCGAGGCCGTGGAAAAGAACAAGAGACTGATCACCGCCGACCAGAGAGAGTACCAGCAA
GAGCTGAAGAAAAACTACAACAAGCTCAAAGAAAATCTGCGGCCCATGATCGAGAGAAAGATCCC
CGAGCTGTACAAGCCCATCTTCCGCGTGGAAAGCCAGAAGCGGGACTCCTTTCACAGATCCAGCT
TTAGGAAGTGCGAGACACAGCTGTCCCAGGGCTCT
(SEQ ID NO: 22)
939 CfaC-CFN-DOCK8 (2^{nd} DOCK8 fragment; C-terminal half)
MVKIISRKSLGTQNVYDIGVEKDHNFLLKNGLVASNCFNSGSSDAPSSPAAPRPASKKHFHEELA
LQMVVSTGMVRETVFKYAWFFFELLVKSMAQHVHNMDKRDSFRRTRFSDRFMDDITTIVNVVTSE
IAALLVKPQKENEQAEKMNISLAFFLYDLLSLMDRGFVFNLIRHYCSQLSAKLSNLPTLISMRLE
FLRILCSHEHYLNLNLFFMNADTAPTSPCPSISSQNSSSCSSFQDQKIASMFDLTSEYRQQHFLT
GLLFTELAAALDAEGEGISKVQRKAVSAIHSLLSSHDLDPRCVKPEVKVKIAALYLPLVGIILDA
LPQLCDFTVADTRRYRTSGSDEEQEGAGAINQNVALAIAGNNFNLKTSGIVLSSLPYKQYNMLNA
DTTRNLMICFLWIMKNADQSLIRKWIADLPSTQLNRILDLLFICVLCFEYKGKQSSDKVSTQVLQ
KSRDVKARLEEALLRGEGARGEMMRRRAPGNDRFPGLNENLRWKKEQTHWRQANEKLDKTKAELD
QEALISGNLATEAHLIILDMQENIIQASSALDCKDSLLGGVLRVLVNSLNCDQSTTYLTHCFATL
RALIAKFGDLLFEEEVEQCFDLCHQVLHHCSSSMDVTRSQACATLYLLMRFSFGATSNFARVKMQ
VTMSLASLVGRAPDFNEEHLRRSLRTILAYSEEDTAMQMTPFPTQVEELLCNLNSILYDTVKMRE
FQEDPEMLMDLMYRIAKSYQASPDLRLTWLQNMAEKHTKKKCYTEAAMCLVHAAALVAEYLSMLE
DHSYLPVGSVSFQNISSNVLEESVVSEDTLSPDEDGVCAGQYFTESGLVGLLEQAAELFSTGGLY
ETVNEVYKLVIPILEAHREFRKLTLTHSKLQRAFDSIVNKDHKRMFGTYFRVGFFGSKFGDLDEQ
EFVYKEPAITKLPEISHRLEAFYGQCFGAEFVEVIKDSTPVDKTKLDPNKAYIQITFVEPYFDEY
EMKDRVTYFEKNFNLRRFMYTTPFTLEGRPRGELHEQYRRNTVLTTMHAFPYIKTRISVIQKEEF
VLTPIEVAIEDMKKKTLQLAVAINQEPPDAKMLQMVLQGSVGATVNQGPLEVAQVFLAEIPADPK
LYRHHNKLRLCFKEFIMRCGEAVEKNKRLITADQREYQQELKKNYNKLKENLRPMIERKIPELYK
PIFRVESQKRDSFHRSSFRKCETQLSQGS*
(SEQ ID NO: 23; *- stop codon)
939 DOCK8 (2^{nd} DOCK8 fragment; C-terminal half)
SGSSDAPSSPAAPRPASKKHFHEELALQMVVSTGMVRETVFKYAWFFFELLVKSMAQHVHNMDKR
DSFRRTRFSDRFMDDITTIVNVVTSEIAALLVKPQKENEQAEKMNISLAFFLYDLLSLMDRGFVF
NLIRHYCSQLSAKLSNLPTLISMRLEFLRILCSHEHYLNLNLFFMNADTAPTSPCPSISSQNSSS
CSSFQDQKIASMFDLTSEYRQQHFLTGLLFTELAAALDAEGEGISKVQRKAVSAIHSLLSSHDLD
PRCVKPEVKVKIAALYLPLVGIILDALPQLCDFTVADTRRYRTSGSDEEQEGAGAINQNVALAIA
GNNFNLKTSGIVLSSLPYKQYNMLNADTTRNLMICFLWIMKNADQSLIRKWIADLPSTQLNRILD
LLFICVLCFEYKGKQSSDKVSTQVLQKSRDVKARLEEALLRGEGARGEMMRRRAPGNDRFPGLNE
NLRWKKEQTHWRQANEKLDKTKAELDQEALISGNLATEAHLIILDMQENIIQASSALDCKDSLLG
GVLRVLVNSLNCDQSTTYLTHCFATLRALIAKFGDLLFEEEVEQCFDLCHQVLHHCSSSMDVTRS
QACATLYLLMRFSFGATSNFARVKMQVTMSLASLVGRAPDFNEEHLRRSLRTILAYSEEDTAMQM
TPFPTQVEELLCNLNSILYDTVKMREFQEDPEMLMDLMYRIAKSYQASPDLRLTWLQNMAEKHTK
KKCYTEAAMCLVHAAALVAEYLSMLEDHSYLPVGSVSFQNISSNVLEESVVSEDTLSPDEDGVCA
GQYFTESGLVGLLEQAAELFSTGGLYETVNEVYKLVIPILEAHREFRKLTLTHSKLQRAFDSIVN
KDHKRMFGTYFRVGFFGSKFGDLDEQEFVYKEPAITKLPEISHRLEAFYGQCFGAEFVEVIKDST
PVDKTKLDPNKAYIQITFVEPYFDEYEMKDRVTYFEKNFNLRRFMYTTPFTLEGRPRGELHEQYR
RNTVLTTMHAFPYIKTRISVIQKEEFVLTPIEVAIEDMKKKTLQLAVAINQEPPDAKMLQMVLQG
SVGATVNQGPLEVAQVFLAEIPADPKLYRHHNKLRLCFKEFIMRCGEAVEKNKRLITADQREYQQ
ELKKNYNKLKENLRPMIERKIPELYKPIFRVESQKRDSFHRSSFRKCETQLSQGS*
(SEQ ID NO: 25; *- stop codon)
939 CfaC-CF-DOCK8 (2^{nd} DOCK8 fragment; 3′)
ATGGTGAAGATCATCAGCAGAAAGAGCCTGGGCACCCAGAACGTGTACGACATCGGCGTGGAAAA
GGACCACAACTTTCTGCTCAAGAACGGCCTGGTGGCCAGCAACTGCTTTAGCGGCAGCTCTGATG
CCCCTAGCTCTCCAGCTGCTCCTAGACCTGCCAGCAAGAAGCACTTTCACGAGGAACTGGCCCTG
CAGATGGTCGTGTCTACCGGCATGGTTCGAGAGACAGTGTTTAAGTACGCCTGGTTCTTCTTCGA
ACTGCTGGTCAAGAGCATGGCCCAGCACGTGCACAACATGGACAAGCGGGACAGCTTCCGGCGGA
CCAGATTCAGCGACAGATTCATGGACGACATCACCACCATCGTGAACGTGGTCACCAGCGAGATT
GCTGCCCTGCTGGTTAAGCCCCAGAAAGAGAACGAGCAGGCCGAAAAGATGAATATCTCTCTGGC
CTTCTTCCTCTACGACCTGCTGAGCCTGATGGACCGGGGCTTCGTGTTCAACCTGATCCGGCACT
ACTGCTCCCAGCTGTCTGCCAAGCTGTCCAACCTGCCTACACTGATCTCTATGAGACTCGAGTTC
CTGAGGATCCTGTGCAGCCACGAGCACTACCTGAACCTGAATCTGTTCTTCATGAACGCCGACAC
AGCCCCTACAAGCCCCTGTCCTTCTATCAGCAGCCAGAACAGCAGCAGCTGTAGCAGCTTTCAGG
ATCAGAAAATCGCCAGCATGTTCGATCTGACCAGCGAGTACCGGCAGCAGCACTTTCTGACCGGC
CTGCTGTTTACCGAACTGGCAGCCGCTCTTGATGCCGAAGGCGAGGGAATCAGCAAGGTGCAGCG
GAAAGCCGTGTCTGCCATTCACAGCCTGCTGTCTAGCCACGACCTGGATCCTAGATGCGTGAAGC
CTGAAGTGAAAGTGAAAATCGCTGCCCTGTACCTGCCTCTCGTGGGCATCATTCTGGATGCCCTG
CCTCAGCTGTGTGATTTCACCGTGGCCGACACCAGACGGTACAGAACCTCTGGCTCCGACGAGGA
ACAAGAAGGCGCTGGCGCCATCAACCAGAATGTGGCCCTTGCCATTGCCGGGAACAACTTCAATC
TGAAAACCAGCGGCATCGTGCTGAGCAGCCTGCCTTACAAGCAGTACAACATGCTGAATGCCGAC
ACTACCCGCAACCTGATGATCTGCTTCCTGTGGATTATGAAGAACGCCGATCAGAGCCTGATTCG
GAAGTGGATTGCCGACCTGCCAAGCACTCAGCTGAACCGGATTCTGGACCTCCTGTTCATCTGCG
TGCTGTGCTTCGAGTACAAGGGCAAGCAGTCCAGCGACAAGGTGTCCACTCAGGTGCTGCAGAAA
AGCCGGGATGTGAAGGCCAGGCTGGAAGAGGCACTTCTGAGAGGCGAAGGCGCCAGAGGCGAGAT
GATGAGGCGTAGAGCCCCTGGCAACGATAGATTCCCCGGCCTGAATGAGAACCTGCGGTGGAAGA
AAGAGCAGACCCATTGGAGACAGGCCAACGAGAAACTGGACAAGACCAAGGCCGAACTGGACCAA
GAGGCCCTGATCTCTGGCAACCTGGCCACAGAAGCCCACCTGATTATCCTGGACATGCAAGAGAA
CATCATCCAGGCCAGTAGCGCCCTGGATTGCAAGGATTCTCTGCTCGGAGGCGTGCTGAGGGTGC
TCGTGAATAGCCTGAATTGCGACCAGAGCACCACCTACCTGACACACTGCTTCGCCACACTGAGG
GCCCTGATTGCCAAGTTTGGGGACCTGCTGTTCGAGGAAGAGGTGGAACAGTGCTTCGACCTGTG
TCACCAAGTGCTGCACCACTGCAGCAGCTCCATGGACGTGACAAGATCCCAGGCCTGCGCCACTC
TGTATCTGCTGATGCGGTTTAGCTTCGGCGCCACCAGCAATTTCGCCAGAGTGAAGATGCAAGTG
ACCATGAGCCTGGCCAGCCTCGTTGGAAGGGCCCCTGACTTCAATGAGGAACACCTCAGAAGAAG
CCTGCGGACCATCCTGGCCTACAGCGAAGAGGATACCGCCATGCAGATGACCCCATTTCCAACAC
AAGTGGAAGAACTGCTGTGCAACCTGAATTCCATCCTGTACGATACCGTGAAGATGAGAGAGTTC
CAAGAGGACCCCGAGATGCTGATGGATCTGATGTACCGGATCGCCAAGAGCTACCAGGCCTCTCC
TGATCTGAGACTGACCTGGCTGCAGAACATGGCCGAGAAGCACACCAAGAAGAAGTGCTACACCG
AGGCCGCCATGTGCCTGGTTCATGCTGCTGCACTCGTGGCCGAGTACCTGAGCATGCTGGAAGAT
CACTCCTACCTGCCAGTGGGCAGCGTGTCCTTCCAGAACATCAGCTCCAACGTCCTGGAAGAGTC
CGTGGTGTCCGAGGACACACTGAGCCCAGATGAGGATGGCGTGTGTGCCGGACAGTACTTTACCG
AGTCTGGCCTCGTGGGCCTTCTGGAACAGGCCGCCGAACTGTTTTCTACCGGCGGACTGTACGAG
ACAGTGAACGAAGTGTACAAGCTGGTCATCCCCATCCTGGAAGCCCACAGAGAGTTCAGAAAGCT
GACACTGACCCACTCCAAGCTGCAGCGGGCCTTCGACAGCATTGTGAACAAGGACCACAAGCGGA
TGTTCGGCACCTACTTCAGAGTGGGCTTTTTCGGCAGCAAATTCGGCGACCTGGACGAGCAAGAG
TTCGTGTACAAAGAGCCCGCCATCACCAAGCTGCCTGAGATCTCCCATCGGCTGGAAGCCTTCTA
CGGCCAGTGTTTTGGCGCCGAGTTCGTGGAAGTGATCAAGGACAGCACCCCTGTGGATAAGACCA
AACTGGACCCTAACAAGGCCTACATCCAGATCACCTTCGTGGAACCCTACTTCGACGAGTACGAG
ATGAAGGATAGAGTGACCTACTTTGAGAAGAACTTCAACCTGCGGCGGTTCATGTACACCACACC
TTTTACACTGGAAGGCAGACCCAGGGGCGAGCTGCACGAGCAGTATAGAAGAAACACCGTGCTGA
CCACCATGCACGCATTCCCTTACATCAAGACCCGGATCAGCGTGATCCAGAAAGAAGAGTTTGTC
CTGACACCAATCGAGGTGGCCATCGAGGACATGAAGAAGAAAACCCTCCAGCTGGCCGTGGCCAT
CAATCAAGAACCTCCAGACGCCAAGATGCTCCAGATGGTGCTCCAGGGATCTGTGGGCGCCACAG
TGAATCAGGGACCTCTGGAAGTGGCCCAAGTGTTCCTGGCTGAGATCCCCGCCGATCCTAAGCTG
TACAGACACCATAACAAGCTGCGGCTCTGCTTCAAAGAATTCATCATGAGATGTGGCGAGGCCGT
GGAAAAGAACAAGAGACTGATCACCGCCGACCAGAGAGAGTACCAGCAAGAGCTGAAGAAAAACT
ACAACAAGCTCAAAGAAAATCTGCGGCCCATGATCGAGAGAAAGATCCCCGAGCTGTACAAGCCC
ATCTTCCGCGTGGAAAGCCAGAAGCGGGACTCCTTTCACAGATCCAGCTTTAGGAAGTGCGAGAC
ACAGCTGTCCCAGGGCTCTTGA
(SEQ ID NO: 26)
939 CfaC-CF-DOCK8 (2^{nd} DOCK8 fragment; C-terminal half)
MVKIISRKSLGTQNVYDIGVEKDHNFLLKNGLVASNCFSGSSDAPSSPAAPRPASKKHFHEELAL
QMVVSTGMVRETVFKYAWFFFELLVKSMAQHVHNMDKRDSFRRTRFSDRFMDDITTIVNVVTSEI
AALLVKPQKENEQAEKMNISLAFFLYDLLSLMDRGFVFNLIRHYCSQLSAKLSNLPTLISMRLEF
LRILCSHEHYLNLNLFFMNADTAPTSPCPSISSQNSSSCSSFQDQKIASMFDLTSEYRQQHFLTG
LLFTELAAALDAEGEGISKVQRKAVSAIHSLLSSHDLDPRCVKPEVKVKIAALYLPLVGIILDAL
PQLCDFTVADTRRYRTSGSDEEQEGAGAINQNVALAIAGNNFNLKTSGIVLSSLPYKQYNMLNAD
TTRNLMICFLWIMKNADQSLIRKWIADLPSTQLNRILDLLFICVLCFEYKGKQSSDKVSTQVLQK
SRDVKARLEEALLRGEGARGEMMRRRAPGNDRFPGLNENLRWKKEQTHWRQANEKLDKTKAELDQ
EALISGNLATEAHLIILDMQENIIQASSALDCKDSLLGGVLRVLVNSLNCDQSTTYLTHCFATLR
ALIAKFGDLLFEEEVEQCFDLCHQVLHHCSSSMDVTRSQACATLYLLMRFSFGATSNFARVKMQV
TMSLASLVGRAPDFNEEHLRRSLRTILAYSEEDTAMQMTPFPTQVEELLCNLNSILYDTVKMREF
QEDPEMLMDLMYRIAKSYQASPDLRLTWLQNMAEKHTKKKCYTEAAMCLVHAAALVAEYLSMLED
HSYLPVGSVSFQNISSNVLEESVVSEDTLSPDEDGVCAGQYFTESGLVGLLEQAAELFSTGGLYE
TVNEVYKLVIPILEAHREFRKLTLTHSKLQRAFDSIVNKDHKRMFGTYFRVGFFGSKFGDLDEQE
FVYKEPAITKLPEISHRLEAFYGQCFGAEFVEVIKDSTPVDKTKLDPNKAYIQITFVEPYFDEYE
MKDRVTYFEKNFNLRRFMYTTPFTLEGRPRGELHEQYRRNTVLTTMHAFPYIKTRISVIQKEEFV
LTPIEVAIEDMKKKTLQLAVAINQEPPDAKMLQMVLQGSVGATVNQGPLEVAQVFLAEIPADPKL
YRHHNKLRLCFKEFIMRCGEAVEKNKRLITADQREYQQELKKNYNKLKENLRPMIERKIPELYKP
IFRVESQKRDSFHRSSFRKCETQLSQGS*
(SEQ ID NO: 27; *- stop codon)
EFS-939 DOCK8-CfaN-WPRE (1^{st} DOCK8 fragment; 5′ half)
acgcgccctgtagcggcgcattaagcgcggcgggtgtggtggttacgcgcagcgtgaccgctaca
cttgccagcgccctagcgcccgctcctttcgctttcttcccttcctttctcgccacgttcgccgg
ctttccccgtcaagctctaaatcgggggctccctttagggttccgatttagtgctttacggcacc
tcgaccccaaaaaacttgattagggtgatggttcacgtagtgggccatcgccctgatagacggtt
tttcgccctttgacgttggagtccacgttctttaatagtggactcttgttccaaactggaacaac
actcaaccctatctcggtctattcttttgatttataagggattttgccgatttcggcctattggt
taaaaaatgagctgatttaacaaaaatttaacgcgaattttaacaaaatattaacgcttacaatt
taggtggcacttttcggggaaatgtgcgcggaacccctatttgtttatttttctaaatacattca
aatatgtatccgctcatgagacaataaccctgataaatgcttcaataatagcacctagatcaaga
gacaggatgaggatcgtttcgcatgattgaacaagatggattgcacgcaggttctccggccgctt
gggtggagaggctattcggctatgactgggcacaacagacaatcggctgctctgatgccgccgtg
ttccggctgtcagcgcaggggcgcccggttctttttgtcaagaccgacctgtccggtgccctgaa
tgaactgcaagacgaggcagcgcggctatcgtggctggccacgacgggcgttccttgcgcagctg
tgctcgacgttgtcactgaagcgggaagggactggctgctattgggcgaagtgccggggcaggat
ctcctgtcatctcaccttgctcctgccgagaaagtatccatcatggctgatgcaatgcggcggct
gcatacgcttgatccggctacctgcccattcgaccaccaagcgaaacatcgcatcgagcgagcac
gtactcggatggaagccggtcttgtcgatcaggatgatctggacgaagagcatcaggggctcgcg
ccagccgaactgttcgccaggctcaaggcgagcatgcccgacggcgaggatctcgtcgtgaccca
tggcgatgcctgcttgccgaatatcatggtggaaaatggccgcttttctggattcatcgactgtg
gccggctgggtgtggcggaccgctatcaggacatagcgttggctacccgtgatattgctgaagag
cttggcggcgaatgggctgaccgcttcctcgtgctttacggtatcgccgctcccgattcgcagcg
catcgccttctatcgccttcttgacgagttcttctgaattattaacgcttacaatttcctgatgc
ggtattttctccttacgcatctgtgcggtatttcacaccgcatcaggtggcacttttcggggaaa
tgtgcgcggaacccctatttgtttatttttctaaatacattcaaatatgtatccgctcatgacca
aaatcccttaacgtgagttttcgttccactgagcgtcagaccccgtagaaaagatcaaaggatct
tcttgagatcctttttttctgcgcgtaatctgctgcttgcaaacaaaaaaaccaccgctaccagc
ggtggtttgtttgccggatcaagagctaccaactctttttccgaaggtaactggcttcagcagag
cgcagataccaaatactgttcttctagtgtagccgtagttaggccaccacttcaagaactctgta
gcaccgcctacatacctcgctctgctaatcctgttaccagtggctgctgccagtggcgataagtc
gtgtcttaccgggttggactcaagacgatagttaccggataaggcgcagcggtcgggctgaacgg
gggggtcgtgcacacagcccagcttggagcgaacgacctacaccgaactgagatacctacagcgt
gagctatgagaaagcgccacgcttcccgaagggagaaaggcggacaggtatccggtaagcggcag
ggtcggaacaggagagcgcacgagggagcttccagggggaaacgcctggtatctttatagtcctg
tcgggtttcgccacctctgactigagcgtcgattttigtgatgctcgtcaggggggggagcctal
ggaaaaacgccagcaacgcggcctttttacggttcctggccttttgctggccttttgctcacatg
ttctttcctgcgttatcccctgattctgtggataaccgtattaccgcctttgagtgagctgatac
cgctcgccgcagccgaacgaccgagcgcagcgagtcagtgagcgaggaagcggaagagcgcccaa
tacgcaaaccgcctctccccgcgcgttggccgattcattaatgcagctggcacgacaggtttccc
gactggaaagcgggcagtgagcgcaacgcaattaatgtgagttagctcactcattaggcacccca
ggctttacactttatgcttccggctcgtatgttgtgtggaattgtgagcggataacaatttcaca
caggaaacagctatgaccatgattacgccaagcgcgcaattaaccctcactaaagggaacaaaag
ctggagctgcaagcttggccattgcatacgttgtatccatatcataatatgtacatttatattgg
ctcatgtccaacattaccgccatgttgacattgattattgactagttattaatagtaatcaatta
cggggtcattagttcatagcccatatatggagttccgcgttacataacttacggtaaatggcccg
cctggctgaccgcccaacgacccccgcccattgacgtcaataatgacgtatgttcccatagtaac
gccaatagggactttccattgacgtcaatgggtggagtatttacggtaaactgcccacttggcag
tacatcaagtgtatcatatgccaagtacgccccctattgacgtcaatgacggtaaatggcccgcc
tggcattatgcccagtacatgaccttatgggactttcctacttggcagtacatctacgtattagt
catcgctattaccatggtgatgcggttttggcagtacatcaatgggcgtggatagcggtttgact
cacggggatttccaagtctccaccccattgacgtcaatgggagtttgttttggcaccaaaatcaa
cgggactttccaaaatgtcgtaacaactccgccccattgacgcaaatgggcggtaggcgtgtacg
ggggaggtctatataagcagagctcgtttagtgaaccgGGGTCTCTCTGGTTAGACCAGATCTGA
GCCTGGGAGCTCTCTGGCTAACTAGGGAACCCACTGCTTAAGCCTCAATAAAGCTTGCCTTGAGT
GCTTCAAGTAGTGTGTGCCCGTCTGTTGTGTGACTCTGGTAACTAGAGATCCCTCAGACCCTTTT
AGTCAGTGTGGAAAATCTCTAGCagtggcgcccgaacagggacttgaaagcgaaagggaaaccag
aggagctctcTCGACGCAGGACTCGGCTTGCTGAAGCGCGCACGGCAAGAGGCGAGGGGCGGCGA
CTGGTGAGTACGCCAAAAATTTTGACTAGCGGAGGCTAGAAGGAGAGAGATGGGTGCGAGAGCGT
CAGTATTAAGCGGGGGAGaattagatcgcgatgggaaaaaattcggttaaggccagggggaaaga
aaaaatataaattaaaacatatagtatgggcaagcagggagctagaacgattcgcagttaatcct
ggcctgttagaaacatcagaaggctgtagacaaatactgggacagctacaaccatcccttcagac
aggatcagaagaacttagatcattatataatacagtagcaaccctctattgtgtgcatcaaagga
tagagataaaagacaccaaggaagctttagacaagatagaggaagagcaaaacaaaagtaagacc
accgcacagcaagcGGCCGCTgatcttcagacctggaggaggagatatgagggacaattggagaa
gtgaattatataaatataaagtagtaaaaattgaaccattaggagtagcacccaccaaggcaaag
agaagagtggtgcagagagaaaaaagaGCAGTGGGAATaggagctttgttccttgggttcttggg
agcagcaggaagcactatgggcgcagcgtcaatgacgctgacggtacaggccagacaattattgt
ctggtatagtgcagcagcagaacaatttgctgagggctattgaggcgcaacagcatctgttgcaa
ctcacagtctggggcatcaagcagctccaggcaagaatcctggctgtggaaagatacctaaagga
tcaacagctcctggggatttggggttgctctggaaaactcatttgcaccactgctgtgccttgga
atgctagttggagtaataaatctctggaacagatttggaatcacacgacctggatggagtgggac
agagaaattaacaattacacaagcttaatacactccttaattgaagaatcgcaaaaccagcaaga
aaagaatgaacaagaattattggaattagataaatgggcaagtttgtggaattggtttaacataa
caaattggctgtggtatataaaattattcataatgatagtaggaggcttggtaggtttaagaata
gtttttgctgtactttctatagtgaatagagttaggcagggatattcaccattatcgtttcagac
ccacctcccaaccccgaggggacccgacaggcccgaaggaatagaagaagaaggtggagagagag
acagagacagatccattcgattagtgaacggatCacaaatggcagtattcatccacaattttaaa
agaaaaggggggattggggggtacagtgcaggggaaagaatagtagacataatagcaacagacat
acaaactaaagaattacaaaaacaaattacaaaaattcaaaattttcgggtttattacagggaca
gcagaggctccggtgcccgtcagtgggcagagcgcacatcgcccacagtccccgagaagttgggg
ggaggggtcggcaattgaaccggtgcctagagaaggtggcgcggggtaaactgggaaagtgatgt
cgtgtactggctccgcctttttcccgaggggggggagaaccgtatataagtgcagtagtcgccgt
gaacgttctttttcgcaacgggtttgccgccagaacacaggtgtcgtgaAAATAAGAGAGAAAAG
AAGAGTAAGAAGAAATATAAGAGCCACCATGGCCACACTGCCTAGCGCTGAGAGAAGGGCCTTCG
CTCTGAAGATCAACCGGTACAGCAGCGCCGAGATCCGGAAGCAGTTTACCCTGCCTCCTAACCTG
GGCCAGTACCACAGACAGAGCATCAGCACCAGCGGCTTCCCATCTCTGCAGCTGCCCCAGTTCTA
CGACCCTGTGGAACCCGTGGATTTCGAGGGCCTGCTGATGACCCACCTGAACAGCCTGGATGTGC
AGCTGGCCCAAGAGCTGGGCGACTTCACCGACGATGATCTGGACGTGGTGTTCACCCCTAAAGAG
TGCAGAACCCTGCAGCCTAGCCTGCCTGAAGAAGGCGTGGAACTGGATCCTCACGTGCGGGATTG
TGTGCAGACCTACATCCGCGAGTGGCTGATCGTGAACCGGAAGAATCAGGGCAGCCCAGAGATCT
GCGGCTTCAAGAAAACCGGCAGCCGGAAGGACTTCCACAAGACCCTGCCTAAGCAGACCTTCGAG
AGCGAGACACTGGAATGCTCTGAGCCTGCTGCTCAGGCCGGACCTAGACACCTGAATGTGCTGTG
TGACGTGTCCGGCAAGGGACCTGTGACCGCCTGCGATTTCGACCTGAGAAGTCTGCAGCCCGACA
AGCGGCTGGAAAACCTGCTCCAGCAAGTGTCCGCCGAGGACTTCGAGAAGCAGAACGAGGAAGCC
AGACGGACCAACAGACAGGCCGAGCTGTTTGCTCTGTACCCCAGCGTGGACGAGGAAGATGCCGT
CGAGATTAGACCCGTGCCTGAGTGCCCAAAAGAGCACCTGGGCAACAGAATCCTGGTCAAGCTGC
TGACCCTGAAGTTCGAGATCGAGATTGAGCCCCTGTTCGCCTCTATCGCCCTGTACGACGTGAAA
GAGCGGAAGAAGATCAGCGAGAACTTCCACTGCGACCTGAACTCCGACCAGTTCAAGGGCTTCCT
GAGAGCCCACACACCATCTGTGGCCGCTAGCTCTCAGGCCAGATCTGCCGTGTTCAGCGTGACAT
ACCCCAGCAGCGACATCTACCTGGTGGTCAAGATCGAGAAGGTCCTGCAGCAGGGCGAGATCGGC
GATTGTGCCGAGCCTTACACCGTGATCAAAGAGAGCGACGGCGGCAAGAGCAAAGAGAAGATTGA
GAAGCTGAAGCTGCAGGCCGAGAGCTTCTGTCAGCGGCTGGGCAAGTACAGAATGCCCTTTGCCT
GGGCTCCTATCAGCCTGTCCAGCTTCTTCAACGTGTCCACACTGGAACGGGAAGTGACCGACGTG
GACTCTGTCGTGGGCAGATCTAGCGTGGGCGAGAGAAGGACACTGGCTCAGTCTAGACGGCTGAG
CGAGAGAGCCCTGAGCCTGGAAGAAAATGGCGTGGGCAGCAACTTCAAGACCAGCACTCTGTCCG
TGTCTAGCTTCTTTAAGCAAGAGGGCGACAGACTGAGCGACGAGGACCTGTTCAAGTTCCTGGCC
GACTACAAGCGGAGCAGCTCCCTGCAGAGAAGAGTGAAGTCTATCCCTGGCCTGCTGCGCCTGGA
AATCTCTACAGCCCCTGAGATCATCAACTGCTGTCTGACCCCTGAAATGCTGCCCGTGAAGCCCT
TTCCAGAGAACCGGACCAGACCTCACAAAGAGATCCTGGAATTCCCCACCAGAGAAGTGTACGTG
CCCCACACCGTGTACCGGAACCTGCTGTATGTGTACCCACAGCGGCTGAACTTCGTGAACAAGCT
GGCCTCCGCCAGAAACATCACCATCAAGATCCAGTTTATGTGCGGCGAGGACGCCAGCAACGCCA
TGCCTGTGATCTTCGGCAAGTCTAGCGGCCCTGAGTTCCTGCAAGAGGTGTACACAGCCGTGACC
TACCACAACAAGAGCCCCGACTTCTACGAGGAAGTGAAGATTAAGCTGCCCGCCAAGCTGACCGT
GAATCACCATCTGCTGTTCACCTTCTACCACATCAGCTGCCAGCAGAAACAGGGCGCCTCTGTGG
AAACACTGCTGGGCTATAGCTGGCTGCCCATCCTGCTGAACGAGAGACTGCAGACCGGCAGCTAC
TGTCTGCCTGTGGCTCTGGAAAAGCTGCCACCTAACTACAGCATGCACTCCGCCGAGAAGGTGCC
CCTGCAGAATCCTCCTATTAAGTGGGCCGAGGGCCACAAGGGCGTGTTCAATATCGAGGTGCAGG
CCGTGTCCTCCGTGCACACCCAGGATAACCACCTGGAAAAGTTCTTCACCCTGTGCCACAGCCTC
GAGAGCCAAGTGACATTCCCCATCCGCGTGCTGGACCAGAAAATCTCCGAGATGGCCCTGGAACA
CGAGCTGAAACTGAGCATCATCTGCCTGAATAGCAGCAGACTGGAACCCCTGGTGCTGTTCCTGC
ATCTGGTGCTGGACAAGCTGTTCCAGCTGAGCGTGCAGCCCATGGTTATCGCCGGACAGACCGCC
AACTTCAGCCAGTTCGCCTTTGAGAGCGTGGTGGCCATTGCCAACAGCCTGCACAACAGCAAGGA
CCTGAGCAAGGATCAGCACGGCAGAAACTGCCTGCTGGCCTCTTACGTGCACTACGTGTTCAGAC
TGCCTGAGGTGCAGAGGGACGTGCCAAAATCTGGCGCTCCTACCGCTCTGCTGGACCCCAGAAGC
TATCACACCTACGGCAGAACATCTGCCGCCGCTGTGTCCTCTAAACTCCTGCAGGCTAGAGTGAT
GTCCAGCAGCAACCCTGATCTGGCCGGAACACACAGCGCCGCTGATGAAGAAGTGAAAAACATCA
TGAGCAGCAAGATCGCCGACCGGAACTGCAGCCGGATGAGCTACTACTGCCTGAGCTACGACACC
GAGATCCTGACCGTGGAATACGGCTTCCTGCCTATCGGCAAGATCGTGGAAGAACGGATCGAGTG
CACCGTGTACACCGTGGACAAGAACGGCTTCGTGTACACACAGCCTATCGCTCAGTGGCACAACC
GGGGAGAGCAAGAGGTGTTCGAGTACTGCCTGGAAGATGGCAGCATCATCCGGGCCACCAAGGAC
CACAAGTTCATGACCACCGACGGCCAGATGCTGCCCATCGACGAGATCTTTGAGAGAGGCCTGGA
CCTGAAACAGGTGGACGGACTTCCTtgattccgataatcaacctctggattacaaaatttgtgaa
agattgactggtattcttaactatgttgctccttttacgctatgtggatacgctgctttaatgcc
tttgtatcatgctattgcttcccgtatggctttcattttctcctccttgtataaatcctggttgc
tgtctctttatgaggagttgtggcccgttgtcaggcaacgtggcgtggtgtgcactgtgtttgct
gacgcaacccccactggttggggcattgccaccacctgtcagctcctttccgggactttcgcttt
ccccctccctattgccacggcggaactcatcgccgcctgccttgcccgctgctggacaggggctc
ggctgttgggcactgacaattccgtggtgttgtcggggaaATCATcgtcctttccTtggctgctc
gcctgtgttgccacctggattctgcgcgggacgtccttctgctacgtcccttcggccctcaatcc
agcggaccttccttcccgcggcctgctgccggctctgcggcctcttccgcgtcttcgccttcgcc
ctcagacgagtcggatctccctttgggccgcctccccgcTaaaagaaaaggggggactggaaggg
ctaattcactcccaacgaagacaagatctgctttttgcttgtactGGGTCTCTCTGGTTAGACCA
GATCTGAGCCTGGGAGCTCTCTGGCTAACTAGGGAACCCACTGCTTAAGCCTCAATAAAGCTTGC
CTTGAGTGCTTCAAGTAGTGTGTTGGTTTTTTGTGTGTGCATTGTCTGAGTAGGTGTCATTCTAT
TCTGGGGGGTGGGGTGGGGCAGCACAGCAAGGGGGAGGATTGGGAAGACAATAGCAGGCATGCTG
GGGATGCGGTGGGCTCTATGGagtagtagttcatgtcatcttattattcagtatttataacttgc
aaagaaatgaatatcagagagtgagaggaacttgtttattgcagcttataatggttacaaataaa
gcaatagcatcacaaatttcacaaataaagcatttttttcactgcattctagttgtggtttgtcc
aaactcatcaatgtatcttatcatgtctggctctagctatcccgcccctaactccgcccATCCCG
CCCCTAACTCCGCCCAGTTCCGCCCATTCTCCGCCCCATGGCTCTCACTACTTCTGGAATAGCTC
AGAGGCCGAGGCGGCCTCGGCCTCTGCATAAATAAAAAAAATTAGTCGAGGCTTTTTTGGAGGCC
TAGGgacgtacccaattcgccctatagtgagtcgtattacgcgcgctcactggccgtcgttttac
aacgtcgtgactgggaaaaccctggcgttacccaacttaatcgccttgcagcacatccccctttc
gccagctggcgtaatagcgaagaggcccgcaccgatcgcccttcccaacagttgcgcagcctgaa
tggcgaatggg
(SEQ ID NO: 28)
EFS-939 DOCK8-CfaC-CFN-WPRE (2^{nd} DOCK8 fragment; 3′ half)
acgcgccctgtagcggcgcattaagcgcggcgggtgtggtggttacgcgcagcgtgaccgctaca
cttgccagcgccctagcgcccgctcctttcgctttcttcccttcctttctcgccacgttcgccgg
ctttccccgtcaagctctaaatcgggggctccctttagggttccgatttagtgctttacggcacc
tcgaccccaaaaaacttgattagggtgatggttcacgtagtgggccatcgccctgatagacggtt
tttcgccctttgacgttggagtccacgttctttaatagtggactcttgttccaaactggaacaac
actcaaccctatctcggtctattcttttgatttataagggattttgccgatttcggcctattggt
taaaaaatgagctgatttaacaaaaatttaacgcgaattttaacaaaatattaacgcttacaatt
taggtggcacttttcggggaaatgtgcgcggaacccctatttgtttatttttctaaatacattca
aatatgtatccgctcatgagacaataaccctgataaatgcttcaataatagcacctagatcaaga
gacaggatgaggatcgtttcgcatgattgaacaagatggattgcacgcaggttctccggccgctt
gggtggagaggctattcggctatgactgggcacaacagacaatcggctgctctgatgccgccgtg
ttccggctgtcagcgcaggggcgcccggttctttttgtcaagaccgacctgtccggtgccctgaa
tgaactgcaagacgaggcagcgcggctatcgiggciggccacgacgggcgticctigcgcagcig
igctcgacgitgtcactgaagcgggaagggactggctgctattgggcgaagtgccggggcaggat
ctcctgtcatctcaccttgctcctgccgagaaagtatccatcatggctgatgcaatgcggcggct
gcatacgcttgatccggctacctgcccattcgaccaccaagcgaaacatcgcatcgagcgagcac
gtactcggatggaagccggtcttgtcgatcaggatgatctggacgaagagcatcaggggctcgcg
ccagccgaactgttcgccaggctcaaggcgagcatgcccgacggcgaggatctcgtcgtgaccca
tggcgatgcctgcttgccgaatatcatggtggaaaatggccgcttttctggattcatcgactgtg
gccggctgggtgtggcggaccgctatcaggacatagcgttggctacccgtgatattgctgaagag
cttggcggcgaatgggctgaccgcttcctcgtgctttacggtatcgccgctcccgattcgcagcg
catcgccttctatcgccttcttgacgagttcttctgaattattaacgcttacaatttcctgatgc
ggtattttctccttacgcatctgtgcggtatttcacaccgcatcaggtggcacttttcggggaaa
tgtgcgcggaacccctatttgtttatttttctaaatacattcaaatatgtatccgctcatgacca
aaatcccttaacgtgagttttcgttccactgagcgtcagaccccgtagaaaagatcaaaggatct
tcttgagatcctttttttctgcgcgtaatctgctgcttgcaaacaaaaaaaccaccgctaccagc
ggtggtttgtttgccggatcaagagctaccaactctttttccgaaggtaactggcttcagcagag
cgcagataccaaatactgttcttctagtgtagccgtagttaggccaccacttcaagaactctgta
gcaccgcctacatacctcgctctgctaatcctgttaccagtggctgctgccagtggcgataagtc
gtgtcttaccgggttggactcaagacgatagttaccggataaggcgcagcggtcgggctgaacgg
gggggtcgtgcacacagcccagcttggagcgaacgacctacaccgaactgagatacctacagcgt
gagctatgagaaagcgccacgcttcccgaagggagaaaggcggacaggtatccggtaagcggcag
ggtcggaacaggagagcgcacgagggagcttccagggggaaacgcctggtatctttatagtcctg
tcgggtttcgccacctctgacttgagcgtcgatttttgtgatgctcgtcaggggggcggagccta
tggaaaaacgccagcaacgcggcctttttacggttcctggccttttgctggccttttgctcacat
gttctttcctgcgttatcccctgattctgtggataaccgtattaccgcctttgagtgagctgata
ccgctcgccgcagccgaacgaccgagcgcagcgagtcagtgagcgaggaagcggaagagcgccca
atacgcaaaccgcctctccccgcgcgttggccgattcattaatgcagctggcacgacaggtttcc
cgactggaaagcgggcagtgagcgcaacgcaattaatgtgagttagctcactcattaggcacccc
aggctttacactttatgcttccggctcgtatgttgtgtggaattgtgagcggataacaatttcac
acaggaaacagctatgaccatgattacgccaagcgcgcaattaaccctcactaaagggaacaaaa
gctggagctgcaagcttggccattgcatacgttgtatccatatcataatatgtacatttatattg
gctcatgtccaacattaccgccatgttgacattgattattgactagttattaatagtaatcaatt
acggggtcattagttcatagcccatatatggagttccgcgttacataacttacggtaaatggccc
gcctggctgaccgcccaacgacccccgcccattgacgtcaataatgacgtatgttcccatagtaa
cgccaatagggactttccattgacgtcaatgggtggagtatttacggtaaactgcccacttggca
gtacatcaagtgtatcatatgccaagtacgccccctattgacgtcaatgacggtaaatggcccgc
ctggcattatgcccagtacatgaccttatgggactttcctacttggcagtacatctacgtattag
tcatcgctattaccatggtgatgcggttttggcagtacatcaatgggcgtggatagcggtttgac
tcacggggatttccaagtctccaccccattgacgtcaatgggagtttgttttggcaccaaaatca
acgggactttccaaaatgtcgtaacaactccgccccattgacgcaaatgggcggtaggcgtgtac
ggtgggaggtctatataagcagagctcgtttagtgaaccgGGGTCTCTCTGGTTAGACCAGATCT
GAGCCTGGGAGCTCTCTGGCTAACTAGGGAACCCACTGCTTAAGCCTCAATAAAGCTTGCCTTGA
GTGCTTCAAGTAGTGTGTGCCCGTCTGTTGTGTGACTCTGGTAACTAGAGATCCCTCAGACCCTT
TTAGTCAGTGTGGAAAATCTCTAGCagtggcgcccgaacagggacttgaaagcgaaagggaaacc
agaggagctctcTCGACGCAGGACTCGGCTTGCTGAAGCGCGCACGGCAAGAGGCGAGGGGCGGC
GACTGGTGAGTACGCCAAAAATTTTGACTAGCGGAGGCTAGAAGGAGAGAGATGGGTGCGAGAGC
GTCAGTATTAAGCGGGGGAGaattagatcgcgatgggaaaaaattcggttaaggccagggggaaa
gaaaaaatataaattaaaacatatagtatgggcaagcagggagctagaacgattcgcagttaatc
ctggcctgttagaaacatcagaaggctgtagacaaatactgggacagctacaaccatcccttcag
acaggatcagaagaacttagatcattatataatacagtagcaaccctctattgtgtgcatcaaag
gatagagataaaagacaccaaggaagctttagacaagatagaggaagagcaaaacaaaagtaaga
ccaccgcacagcaagcGGCCGCTgatcttcagacctggaggaggagatatgagggacaattggag
aagtgaattatataaatataaagtagtaaaaattgaaccattaggagtagcacccaccaaggcaa
agagaagagtggtgcagagagaaaaaagaGCAGTGGGAATaggagctttgttccttgggttcttg
ggagcagcaggaagcactatgggcgcagcgtcaatgacgctgacggtacaggccagacaattatt
gtctggtatagtgcagcagcagaacaatttgctgagggctattgaggcgcaacagcatctgttgc
aactcacagtctggggcatcaagcagctccaggcaagaatcctggctgtggaaagatacctaaag
gatcaacagctcctggggatttggggttgctctggaaaactcatttgcaccactgctgtgccttg
gaatgctagttggagtaataaatctctggaacagatttggaatcacacgacctggatggagtggg
acagagaaattaacaattacacaagcttaatacactccttaattgaagaatcgcaaaaccagcaa
gaaaagaatgaacaagaattattggaattagataaatgggcaagtttgtggaattggtttaacat
aacaaattggctgtggtatataaaattattcataatgatagtaggaggcttggtaggtttaagaa
tagtttttgctgtactttctatagtgaatagagttaggcagggatattcaccattatcgtttcag
acccacctcccaaccccgaggggacccgacaggcccgaaggaatagaagaagaaggtggagagag
agacagagacagatccattcgattagtgaacggatCacaaatggcagtattcatccacaatttta
aaagaaaaggggggattggggggtacagtgcaggggaaagaatagtagacataatagcaacagac
atacaaactaaagaattacaaaaacaaattacaaaaattcaaaattttcgggtttattacaggga
cagcagaggctccggtgcccgtcagtgggcagagcgcacatcgcccacagtccccgagaagttgg
ggggaggggtcggcaattgaaccggtgcctagagaaggtggcgcggggtaaactgggaaagtgat
gtcgtgtactggctccgcctttttcccgaggggggggagaaccgtatataagtgcagtagtcgcc
gtgaacgttctttttcgcaacgggtttgccgccagaacacaggtgtcgtgaAAATAAGAGAGAAA
AGAAGAGTAAGAAGAAATATAAGAGCCACCatgGTGAAGATCATCAGCAGAAAGAGCCTGGGCAC
CCAGAACGTGTACGACATCGGCGTGGAAAAGGACCACAACTTTCTGCTCAAGAACGGCCTGGTGG
CCAGCAACTGCTTTAATAGCGGCAGCTCTGATGCCCCTAGCTCTCCAGCTGCTCCTAGACCTGCC
AGCAAGAAGCACTTTCACGAGGAACTGGCCCTGCAGATGGTCGTGTCTACCGGCATGGTTCGAGA
GACAGTGTTTAAGTACGCCTGGTTCTTCTTCGAACTGCTGGTCAAGAGCATGGCCCAGCACGTGC
ACAACATGGACAAGCGGGACAGCTTCCGGCGGACCAGATTCAGCGACAGATTCATGGACGACATC
ACCACCATCGTGAACGTGGTCACCAGCGAGATTGCTGCCCTGCTGGTTAAGCCCCAGAAAGAGAA
CGAGCAGGCCGAAAAGATGAATATCTCTCTGGCCTTCTTCCTCTACGACCTGCTGAGCCTGATGG
ACCGGGGCTTCGTGTTCAACCTGATCCGGCACTACTGCTCCCAGCTGTCTGCCAAGCTGTCCAAC
CTGCCTACACTGATCTCTATGAGACTCGAGTTCCTGAGGATCCTGTGCAGCCACGAGCACTACCT
GAACCTGAATCTGTTCTTCATGAACGCCGACACAGCCCCTACAAGCCCCTGTCCTTCTATCAGCA
GCCAGAACAGCAGCAGCTGTAGCAGCTTTCAGGATCAGAAAATCGCCAGCATGTTCGATCTGACC
AGCGAGTACCGGCAGCAGCACTTTCTGACCGGCCTGCTGTTTACCGAACTGGCAGCCGCTCTTGA
TGCCGAAGGCGAGGGAATCAGCAAGGTGCAGCGGAAAGCCGTGTCTGCCATTCACAGCCTGCTGT
CTAGCCACGACCTGGATCCTAGATGCGTGAAGCCTGAAGTGAAAGTGAAAATCGCTGCCCTGTAC
CTGCCTCTCGTGGGCATCATTCTGGATGCCCTGCCTCAGCTGTGTGATTTCACCGTGGCCGACAC
CAGACGGTACAGAACCTCTGGCTCCGACGAGGAACAAGAAGGCGCTGGCGCCATCAACCAGAATG
TGGCCCTTGCCATTGCCGGGAACAACTTCAATCTGAAAACCAGCGGCATCGTGCTGAGCAGCCTG
CCTTACAAGCAGTACAACATGCTGAATGCCGACACTACCCGCAACCTGATGATCTGCTTCCTGTG
GATTATGAAGAACGCCGATCAGAGCCTGATTCGGAAGTGGATTGCCGACCTGCCAAGCACTCAGC
TGAACCGGATTCTGGACCTCCTGTTCATCTGCGTGCTGTGCTTCGAGTACAAGGGCAAGCAGTCC
AGCGACAAGGTGTCCACTCAGGTGCTGCAGAAAAGCCGGGATGTGAAGGCCAGGCTGGAAGAGGC
ACTTCTGAGAGGCGAAGGCGCCAGAGGCGAGATGATGAGGCGTAGAGCCCCTGGCAACGATAGAT
TCCCCGGCCTGAATGAGAACCTGCGGTGGAAGAAAGAGCAGACCCATTGGAGACAGGCCAACGAG
AAACTGGACAAGACCAAGGCCGAACTGGACCAAGAGGCCCTGATCTCTGGCAACCTGGCCACAGA
AGCCCACCTGATTATCCTGGACATGCAAGAGAACATCATCCAGGCCAGTAGCGCCCTGGATTGCA
AGGATTCTCTGCTCGGAGGCGTGCTGAGGGTGCTCGTGAATAGCCTGAATTGCGACCAGAGCACC
ACCTACCTGACACACTGCTTCGCCACACTGAGGGCCCTGATTGCCAAGTTTGGGGACCTGCTGTT
CGAGGAAGAGGTGGAACAGTGCTTCGACCTGTGTCACCAAGTGCTGCACCACTGCAGCAGCTCCA
TGGACGTGACAAGATCCCAGGCCTGCGCCACTCTGTATCTGCTGATGCGGTTTAGCTTCGGCGCC
ACCAGCAATTTCGCCAGAGTGAAGATGCAAGTGACCATGAGCCTGGCCAGCCTCGTTGGAAGGGC
CCCTGACTTCAATGAGGAACACCTCAGAAGAAGCCTGCGGACCATCCTGGCCTACAGCGAAGAGG
ATACCGCCATGCAGATGACCCCATTTCCAACACAAGTGGAAGAACTGCTGTGCAACCTGAATTCC
ATCCTGTACGATACCGTGAAGATGAGAGAGTTCCAAGAGGACCCCGAGATGCTGATGGATCTGAT
GTACCGGATCGCCAAGAGCTACCAGGCCTCTCCTGATCTGAGACTGACCTGGCTGCAGAACATGG
CCGAGAAGCACACCAAGAAGAAGTGCTACACCGAGGCCGCCATGTGCCTGGTTCATGCTGCTGCA
CTCGTGGCCGAGTACCTGAGCATGCTGGAAGATCACTCCTACCTGCCAGTGGGCAGCGTGTCCTT
CCAGAACATCAGCTCCAACGTCCTGGAAGAGTCCGTGGTGTCCGAGGACACACTGAGCCCAGATG
AGGATGGCGTGTGTGCCGGACAGTACTTTACCGAGTCTGGCCTCGTGGGCCTTCTGGAACAGGCC
GCCGAACTGTTTTCTACCGGCGGACTGTACGAGACAGTGAACGAAGTGTACAAGCTGGTCATCCC
CATCCTGGAAGCCCACAGAGAGTTCAGAAAGCTGACACTGACCCACTCCAAGCTGCAGCGGGCCT
TCGACAGCATTGTGAACAAGGACCACAAGCGGATGTTCGGCACCTACTTCAGAGTGGGCTTTTTC
GGCAGCAAATTCGGCGACCTGGACGAGCAAGAGTTCGTGTACAAAGAGCCCGCCATCACCAAGCT
GCCTGAGATCTCCCATCGGCTGGAAGCCTTCTACGGCCAGTGTTTTGGCGCCGAGTTCGTGGAAG
TGATCAAGGACAGCACCCCTGTGGATAAGACCAAACTGGACCCTAACAAGGCCTACATCCAGATC
ACCTTCGTGGAACCCTACTTCGACGAGTACGAGATGAAGGATAGAGTGACCTACTTTGAGAAGAA
CTTCAACCTGCGGCGGTTCATGTACACCACACCTTTTACACTGGAAGGCAGACCCAGGGGCGAGC
TGCACGAGCAGTATAGAAGAAACACCGTGCTGACCACCATGCACGCATTCCCTTACATCAAGACC
CGGATCAGCGTGATCCAGAAAGAAGAGTTTGTCCTGACACCAATCGAGGTGGCCATCGAGGACAT
GAAGAAGAAAACCCTCCAGCTGGCCGTGGCCATCAATCAAGAACCTCCAGACGCCAAGATGCTCC
AGATGGTGCTCCAGGGATCTGTGGGCGCCACAGTGAATCAGGGACCTCTGGAAGTGGCCCAAGTG
TTCCTGGCTGAGATCCCCGCCGATCCTAAGCTGTACAGACACCATAACAAGCTGCGGCTCTGCTT
CAAAGAATTCATCATGAGATGTGGCGAGGCCGTGGAAAAGAACAAGAGACTGATCACCGCCGACC
AGAGAGAGTACCAGCAAGAGCTGAAGAAAAACTACAACAAGCTCAAAGAAAATCTGCGGCCCATG
ATCGAGAGAAAGATCCCCGAGCTGTACAAGCCCATCTTCCGCGTGGAAAGCCAGAAGCGGGACTC
CTTTCACAGATCCAGCTTTAGGAAGTGCGAGACACAGCTGTCCCAGGGCTCTTGAttccgataat
caacctctggattacaaaatttgtgaaagattgactggtattcttaactatgttgctccttttac
gctatgtggatacgctgctttaatgcctttgtatcatgctattgcttcccgtatggctttcattt
tctcctccttgtataaatcctggttgctgtctctttatgaggagttgtggcccgttgtcaggcaa
cgtggcgtggtgtgcactgtgtttgctgacgcaacccccactggttggggcattgccaccacctg
tcagctcctttccgggactttcgctttccccctccctattgccacggcggaactcatcgccgcct
gccttgcccgctgctggacaggggctcggctgttgggcactgacaattccgtggtgttgtcgggg
aaATCATcgtcctttccTtggctgctcgcctgtgttgccacctggattctgcgcgggacgtcctt
ctgctacgtcccttcggccctcaatccagcggaccttccttcccgggcctgctgccggctctgcg
gcctcttccgcgtcttcgccttcgccctcagacgagtcggatctccctttgggccgcctccccgc
Taaaagaaaaggggggactggaagggctaattcactcccaacgaagacaagatctgctttttgct
tgtactGGGTCTCTCTGGTTAGACCAGATCTGAGCCTGGGAGCTCTCTGGCTAACTAGGGAACCC
ACTGCTTAAGCCTCAATAAAGCTTGCCTTGAGTGCTTCAAGTAGTGTGTTGGTTTTTTGTGTGTG
CATTGTCTGAGTAGGTGTCATTCTATTCTGGGGGGTGGGGTGGGGCAGCACAGCAAGGGGGAGGA
TTGGGAAGACAATAGCAGGCATGCTGGGGATGCGGTGGGCTCTATGGagtagtagttcatgtcat
cttattattcagtatttataacttgcaaagaaatgaatatcagagagtgagaggaacttgtttat
tgcagcttataatggttacaaataaagcaatagcatcacaaatttcacaaataaagcattttttt
cactgcattctagttgtggtttgtccaaactcatcaatgtatcttatcatgtctggctctagcta
tcccgcccctaactccgcccATCCCGCCCCTAACTCCGCCCAGTTCCGCCCATTCTCCGCCCCAT
GGCTCTCACTACTTCTGGAATAGCTCAGAGGCCGAGGCGGCCTCGGCCTCTGCATAAATAAAAAA
AATTAGTCGAGGCTTTTTTGGAGGCCTAGGgacgtacccaattcgccctatagtgagtcgtatta
cgcgcgctcactggccgtcgttttacaacgtcgtgactgggaaaaccctggcgttacccaactta
atcgccttgcagcacatccccctttcgccagctggcgtaatagcgaagaggcccgcaccgatcgc
ccttcccaacagttgcgcagcctgaatggcgaatggg
(SEQ ID NO: 29)
EFS-939 DOCK8-CfaC-CF-WPRE (2^{nd} DOCK8 fragment;3′ half)
acgcgccctgtagcggcgcattaagcgcggcgggtgtggtggttacgcgcagcgtgaccgctaca
cttgccagcgccctagcgcccgctcctttcgctttcttcccttcctttctcgccacgttcgccgg
ctttccccgtcaagctctaaatcgggggctccctttagggttccgatttagtgctttacggcacc
tcgaccccaaaaaacttgattagggtgatggttcacgtagtgggccatcgccctgatagacggtt
tttcgccctttgacgttggagtccacgttctttaatagtggactcttgttccaaactggaacaac
actcaaccctatctcggtctattcttttgatttataagggattttgccgatttcggcctattggt
taaaaaatgagctgatttaacaaaaatttaacgcgaattttaacaaaatattaacgcttacaatt
taggtggcacttttcggggaaatgtgcgcggaacccctatttgtttatttttctaaatacattca
aatatgtatccgctcatgagacaataaccctgataaatgcttcaataatagcacctagatcaaga
gacaggatgaggatcgtttcgcatgattgaacaagatggattgcacgcaggttctccggccgctt
gggtggagaggctattcggctatgactgggcacaacagacaatcggctgctctgatgccgccgtg
ttccggctgtcagcgcaggggcgcccggttctttttgtcaagaccgacctgtccggtgccctgaa
tgaactgcaagacgaggcagcgcggctatcgtggctggccacgacgggcgttccttgcgcagctg
tgctcgacgttgtcactgaagcgggaagggactggctgctattgggcgaagtgccggggcaggat
ctcctgtcatctcaccttgctcctgccgagaaagtatccatcatggctgatgcaatgcggcggct
gcatacgcttgatccggctacctgcccattcgaccaccaagcgaaacatcgcatcgagcgagcac
gtactcggatggaagccggtcttgtcgatcaggatgatctggacgaagagcatcaggggctcgcg
ccagccgaactgttcgccaggctcaaggcgagcatgcccgacggcgaggatctcgtcgtgaccca
tggcgatgcctgcttgccgaatatcatggtggaaaatggccgcttttctggattcatcgactgtg
gccggctgggtgtggcggaccgctatcaggacatagcgttggctacccgtgatattgctgaagag
cttggcggcgaatgggctgaccgcttcctcgtgctttacggtatcgccgctcccgattcgcagcg
catcgccttctatcgccttcttgacgagttcttctgaattattaacgcttacaatttcctgatgc
ggtattttctccttacgcatctgtgcggtatttcacaccgcatcaggtggcacttttcggggaaa
tgtgcgcggaacccctatttgtttatttttctaaatacattcaaatatgtatccgctcatgacca
aaatcccttaacgtgagttttcgttccactgagcgtcagaccccgtagaaaagatcaaaggatct
tcttgagatcctttttttcigcgcgtaatctgctgcttgcaaacaaaaaaaccaccgctaccagc
ggiggtutgittgccggatcaagagctaccaactctttttccgaaggtaactggcttcagcagag
cgcagataccaaatactgttcttctagtgtagccgtagttaggccaccacttcaagaactctgta
gcaccgcctacatacctcgctctgctaatcctgttaccagtggctgctgccagtggcgataagtc
gtgtcttaccgggttggactcaagacgatagttaccggataaggcgcagcggtcgggctgaacgg
gggggtcgtgcacacagcccagcttggagcgaacgacctacaccgaactgagatacctacagcgt
gagctatgagaaagcgccacgcttcccgaagggagaaaggcggacaggtatccggtaagcggcag
ggtcggaacaggagagcgcacgagggagcttccagggggaaacgcctggtatctttatagtcctg
tcgggtttcgccacctctgacttgagcgtcgatttttgtgatgctcgtcaggggggcggagccta
tggaaaaacgccagcaacgcggcctttttacggttcctggccttttgctggccttttgctcacat
gttctttcctgcgttatcccctgattctgtggataaccgtattaccgcctttgagtgagctgata
ccgctcgccgcagccgaacgaccgagcgcagcgagtcagtgagcgaggaagcggaagagcgccca
atacgcaaaccgcctctccccgcgcgttggccgattcattaatgcagctggcacgacaggtttcc
cgactggaaagcgggcagtgagcgcaacgcaattaatgtgagttagctcactcattaggcacccc
aggctttacactttatgcttccggctcgtatgttgtgtggaattgtgagcggataacaatttcac
acaggaaacagctatgaccatgattacgccaagcgcgcaattaaccctcactaaagggaacaaaa
gctggagctgcaagcttggccattgcatacgttgtatccatatcataatatgtacatttatattg
gctcatgtccaacattaccgccatgttgacattgattattgactagttattaatagtaatcaatt
acggggtcattagttcatagcccatatatggagttccgcgttacataacttacggtaaatggccc
gcctggctgaccgcccaacgacccccgcccattgacgtcaataatgacgtatgttcccatagtaa
cgccaatagggactttccattgacgtcaatgggtggagtatttacggtaaactgcccacttggca
gtacatcaagtgtatcatatgccaagtacgccccctattgacgtcaatgacggtaaatggcccgc
ctggcattatgcccagtacatgaccttatgggactttcctacttggcagtacatctacgtattag
tcatcgctattaccatggtgatgcggttttggcagtacatcaatgggcgtggatagcggtttgac
tcacggggatttccaagtctccaccccattgacgtcaatgggagtttgttttggcaccaaaatca
acgggactttccaaaatgtcgtaacaactccgccccattgacgcaaatgggcggtaggcgtgtac
ggtgggaggtctatataagcagagctcgtttagtgaaccgGGGTCTCTCTGGTTAGACCAGATCT
GAGCCTGGGAGCTCTCTGGCTAACTAGGGAACCCACTGCTTAAGCCTCAATAAAGCTTGCCTTGA
GTGCTTCAAGTAGTGTGTGCCCGTCTGTTGTGTGACTCTGGTAACTAGAGATCCCTCAGACCCTT
TTAGTCAGTGTGGAAAATCTCTAGCagtggcgcccgaacagggacttgaaagcgaaagggaaacc
agaggagctctcTCGACGCAGGACTCGGCTTGCTGAAGCGCGCACGGCAAGAGGCGAGGGGCGGC
GACTGGTGAGTACGCCAAAAATTTTGACTAGCGGAGGCTAGAAGGAGAGAGATGGGTGCGAGAGC
GTCAGTATTAAGCGGGGGAGaattagatcgcgatgggaaaaaattcggttaaggccagggggaaa
gaaaaaatataaattaaaacatatagtatgggcaagcagggagctagaacgattcgcagttaatc
ctggcctgttagaaacatcagaaggctgtagacaaatactgggacagctacaaccatcccttcag
acaggatcagaagaacttagatcattatataatacagtagcaaccctctattgtgtgcatcaaag
gatagagataaaagacaccaaggaagctttagacaagatagaggaagagcaaaacaaaagtaaga
ccaccgcacagcaagcGGCCGCTgatcttcagacctggaggaggagatatgagggacaattggag
aagtgaattatataaatataaagtagtaaaaattgaaccattaggagtagcacccaccaaggcaa
agagaagagtggtgcagagagaaaaaagaGCAGTGGGAATaggagctttgttccttgggttcttg
ggagcagcaggaagcactatgggcgcagcgtcaatgacgctgacggtacaggccagacaattatt
gtctggtatagtgcagcagcagaacaatttgctgagggctattgaggcgcaacagcatctgttgc
aactcacagtctggggcatcaagcagciccaggcaagaatcctggctglggaaagatacctaaag
gatcaacagctcctggggatttggggitgctctggaaaactcatttgcaccactgctgtgccttg
gaatgctagttggagtaataaatctctggaacagatttggaatcacacgacctggatggagtggg
acagagaaattaacaattacacaagcttaatacactccttaattgaagaatcgcaaaaccagcaa
gaaaagaatgaacaagaattattggaattagataaatgggcaagtttgtggaattggtttaacat
aacaaattggctgtggtatataaaattattcataatgatagtaggaggcttggtaggtttaagaa
tagtttttgctgtactttctatagtgaatagagttaggcagggatattcaccattatcgtttcag
acccacctcccaaccccgaggggacccgacaggcccgaaggaatagaagaagaaggtggagagag
agacagagacagatccattcgattagtgaacggatCacaaatggcagtattcatccacaatttta
aaagaaaaggggggattggggggtacagtgcaggggaaagaatagtagacataatagcaacagac
atacaaactaaagaattacaaaaacaaattacaaaaattcaaaattttcgggtttattacaggga
cagcagaggctccggtgcccgtcagtgggcagagcgcacatcgcccacagtccccgagaagttgg
ggggaggggtcggcaattgaaccggtgcctagagaaggtggcgcggggtaaactgggaaagtgat
gtcgtgtactggctccgcctttttcccgaggggggggagaaccgtatataagtgcagtagtcgcc
gtgaacgttctttttcgcaacgggtttgccgccagaacacaggtgtcgtgaAAATAAGAGAGAAA
AGAAGAGTAAGAAGAAATATAAGAGCCACCatgGTGAAGATCATCAGCAGAAAGAGCCTGGGCAC
CCAGAACGTGTACGACATCGGCGTGGAAAAGGACCACAACTTTCTGCTCAAGAACGGCCTGGTGG
CCAGCAACTGCTTTAGCGGCAGCTCTGATGCCCCTAGCTCTCCAGCTGCTCCTAGACCTGCCAGC
AAGAAGCACTTTCACGAGGAACTGGCCCTGCAGATGGTCGTGTCTACCGGCATGGTTCGAGAGAC
AGTGTTTAAGTACGCCTGGTTCTTCTTCGAACTGCTGGTCAAGAGCATGGCCCAGCACGTGCACA
ACATGGACAAGCGGGACAGCTTCCGGCGGACCAGATTCAGCGACAGATTCATGGACGACATCACC
ACCATCGTGAACGTGGTCACCAGCGAGATTGCTGCCCTGCTGGTTAAGCCCCAGAAAGAGAACGA
GCAGGCCGAAAAGATGAATATCTCTCTGGCCTTCTTCCTCTACGACCTGCTGAGCCTGATGGACC
GGGGCTTCGTGTTCAACCTGATCCGGCACTACTGCTCCCAGCTGTCTGCCAAGCTGTCCAACCTG
CCTACACTGATCTCTATGAGACTCGAGTTCCTGAGGATCCTGTGCAGCCACGAGCACTACCTGAA
CCTGAATCTGTTCTTCATGAACGCCGACACAGCCCCTACAAGCCCCTGTCCTTCTATCAGCAGCC
AGAACAGCAGCAGCTGTAGCAGCTTTCAGGATCAGAAAATCGCCAGCATGTTCGATCTGACCAGC
GAGTACCGGCAGCAGCACTTTCTGACCGGCCTGCTGTTTACCGAACTGGCAGCCGCTCTTGATGC
CGAAGGCGAGGGAATCAGCAAGGTGCAGCGGAAAGCCGTGTCTGCCATTCACAGCCTGCTGTCTA
GCCACGACCTGGATCCTAGATGCGTGAAGCCTGAAGTGAAAGTGAAAATCGCTGCCCTGTACCTG
CCTCTCGTGGGCATCATTCTGGATGCCCTGCCTCAGCTGTGTGATTTCACCGTGGCCGACACCAG
ACGGTACAGAACCTCTGGCTCCGACGAGGAACAAGAAGGCGCTGGCGCCATCAACCAGAATGTGG
CCCTTGCCATTGCCGGGAACAACTTCAATCTGAAAACCAGCGGCATCGTGCTGAGCAGCCTGCCT
TACAAGCAGTACAACATGCTGAATGCCGACACTACCCGCAACCTGATGATCTGCTTCCTGTGGAT
TATGAAGAACGCCGATCAGAGCCTGATTCGGAAGTGGATTGCCGACCTGCCAAGCACTCAGCTGA
ACCGGATTCTGGACCTCCTGTTCATCTGCGTGCTGTGCTTCGAGTACAAGGGCAAGCAGTCCAGC
GACAAGGTGTCCACTCAGGTGCTGCAGAAAAGCCGGGATGTGAAGGCCAGGCTGGAAGAGGCACT
TCTGAGAGGCGAAGGCGCCAGAGGCGAGATGATGAGGCGTAGAGCCCCTGGCAACGATAGATTCC
CCGGCCTGAATGAGAACCTGCGGTGGAAGAAAGAGCAGACCCATTGGAGACAGGCCAACGAGAAA
CTGGACAAGACCAAGGCCGAACTGGACCAAGAGGCCCTGATCTCTGGCAACCTGGCCACAGAAGC
CCACCTGATTATCCTGGACATGCAAGAGAACATCATCCAGGCCAGTAGCGCCCTGGATTGCAAGG
ATTCTCTGCTCGGAGGCGTGCTGAGGGTGCTCGTGAATAGCCTGAATTGCGACCAGAGCACCACC
TACCTGACACACTGCTTCGCCACACTGAGGGCCCTGATTGCCAAGTTTGGGGACCTGCTGTTCGA
GGAAGAGGTGGAACAGTGCTTCGACCTGTGTCACCAAGTGCTGCACCACTGCAGCAGCTCCATGG
ACGTGACAAGATCCCAGGCCTGCGCCACTCTGTATCTGCTGATGCGGTTTAGCTTCGGCGCCACC
AGCAATTTCGCCAGAGTGAAGATGCAAGTGACCATGAGCCTGGCCAGCCTCGTTGGAAGGGCCCC
TGACTTCAATGAGGAACACCTCAGAAGAAGCCTGCGGACCATCCTGGCCTACAGCGAAGAGGATA
CCGCCATGCAGATGACCCCATTTCCAACACAAGTGGAAGAACTGCTGTGCAACCTGAATTCCATC
CTGTACGATACCGTGAAGATGAGAGAGTTCCAAGAGGACCCCGAGATGCTGATGGATCTGATGTA
CCGGATCGCCAAGAGCTACCAGGCCTCTCCTGATCTGAGACTGACCTGGCTGCAGAACATGGCCG
AGAAGCACACCAAGAAGAAGTGCTACACCGAGGCCGCCATGTGCCTGGTTCATGCTGCTGCACTC
GTGGCCGAGTACCTGAGCATGCTGGAAGATCACTCCTACCTGCCAGTGGGCAGCGTGTCCTTCCA
GAACATCAGCTCCAACGTCCTGGAAGAGTCCGTGGTGTCCGAGGACACACTGAGCCCAGATGAGG
ATGGCGTGTGTGCCGGACAGTACTTTACCGAGTCTGGCCTCGTGGGCCTTCTGGAACAGGCCGCC
GAACTGTTTTCTACCGGCGGACTGTACGAGACAGTGAACGAAGTGTACAAGCTGGTCATCCCCAT
CCTGGAAGCCCACAGAGAGTTCAGAAAGCTGACACTGACCCACTCCAAGCTGCAGCGGGCCTTCG
ACAGCATTGTGAACAAGGACCACAAGCGGATGTTCGGCACCTACTTCAGAGTGGGCTTTTTCGGC
AGCAAATTCGGCGACCTGGACGAGCAAGAGTTCGTGTACAAAGAGCCCGCCATCACCAAGCTGCC
TGAGATCTCCCATCGGCTGGAAGCCTTCTACGGCCAGTGTTTTGGCGCCGAGTTCGTGGAAGTGA
TCAAGGACAGCACCCCTGTGGATAAGACCAAACTGGACCCTAACAAGGCCTACATCCAGATCACC
TTCGTGGAACCCTACTTCGACGAGTACGAGATGAAGGATAGAGTGACCTACTTTGAGAAGAACTT
CAACCTGCGGCGGTTCATGTACACCACACCTTTTACACTGGAAGGCAGACCCAGGGGCGAGCTGC
ACGAGCAGTATAGAAGAAACACCGTGCTGACCACCATGCACGCATTCCCTTACATCAAGACCCGG
ATCAGCGTGATCCAGAAAGAAGAGTTTGTCCTGACACCAATCGAGGTGGCCATCGAGGACATGAA
GAAGAAAACCCTCCAGCTGGCCGTGGCCATCAATCAAGAACCTCCAGACGCCAAGATGCTCCAGA
TGGTGCTCCAGGGATCTGTGGGCGCCACAGTGAATCAGGGACCTCTGGAAGTGGCCCAAGTGTTC
CTGGCTGAGATCCCCGCCGATCCTAAGCTGTACAGACACCATAACAAGCTGCGGCTCTGCTTCAA
AGAATTCATCATGAGATGTGGCGAGGCCGTGGAAAAGAACAAGAGACTGATCACCGCCGACCAGA
GAGAGTACCAGCAAGAGCTGAAGAAAAACTACAACAAGCTCAAAGAAAATCTGCGGCCCATGATC
GAGAGAAAGATCCCCGAGCTGTACAAGCCCATCTTCCGCGTGGAAAGCCAGAAGCGGGACTCCTT
TCACAGATCCAGCTTTAGGAAGTGCGAGACACAGCTGTCCCAGGGCTCTTGAttccgataatcaa
cctctggattacaaaatttgtgaaagattgactggtattcttaactatgttgctccttttacgct
atgtggatacgctgctttaatgcctttgtatcatgctattgcttcccgtatggctttcatttict
ccicctigtataaatcctggitgctgictctttatgaggagligtggcccgtigtcaggcaacgi
ggcgtggtgtgcactgtgtttgctgacgcaacccccactggttggggcattgccaccacctgtca
gctcctttccgggactttcgctttccccctccctattgccacggcggaactcatcgccgcctgcc
ttgcccgctgctggacaggggctcggctgttgggcactgacaattccgtggtgttgtcggggaaA
TCATcgtcctttccTtggctgctcgcctgtgttgccacctggattctgcgcgggacgtccttctg
ctacgtcccttcggccctcaatccagcggaccttccttcccgcggcctgctgccggctctgcggc
ctcttccgcgtcttcgccttcgccctcagacgagtcggatctccctttgggccgcctccccgcTa
aaagaaaaggggggactggaagggctaattcactcccaacgaagacaagatctgctttttgcttg
tactGGGTCTCTCTGGTTAGACCAGATCTGAGCCTGGGAGCTCTCTGGCTAACTAGGGAACCCAC
TGCTTAAGCCTCAATAAAGCTTGCCTTGAGTGCTTCAAGTAGTGTGTTGGTTTTTTGTGTGTGCA
TTGTCTGAGTAGGTGTCATTCTATTCTGGGGGGTGGGGTGGGGCAGCACAGCAAGGGGGAGGATT
GGGAAGACAATAGCAGGCATGCTGGGGATGCGGTGGGCTCTATGGagtagtagttcatgtcatct
tattattcagtatttataacttgcaaagaaatgaatatcagagagtgagaggaacttgtttattg
cagcttataatggttacaaataaagcaatagcatcacaaatttcacaaataaagcatttttttca
ctgcattctagttgtggtttgtccaaactcatcaatgtatcttatcatgtctggctctagctatc
ccgcccctaactccgcccATCCCGCCCCTAACTCCGCCCAGTTCCGCCCATTCTCCGCCCCATGG
CTCTCACTACTTCTGGAATAGCTCAGAGGCCGAGGCGGCCTCGGCCTCTGCATAAATAAAAAAAA
TTAGTCGAGGCTTTTTTGGAGGCCTAGGgacgtacccaattcgccctatagtgagtcgtattacg
cgcgctcactggccgtcgttttacaacgtcgtgactgggaaaaccctggcgttacccaacttaat
cgccttgcagcacatccccctttcgccagctggcgtaatagcgaagaggcccgcaccgatcgccc
ttcccaacagttgcgcagcctgaatggcgaatggg
(SEQ ID NO: 30)
1267 DOCK8-CfaN (1^{st} DOCK8 fragment; 5′ half)
acgcgccctgtagcggcgcattaagcgcggcgggtgtggtggttacgcgcagcgtgaccgctaca
cttgccagcgccctagcgcccgctcctttcgctttcttcccttcctttctcgccacgttcgccgg
ctttccccgtcaagctctaaatcgggggctccctttagggttccgatttagtgctttacggcacc
tcgaccccaaaaaacttgattagggtgatggttcacgtagtgggccatcgccctgatagacggtt
tttcgccctttgacgttggagtccacgttctttaatagtggactcttgttccaaactggaacaac
actcaaccctatctcggtctattcttttgatttataagggattttgccgatttcggcctattggt
taaaaaatgagctgatttaacaaaaatttaacgcgaattttaacaaaatattaacgcttacaatt
taggtggcacttttcggggaaatgtgcgcggaacccctatttgtttatttttctaaatacattca
aatatgtatccgctcatgagacaataaccctgataaatgcttcaataatagcacctagatcaaga
gacaggatgaggatcgtttcgcatgattgaacaagatggattgcacgcaggttctccggccgctt
gggtggagaggctattcggctatgactgggcacaacagacaatcggctgctctgatgccgccgtg
ttccggctgtcagcgcaggggcgcccggttctttttgtcaagaccgacctgtccggtgccctgaa
tgaactgcaagacgaggcagcgcggctatcgtggctggccacgacgggcgttccttgcgcagctg
tgctcgacgttgtcactgaagcgggaagggactggctgctattgggcgaagtgccggggcaggat
ctcctgtcatctcaccttgctcctgccgagaaagtatccatcatggctgatgcaatgcggcggct
gcatacgcttgatccggctacctgcccattcgaccaccaagcgaaacatcgcatcgagcgagcac
gtactcggatggaagccggtcttgtcgatcaggatgatctggacgaagagcatcaggggctcgcg
ccagccgaactgttcgccaggctcaaggcgagcatgcccgacggcgaggatctcgtcgtgaccca
tggcgatgcctgcttgccgaatatcatggtggaaaatggccgcttttctggattcatcgactgtg
gccggctgggtgtggcggaccgctatcaggacatagcgttggctacccgtgatattgctgaagag
cttggcggcgaatgggctgaccgcttcctcgtgctttacggtatcgccgctcccgattcgcagcg
catcgccttctatcgccttcttgacgagttcttctgaattattaacgcttacaatttcctgatgc
ggtattttctccttacgcatctgtgcggtatttcacaccgcatcaggtggcacttttcggggaaa
tgtgcgcggaacccctatttgtttatttttctaaatacattcaaatatgtatccgctcatgacca
aaatcccttaacgtgagttttcgttccactgagcgtcagaccccgtagaaaagatcaaaggatct
tcttgagatcctttttttctgcgcgtaatctgctgcttgcaaacaaaaaaaccaccgctaccagc
ggtggtttgtttgccggatcaagagctaccaactctttttccgaaggtaactggcttcagcagag
cgcagataccaaatactgttcttctagtgtagccgtagttaggccaccacttcaagaactctgta
gcaccgcctacatacctcgctctgctaatcctgttaccagtggctgctgccagtggcgataagtc
gtgtcttaccgggttggactcaagacgatagttaccggataaggcgcagcggtcgggctgaacgg
gggggtcgtgcacacagcccagcttggagcgaacgacctacaccgaactgagatacctacagcgt
gagctatgagaaagcgccacgcttcccgaagggagaaaggcggacaggtatccggtaagcggcag
ggtcggaacaggagagcgcacgagggagcttccagggggaaacgcctggtatctttatagtcctg
tcgggtttcgccacctctgacttgagcgtcgatttttgtgatgctcgtcaggggggcggagccta
tggaaaaacgccagcaacgcggcctttttacggttcctggccttttgctggccttttgctcacat
gttctttcctgcgttatcccctgattctgtggataaccgtattaccgcctttgagtgagctgata
ccgctcgccgcagccgaacgaccgagcgcagcgagtcagtgagcgaggaagcggaagagcgccca
atacgcaaaccgcctctccccgcgcgttggccgattcattaatgcagctggcacgacaggtttcc
cgactggaaagcgggcagigagcgcaacgcaattaatgtgagttagctcactcattaggcacccc
aggcittacactttatgcttccggctcgtatgttgtgtggaattgtgagcggataacaatttcac
acaggaaacagctatgaccatgattacgccaagcgcgcaattaaccctcactaaagggaacaaaa
gctggagctgcaagcttggccattgcatacgttgtatccatatcataatatgtacatttatattg
gctcatgtccaacattaccgccatgttgacattgattattgactagttattaatagtaatcaatt
acggggtcattagttcatagcccatatatggagttccgcgttacataacttacggtaaatggccc
gcctggctgaccgcccaacgacccccgcccattgacgtcaataatgacgtatgttcccatagtaa
cgccaatagggactttccattgacgtcaatgggtggagtatttacggtaaactgcccacttggca
gtacatcaagtgtatcatatgccaagtacgccccctattgacgtcaatgacggtaaatggcccgc
ctggcattatgcccagtacatgaccttatgggactttcctacttggcagtacatctacgtattag
tcatcgctattaccatggtgatgcggttttggcagtacatcaatgggcgtggatagcggtttgac
tcacggggatttccaagtctccaccccattgacgtcaatgggagtttgttttggcaccaaaatca
acgggactttccaaaatgtcgtaacaactccgccccattgacgcaaatgggcggtaggcgtgtac
ggtgggaggtctatataagcagagctcgtttagtgaaccgGGGTCTCTCTGGTTAGACCAGATCT
GAGCCTGGGAGCTCTCTGGCTAACTAGGGAACCCACTGCTTAAGCCTCAATAAAGCTTGCCTTGA
GTGCTTCAAGTAGTGTGTGCCCGTCTGTTGTGTGACTCTGGTAACTAGAGATCCCTCAGACCCTT
TTAGTCAGTGTGGAAAATCTCTAGCagtggcgcccgaacagggacttgaaagcgaaagggaaacc
agaggagctctcTCGACGCAGGACTCGGCTTGCTGAAGCGCGCACGGCAAGAGGCGAGGGGCGGC
GACTGGTGAGTACGCCAAAAATTTTGACTAGCGGAGGCTAGAAGGAGAGAGATGGGTGCGAGAGC
GTCAGTATTAAGCGGGGGAGaattagatcgcgatgggaaaaaattcggttaaggccagggggaaa
gaaaaaatataaattaaaacatatagtatgggcaagcagggagctagaacgattcgcagttaatc
ctggcctgttagaaacatcagaaggctgtagacaaatactgggacagctacaaccatcccttcag
acaggatcagaagaacttagatcattatataatacagtagcaaccctctattgtgtgcatcaaag
gatagagataaaagacaccaaggaagctttagacaagatagaggaagagcaaaacaaaagtaaga
ccaccgcacagcaagcGGCCGCTgatcttcagacctggaggaggagatatgagggacaattggag
aagtgaattatataaatataaagtagtaaaaattgaaccattaggagtagcacccaccaaggcaa
agagaagagtggtgcagagagaaaaaagaGCAGTGGGAATaggagctttgttccttgggttcttg
ggagcagcaggaagcactatgggcgcagcgtcaatgacgctgacggtacaggccagacaattatt
gtctggtatagtgcagcagcagaacaatttgctgagggctattgaggcgcaacagcatctgttgc
aactcacagtctggggcatcaagcagctccaggcaagaatcctggctgtggaaagatacctaaag
gatcaacagctcctggggatttggggttgctctggaaaactcatttgcaccactgctgtgccttg
gaatgctagttggagtaataaatctctggaacagatttggaatcacacgacctggatggagtggg
acagagaaattaacaattacacaagcttaatacactccttaattgaagaatcgcaaaaccagcaa
gaaaagaatgaacaagaattattggaattagataaatgggcaagtttgtggaattggtttaacat
aacaaattggctgtggtatataaaattattcataatgatagtaggaggcttggtaggtttaagaa
tagtttttgctgtactttctatagtgaatagagttaggcagggatattcaccattatcgtttcag
acccacctcccaaccccgaggggacccgacaggcccgaaggaatagaagaagaaggtggagagag
agacagagacagatccattcgattagtgaacggatCacaaatggcagtattcatccacaatttta
aaagaaaaggggggattggggggtacagtgcaggggaaagaatagtagacataatagcaacagac
atacaaactaaagaattacaaaaacaaattacaaaaattcaaaattttcgggtttattacaggga
cagcagaggctccggtgcccgtcagtgggcagagcgcacatcgcccacagtccccgagaagttgg
ggggaggggtcggcaattgaaccggtgcctagagaaggtggcgcggggtaaactgggaaagtgat
gtcgtgtactggctccgcctttttcccgaggggggggagaaccgtatataagigcagtagtcgcc
gtgaacgttctttttcgcaacgggittgccgccagaacacaggtgtcgtgaAAATAAGAGAGAAA
AGAAGAGTAAGAAGAAATATAAGAGCCACCatgGTGAAGATCATCAGCAGAAAGAGCCTGGGCAC
CCAGAACGTGTACGACATCGGCGTGGAAAAGGACCACAACTTTCTGCTCAAGAACGGCCTGGTGG
CCAGCAACTGCTTTAGCGGCAGCTCTGATGCCCCTAGCTCTCCAGCTGCTCCTAGACCTGCCAGC
AAGAAGCACTTTCACGAGGAACTGGCCCTGCAGATGGTCGTGTCTACCGGCATGGTTCGAGAGAC
AGTGTTTAAGTACGCCTGGTTCTTCTTCGAACTGCTGGTCAAGAGCATGGCCCAGCACGTGCACA
ACATGGACAAGCGGGACAGCTTCCGGCGGACCAGATTCAGCGACAGATTCATGGACGACATCACC
ACCATCGTGAACGTGGTCACCAGCGAGATTGCTGCCCTGCTGGTTAAGCCCCAGAAAGAGAACGA
GCAGGCCGAAAAGATGAATATCTCTCTGGCCTTCTTCCTCTACGACCTGCTGAGCCTGATGGACC
GGGGCTTCGTGTTCAACCTGATCCGGCACTACTGCTCCCAGCTGTCTGCCAAGCTGTCCAACCTG
CCTACACTGATCTCTATGAGACTCGAGTTCCTGAGGATCCTGTGCAGCCACGAGCACTACCTGAA
CCTGAATCTGTTCTTCATGAACGCCGACACAGCCCCTACAAGCCCCTGTCCTTCTATCAGCAGCC
AGAACAGCAGCAGCTGTAGCAGCTTTCAGGATCAGAAAATCGCCAGCATGTTCGATCTGACCAGC
GAGTACCGGCAGCAGCACTTTCTGACCGGCCTGCTGTTTACCGAACTGGCAGCCGCTCTTGATGC
CGAAGGCGAGGGAATCAGCAAGGTGCAGCGGAAAGCCGTGTCTGCCATTCACAGCCTGCTGTCTA
GCCACGACCTGGATCCTAGATGCGTGAAGCCTGAAGTGAAAGTGAAAATCGCTGCCCTGTACCTG
CCTCTCGTGGGCATCATTCTGGATGCCCTGCCTCAGCTGTGTGATTTCACCGTGGCCGACACCAG
ACGGTACAGAACCTCTGGCTCCGACGAGGAACAAGAAGGCGCTGGCGCCATCAACCAGAATGTGG
CCCTTGCCATTGCCGGGAACAACTTCAATCTGAAAACCAGCGGCATCGTGCTGAGCAGCCTGCCT
TACAAGCAGTACAACATGCTGAATGCCGACACTACCCGCAACCTGATGATCTGCTTCCTGTGGAT
TATGAAGAACGCCGATCAGAGCCTGATTCGGAAGTGGATTGCCGACCTGCCAAGCACTCAGCTGA
ACCGGATTCTGGACCTCCTGTTCATCTGCGTGCTGTGCTTCGAGTACAAGGGCAAGCAGTCCAGC
GACAAGGTGTCCACTCAGGTGCTGCAGAAAAGCCGGGATGTGAAGGCCAGGCTGGAAGAGGCACT
TCTGAGAGGCGAAGGCGCCAGAGGCGAGATGATGAGGCGTAGAGCCCCTGGCAACGATAGATTCC
CCGGCCTGAATGAGAACCTGCGGTGGAAGAAAGAGCAGACCCATTGGAGACAGGCCAACGAGAAA
CTGGACAAGACCAAGGCCGAACTGGACCAAGAGGCCCTGATCTCTGGCAACCTGGCCACAGAAGC
CCACCTGATTATCCTGGACATGCAAGAGAACATCATCCAGGCCAGTAGCGCCCTGGATTGCAAGG
ATTCTCTGCTCGGAGGCGTGCTGAGGGTGCTCGTGAATAGCCTGAATTGCGACCAGAGCACCACC
TACCTGACACACTGCTTCGCCACACTGAGGGCCCTGATTGCCAAGTTTGGGGACCTGCTGTTCGA
GGAAGAGGTGGAACAGTGCTTCGACCTGTGTCACCAAGTGCTGCACCACTGCAGCAGCTCCATGG
ACGTGACAAGATCCCAGGCCTGCGCCACTCTGTATCTGCTGATGCGGTTTAGCTTCGGCGCCACC
AGCAATTTCGCCAGAGTGAAGATGCAAGTGACCATGAGCCTGGCCAGCCTCGTTGGAAGGGCCCC
TGACTTCAATGAGGAACACCTCAGAAGAAGCCTGCGGACCATCCTGGCCTACAGCGAAGAGGATA
CCGCCATGCAGATGACCCCATTTCCAACACAAGTGGAAGAACTGCTGTGCAACCTGAATTCCATC
CTGTACGATACCGTGAAGATGAGAGAGTTCCAAGAGGACCCCGAGATGCTGATGGATCTGATGTA
CCGGATCGCCAAGAGCTACCAGGCCTCTCCTGATCTGAGACTGACCTGGCTGCAGAACATGGCCG
AGAAGCACACCAAGAAGAAGTGCTACACCGAGGCCGCCATGTGCCTGGTTCATGCTGCTGCACTC
GTGGCCGAGTACCTGAGCATGCTGGAAGATCACTCCTACCTGCCAGTGGGCAGCGTGTCCTTCCA
GAACATCAGCTCCAACGTCCTGGAAGAGTCCGTGGTGTCCGAGGACACACTGAGCCCAGATGAGG
ATGGCGTGTGTGCCGGACAGTACTTTACCGAGTCTGGCCTCGTGGGCCTTCTGGAACAGGCCGCC
GAACTGTTTTCTACCGGCGGACTGTACGAGACAGTGAACGAAGTGTACAAGCTGGTCATCCCCAT
CCTGGAAGCCCACAGAGAGTTCAGAAAGCTGACACTGACCCACTCCAAGCTGCAGCGGGCCTTCG
ACAGCATTGTGAACAAGGACCACAAGCGGATGTTCGGCACCTACTTCAGAGTGGGCTTTTTCGGC
AGCAAATTCGGCGACCTGGACGAGCAAGAGTTCGTGTACAAAGAGCCCGCCATCACCAAGCTGCC
TGAGATCTCCCATCGGCTGGAAGCCTTCTACGGCCAGTGTTTTGGCGCCGAGTTCGTGGAAGTGA
TCAAGGACAGCACCCCTGTGGATAAGACCAAACTGGACCCTAACAAGGCCTACATCCAGATCACC
TTCGTGGAACCCTACTTCGACGAGTACGAGATGAAGGATAGAGTGACCTACTTTGAGAAGAACTT
CAACCTGCGGCGGTTCATGTACACCACACCTTTTACACTGGAAGGCAGACCCAGGGGCGAGCTGC
ACGAGCAGTATAGAAGAAACACCGTGCTGACCACCATGCACGCATTCCCTTACATCAAGACCCGG
ATCAGCGTGATCCAGAAAGAAGAGTTTGTCCTGACACCAATCGAGGTGGCCATCGAGGACATGAA
GAAGAAAACCCTCCAGCTGGCCGTGGCCATCAATCAAGAACCTCCAGACGCCAAGATGCTCCAGA
TGGTGCTCCAGGGATCTGTGGGCGCCACAGTGAATCAGGGACCTCTGGAAGTGGCCCAAGTGTTC
CTGGCTGAGATCCCCGCCGATCCTAAGCTGTACAGACACCATAACAAGCTGCGGCTCTGCTTCAA
AGAATTCATCATGAGATGTGGCGAGGCCGTGGAAAAGAACAAGAGACTGATCACCGCCGACCAGA
GAGAGTACCAGCAAGAGCTGAAGAAAAACTACAACAAGCTCAAAGAAAATCTGCGGCCCATGATC
GAGAGAAAGATCCCCGAGCTGTACAAGCCCATCTTCCGCGTGGAAAGCCAGAAGCGGGACTCCTT
TCACAGATCCAGCTTTAGGAAGTGCGAGACACAGCTGTCCCAGGGCTCTTGAttccgataatcaa
cctctggattacaaaatttgtgaaagattgactggtattcttaactatgttgctccttttacgct
atgtggatacgctgctttaatgcctttgtatcatgctattgcttcccgtatggctttcattttct
cctccttgtataaatcctggttgctgtctctttatgaggagttgtggcccgttgtcaggcaacgt
ggcgtggtgtgcactgtgtttgctgacgcaacccccactggttggggcattgccaccacctgtca
gctcctttccgggactttcgctttccccctccctattgccacggcggaactcatcgccgcctgcc
ttgcccgctgctggacaggggctcggctgttgggcactgacaattccgtggtgttgtcggggaaA
TCATcgtcctttccTtggctgctcgcctgtgttgccacctggattctgcgcgggacgtccttctg
ctacgtcccttcggccctcaatccagcggaccttccttcccgcggcctgctgccggctctgcggc
ctcttccgcgtcttcgccttcgccctcagacgagtcggatctccctttgggccgcctccccgcTa
aaagaaaaggggggactggaagggctaattcactcccaacgaagacaagatctgctttttgcttg
tactGGGTCTCTCTGGTTAGACCAGATCTGAGCCTGGGAGCTCTCTGGCTAACTAGGGAACCCAC
TGCTTAAGCCTCAATAAAGCTTGCCTTGAGTGCTTCAAGTAGTGTGTTGGTTTTTTGTGTGTGCA
TTGTCTGAGTAGGTGTCATTCTATTCTGGGGGGTGGGGTGGGGCAGCACAGCAAGGGGGAGGATT
GGGAAGACAATAGCAGGCATGCTGGGGATGCGGTGGGCTCTATGGagtagtagttcatgtcatct
tattattcagtatttataacttgcaaagaaatgaatatcagagagtgagaggaacttgtttattg
cagcttataatggttacaaataaagcaatagcatcacaaatttcacaaataaagcatttttttca
ctgcattctagttgtggtttgtccaaactcatcaatgtatcttatcatgtctggctctagctatc
ccgcccctaactccgcccATCCCGCCCCTAACTCCGCCCAGTTCCGCCCATTCTCCGCCCCATGG
CTCTCACTACTTCTGGAATAGCTCAGAGGCCGAGGCGGCCTCGGCCTCTGCATAAATAAAAAAAA
TTAGTCGAGGCTTTTTTGGAGGCCTAGGgacgtacccaattcgccctatagtgagtcgtattacg
cgcgctcactggccgtcgttttacaacgtcgtgactgggaaaaccctggcgttacccaacttaat
cgccttgcagcacatccccctttcgccagctggcgtaatagcgaagaggcccgcaccgatcgccc
ttcccaacagttgcgcagcctgaatggcgaatggg
(SEQ ID NO: 31)
1267 DOCK8 (1^{st} DOCK8 fragment; 5′ half)
ATGGCCACACTGCCTAGCGCTGAGAGAAGGGCCTTCGCTCTGAAGATCAACCGGTACAGCAGCGC
CGAGATCCGGAAGCAGTTTACCCTGCCTCCTAACCTGGGCCAGTACCACAGACAGAGCATCAGCA
CCAGCGGCTTCCCATCTCTGCAGCTGCCCCAGTTCTACGACCCTGTGGAACCCGTGGATTTCGAG
GGCCTGCTGATGACCCACCTGAACAGCCTGGATGTGCAGCTGGCCCAAGAGCTGGGCGACTTCAC
CGACGATGATCTGGACGTGGTGTTCACCCCTAAAGAGTGCAGAACCCTGCAGCCTAGCCTGCCTG
AAGAAGGCGTGGAACTGGATCCTCACGTGCGGGATTGTGTGCAGACCTACATCCGCGAGTGGCTG
ATCGTGAACCGGAAGAATCAGGGCAGCCCAGAGATCTGCGGCTTCAAGAAAACCGGCAGCCGGAA
GGACTTCCACAAGACCCTGCCTAAGCAGACCTTCGAGAGCGAGACACTGGAATGCTCTGAGCCTG
CTGCTCAGGCCGGACCTAGACACCTGAATGTGCTGTGTGACGTGTCCGGCAAGGGACCTGTGACC
GCCTGCGATTTCGACCTGAGAAGTCTGCAGCCCGACAAGCGGCTGGAAAACCTGCTCCAGCAAGT
GTCCGCCGAGGACTTCGAGAAGCAGAACGAGGAAGCCAGACGGACCAACAGACAGGCCGAGCTGT
TTGCTCTGTACCCCAGCGTGGACGAGGAAGATGCCGTCGAGATTAGACCCGTGCCTGAGTGCCCA
AAAGAGCACCTGGGCAACAGAATCCTGGTCAAGCTGCTGACCCTGAAGTTCGAGATCGAGATTGA
GCCCCTGTTCGCCTCTATCGCCCTGTACGACGTGAAAGAGCGGAAGAAGATCAGCGAGAACTTCC
ACTGCGACCTGAACTCCGACCAGTTCAAGGGCTTCCTGAGAGCCCACACACCATCTGTGGCCGCT
AGCTCTCAGGCCAGATCTGCCGTGTTCAGCGTGACATACCCCAGCAGCGACATCTACCTGGTGGT
CAAGATCGAGAAGGTCCTGCAGCAGGGCGAGATCGGCGATTGTGCCGAGCCTTACACCGTGATCA
AAGAGAGCGACGGCGGCAAGAGCAAAGAGAAGATTGAGAAGCTGAAGCTGCAGGCCGAGAGCTTC
TGTCAGCGGCTGGGCAAGTACAGAATGCCCTTTGCCTGGGCTCCTATCAGCCTGTCCAGCTTCTT
CAACGTGTCCACACTGGAACGGGAAGTGACCGACGTGGACTCTGTCGTGGGCAGATCTAGCGTGG
GCGAGAGAAGGACACTGGCTCAGTCTAGACGGCTGAGCGAGAGAGCCCTGAGCCTGGAAGAAAAT
GGCGTGGGCAGCAACTTCAAGACCAGCACTCTGTCCGTGTCTAGCTTCTTTAAGCAAGAGGGCGA
CAGACTGAGCGACGAGGACCTGTTCAAGTTCCTGGCCGACTACAAGCGGAGCAGCTCCCTGCAGA
GAAGAGTGAAGTCTATCCCTGGCCTGCTGCGCCTGGAAATCTCTACAGCCCCTGAGATCATCAAC
TGCTGTCTGACCCCTGAAATGCTGCCCGTGAAGCCCTTTCCAGAGAACCGGACCAGACCTCACAA
AGAGATCCTGGAATTCCCCACCAGAGAAGTGTACGTGCCCCACACCGTGTACCGGAACCTGCTGT
ATGTGTACCCACAGCGGCTGAACTTCGTGAACAAGCTGGCCTCCGCCAGAAACATCACCATCAAG
ATCCAGTTTATGTGCGGCGAGGACGCCAGCAACGCCATGCCTGTGATCTTCGGCAAGTCTAGCGG
CCCTGAGTTCCTGCAAGAGGTGTACACAGCCGTGACCTACCACAACAAGAGCCCCGACTTCTACG
AGGAAGTGAAGATTAAGCTGCCCGCCAAGCTGACCGTGAATCACCATCTGCTGTTCACCTTCTAC
CACATCAGCTGCCAGCAGAAACAGGGCGCCTCTGTGGAAACACTGCTGGGCTATAGCTGGCTGCC
CATCCTGCTGAACGAGAGACTGCAGACCGGCAGCTACTGTCTGCCTGTGGCTCTGGAAAAGCTGC
CACCTAACTACAGCATGCACTCCGCCGAGAAGGTGCCCCTGCAGAATCCTCCTATTAAGTGGGCC
GAGGGCCACAAGGGCGTGTTCAATATCGAGGTGCAGGCCGTGTCCTCCGTGCACACCCAGGATAA
CCACCTGGAAAAGTTCTTCACCCTGTGCCACAGCCTCGAGAGCCAAGTGACATTCCCCATCCGCG
TGCTGGACCAGAAAATCTCCGAGATGGCCCTGGAACACGAGCTGAAACTGAGCATCATCTGCCTG
AATAGCAGCAGACTGGAACCCCTGGTGCTGTTCCTGCATCTGGTGCTGGACAAGCTGTTCCAGCT
GAGCGTGCAGCCCATGGTTATCGCCGGACAGACCGCCAACTTCAGCCAGTTCGCCTTTGAGAGCG
TGGTGGCCATTGCCAACAGCCTGCACAACAGCAAGGACCTGAGCAAGGATCAGCACGGCAGAAAC
TGCCTGCTGGCCTCTTACGTGCACTACGTGTTCAGACTGCCTGAGGTGCAGAGGGACGTGCCAAA
ATCTGGCGCTCCTACCGCTCTGCTGGACCCCAGAAGCTATCACACCTACGGCAGAACATCTGCCG
CCGCTGTGTCCTCTAAACTCCTGCAGGCTAGAGTGATGTCCAGCAGCAACCCTGATCTGGCCGGA
ACACACAGCGCCGCTGATGAAGAAGTGAAAAACATCATGAGCAGCAAGATCGCCGACCGGAACTG
CAGCCGGATGAGCTACTACTGTAGCGGCAGCTCTGATGCCCCTAGCTCTCCAGCTGCTCCTAGAC
CTGCCAGCAAGAAGCACTTTCACGAGGAACTGGCCCTGCAGATGGTCGTGTCTACCGGCATGGTT
CGAGAGACAGTGTTTAAGTACGCCTGGTTCTTCTTCGAACTGCTGGTCAAGAGCATGGCCCAGCA
CGTGCACAACATGGACAAGCGGGACAGCTTCCGGCGGACCAGATTCAGCGACAGATTCATGGACG
ACATCACCACCATCGTGAACGTGGTCACCAGCGAGATTGCTGCCCTGCTGGTTAAGCCCCAGAAA
GAGAACGAGCAGGCCGAAAAGATGAATATCTCTCTGGCCTTCTTCCTCTACGACCTGCTGAGCCT
GATGGACCGGGGCTTCGTGTTCAACCTGATCCGGCACTACTGCTCCCAGCTGTCTGCCAAGCTGT
CCAACCTGCCTACACTGATCTCTATGAGACTCGAGTTCCTGAGGATCCTGTGCAGCCACGAGCAC
TACCTGAACCTGAATCTGTTCTTCATGAACGCCGACACAGCCCCTACAAGCCCCTGTCCTTCTAT
CAGCAGCCAGAACAGCAGCAGCTGTAGCAGCTTTCAGGATCAGAAAATCGCCAGCATGTTCGATC
TGACCAGCGAGTACCGGCAGCAGCACTTTCTGACCGGCCTGCTGTTTACCGAACTGGCAGCCGCT
CTTGATGCCGAAGGCGAGGGAATCAGCAAGGTGCAGCGGAAAGCCGTGTCTGCCATTCACAGCCT
GCTGTCTAGCCACGACCTGGATCCTAGATGCGTGAAGCCTGAAGTGAAAGTGAAAATCGCTGCCC
TGTACCTGCCTCTCGTGGGCATCATTCTGGATGCCCTGCCTCAGCTGTGTGATTTCACCGTGGCC
GACACCAGACGGTACAGAACCTCTGGCTCCGACGAGGAACAAGAAGGCGCTGGCGCCATCAACCA
GAATGTGGCCCTTGCCATTGCCGGGAACAAC
(SEQ ID NO: 32)
1267 DOCK8-CfaN (1^{st} DOCK8 fragment; N-terminal half)
MATLPSAERRAFALKINRYSSAEIRKQFTLPPNLGQYHRQSISTSGFPSLQLPQFYDPVEPVDFE
GLLMTHLNSLDVQLAQELGDFTDDDLDVVFTPKECRTLQPSLPEEGVELDPHVRDCVQTYIREWL
IVNRKNQGSPEICGFKKTGSRKDFHKTLPKQTFESETLECSEPAAQAGPRHLNVLCDVSGKGPVT
ACDFDLRSLQPDKRLENLLQQVSAEDFEKQNEEARRTNRQAELFALYPSVDEEDAVEIRPVPECP
KEHLGNRILVKLLTLKFEIEIEPLFASIALYDVKERKKISENFHCDLNSDQFKGFLRAHTPSVAA
SSQARSAVFSVTYPSSDIYLVVKIEKVLQQGEIGDCAEPYTVIKESDGGKSKEKIEKLKLQAESF
CQRLGKYRMPFAWAPISLSSFFNVSTLEREVTDVDSVVGRSSVGERRTLAQSRRLSERALSLEEN
GVGSNFKTSTLSVSSFFKQEGDRLSDEDLFKFLADYKRSSSLQRRVKSIPGLLRLEISTAPEIIN
CCLTPEMLPVKPFPENRTRPHKEILEFPTREVYVPHTVYRNLLYVYPQRLNFVNKLASARNITIK
IQFMCGEDASNAMPVIFGKSSGPEFLQEVYTAVTYHNKSPDFYEEVKIKLPAKLTVNHHLLFTFY
HISCQQKQGASVETLLGYSWLPILLNERLQTGSYCLPVALEKLPPNYSMHSAEKVPLQNPPIKWA
EGHKGVFNIEVQAVSSVHTQDNHLEKFFTLCHSLESQVTFPIRVLDQKISEMALEHELKLSIICL
NSSRLEPLVLFLHLVLDKLFQLSVQPMVIAGQTANFSQFAFESVVAIANSLHNSKDLSKDQHGRN
CLLASYVHYVFRLPEVQRDVPKSGAPTALLDPRSYHTYGRTSAAAVSSKLLQARVMSSSNPDLAG
THSAADEEVKNIMSSKIADRNCSRMSYYCSGSSDAPSSPAAPRPASKKHFHEELALQMVVSTGMV
RETVFKYAWFFFELLVKSMAQHVHNMDKRDSFRRTRFSDRFMDDITTIVNVVTSEIAALLVKPQK
ENEQAEKMNISLAFFLYDLLSLMDRGFVFNLIRHYCSQLSAKLSNLPTLISMRLEFLRILCSHEH
YLNLNLFFMNADTAPTSPCPSISSQNSSSCSSFQDQKIASMFDLTSEYRQQHFLTGLLFTELAAA
LDAEGEGISKVQRKAVSAIHSLLSSHDLDPRCVKPEVKVKIAALYLPLVGIILDALPQLCDFTVA
DTRRYRTSGSDEEQEGAGAINQNVALAIAGNNCLSYDTEILTVEYGFLPIGKIVEERIECTVYTV
DKNGFVYTQPIAQWHNRGEQEVFEYCLEDGSIIRATKDHKFMTTDGQMLPIDEIFERGLDLKQVD
GLP*
(SEQ ID NO: 33; *- stop codon)
1267 DOCK8 (1^{st} DOCK8 fragment; N-terminal half)
MATLPSAERRAFALKINRYSSAEIRKQFTLPPNLGQYHRQSISTSGFPSLQLPQFYDPVEPVDFE
GLLMTHLNSLDVQLAQELGDFTDDDLDVVFTPKECRTLQPSLPEEGVELDPHVRDCVQTYIREWL
IVNRKNQGSPEICGFKKTGSRKDFHKTLPKQTFESETLECSEPAAQAGPRHLNVLCDVSGKGPVT
ACDFDLRSLQPDKRLENLLQQVSAEDFEKQNEEARRTNRQAELFALYPSVDEEDAVEIRPVPECP
KEHLGNRILVKLLTLKFEIEIEPLFASIALYDVKERKKISENFHCDLNSDQFKGFLRAHTPSVAA
SSQARSAVFSVTYPSSDIYLVVKIEKVLQQGEIGDCAEPYTVIKESDGGKSKEKIEKLKLQAESF
CQRLGKYRMPFAWAPISLSSFFNVSTLEREVTDVDSVVGRSSVGERRTLAQSRRLSERALSLEEN
GVGSNFKTSTLSVSSFFKQEGDRLSDEDLFKFLADYKRSSSLQRRVKSIPGLLRLEISTAPEIIN
CCLTPEMLPVKPFPENRTRPHKEILEFPTREVYVPHTVYRNLLYVYPQRLNFVNKLASARNITIK
IQFMCGEDASNAMPVIFGKSSGPEFLQEVYTAVTYHNKSPDFYEEVKIKLPAKLTVNHHLLFTFY
HISCQQKQGASVETLLGYSWLPILLNERLQTGSYCLPVALEKLPPNYSMHSAEKVPLQNPPIKWA
EGHKGVFNIEVQAVSSVHTQDNHLEKFFTLCHSLESQVTFPIRVLDQKISEMALEHELKLSIICL
NSSRLEPLVLFLHLVLDKLFQLSVQPMVIAGQTANFSQFAFESVVAIANSLHNSKDLSKDQHGRN
CLLASYVHYVFRLPEVQRDVPKSGAPTALLDPRSYHTYGRTSAAAVSSKLLQARVMSSSNPDLAG
THSAADEEVKNIMSSKIADRNCSRMSYYCSGSSDAPSSPAAPRPASKKHFHEELALQMVVSTGMV
RETVFKYAWFFFELLVKSMAQHVHNMDKRDSFRRTRFSDRFMDDITTIVNVVTSEIAALLVKPQK
ENEQAEKMNISLAFFLYDLLSLMDRGFVFNLIRHYCSQLSAKLSNLPTLISMRLEFLRILCSHEH
YLNLNLFFMNADTAPTSPCPSISSQNSSSCSSFQDQKIASMFDLTSEYRQQHFLTGLLFTELAAA
LDAEGEGISKVQRKAVSAIHSLLSSHDLDPRCVKPEVKVKIAALYLPLVGIILDALPQLCDFTVA
DTRRYRTSGSDEEQEGAGAINQNVALAIAGNN
(SEQ ID NO: 34)
1267 CfaC-CFN-DOCK8 (2^{nd} DOCK8 fragment; 3′ half)
ATGGTGAAGATCATCAGCAGAAAGAGCCTGGGCACCCAGAACGTGTACGACATCGGCGTGGAAAA
GGACCACAACTTTCTGCTCAAGAACGGCCTGGTGGCCAGCAACTGCTTCAATCTGAAAACCAGCG
GCATCGTGCTGAGCAGCCTGCCTTACAAGCAGTACAACATGCTGAATGCCGACACTACCCGCAAC
CTGATGATCTGCTTCCTGTGGATTATGAAGAACGCCGATCAGAGCCTGATTCGGAAGTGGATTGC
CGACCTGCCAAGCACTCAGCTGAACCGGATTCTGGACCTCCTGTTCATCTGCGTGCTGTGCTTCG
AGTACAAGGGCAAGCAGTCCAGCGACAAGGTGTCCACTCAGGTGCTGCAGAAAAGCCGGGATGTG
AAGGCCAGGCTGGAAGAGGCACTTCTGAGAGGCGAAGGCGCCAGAGGCGAGATGATGAGGCGTAG
AGCCCCTGGCAACGATAGATTCCCCGGCCTGAATGAGAACCTGCGGTGGAAGAAAGAGCAGACCC
ATTGGAGACAGGCCAACGAGAAACTGGACAAGACCAAGGCCGAACTGGACCAAGAGGCCCTGATC
TCTGGCAACCTGGCCACAGAAGCCCACCTGATTATCCTGGACATGCAAGAGAACATCATCCAGGC
CAGTAGCGCCCTGGATTGCAAGGATTCTCTGCTCGGAGGCGTGCTGAGGGTGCTCGTGAATAGCC
TGAATTGCGACCAGAGCACCACCTACCTGACACACTGCTTCGCCACACTGAGGGCCCTGATTGCC
AAGTTTGGGGACCTGCTGTTCGAGGAAGAGGTGGAACAGTGCTTCGACCTGTGTCACCAAGTGCT
GCACCACTGCAGCAGCTCCATGGACGTGACAAGATCCCAGGCCTGCGCCACTCTGTATCTGCTGA
TGCGGTTTAGCTTCGGCGCCACCAGCAATTTCGCCAGAGTGAAGATGCAAGTGACCATGAGCCTG
GCCAGCCTCGTTGGAAGGGCCCCTGACTTCAATGAGGAACACCTCAGAAGAAGCCTGCGGACCAT
CCTGGCCTACAGCGAAGAGGATACCGCCATGCAGATGACCCCATTTCCAACACAAGTGGAAGAAC
TGCTGTGCAACCTGAATTCCATCCTGTACGATACCGTGAAGATGAGAGAGTTCCAAGAGGACCCC
GAGATGCTGATGGATCTGATGTACCGGATCGCCAAGAGCTACCAGGCCTCTCCTGATCTGAGACT
GACCTGGCTGCAGAACATGGCCGAGAAGCACACCAAGAAGAAGTGCTACACCGAGGCCGCCATGT
GCCTGGTTCATGCTGCTGCACTCGTGGCCGAGTACCTGAGCATGCTGGAAGATCACTCCTACCTG
CCAGTGGGCAGCGTGTCCTTCCAGAACATCAGCTCCAACGTCCTGGAAGAGTCCGTGGTGTCCGA
GGACACACTGAGCCCAGATGAGGATGGCGTGTGTGCCGGACAGTACTTTACCGAGTCTGGCCTCG
TGGGCCTTCTGGAACAGGCCGCCGAACTGTTTTCTACCGGCGGACTGTACGAGACAGTGAACGAA
GTGTACAAGCTGGTCATCCCCATCCTGGAAGCCCACAGAGAGTTCAGAAAGCTGACACTGACCCA
CTCCAAGCTGCAGCGGGCCTTCGACAGCATTGTGAACAAGGACCACAAGCGGATGTTCGGCACCT
ACTTCAGAGTGGGCTTTTTCGGCAGCAAATTCGGCGACCTGGACGAGCAAGAGTTCGTGTACAAA
GAGCCCGCCATCACCAAGCTGCCTGAGATCTCCCATCGGCTGGAAGCCTTCTACGGCCAGTGTTT
TGGCGCCGAGTTCGTGGAAGTGATCAAGGACAGCACCCCTGTGGATAAGACCAAACTGGACCCTA
ACAAGGCCTACATCCAGATCACCTTCGTGGAACCCTACTTCGACGAGTACGAGATGAAGGATAGA
GTGACCTACTTTGAGAAGAACTTCAACCTGCGGCGGTTCATGTACACCACACCTTTTACACTGGA
AGGCAGACCCAGGGGCGAGCTGCACGAGCAGTATAGAAGAAACACCGTGCTGACCACCATGCACG
CATTCCCTTACATCAAGACCCGGATCAGCGTGATCCAGAAAGAAGAGTTTGTCCTGACACCAATC
GAGGTGGCCATCGAGGACATGAAGAAGAAAACCCTCCAGCTGGCCGTGGCCATCAATCAAGAACC
TCCAGACGCCAAGATGCTCCAGATGGTGCTCCAGGGATCTGTGGGCGCCACAGTGAATCAGGGAC
CTCTGGAAGTGGCCCAAGTGTTCCTGGCTGAGATCCCCGCCGATCCTAAGCTGTACAGACACCAT
AACAAGCTGCGGCTCTGCTTCAAAGAATTCATCATGAGATGTGGCGAGGCCGTGGAAAAGAACAA
GAGACTGATCACCGCCGACCAGAGAGAGTACCAGCAAGAGCTGAAGAAAAACTACAACAAGCTCA
AAGAAAATCTGCGGCCCATGATCGAGAGAAAGATCCCCGAGCTGTACAAGCCCATCTTCCGCGTG
GAAAGCCAGAAGCGGGACTCCTTTCACAGATCCAGCTTTAGGAAGTGCGAGACACAGCTGTCCCA
GGGCTCTTGA
(SEQ ID NO: 35)
1267 DOCK8 (2^{nd} DOCK8 fragment; 3′ half)
CTGAAAACCAGCGGCATCGTGCTGAGCAGCCTGCCTTACAAGCAGTACAACATGCTGAATGCCGA
CACTACCCGCAACCTGATGATCTGCTTCCTGTGGATTATGAAGAACGCCGATCAGAGCCTGATTC
GGAAGTGGATTGCCGACCTGCCAAGCACTCAGCTGAACCGGATTCTGGACCTCCTGTTCATCTGC
GTGCTGTGCTTCGAGTACAAGGGCAAGCAGTCCAGCGACAAGGTGTCCACTCAGGTGCTGCAGAA
AAGCCGGGATGTGAAGGCCAGGCTGGAAGAGGCACTTCTGAGAGGCGAAGGCGCCAGAGGCGAGA
TGATGAGGCGTAGAGCCCCTGGCAACGATAGATTCCCCGGCCTGAATGAGAACCTGCGGTGGAAG
AAAGAGCAGACCCATTGGAGACAGGCCAACGAGAAACTGGACAAGACCAAGGCCGAACTGGACCA
AGAGGCCCTGATCTCTGGCAACCTGGCCACAGAAGCCCACCTGATTATCCTGGACATGCAAGAGA
ACATCATCCAGGCCAGTAGCGCCCTGGATTGCAAGGATTCTCTGCTCGGAGGCGTGCTGAGGGTG
CTCGTGAATAGCCTGAATTGCGACCAGAGCACCACCTACCTGACACACTGCTTCGCCACACTGAG
GGCCCTGATTGCCAAGTTTGGGGACCTGCTGTTCGAGGAAGAGGTGGAACAGTGCTTCGACCTGT
GTCACCAAGTGCTGCACCACTGCAGCAGCTCCATGGACGTGACAAGATCCCAGGCCTGCGCCACT
CTGTATCTGCTGATGCGGTTTAGCTTCGGCGCCACCAGCAATTTCGCCAGAGTGAAGATGCAAGT
GACCATGAGCCTGGCCAGCCTCGTTGGAAGGGCCCCTGACTTCAATGAGGAACACCTCAGAAGAA
GCCTGCGGACCATCCTGGCCTACAGCGAAGAGGATACCGCCATGCAGATGACCCCATTTCCAACA
CAAGTGGAAGAACTGCTGTGCAACCTGAATTCCATCCTGTACGATACCGTGAAGATGAGAGAGTT
CCAAGAGGACCCCGAGATGCTGATGGATCTGATGTACCGGATCGCCAAGAGCTACCAGGCCTCTC
CTGATCTGAGACTGACCTGGCTGCAGAACATGGCCGAGAAGCACACCAAGAAGAAGTGCTACACC
GAGGCCGCCATGTGCCTGGTTCATGCTGCTGCACTCGTGGCCGAGTACCTGAGCATGCTGGAAGA
TCACTCCTACCTGCCAGTGGGCAGCGTGTCCTTCCAGAACATCAGCTCCAACGTCCTGGAAGAGT
CCGTGGTGTCCGAGGACACACTGAGCCCAGATGAGGATGGCGTGTGTGCCGGACAGTACTTTACC
GAGTCTGGCCTCGTGGGCCTTCTGGAACAGGCCGCCGAACTGTTTTCTACCGGCGGACTGTACGA
GACAGTGAACGAAGTGTACAAGCTGGTCATCCCCATCCTGGAAGCCCACAGAGAGTTCAGAAAGC
TGACACTGACCCACTCCAAGCTGCAGCGGGCCTTCGACAGCATTGTGAACAAGGACCACAAGCGG
ATGTTCGGCACCTACTTCAGAGTGGGCTTTTTCGGCAGCAAATTCGGCGACCTGGACGAGCAAGA
GTTCGTGTACAAAGAGCCCGCCATCACCAAGCTGCCTGAGATCTCCCATCGGCTGGAAGCCTTCT
ACGGCCAGTGTTTTGGCGCCGAGTTCGTGGAAGTGATCAAGGACAGCACCCCTGTGGATAAGACC
AAACTGGACCCTAACAAGGCCTACATCCAGATCACCTTCGTGGAACCCTACTTCGACGAGTACGA
GATGAAGGATAGAGTGACCTACTTTGAGAAGAACTTCAACCTGCGGCGGTTCATGTACACCACAC
CTTTTACACTGGAAGGCAGACCCAGGGGCGAGCTGCACGAGCAGTATAGAAGAAACACCGTGCTG
ACCACCATGCACGCATTCCCTTACATCAAGACCCGGATCAGCGTGATCCAGAAAGAAGAGTTTGT
CCTGACACCAATCGAGGTGGCCATCGAGGACATGAAGAAGAAAACCCTCCAGCTGGCCGTGGCCA
TCAATCAAGAACCTCCAGACGCCAAGATGCTCCAGATGGTGCTCCAGGGATCTGTGGGCGCCACA
GTGAATCAGGGACCTCTGGAAGTGGCCCAAGTGTTCCTGGCTGAGATCCCCGCCGATCCTAAGCT
GTACAGACACCATAACAAGCTGCGGCTCTGCTTCAAAGAATTCATCATGAGATGTGGCGAGGCCG
TGGAAAAGAACAAGAGACTGATCACCGCCGACCAGAGAGAGTACCAGCAAGAGCTGAAGAAAAAC
TACAACAAGCTCAAAGAAAATCTGCGGCCCATGATCGAGAGAAAGATCCCCGAGCTGTACAAGCC
CATCTTCCGCGTGGAAAGCCAGAAGCGGGACTCCTTTCACAGATCCAGCTTTAGGAAGTGCGAGA
CACAGCTGTCCCAGGGCTCTTGA
(SEQ ID NO: 36)
1267 DOCK8 (2^{nd} DOCK8 fragment; C-terminal half)
LKTSGIVLSSLPYKQYNMLNADTTRNLMICFLWIMKNADQSLIRKWIADLPSTQLNRILDLLFIC
VLCFEYKGKQSSDKVSTQVLQKSRDVKARLEEALLRGEGARGEMMRRRAPGNDRFPGLNENLRWK
KEQTHWRQANEKLDKTKAELDQEALISGNLATEAHLIILDMQENIIQASSALDCKDSLLGGVLRV
LVNSLNCDQSTTYLTHCFATLRALIAKFGDLLFEEEVEQCFDLCHQVLHHCSSSMDVTRSQACAT
LYLLMRFSFGATSNFARVKMQVTMSLASLVGRAPDFNEEHLRRSLRTILAYSEEDTAMQMTPFPT
QVEELLCNLNSILYDTVKMREFQEDPEMLMDLMYRIAKSYQASPDLRLTWLQNMAEKHTKKKCYT
EAAMCLVHAAALVAEYLSMLEDHSYLPVGSVSFQNISSNVLEESVVSEDTLSPDEDGVCAGQYFT
ESGLVGLLEQAAELFSTGGLYETVNEVYKLVIPILEAHREFRKLTLTHSKLQRAFDSIVNKDHKR
MFGTYFRVGFFGSKFGDLDEQEFVYKEPAITKLPEISHRLEAFYGQCFGAEFVEVIKDSTPVDKT
KLDPNKAYIQITFVEPYFDEYEMKDRVTYFEKNFNLRRFMYTTPFTLEGRPRGELHEQYRRNTVL
TTMHAFPYIKTRISVIQKEEFVLTPIEVAIEDMKKKTLQLAVAINQEPPDAKMLQMVLQGSVGAT
VNQGPLEVAQVFLAEIPADPKLYRHHNKLRLCFKEFIMRCGEAVEKNKRLITADQREYQQELKKN
YNKLKENLRPMIERKIPELYKPIFRVESQKRDSFHRSSFRKCETQLSQGS*
(SEQ ID NO: 37; *- stop codon)
EFS-1267-DOCK8-CfaN-WPRE (1^{st} DOCK8 fragment; 5′ half)
acgcgccctgtagcggcgcattaagcgcggcgggtgtggtggttacgcgcagcgtgaccgctaca
cttgccagcgccctagcgcccgctcctttcgctttcttcccttcctttctcgccacgttcgccgg
ctttccccgtcaagctctaaatcgggggctccctttagggttccgatttagtgctttacggcacc
tcgaccccaaaaaacttgattagggtgatggttcacgtagtgggccatcgccctgatagacggtt
tttcgccctttgacgttggagtccacgttctttaatagtggactcttgttccaaactggaacaac
actcaaccctatctcggtctattcttttgatttataagggattttgccgatttcggcctattggt
taaaaaatgagctgatttaacaaaaatttaacgcgaattttaacaaaatattaacgcttacaatt
taggtggcacttttcggggaaatgtgcgcggaacccctatttgtttatttttctaaatacattca
aatatgtatccgctcatgagacaataaccctgataaatgcttcaataatagcacctagatcaaga
gacaggatgaggatcgtttcgcatgattgaacaagatggattgcacgcaggttctccggccgctt
gggtggagaggctattcggctatgactgggcacaacagacaatcggctgctctgatgccgccgtg
ttccggctgtcagcgcaggggcgcccggttctttttgtcaagaccgacctgtccggtgccctgaa
tgaactgcaagacgaggcagcgcggctatcgtggctggccacgacgggcgttccttgcgcagctg
tgctcgacgttgtcactgaagcgggaagggactggctgctattgggcgaagtgccggggcaggat
ctcctgtcatctcaccttgctcctgccgagaaagtatccatcatggctgatgcaatgcggggctg
catacgcttgatccggctacctgcccattcgaccaccaagcgaaacatcgcatcgagcgagcacg
tactcggatggaagccggtcttgtcgatcaggatgatctggacgaagagcatcaggggctcgcgc
cagccgaactgttcgccaggctcaaggcgagcatgcccgacggcgaggatctcgtcgtgacccat
ggcgatgcctgcttgccgaatatcatggtggaaaatggccgcttttctggattcatcgactgtgg
ccggctgggtgtggcggaccgctatcaggacatagcgttggctacccgtgatattgctgaagagc
ttggcggcgaatgggctgaccgcttcctcgtgctttacggtatcgccgctcccgattcgcagcgc
atcgccttctatcgccttcttgacgagttcttctgaattattaacgcttacaatttcctgatgcg
gtattttctccttacgcatctgtgcggtatttcacaccgcatcaggtggcacttttcggggaaat
gtgcgcggaacccctatttgtttatttttctaaatacattcaaatatgtatccgctcatgaccaa
aatcccttaacgtgagttttcgttccactgagcgtcagaccccgtagaaaagatcaaaggatctt
cttgagatcctttttttctgcgcgtaatctgctgcttgcaaacaaaaaaaccaccgctaccagcg
gtggtttgtttgccggatcaagagctaccaactctttttccgaaggtaactggcttcagcagagc
gcagataccaaatactgttcttctagtgtagccgtagttaggccaccacttcaagaactctgtag
caccgcctacatacctcgctctgctaatcctgttaccagtggctgctgccagtggcgataagtcg
tgtcttaccgggttggactcaagacgatagttaccggataaggcgcagcggtcgggctgaacggg
ggggtcgtgcacacagcccagcttggagcgaacgacctacaccgaactgagatacctacagcgtg
agctatgagaaagcgccacgcttcccgaagggagaaaggcggacaggtatccggtaagcggcagg
gtcggaacaggagagcgcacgagggagcttccagggggaaacgcctggtatctttatagtcctgt
cgggtttcgccacctctgacttgagcgtcgatttttgtgatgctcgtcaggggggcggagcctat
ggaaaaacgccagcaacgcggcctttttacggttcctggccttttgctggccttttgctcacatg
ttctttcctgcgttatcccctgattctgtggataaccgtattaccgcctttgagtgagctgatac
cgctcgccgcagccgaacgaccgagcgcagcgagtcagtgagcgaggaagcggaagagcgcccaa
tacgcaaaccgcctctccccgcgcgttggccgattcattaatgcagctggcacgacaggtttccc
gactggaaagcgggcagtgagcgcaacgcaattaatgtgagttagctcactcattaggcacccca
ggctttacactttatgttccggctcgtatgttgtgtggaattgtgagcggataacaatttcacac
aggaaacagctatgaccatgattacgccaagcgcgcaattaaccctcactaaagggaacaaaagc
tggagctgcaagcttggccattgcatacgttgtatccatatcataatatgtacatttatattggc
tcatgtccaacattaccgccatgttgacattgattattgactagttattaatagtaatcaattac
ggggtcattagttcatagcccatatatggagttccgcgttacataacttacggtaaatggcccgc
ctggctgaccgcccaacgacccccgcccattgacgtcaataatgacgtatgttcccatagtaacg
ccaatagggactttccattgacgtcaatgggtggagtatttacggtaaactgcccacttggcagt
acatcaagtgtatcatatgccaagtacgccccctattgacgicaatgacggtaaatggcccgcct
ggcattalgcccaglacatgaccttatgggactttcctacttggcagtacatctacgtattagtc
atcgctattaccatggtgatgcggttttggcagtacatcaatgggcgtggatagcggtttgactc
acggggatttccaagtctccaccccattgacgtcaatgggagtttgttttggcaccaaaatcaac
gggactttccaaaatgtcgtaacaactccgccccattgacgcaaatgggcggtaggcgtgtacgg
tgggaggtctatataagcagagctcgtttagtgaaccgGGGTCTCTCTGGTTAGACCAGATCTGA
GCCTGGGAGCTCTCTGGCTAACTAGGGAACCCACTGCTTAAGCCTCAATAAAGCTTGCCTTGAGT
GCTTCAAGTAGTGTGTGCCCGTCTGTTGTGTGACTCTGGTAACTAGAGATCCCTCAGACCCTTTT
AGTCAGTGTGGAAAATCTCTAGCagtggcgcccgaacagggacttgaaagcgaaagggaaaccag
aggagctctcTCGACGCAGGACTCGGCTTGCTGAAGCGCGCACGGCAAGAGGCGAGGGGCGGCGA
CTGGTGAGTACGCCAAAAATTTTGACTAGCGGAGGCTAGAAGGAGAGAGATGGGTGCGAGAGCGT
CAGTATTAAGCGGGGGAGaattagatcgcgatgggaaaaaattcggttaaggccagggggaaaga
aaaaatataaattaaaacatatagtatgggcaagcagggagctagaacgattcgcagttaatcct
ggcctgttagaaacatcagaaggctgtagacaaatactgggacagctacaaccatcccttcagac
aggatcagaagaacttagatcattatataatacagtagcaaccctctattgtgtgcatcaaagga
tagagataaaagacaccaaggaagctttagacaagatagaggaagagcaaaacaaaagtaagacc
accgcacagcaagcGGCCGCTgatcttcagacctggaggaggagatatgagggacaattggagaa
gtgaattatataaatataaagtagtaaaaattgaaccattaggagtagcacccaccaaggcaaag
agaagagtggtgcagagagaaaaaagaGCAGTGGGAATaggagctttgttccttgggttcttggg
agcagcaggaagcactatgggcgcagcgtcaatgacgctgacggtacaggccagacaattattgt
ctggtatagtgcagcagcagaacaatttgctgagggctattgaggcgcaacagcatctgttgcaa
ctcacagtctggggcatcaagcagctccaggcaagaatcctggctgtggaaagatacctaaagga
tcaacagctcctggggatttggggttgctctggaaaactcatttgcaccactgctgtgccttgga
atgctagttggagtaataaatctctggaacagatttggaatcacacgacctggatggagtgggac
agagaaattaacaattacacaagcttaatacactccttaattgaagaatcgcaaaaccagcaaga
aaagaatgaacaagaattattggaattagataaatgggcaagtttgtggaattggtttaacataa
caaattggctgtggtatataaaattattcataatgatagtaggaggcttggtaggtttaagaata
gtttttgctgtactttctatagtgaatagagttaggcagggatattcaccattatcgtttcagac
ccacctcccaaccccgaggggacccgacaggcccgaaggaatagaagaagaaggtggagagagag
acagagacagatccattcgattagtgaacggatCacaaatggcagtattcatccacaattttaaa
agaaaaggggggattggggggtacagtgcaggggaaagaatagtagacataatagcaacagacat
acaaactaaagaattacaaaaacaaattacaaaaattcaaaattttcgggtttattacagggaca
gcagaggctccggtgcccgtcagtgggcagagcgcacatcgcccacagtccccgagaagttgggg
ggaggggtcggcaattgaaccggtgcctagagaaggtggcgcggggtaaactgggaaagtgatgt
cgtgtactggctccgcctttttcccgaggggggggagaaccgtatataagtgcagtagtcgccgt
gaacgttctttttcgcaacgggtttgccgccagaacacaggtgtcgtgaAAATAAGAGAGAAAAG
AAGAGTAAGAAGAAATATAAGAGCCACCATGGCCACACTGCCTAGCGCTGAGAGAAGGGCCTTCG
CTCTGAAGATCAACCGGTACAGCAGCGCCGAGATCCGGAAGCAGTTTACCCTGCCTCCTAACCTG
GGCCAGTACCACAGACAGAGCATCAGCACCAGCGGCTTCCCATCTCTGCAGCTGCCCCAGTTCTA
CGACCCTGTGGAACCCGTGGATTTCGAGGGCCTGCTGATGACCCACCTGAACAGCCTGGATGTGC
AGCTGGCCCAAGAGCTGGGCGACTTCACCGACGATGATCTGGACGTGGTGTTCACCCCTAAAGAG
TGCAGAACCCTGCAGCCTAGCCTGCCTGAAGAAGGCGTGGAACTGGATCCTCACGTGCGGGATTG
TGTGCAGACCTACATCCGCGAGTGGCTGATCGTGAACCGGAAGAATCAGGGCAGCCCAGAGATCT
GCGGCTTCAAGAAAACCGGCAGCCGGAAGGACTTCCACAAGACCCTGCCTAAGCAGACCTTCGAG
AGCGAGACACTGGAATGCTCTGAGCCTGCTGCTCAGGCCGGACCTAGACACCTGAATGTGCTGTG
TGACGTGTCCGGCAAGGGACCTGTGACCGCCTGCGATTTCGACCTGAGAAGTCTGCAGCCCGACA
AGCGGCTGGAAAACCTGCTCCAGCAAGTGTCCGCCGAGGACTTCGAGAAGCAGAACGAGGAAGCC
AGACGGACCAACAGACAGGCCGAGCTGTTTGCTCTGTACCCCAGCGTGGACGAGGAAGATGCCGT
CGAGATTAGACCCGTGCCTGAGTGCCCAAAAGAGCACCTGGGCAACAGAATCCTGGTCAAGCTGC
TGACCCTGAAGTTCGAGATCGAGATTGAGCCCCTGTTCGCCTCTATCGCCCTGTACGACGTGAAA
GAGCGGAAGAAGATCAGCGAGAACTTCCACTGCGACCTGAACTCCGACCAGTTCAAGGGCTTCCT
GAGAGCCCACACACCATCTGTGGCCGCTAGCTCTCAGGCCAGATCTGCCGTGTTCAGCGTGACAT
ACCCCAGCAGCGACATCTACCTGGTGGTCAAGATCGAGAAGGTCCTGCAGCAGGGCGAGATCGGC
GATTGTGCCGAGCCTTACACCGTGATCAAAGAGAGCGACGGCGGCAAGAGCAAAGAGAAGATTGA
GAAGCTGAAGCTGCAGGCCGAGAGCTTCTGTCAGCGGCTGGGCAAGTACAGAATGCCCTTTGCCT
GGGCTCCTATCAGCCTGTCCAGCTTCTTCAACGTGTCCACACTGGAACGGGAAGTGACCGACGTG
GACTCTGTCGTGGGCAGATCTAGCGTGGGCGAGAGAAGGACACTGGCTCAGTCTAGACGGCTGAG
CGAGAGAGCCCTGAGCCTGGAAGAAAATGGCGTGGGCAGCAACTTCAAGACCAGCACTCTGTCCG
TGTCTAGCTTCTTTAAGCAAGAGGGCGACAGACTGAGCGACGAGGACCTGTTCAAGTTCCTGGCC
GACTACAAGCGGAGCAGCTCCCTGCAGAGAAGAGTGAAGTCTATCCCTGGCCTGCTGCGCCTGGA
AATCTCTACAGCCCCTGAGATCATCAACTGCTGTCTGACCCCTGAAATGCTGCCCGTGAAGCCCT
TTCCAGAGAACCGGACCAGACCTCACAAAGAGATCCTGGAATTCCCCACCAGAGAAGTGTACGTG
CCCCACACCGTGTACCGGAACCTGCTGTATGTGTACCCACAGCGGCTGAACTTCGTGAACAAGCT
GGCCTCCGCCAGAAACATCACCATCAAGATCCAGTTTATGTGCGGCGAGGACGCCAGCAACGCCA
TGCCTGTGATCTTCGGCAAGTCTAGCGGCCCTGAGTTCCTGCAAGAGGTGTACACAGCCGTGACC
TACCACAACAAGAGCCCCGACTTCTACGAGGAAGTGAAGATTAAGCTGCCCGCCAAGCTGACCGT
GAATCACCATCTGCTGTTCACCTTCTACCACATCAGCTGCCAGCAGAAACAGGGCGCCTCTGTGG
AAACACTGCTGGGCTATAGCTGGCTGCCCATCCTGCTGAACGAGAGACTGCAGACCGGCAGCTAC
TGTCTGCCTGTGGCTCTGGAAAAGCTGCCACCTAACTACAGCATGCACTCCGCCGAGAAGGTGCC
CCTGCAGAATCCTCCTATTAAGTGGGCCGAGGGCCACAAGGGCGTGTTCAATATCGAGGTGCAGG
CCGTGTCCTCCGTGCACACCCAGGATAACCACCTGGAAAAGTTCTTCACCCTGTGCCACAGCCTC
GAGAGCCAAGTGACATTCCCCATCCGCGTGCTGGACCAGAAAATCTCCGAGATGGCCCTGGAACA
CGAGCTGAAACTGAGCATCATCTGCCTGAATAGCAGCAGACTGGAACCCCTGGTGCTGTTCCTGC
ATCTGGTGCTGGACAAGCTGTTCCAGCTGAGCGTGCAGCCCATGGTTATCGCCGGACAGACCGCC
AACTTCAGCCAGTTCGCCTTTGAGAGCGTGGTGGCCATTGCCAACAGCCTGCACAACAGCAAGGA
CCTGAGCAAGGATCAGCACGGCAGAAACTGCCTGCTGGCCTCTTACGTGCACTACGTGTTCAGAC
TGCCTGAGGTGCAGAGGGACGTGCCAAAATCTGGCGCTCCTACCGCTCTGCTGGACCCCAGAAGC
TATCACACCTACGGCAGAACATCTGCCGCCGCTGTGTCCTCTAAACTCCTGCAGGCTAGAGTGAT
GTCCAGCAGCAACCCTGATCTGGCCGGAACACACAGCGCCGCTGATGAAGAAGTGAAAAACATCA
TGAGCAGCAAGATCGCCGACCGGAACTGCAGCCGGATGAGCTACTACTGTAGCGGCAGCTCTGAT
GCCCCTAGCTCTCCAGCTGCTCCTAGACCTGCCAGCAAGAAGCACTTTCACGAGGAACTGGCCCT
GCAGATGGTCGTGTCTACCGGCATGGTTCGAGAGACAGTGTTTAAGTACGCCTGGTTCTTCTTCG
AACTGCTGGTCAAGAGCATGGCCCAGCACGTGCACAACATGGACAAGCGGGACAGCTTCCGGCGG
ACCAGATTCAGCGACAGATTCATGGACGACATCACCACCATCGTGAACGTGGTCACCAGCGAGAT
TGCTGCCCTGCTGGTTAAGCCCCAGAAAGAGAACGAGCAGGCCGAAAAGATGAATATCTCTCTGG
CCTTCTTCCTCTACGACCTGCTGAGCCTGATGGACCGGGGCTTCGTGTTCAACCTGATCCGGCAC
TACTGCTCCCAGCTGTCTGCCAAGCTGTCCAACCTGCCTACACTGATCTCTATGAGACTCGAGTT
CCTGAGGATCCTGTGCAGCCACGAGCACTACCTGAACCTGAATCTGTTCTTCATGAACGCCGACA
CAGCCCCTACAAGCCCCTGTCCTTCTATCAGCAGCCAGAACAGCAGCAGCTGTAGCAGCTTTCAG
GATCAGAAAATCGCCAGCATGTTCGATCTGACCAGCGAGTACCGGCAGCAGCACTTTCTGACCGG
CCTGCTGTTTACCGAACTGGCAGCCGCTCTTGATGCCGAAGGCGAGGGAATCAGCAAGGTGCAGC
GGAAAGCCGTGTCTGCCATTCACAGCCTGCTGTCTAGCCACGACCTGGATCCTAGATGCGTGAAG
CCTGAAGTGAAAGTGAAAATCGCTGCCCTGTACCTGCCTCTCGTGGGCATCATTCTGGATGCCCT
GCCTCAGCTGTGTGATTTCACCGTGGCCGACACCAGACGGTACAGAACCTCTGGCTCCGACGAGG
AACAAGAAGGCGCTGGCGCCATCAACCAGAATGTGGCCCTTGCCATTGCCGGGAACAACTGCCTG
AGCTACGACACCGAGATCCTGACCGTGGAATACGGCTTCCTGCCTATCGGCAAGATCGTGGAAGA
ACGGATCGAGTGCACCGTGTACACCGTGGACAAGAACGGCTTCGTGTACACACAGCCTATCGCTC
AGTGGCACAACCGGGGAGAGCAAGAGGTGTTCGAGTACTGCCTGGAAGATGGCAGCATCATCCGG
GCCACCAAGGACCACAAGTTCATGACCACCGACGGCCAGATGCTGCCCATCGACGAGATCTTTGA
GAGAGGCCTGGACCTGAAACAGGTGGACGGACTTCCTtgattccgataatcaacctctggattac
aaaatttgtgaaagattgactggtattcttaactatgttgctccttttacgctatgtggatacgc
tgctttaatgcctttgtatcatgctattgcttcccgtatggctttcattttctcctccttgtata
aatcctggttgctgtctctttatgaggagttgtggcccgttgtcaggcaacgtggcgtggtgtgc
actgtgtttgctgacgcaacccccactggttggggcattgccaccacctgtcagctcctttccgg
gactttcgctttccccctccctattgccacggcggaactcatcgccgcctgccttgcccgctgct
ggacaggggctcggctgttgggcactgacaattccgtggtgttgtcggggaaATCATcgtccttt
ccTtggctgctcgcctgtgttgccacctggattctgcgcgggacgtccttctgctacgtcccttc
ggccctcaatccagcggaccttccttcccgcggcctgctgccggctctgcggcctcttccgcgtc
ttcgccttcgccctcagacgagtcggatctccctttgggccgcctccccgcTaaaagaaaagggg
ggactggaagggctaattcactcccaacgaagacaagatctgctttttgcttgtactGGGTCTCT
CTGGTTAGACCAGATCTGAGCCTGGGAGCTCTCTGGCTAACTAGGGAACCCACTGCTTAAGCCTC
AATAAAGCTTGCCTTGAGTGCTTCAAGTAGTGTGTTGGTTTTTTGTGTGTGCATTGTCTGAGTAG
GTGTCATTCTATTCTGGGGGGTGGGGTGGGGCAGCACAGCAAGGGGGAGGATTGGGAAGACAATA
GCAGGCATGCTGGGGATGCGGTGGGCTCTATGGagtagtagttcatgtcatcttattattcagta
tttataacttgcaaagaaatgaatatcagagagtgagaggaacttgtttattgcagcttataatg
gttacaaataaagcaatagcatcacaaatttcacaaataaagcatttttttcactgcattctagt
tgtggtttgtccaaactcatcaatgtatcttatcatgtctggctctagclatcccgcccctaact
ccgcccATCCCGCCCCTAACTCCGCCCAGTTCCGCCCATTCTCCGCCCCATGGCTCTCACTACTT
CTGGAATAGCTCAGAGGCCGAGGCGGCCTCGGCCTCTGCATAAATAAAAAAAATTAGTCGAGGCT
TTTTTGGAGGCCTAGGgacgtacccaattcgccctatagtgagtcgtattacgcgcgctcactgg
ccgtcgttttacaacgtgtgactgggaaaaccctggcgttacccaacttaatcgccttgcagcac
atccccctttcgccagctggcgtaatagcgaagaggcccgcaccgatcgcccttcccaacagttg
cgcagcctgaatggcgaatggg
(SEQ ID NO: 38)
EFS-1267-DOCK8-CfaC-CFN-WPRE (2^{nd} DOCK8 fragment; 3′ half)
acgcgccctgtagcggcgcattaagcgcggcgggtgtggtggtlacgcgcagcgtgaccgctaca
cttgccagcgccctagcgcccgctcctttcgctttcttcccttcctttctcgccacgttcgccgg
ctttccccgtcaagctctaaatcgggggctccctttagggttccgatttagtgctttacggcacc
tcgaccccaaaaaacttgattagggtgatggttcacgtagtgggccatcgccctgatagacggtt
tttcgccctttgacgttggagtccacgttctttaatagtggactcttgttccaaactggaacaac
actcaaccctatctcggtctattcttttgatttataagggattttgccgatttcggcctattggt
taaaaaatgagctgatttaacaaaaatttaacgcgaattttaacaaaatattaacgcttacaatt
taggtggcacttttcggggaaatgtgcgcggaacccctatttgtttatttttctaaatacattca
aatatgtatccgctcatgagacaataaccctgataaatgcttcaataatagcacctagatcaaga
gacaggatgaggatcgtttcgcatgattgaacaagatggattgcacgcaggttctccggccgctt
gggtggagaggctattcggctatgactgggcacaacagacaatcggctgctctgatgccgccgtg
ttccggctgtcagcgcaggggcgcccggttctttttgtcaagaccgacctgtccggtgccctgaa
tgaactgcaagacgaggcagcgcggctatcgtggctggccacgacgggcgttccttgcgcagctg
tgctcgacgttgtcactgaagcgggaagggactggctgctattgggcgaagtgccggggcaggat
ctcctgtcatctcaccttgctcctgccgagaaagtatccatcatggctgatgcaatgcggcggct
gcatacgcttgatccggctacctgcccattcgaccaccaagcgaaacatcgcatcgagcgagcac
gtactcggatggaagccggtcttgtcgatcaggatgatctggacgaagagcatcaggggctcgcg
ccagccgaactgttcgccaggctcaaggcgagcatgcccgacggcgaggatctcgtcgtgaccca
tggcgatgcctgcttgccgaatatcatggtggaaaatggccgcttttctggattcatcgactgtg
gccggctgggtgtggcggaccgctatcaggacatagcgttggctacccgtgatattgctgaagag
cttggcggcgaatgggctgaccgcttcctcgtgctttacggtatcgccgctcccgattcgcagcg
catcgccttctatcgccttcttgacgagttcttctgaattattaacgcttacaatttcctgatgc
ggtattttctccttacgcatctgtgcggtatttcacaccgcatcaggtggcacttttcggggaaa
tgtgcgcggaacccctatttgtttatttttctaaatacattcaaatatgtatccgctcatgacca
aaatcccttaacgtgagttttcgttccactgagcgtcagaccccgtagaaaagatcaaaggatct
tcttgagatcctttttttctgcgcgtaatctgctgcttgcaaacaaaaaaaccaccgctaccagc
ggtggtttgtttgccggatcaagagctaccaactctttttccgaaggtaactggcttcagcagag
cgcagataccaaatactgttcttctagtgtagccgtagttaggccaccacttcaagaactctgta
gcaccgcctacatacctcgctctgctaatcctgttaccagtggctgctgccagtggcgataagtc
gtgtcttaccgggttggactcaagacgatagttaccggataaggcgcagcggtcgggctgaacgg
gggggtcgtgcacacagcccagcttggagcgaacgacctacaccgaactgagatacctacagcgt
gagctatgagaaagcgccacgcttcccgaagggagaaaggcggacaggtatccggtaagcggcag
ggtcggaacaggagagcgcacgagggagcttccagggggaaacgcctggtatctttatagtcctg
tcgggtttcgccacctctgacttgagcgtcgatttttgtgatgctcgtcaggggggcggagccta
tggaaaaacgccagcaacgcggcctttttacggttcctggccttttgctggccttttgctcacat
gttctttcctgcgttatcccctgattctgtggataaccgtattaccgcctttgagtgagctgata
ccgctcgccgcagccgaacgaccgagcgcagcgagtcagtgagcgaggaagcggaagagcgccca
atacgcaaaccgcctctccccgcgcgttggccgattcattaatgcagctggcacgacaggtttcc
cgactggaaagcgggcagtgagcgcaacgcaattaatgtgagttagctcactcattaggcacccc
aggctttacactttatgcttccggctcgtatgttgtgtggaattgtgagcggataacaatttcac
acaggaaacagctatgaccatgattacgccaagcgcgcaattaaccctcactaaagggaacaaaa
gctggagctgcaagcttggccattgcatacgttgtatccatatcataatatgtacatttatattg
gctcatgtccaacattaccgccatgttgacattgattattgactagttattaatagtaatcaatt
acggggtcattagttcatagcccatatatggagttccgcgttacataacttacggtaaatggccc
gcctggctgaccgcccaacgacccccgcccattgacgtcaataatgacgtatgttcccatagtaa
cgccaatagggactttccattgacgtcaatgggtggagtatttacggtaaactgcccacttggca
gtacatcaagtgtatcatatgccaagtacgccccctattgacgtcaatgacggtaaatggcccgc
ctggcattatgcccagtacatgaccttatgggactttcctacttggcagtacatctacgtattag
tcatcgctattaccatggtgatgcggttttggcagtacatcaatgggcgtggatagcggtttgac
tcacggggatttccaagtctccaccccattgacgtcaatgggagtttgttttggcaccaaaatca
acgggactttccaaaatgtcgtaacaactccgccccattgacgcaaatgggcggtaggcgtgtac
ggtgggaggtctatataagcagagctcgtttagtgaaccgGGGTCTCTCTGGTTAGACCAGATCT
GAGCCTGGGAGCTCTCTGGCTAACTAGGGAACCCACTGCTTAAGCCTCAATAAAGCTTGCCTTGA
GTGCTTCAAGTAGTGTGTGCCCGTCTGTTGTGTGACTCTGGTAACTAGAGATCCCTCAGACCCTT
TTAGTCAGTGTGGAAAATCTCTAGCagtggcgcccgaacagggacttgaaagcgaaagggaaacc
agaggagctctcTCGACGCAGGACTCGGCTTGCTGAAGCGCGCACGGCAAGAGGCGAGGGGCGGC
GACTGGTGAGTACGCCAAAAATTTTGACTAGCGGAGGCTAGAAGGAGAGAGATGGGTGCGAGAGC
GTCAGTATTAAGCGGGGGAGaattagatcgcgatgggaaaaaattcggttaaggccagggggaaa
gaaaaaatataaattaaaacatatagtatgggcaagcagggagctagaacgattcgcagttaatc
ctggcctgttagaaacatcagaaggctgtagacaaatactgggacagctacaaccatcccttcag
acaggatcagaagaacttagatcattatataatacagtagcaaccctctattgtgtgcatcaaag
gatagagataaaagacaccaaggaagctttagacaagatagaggaagagcaaaacaaaagtaaga
ccaccgcacagcaagcGGCCGCTgatcttcagacctggaggaggagatatgagggacaattggag
aagtgaattatataaatataaagtagtaaaaattgaaccattaggagtagcacccaccaaggcaa
agagaagagtggtgcagagagaaaaaagaGCAGTGGGAATaggagctttgttccttgggttcttg
ggagcagcaggaagcactatgggcgcagcgtcaatgacgctgacggtacaggccagacaattatt
gtctggtatagtgcagcagcagaacaatttgctgagggctattgaggcgcaacagcatctgttgc
aactcacagtctggggcatcaagcagctccaggcaagaatcctggctgtggaaagatacctaaag
gatcaacagctcctggggatttggggttgctctggaaaactcatttgcaccactgctgtgccttg
gaatgctagttggagtaataaatctctggaacagatttggaatcacacgacctggatggagtggg
acagagaaattaacaattacacaagcttaatacactccttaattgaagaatcgcaaaaccagcaa
gaaaagaatgaacaagaattattggaattagataaatgggcaagtttgtggaattggtttaacat
aacaaattggctgtggtatataaaattattcataatgatagtaggaggcttggtaggtttaagaa
tagtttttgctgtactttctatagtgaatagagttaggcagggatattcaccattatcgtttcag
acccacctcccaaccccgaggggacccgacaggcccgaaggaatagaagaagaaggtggagagag
agacagagacagatccattcgattagtgaacggatCacaaatggcagtattcatccacaatttta
aaagaaaaggggggattggggggtacagtgcaggggaaagaatagtagacataatagcaacagac
atacaaactaaagaattacaaaaacaaattacaaaaattcaaaattttcgggtttattacaggga
cagcagaggctccggtgcccgtcagtgggcagagcgcacatcgcccacagtccccgagaagttgg
ggggaggggtcggcaattgaaccggtgcctagagaaggtggcgcggggtaaactgggaaagtgat
gtcgtgtactggctccgcctttttcccgaggggggggagaaccgtatataagtgcagtagtcgcc
gtgaacgttctttttcgcaacgggtttgccgccagaacacaggtgtcgtgaAAATAAGAGAGAAA
AGAAGAGTAAGAAGAAATATAAGAGCCACCatgGTGAAGATCATCAGCAGAAAGAGCCTGGGCAC
CCAGAACGTGTACGACATCGGCGTGGAAAAGGACCACAACTTTCTGCTCAAGAACGGCCTGGTGG
CCAGCAACTGCTTCAATCTGAAAACCAGCGGCATCGTGCTGAGCAGCCTGCCTTACAAGCAGTAC
AACATGCTGAATGCCGACACTACCCGCAACCTGATGATCTGCTTCCTGTGGATTATGAAGAACGC
CGATCAGAGCCTGATTCGGAAGTGGATTGCCGACCTGCCAAGCACTCAGCTGAACCGGATTCTGG
ACCTCCTGTTCATCTGCGTGCTGTGCTTCGAGTACAAGGGCAAGCAGTCCAGCGACAAGGTGTCC
ACTCAGGTGCTGCAGAAAAGCCGGGATGTGAAGGCCAGGCTGGAAGAGGCACTTCTGAGAGGCGA
AGGCGCCAGAGGCGAGATGATGAGGCGTAGAGCCCCTGGCAACGATAGATTCCCCGGCCTGAATG
AGAACCTGCGGTGGAAGAAAGAGCAGACCCATTGGAGACAGGCCAACGAGAAACTGGACAAGACC
AAGGCCGAACTGGACCAAGAGGCCCTGATCTCTGGCAACCTGGCCACAGAAGCCCACCTGATTAT
CCTGGACATGCAAGAGAACATCATCCAGGCCAGTAGCGCCCTGGATTGCAAGGATTCTCTGCTCG
GAGGCGTGCTGAGGGTGCTCGTGAATAGCCTGAATTGCGACCAGAGCACCACCTACCTGACACAC
TGCTTCGCCACACTGAGGGCCCTGATTGCCAAGTTTGGGGACCTGCTGTTCGAGGAAGAGGTGGA
ACAGTGCTTCGACCTGTGTCACCAAGTGCTGCACCACTGCAGCAGCTCCATGGACGTGACAAGAT
CCCAGGCCTGCGCCACTCTGTATCTGCTGATGCGGTTTAGCTTCGGCGCCACCAGCAATTTCGCC
AGAGTGAAGATGCAAGTGACCATGAGCCTGGCCAGCCTCGTTGGAAGGGCCCCTGACTTCAATGA
GGAACACCTCAGAAGAAGCCTGCGGACCATCCTGGCCTACAGCGAAGAGGATACCGCCATGCAGA
TGACCCCATTTCCAACACAAGTGGAAGAACTGCTGTGCAACCTGAATTCCATCCTGTACGATACC
GTGAAGATGAGAGAGTTCCAAGAGGACCCCGAGATGCTGATGGATCTGATGTACCGGATCGCCAA
GAGCTACCAGGCCTCTCCTGATCTGAGACTGACCTGGCTGCAGAACATGGCCGAGAAGCACACCA
AGAAGAAGTGCTACACCGAGGCCGCCATGTGCCTGGTTCATGCTGCTGCACTCGTGGCCGAGTAC
CTGAGCATGCTGGAAGATCACTCCTACCTGCCAGTGGGCAGCGTGTCCTTCCAGAACATCAGCTC
CAACGTCCTGGAAGAGTCCGTGGTGTCCGAGGACACACTGAGCCCAGATGAGGATGGCGTGTGTG
CCGGACAGTACTTTACCGAGTCTGGCCTCGTGGGCCTTCTGGAACAGGCCGCCGAACTGTTTTCT
ACCGGCGGACTGTACGAGACAGTGAACGAAGTGTACAAGCTGGTCATCCCCATCCTGGAAGCCCA
CAGAGAGTTCAGAAAGCTGACACTGACCCACTCCAAGCTGCAGCGGGCCTTCGACAGCATTGTGA
ACAAGGACCACAAGCGGATGTTCGGCACCTACTTCAGAGTGGGCTTTTTCGGCAGCAAATTCGGC
GACCTGGACGAGCAAGAGTTCGTGTACAAAGAGCCCGCCATCACCAAGCTGCCTGAGATCTCCCA
TCGGCTGGAAGCCTTCTACGGCCAGTGTTTTGGCGCCGAGTTCGTGGAAGTGATCAAGGACAGCA
CCCCTGTGGATAAGACCAAACTGGACCCTAACAAGGCCTACATCCAGATCACCTTCGTGGAACCC
TACTTCGACGAGTACGAGATGAAGGATAGAGTGACCTACTTTGAGAAGAACTTCAACCTGCGGCG
GTTCATGTACACCACACCTTTTACACTGGAAGGCAGACCCAGGGGCGAGCTGCACGAGCAGTATA
GAAGAAACACCGTGCTGACCACCATGCACGCATTCCCTTACATCAAGACCCGGATCAGCGTGATC
CAGAAAGAAGAGTTTGTCCTGACACCAATCGAGGTGGCCATCGAGGACATGAAGAAGAAAACCCT
CCAGCTGGCCGTGGCCATCAATCAAGAACCTCCAGACGCCAAGATGCTCCAGATGGTGCTCCAGG
GATCTGTGGGCGCCACAGTGAATCAGGGACCTCTGGAAGTGGCCCAAGTGTTCCTGGCTGAGATC
CCCGCCGATCCTAAGCTGTACAGACACCATAACAAGCTGCGGCTCTGCTTCAAAGAATTCATCAT
GAGATGTGGCGAGGCCGTGGAAAAGAACAAGAGACTGATCACCGCCGACCAGAGAGAGTACCAGC
AAGAGCTGAAGAAAAACTACAACAAGCTCAAAGAAAATCTGCGGCCCATGATCGAGAGAAAGATC
CCCGAGCTGTACAAGCCCATCTTCCGCGTGGAAAGCCAGAAGCGGGACTCCTTTCACAGATCCAG
CTTTAGGAAGTGCGAGACACAGCTGTCCCAGGGCTCTTGAttccgataatcaacctctggattac
aaaatttgtgaaagattgactggtattcttaactatgttgctccttttacgctatgtggatacgc
tgctttaatgcctttgtatcatgctattgcttcccgtatggctttcattttctcctccttgtata
aatcctggttgctgictcittatgaggagtigtggcccgtigtcaggcaacgtggcgtggigigc
actgtgtutgctgacgcaacccccactggttggggcattgccaccacctgtcagctcctttccgg
gactttcgctttccccctccctattgccacggcggaactcatcgccgcctgccttgcccgctgct
ggacaggggctcggctgttgggcactgacaattccgtggtgttgtcggggaaATCAtcgtccttt
ccTtggctgctcgcctgtgttgccacctggattctgcgcgggacgtccttctgctacgtcccttc
ggccctcaatccagcggaccttccttcccgcggcctgctgccggctctgcggcctcttccgcgtc
ttcgccttcgccctcagacgagtcggatctccctttgggccgcctccccgcTaaaagaaaagggg
ggactggaagggctaattcactcccaacgaagacaagatctgctttttgcttgtactGGGTCTCT
CTGGTTAGACCAGATCTGAGCCTGGGAGCTCTCTGGCTAACTAGGGAACCCACTGCTTAAGCCTC
AATAAAGCTTGCCTTGAGTGCTTCAAGTAGTGTGTTGGTTTTTTGTGTGTGCATTGTCTGAGTAG
GTGTCATTCTATTCTGGGGGGTGGGGTGGGGCAGCACAGCAAGGGGGAGGATTGGGAAGACAATA
GCAGGCATGCTGGGGATGCGGTGGGCTCTATGGagtagtagttcatgtcatcttattattcagta
tttataacttgcaaagaaatgaatatcagagagtgagaggaacttgtttattgcagcttataatg
gttacaaataaagcaatagcatcacaaatttcacaaataaagcatttttttcactgcattctagt
tgtggtttgtccaaactcatcaatgtatcttatcatgtctggctctagctatcccgcccctaact
ccgcccATCCCGCCCCTAACTCCGCCCAGTTCCGCCCATTCTCCGCCCCATGGCTCTCACTACTT
CTGGAATAGCTCAGAGGCCGAGGCGGCCTCGGCCTCTGCATAAATAAAAAAAATTAGTCGAGGCT
TTTTTGGAGGCCTAGGgacgtacccaattcgccctatagtgagtcgtattacgcgcgctcactgg
ccgtcgttttacaacgtcgtgactgggaaaaccctggcgttacccaacttaatcgccttgcagca
catccccctttcgccagctggcgtaatagcgaagaggcccgcaccgatcgcccttcccaacagtt
gcgcagcctgaatggcgaatggg
(SEQ ID NO: 39)
1267 CfaC-CFN-DOCK8 (2^{nd} fragment; C-terminal half)
MVKIISRKSLGTQNVYDIGVEKDHNFLLKNGLVASNCFNLKTSGIVLSSLPYKQYNMLNADTTRN
LMICFLWIMKNADQSLIRKWIADLPSTQLNRILDLLFICVLCFEYKGKQSSDKVSTQVLQKSRDV
KARLEEALLRGEGARGEMMRRRAPGNDRFPGLNENLRWKKEQTHWRQANEKLDKTKAELDQEALI
SGNLATEAHLIILDMQENIIQASSALDCKDSLLGGVLRVLVNSLNCDQSTTYLTHCFATLRALIA
KFGDLLFEEEVEQCFDLCHQVLHHCSSSMDVTRSQACATLYLLMRFSFGATSNFARVKMQVTMSL
ASLVGRAPDFNEEHLRRSLRTILAYSEEDTAMQMTPFPTQVEELLCNLNSILYDTVKMREFQEDP
EMLMDLMYRIAKSYQASPDLRLTWLQNMAEKHTKKKCYTEAAMCLVHAAALVAEYLSMLEDHSYL
PVGSVSFQNISSNVLEESVVSEDTLSPDEDGVCAGQYFTESGLVGLLEQAAELFSTGGLYETVNE
VYKLVIPILEAHREFRKLTLTHSKLQRAFDSIVNKDHKRMFGTYFRVGFFGSKFGDLDEQEFVYK
EPAITKLPEISHRLEAFYGQCFGAEFVEVIKDSTPVDKTKLDPNKAYIQITFVEPYFDEYEMKDR
VTYFEKNFNLRRFMYTTPFTLEGRPRGELHEQYRRNTVLTTMHAFPYIKTRISVIQKEEFVLTPI
EVAIEDMKKKTLQLAVAINQEPPDAKMLQMVLQGSVGATVNQGPLEVAQVFLAEIPADPKLYRHH
NKLRLCFKEFIMRCGEAVEKNKRLITADQREYQQELKKNYNKLKENLRPMIERKIPELYKPIFRV
ESQKRDSFHRSSFRKCETQLSQGS*
(SEQ ID NO: 42;*-stop codon)

### Methods and Results:

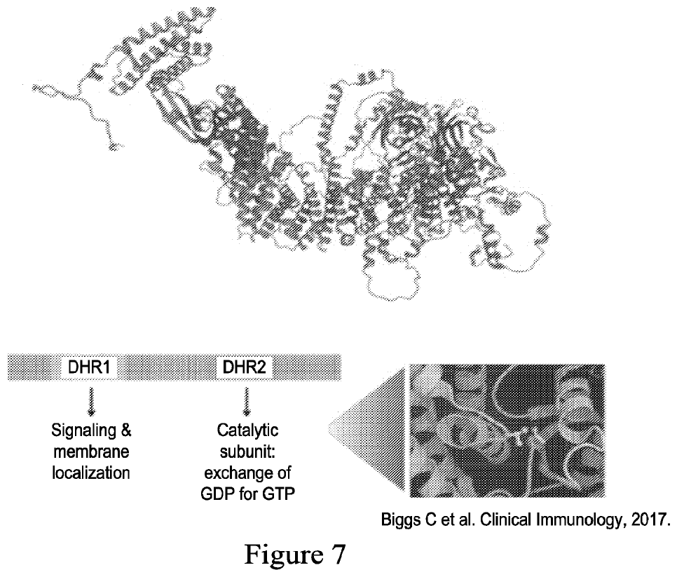

To assess if splicing of the DOCK8 protein would be feasible by inteins, additional plasmids were cloned containing two halves of the DOCK8 protein with their respective inteins. (See Table 1 for the amino acid and nucleic acid sequences of DOCK8 plasmids and components thereof.) However, in contrast to GFP, the DOCK8 protein is large and complex and is known to have two domains (DHR1 and DHR2) critical for protein function. (FIG. 7; Biggs et al (2017) DOCK8 deficiency: Insights into pathophysiology, clinical features and management. Clin Immunol. 181:75-82.) Therefore, intein split sites were identified in the region between the two Dock Homology Regions at amino acids 939 or 1267 in external loops that do not obviously contribute to intra-protein interactions (FIG. 8).

For the 939 CfaC intein, both the C-F-N and C-F extein linkers were tested; for the 1267 CfaC intein, only the C-F-N extein linker was tested since that particular split site naturally starts with an N (asparagine).

The intein plasmids used were:
939 DOCK8-CfaN (SEQ ID NO: 5): This is the N-terminal intein sequence containing the first fragment (half) of the human DOCK8 codon-optimized cDNA followed by the CfaN intein sequence. The split site is just before the 939′ amino acid of DOCK8 so the DOCK8 cDNA for this sequence ends at the codon encoding amino acid 938.
939 CfaC-CFN-DOCK8 (SEQ ID NO: 7): This is the C terminal DOCK8 intein starting with the CfaC intein sequence followed by the second fragment (half) of the human DOCK8 codon-optimized cDNA sequence. Note, the CfaC intein has a required linker sequence between the intein and the extein that can be either -CF- (cysteine-phenylalanine) or -CFN- (cysteine-phenylalanine-asparagine). Since the efficiency of each of these linkers is unknown for intein splicing, both versions were designed and tested.
939 CfaC-CF-DOCK8 (SEQ ID NO: 9): Same as 939 CfaC-CFN-DOCK8 except with the -CF- (cysteine-phenylalanine linker).
1267 DOCK8-CfaN: (SEQ ID NO: 10) This is the N-terminal intein sequence containing the first fragment (half) of the human DOCK8 codon-optimized cDNA followed by the CfaN intein sequence. The split site for this cassette is just after the 1267th amino acid of DOCK8.
1267 CfaC-CFN-DOCK8: (SEQ ID NO: 12) This is the C terminal DOCK8 intein starting with the CfaC intein sequence followed by the second fragment (half) of the human DOCK8 codon-optimized cDNA sequence. Note, we chose this split site because the first amino acid of the C terminal half of the protein naturally starts with an phenylalanine followed by an asparagine, so we only needed to add cysteine to create a -CFN- linker.

Intein plasmids were tested in the Jurkat T cell line, which does not express DOCK8. DOCK8 inteins were introduced to Jurkat cells via electroporation using the Lonza 4D Nucleofector. 1 ug of each plasmid listed above were used in each experimental condition. One day after electroporation, protein lysates were collected, quantified, and analyzed for DOCK8 protein expression by immunoblot.

For both the 939 and 1267 split sites, only samples that receive both the CfaN and CfaC components of the DOCK8 inteins form DOCK8 protein visible at 190 kDa. Control samples for the Western blot include T cells and K562 cell lines, both of which are positive for DOCK8 and untreated Jurkat cells, which do not express DOCK8 at baseline.

As shown in FIG. 9, untreated Jurkat cells are negative for DOCK8, while the positive controls, which include primary human T cells and K562 cells express DOCK8. Jurkat cells that received only half of the DOCK8 protein are negative by immunoblot, while samples receiving both the CfaN and CfaC intein plasmids have restored DOCK8 expression. In addition, both the CfaC-C-F and CfaC-C-F-N plasmids appear to restore DOCK8 expression equally at the 939 DOCK8 split site. As both the 939 CfaC-CF-DOCK8-bGHpA (Seq ID No 7) and 939 CfaC-CFN-DOCK8-bGHpA (Seq ID No 9) perform similarly in these experiments, moving forward the CF linker (Seq ID No 7) was used, given this would result in fewer added amino acids to the DOCK8 protein.

In all, these experiments demonstrate the ability of inteins to restore expression of the DOCK8 protein. Additional work using inteins delivered by lentiviral vectors was performed in primary hematopoietic stem cells.

### Example 5: Integration of DOCK8 cDNA Split Inteins in Stem Cells Using Lentiviral Vectors

Objectives: To develop a human stem cell transfer method for the treatment of DOCK8 deficiency using Lentiviral vector transduction of human hematopoietic stem cells approach.

Materials: Lentiviral vectors and sequences thereof are provided in Table 1 (Example 4 above) and FIGS. 14, 16, 18, and 20. In these experiments the nucleotide sequence for both DOCK8 and the intein halves were codon-optimized using GeneArt algorithms.

PBSC cells used herein comprise a mix of stem and progenitor cells, also called HSPC—Hematopoietic Stem and Progenitor Cells.

### Methods and Results:

Jurkat T cells were transduced with either mCCL-EFS-939DOCK8-CfaN-WPRE (SEQ ID NO: 6) with mCCL-EFS-939Dock8-CfaC-CF-WPRE (SEQ ID NO: 8), or with mCCL-EFS-1267Dock8-CfaN-WPRE (SEQ ID NO: 11) with mCCL-EFS-1267Dock8-CfaC-CFN-WPRE (SEQ ID NO: 13) at a range of concentrations from 2e5 TU/mL up to 6e6 TU/mL. Two weeks post-transduction, genomic DNA was isolated from cells and vector copy number (VCN) was quantified by droplet digital PCR (ddPCR).

FIG. 21 shows that VCNs increased in a dose dependent manner based on the amount of vector added and ranged from 1.56 up to 16.1.

To gage if the concentration of Lentiviral vectors influenced expression level of DOCK8, flow cytometric analysis of DOCK8 expression in Jurkat T cells transduced with either the 939 or 1267 intein lentiviral vector pairs at a range of concentrations from 2e5 to 6e6 TU/mL was performed.

The results show that there is a dose dependent response based on the amount of lentiviral vector used in terms of both VCN quantified 2 weeks after transduction by ddPCR or DOCK8 expression measured by flow cytometry. DOCK8 positive cells range from 23% to 93% from the lowest to the highest concentrations of virus shown in FIGS. 22A-22B.

Transduced Jurkat cells were also assessed for DOCK8 expression by Western blot (FIG. 23). Mock treated Jurkat cells do not have detected DOCK8 protein (190 kDa) while cells transduced with the 939 or 1267 lentiviral pairs have detectable DOCK8 expression. CD4 T cells are included as a positive control.

FIG. 24A shows healthy donor derived peripheral blood stem cells (PBSC) were pre-stimulated for either one or two days in XVIVO15 with 50 ng/mL hSCF, 50 ng/mL hFlt3ligand, and 50 ng/mL hTPO. After 1 or 2 days of pre-stimulation, PBSC were transduced with the mCCL-EFS-939DOCK8-CfaN-WPRE (SEQ ID NO: 6) and mCCL-EFS-939Dock8-CfaC-CF-WPRE (SEQ ID NO: 8) lentiviral vectors at a range of TU/mL, with or without LentiBoost (Sirion Biotech). Vector copy number was quantified by ddPCR and shown above. With the addition of LentiBoost, transduction efficiency is improved and VCNs reach >1.5.

FIG. 24B shows similar results in PBSC transduced with mCCL-EFS-1267Dock8-CfaN-WPRE (SEQ ID NO: 11) and mCCL-EFS-1267Dock8-CfaC-CFN-WPRE (SEQ ID NO: 13) lentiviral vectors at a range of concentrations from 6e6 to 2e8 TU/mL with LentiBoost. The results show that increasing amounts of lentiviral vector improves VCN.

DOCK8 promotes STAT3 phosphorylation and translocation to the nucleus. Therefore, gene modified cells that properly express DOCK8, as has been shown here, should also have normal phosphorylation of STAT3 upon immune stimulation as detected by Western blot and flow cytometry.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the systems for expressing DOCK8 and uses thereof.

What is claimed is:

1. A system for expressing human DOCK8 protein in a cell, said system comprising:
a first nucleic acid encoding a first fragment of a human DOCK8 protein fused at the C terminal to an N-intein; and
a second nucleic acid encoding a second fragment of the human DOCK8 protein fused at the N terminal with a C-intein;
wherein the first fragment of the DOCK8 protein ligated to the second fragment of the DOCK8 protein forms a full-length human DOCK8 protein,
wherein the first fragment of the DOCK8 protein and the second fragment of the DOCK8 protein are selected to provide a split between said first fragment and said second fragment in an external loop that does not contribute to intra-protein interactions, wherein said first fragment comprises the DOCK homology region 1 (DHR1) and said second fragment comprises the DOCK homology region 2 (DHR2), and
wherein said first nucleic acid further comprises a promoter located 5′ of said nucleic acid encoding the first fragment of DOCK8 and said second nucleic acid further comprises a promoter located 5′ of said nucleic acid encoding the C-intein, wherein optionally said promoter comprises EFS.

2. The system according to claim 1, wherein
the amino acid sequence of said first fragment of DOCK8 is set forth in SEQ ID NO: 18 and the amino acid sequence of said second fragment of DOCK8 is set forth in SEQ ID NO: 25, or
the amino acid sequence of said first fragment of DOCK8 is set forth in SEQ ID NO: 34 and the amino acid sequence of said second fragment of DOCK8 is set forth in SEQ ID NO: 37.

3. The system according to claim 1, wherein said first nucleic acid encoding a first fragment of a human DOCK8 is codon-optimized and wherein said second nucleic acid encoding a second fragment of the human DOCK8 protein is codon-optimized.

4. The system according to claim 1, wherein said second fragment of the DOCK8 protein comprise an extein linker where said linker comprises C-F or C-F-N.

5. The system according to claim 1, wherein the N-intein and C-intein comprise a Cfa split intein, wherein the N-intein comprises a CfaN sequence and the C-intein comprise a CfaC sequence, wherein optionally said nucleotide sequence encoding said CfaN amino acid sequence and said nucleotide sequence encoding said CfaC amino acid sequence are codon optimized for expression in a human cell.

6. The system according to claim 5, wherein
the amino acid sequence of said first fragment of the human DOCK8 protein fused at the C terminal to an N-intein is set forth in SEQ ID NO: 17 and the amino acid sequence of said second fragment of the human DOCK8 protein fused at the N terminal with a C-intein is set forth in SEQ ID NO: 23 or SEQ ID NO: 27; or
the amino acid sequence of said first fragment of the human DOCK8 protein fused at the C terminal to an N-intein is set forth in SEQ ID NO: 33 and the amino acid sequence of said second fragment of the human DOCK8 protein fused at the N terminal with a C-intein is set forth in SEQ ID NO: 42.

7. The system according to claim 1, wherein the first nucleic acid is comprised in a first lentiviral vector (first LV) and the second nucleic acid is comprised in a second lentiviral vector (second LV).

8. The system according to claim 7, wherein said first LV and said second LV are each TAT-independent and self-inactivating (SIN) lentiviral vectors.

9. The system according to claim 7, wherein said first nucleic acid further comprises a WPRE element located 3′ of said nucleic acid encoding the N-intein and said second nucleic acid further comprises a WPRE element located 3′ of said nucleic acid encoding the second fragment of DOCK8 protein.

10. The system according to claim 9, wherein the nucleic acid sequence of said first nucleic acid is set forth in SEQ ID NO: 28 and the nucleic acid sequence of said second nucleic acid is set forth in SEQ ID NO: 29 or SEQ ID NO: 30.

11. The system according to claim 9, wherein the nucleic acid sequence of said first nucleic acid is set forth in SEQ ID NO: 38 and the nucleic acid sequence of said second nucleic acid is set forth in SEQ ID NO: 39.

12. A pair of recombinant lentiviral vectors (LVs) comprising:
a first LV comprising a first nucleic acid encoding a first fragment of a human DOCK8 protein fused at the C termina to an N-intein; and
a second LV comprising a second nucleic acid encoding a second fragment of the human DOCK8 protein fused at the N terminal with a C-intein;
wherein the first fragment of the DOCK8 protein ligated to the second fragment of the DOCK8 protein forms a full-length DOCK8 protein,
wherein the first fragment of the DOCK8 protein and the second fragment of the DOCK8 protein are selected to provide a split between said first fragment and said second fragment in an external loop that does not contribute to intra-protein interactions,
wherein said first fragment comprises the DOCK homology region 1 (DHR1) and said second fragment comprises the DOCK homology region 2 (DHR2), and
wherein said first nucleic acid further comprises a promoter located 5′ of said nucleic acid encoding the first fragment of DOCK8 and said second nucleic acid further comprises a promoter located 5′ of said nucleic acid encoding the C-intein.

13. The pair of lentiviral vectors of claim 12, wherein
the amino acid sequence of said first fragment of DOCK8 is set forth in SEQ ID NO: 18 and the amino acid sequence of said second fragment of DOCK8 is set forth in SEQ ID NO: 25, or
the amino acid sequence of said first fragment of DOCK8 is set forth in SEQ ID NO: 34 and the amino acid sequence of said second fragment of DOCK8 is set forth in SEQ ID NO: 37.

14. The pair of lentiviral vectors according to claim 12, wherein said first nucleic acid encoding a first fragment of a human DOCK8 is codon-optimized and wherein said second nucleic acid encoding a second fragment of the human DOCK8 protein is codon-optimized.

15. The pair of lentiviral vectors according to claim 12, wherein said second fragment of the DOCK8 protein comprise an extein linker where said linker comprises C-F or C-F-N.

16. The pair of lentiviral vectors according to claim 12, wherein the N-intein and C-intein comprise a Cfa split intein, wherein the N-intein comprises a CfaN sequence and the C-intein comprise a CfaC sequence, wherein optionally said nucleotide sequence encoding said CfaN amino acid sequence and said nucleotide sequence encoding said CfaC amino acid sequence are codon optimized for expression in a human cell.

17. The pair of lentiviral vectors according to claim 16, wherein
the amino acid sequence of said first fragment of the human DOCK8 protein fused at the C terminal to an N-intein is set forth in SEQ ID NO: 17 and the amino acid sequence of said second fragment of the human DOCK8 protein fused at the N terminal with a C-intein is set forth in SEQ ID NO: 23 or SEQ ID NO: 27; or
the amino acid sequence of said first fragment of the human DOCK8 protein fused at the C terminal to an N-intein is set forth in SEQ ID NO: 33 and the amino acid sequence of said second fragment of the human DOCK8 protein fused at the N terminal with a C-intein is set forth in SEQ ID NO: 42.

18. The pair of lentiviral vectors according to claim 12, wherein said first nucleic acid further comprises a WPRE element located 3′ of said nucleic acid encoding the N-intein and said second nucleic acid further comprises a WPRE element located 3′ of said nucleic acid encoding the second fragment of DOCK8 protein.

19. The pair of lentiviral vectors according to claim 18, wherein the nucleic acid sequence of said first nucleic acid is set forth in SEQ ID NO: 28 and the nucleic acid sequence of said second nucleic acid is set forth in SEQ ID NO: 29 or SEQ ID NO: 30.

20. The pair of lentiviral vectors according to claim 18, wherein the nucleic acid sequence of said first nucleic acid is set forth in SEQ ID NO: 38 and the nucleic acid sequence of said second nucleic acid is set forth in SEQ ID NO: 39.

21. The pair of lentiviral vectors of claim 12, wherein said first LV and said second LV are each TAT-independent and self-inactivating (SIN) lentiviral vectors.

22. A host cell transduced with the pair of lentiviral vectors according to claim 12.

23. The host cell according to claim 22, wherein the cell is a stem cell derived from bone marrow, umbilical cord blood, or from peripheral blood, or any combination thereof, or is a hematopoietic progenitor cell.

24. The host cell according to claim 23, wherein the human hematopoietic progenitor cell is a CD34+ cell.

25. A method of treating a DOCK8 deficiency in a human subject, said method comprising:
transducing a stem cell and/or progenitor cell from said subject with a pair of lentiviral vectors according to claim 12 to produce transduced cells expressing functional DOCK8 protein; and
transplanting said transduced cell or cells derived therefrom into said subject, where said cells therefrom express functional DOCK8 protein.

26. The method of claim 25, wherein the DOCK8 protein is constitutively expressed.

27. The method of claim 25, wherein said cell is a stem cell or human hematopoietic progenitor cell or a combination thereof.

28. The method of claim 27, wherein said stem cell is derived from bone marrow.

29. The method according to claim 27, wherein said human hematopoietic progenitor cell is a CD34+ cell.
